(19) United States
(12) Patent Application Publication
(10) Pub. No.: US 2009/0082400 A1
(43) Pub. Date: Mar. 26, 2009

(54) SOLUBLE PYRONE ANALOGS METHODS AND COMPOSITIONS

(76) Inventors: Ving LEE, Los Altos, CA (US); Wendye Robbins, San Francisco, CA (US)

Correspondence Address: WILSON SONSINI GOODRICH & ROSATI, 650 PAGE MILL ROAD, PALO ALTO, CA, 94304-1050, US

(21) Appl. No.: 12/182,979

(22) Filed: Jul. 30, 2008

Related U.S. Application Data
(60) Provisional application No. 60/953,186, filed on Jul. 31, 2007.
(60) Provisional application No. 61/076,612, filed on Jun. 27, 2008.

(51) Int. Cl. A61K 31/453; A61K 31/352; A61P 37/00; A61P 29/00

(52) U.S. Cl. 514/326; 514/456

(57) ABSTRACT

Methods and compositions are described that comprise pyrone analogs such as flavonoids and cyclodextrins including quercetin and quercetin derivatives and sulfoalkyl ether cyclodextrins. In some cases the compounds of the invention are administered with a therapeutic agent such as an analgesic. In some cases, treatment with the compositions of the invention can result in the modulation of central nervous system and/or fetal effects of substances. Methods and compositions are described for the modulation of efflux transporter activity to increase the efflux of drugs and other compositions out of a physiological compartment and into an external environment. In particular, the methods and compositions disclosed herein provide for the increase of efflux transporter activity at blood-brain, blood-CSF and placental-maternal barriers to increase the efflux of drugs and other compositions from physiological compartments, including central nervous system and fetal compartments.

### CROSS-REFERENCE

This application claims the benefit of U.S. Provisional Application No. 60/953,186, filed Jul. 31, 2007; and U.S. Provisional Application No. 61/076,612, filed Jun. 27, 2008; which are incorporated herein by reference in their entirety.

### BACKGROUND OF THE INVENTION

Flavonoids are widely distributed in vegetables and plants. Flavonoids and other pyrone analogs such as quercetin have been shown to possess a wide array of biological effects that can be beneficial to health, including antioxidative, free radical scavenging, anticancer, and antiviral properties. Flavonoids can also enhance the effectiveness and/or reduce the side effects of therapeutic agents, for example, analgesics when administered in combination with such agents (see U.S. patent application Ser. No. 11/281,771, 11/281,984, and 11/553,924).

Quercetin, as well as other useful flavonoids, however, is only sparingly soluble in water, which limits its absorption, for example, upon oral administration. Flavonoids can also be chemically unstable, for example in aqueous alkaline medium and can undergo extensive metabolism in the gut and the liver following absorption. All these factors lead to a low oral bioavailability of flavonoids such as quercetin. Therefore, aqueous compositions with high concentrations of flavonoids would be effective pharmaceutical formulations for oral and intravenous administration.

### SUMMARY OF THE INVENTION

One aspect of the invention is a method for producing a stable aqueous composition comprising a pyrone analog such as a flavonoid comprising mixing a cyclodextrin and the pyrone analog such as a flavonoid in an aqueous medium at a pH greater than about 11 and subsequently lowering the pH to less than about 9. In some embodiments the mixing of the cyclodextrin and the pyrone analog such as a flavonoid is at a pH greater than 12, and the pH is subsequently lowered to less than 8.5. In some embodiments the cyclodextrin is sulfobutylether-7-β-cyclodextrin. In some embodiments the pyrone analog such as a flavonoid is quercetin, galangin, fisetin, or kaempferol. In some embodiments the flavonoid is quercetin. In some embodiments the flavonoid is fisetin. In some embodiments the flavonoid is 5,7-dideoxyquercetin.

In some embodiments the pyrone analog such as a flavonoid has acidic protons. In some embodiments the pyrone analog such as a flavonoid has 3, 4, 5 or 6 acidic protons. In some embodiments the pyrone analog such as a flavonoid has 5 or 6 acidic protons. In some embodiments the pyrone analog such as a flavonoid has an aromatic hydroxyl (—OH) group. In some embodiments the pyrone analog such as a flavonoid has 3, 4, 5 or 6 aromatic hydroxyl (—OH) groups. In some embodiments the pyrone analog such as a flavonoid has 5 or 6 aromatic hydroxyl (—OH) groups.

In some embodiments the pyrone analog such as a flavonoid is substantially insoluble in water. In some embodiments the pyrone analog such as a flavonoid is sparingly soluble in water.

In some embodiments sodium hydroxide is added in order to bring the pH of the aqueous solution to greater than about 11. In some embodiments hydrochloric acid is used to lower the pH.

In some embodiments the method is carried out at a temperature below 30° C. In some embodiments the method is carried out at a temperature below 26° C.

In some embodiments the time that the reaction is above pH 9 is less than 20 minutes. In some embodiments the time that the reaction is above pH 9 is less than 10 minutes. In some embodiments the time that the reaction is above pH 9 is less than 5 minutes.

In some embodiments the concentration of the pyrone analog such as a flavonoid in the aqueous composition of the invention is greater than about 0.5 mM. In some embodiments the concentration of the pyrone analog such as a flavonoid in the aqueous composition is greater than about 5 mM. In some embodiments the concentration of the pyrone analog such as a flavonoid in the aqueous composition is greater than about 10 mM. In some embodiments the concentration of the pyrone analog such as a flavonoid in the aqueous composition is greater than about 30 mM. In some embodiments the concentration of the pyrone analog such as a flavonoid in the aqueous composition is greater than about 50 mM. In some embodiments the concentration of the pyrone analog such as a flavonoid in the aqueous composition is greater than about 80 mM.

One aspect of the invention is method for producing a composition comprising a pyrone analog such as a flavonoid in an aqueous solution comprising: dissolving a cyclodextrin in an aqueous solution; adding the pyrone analog such as a flavonoid to the aqueous solution; raising the pH of the aqueous solution to above about pH 11 while mixing the cyclodextrin and pyrone analog such as a flavonoid; and lowering the pH of the aqueous solution to below about pH 9.

In some embodiments the steps are carried out in the order listed. In some embodiments the cyclodextrin is sulfobutylether-7-β-cyclodextrin. In some embodiments the pyrone analog such as a flavonoid is quercetin, galangin, fisetin, or kaempferol. In some embodiments the pyrone analog such as a flavonoid is quercetin. In some embodiments the pyrone analog such as a flavonoid is fisetin. In some embodiments the pyrone analog such as a flavonoid is 5,7-dideoxyquercetin. In some embodiments the cyclodextrin is sulfobutylether-7-β-cyclodextrin, and the pyrone analog such as a flavonoid is quercetin. In some embodiments the cyclodextrin is sulfobutylether-7-β-cyclodextrin, and the pyrone analog such as a flavonoid is fisetin. In some embodiments the cyclodextrin is sulfobutylether-7-β-cyclodextrin, and the pyrone analog such as a flavonoid is 5,7-dideoxyquercetin.

In some embodiments, in step (c) the pH is raised to above about pH 12. In some embodiments, in step (d) the pH is lowered to below about pH 8.5. In some embodiments the method is carried out at a temperature below 30° C. In some embodiments the method is carried out at a temperature below 26° C.

In some embodiments the time that the reaction is above pH 9 is less than 20 minutes. In some embodiments the time that the reaction is above pH 9 is less than 10 minutes. In some embodiments the time that the reaction is above pH 9 is less than 5 minutes.

One aspect of the invention is a composition produced by carrying out s 1 or 27.

One aspect of the invention is a composition comprising a pyrone analog such as a flavonoid and a sulfo-alkyl ether substituted cyclodextrin and an aqueous carrier wherein the pyrone analog such as a flavonoid is present in a concentration greater than 0.5 mM.

In some embodiments the pyrone analog such as a flavonoid is present in a concentration greater than about 1 mM. In some embodiments the pyrone analog such as a flavonoid is present in a concentration greater than about 10 mM. In some embodiments the pyrone analog such as a flavonoid is present in a concentration greater than about 30 mM. In some embodiments the pyrone analog such as a flavonoid is present in a concentration greater than about 50 mM. In some embodiments the pyrone analog such as a flavonoid is present in a concentration greater than about 80 mM. In some embodiments the pyrone analog such as a flavonoid is present in a concentration of about 33 mM.

In some embodiments the sulfo-alkyl ether substituted cyclodextrin is a sulfobutyl ether substituted cyclodextrin. In some embodiments the sulfo-alkyl ether substituted cyclodextrin is sulfobutylether-7-β-cyclodextrin. In some embodiments the sulfo-alkyl ether substituted cyclodextrin is Captisol™.

In some embodiments the pyrone analog such as a flavonoid is selected from the group consisting of quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, 5,7-dideoxyquercetin (3,3′,4′-trihydroxyflavone), and epicatechin. In some embodiments the pyrone analog such as a flavonoid is quercetin, galangin, fisetin, or kaempferol. In some embodiments the pyrone analog such as a flavonoid is quercetin. In some embodiments the pyrone analog such as a flavonoid is fisetin. In some embodiments the pyrone analog such as a flavonoid is 5,7-dideoxyquercetin. In some embodiments the pyrone analog such as a flavonoid has 3, 4, 5 or 6 acidic protons. In some embodiments the pyrone analog such as a flavonoid has 5 or 6 acidic protons.

In some embodiments the molar ratio of pyrone analog such as a flavonoid to cyclodextrin is from 1:10 to 10:1. In some embodiments the molar ratio of pyrone analog such as a flavonoid to cyclodextrin is from about 1:2 to about 1:4. In some embodiments the composition is suitable for intravenous administration. In some embodiments the pH of the composition is between 6.5 and about 9. In some embodiments the pH of the composition is between 7.2 and about 8.4. In some embodiments the pH of the composition is between 7.6 and about 8.0. In some embodiments the pH of the composition is about 7.9.

In some embodiments the amount of pyrone analog such as a flavonoid in solution is from about 1 mg/ml to 50 mg/ml. In some embodiments the amount of pyrone analog such as a flavonoid in solution is about 10 mg/ml.

In some embodiments, the composition comprises quercetin in an amount of from about 0.1% to about 1% (w/v); a sulfobutylether-7-β-cyclodextrin in an amount of from about 0.1% to about 5% (w/v); water; and a pH adjusting agent sufficient to adjust the pH of the formulation to from about to 6.5 to about 8. In some embodiments the composition further comprises a co-solvent in an amount of from about 1% to about 35% (w/v). In some embodiments the co-solvent is an alcohol. In some embodiments the composition further comprises an effective amount of an antimicrobial preservative. In some embodiments the composition further comprises an effective amount of an antioxidant.

One aspect of the invention is a composition comprising quercetin and an aqueous carrier wherein the quercetin is soluble at a concentration greater than 0.5 mM at a pH below about 9. In some embodiments the pyrone analog such as a flavonoid is soluble at a concentration greater than about 1 mM at a pH below about 9. In some embodiments the pyrone analog such as a flavonoid is soluble at a concentration greater than about 10 mM at a pH below about 9. In some embodiments the pyrone analog such as a flavonoid is soluble at a concentration greater than about 30 mM at a pH below about 9. In some embodiments the pyrone analog such as a flavonoid is soluble at a concentration greater than about 50 mM at a pH below about 9. In some embodiments the pyrone analog such as a flavonoid is soluble at a concentration greater than about 80 mM. In some embodiments the pyrone analog such as a flavonoid is soluble at a concentration of about 33 mM at a pH below about 9.

One aspect of the invention is a solid pharmaceutical formulation that is made using the methods described above. In some embodiments the cyclodextrin is sulfobutylether-7-β-cyclodextrin. In some embodiments the cyclodextrin is Captisol™. In some embodiments the pyrone analog such as a flavonoid is selected from the group consisting of quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, 5,7-dideoxyquercetin (3,3′,4′-trihydroxyflavone), and epicatechin. In some embodiments the pyrone analog such as a flavonoid is quercetin, galangin, fisetin, or kaempferol. In some embodiments the pyrone analog such as a flavonoid is quercetin. In some embodiments the pyrone analog such as a flavonoid is fisetin. In some embodiments the pyrone analog such as a flavonoid is 5,7-dideoxyquercetin.

In some embodiments the formulation is suitable for oral administration. In some embodiments water is substantially removed from the composition in order to make the solid formulation. In some embodiments the removal of water is performed by a process comprising freeze-drying or lyophilization.

In some embodiments the formulation is suitable for intravenous administration. In some embodiments the molar ratio of quercetin to sulfobutylether-7-β-cyclodextrin is between about 1:1 to about 1:5. In some embodiments the molar ratio of quercetin to sulfobutylether-7-β-cyclodextrin is between about 1:2 to about 1:4. In some embodiments the weight ratio of quercetin to the sulfobutylether-7-β-cyclodextrin is between about 1:10 to about 1:40. In some embodiments the weight ratio of quercetin to sulfobutylether-7-β-cyclodextrin is between about 1:15 to about 1:20.

One aspect of the invention is a kit comprising: a) an container comprising a composition made by a method of the invention, and b) instructions for using the formulation to treat a disorder. In some embodiments of the kit the formulation is suitable for intravenous administration. In some embodiments of the kit the formulation is suitable for oral administration.

One aspect of the invention is a pharmaceutical composition comprising a therapeutic agent, a pyrone analog such as a flavonoid, a sulfo-alkyl ether substituted cyclodextrin and a carrier. In some embodiments the pyrone analog such as a flavonoid is selected from the group consisting of quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, 5,7-dideoxyquercetin (3,3′,4′-trihydroxyflavone), and epicatechin. In some embodiments the pyrone analog such as a flavonoid is quercetin, galangin, fisetin, or kaempferol. In some embodiments the pyrone analog such as a flavonoid is quercetin. In some embodiments the pyrone analog such as a flavonoid is fisetin. In some embodiments the pyrone analog such as a flavonoid is 5,7-dideoxyquercetin.

In some embodiments the cyclodextrin is sulfobutylether-7-β-cyclodextrin.

In some embodiments the therapeutic agent is an analgesic agent. In some embodiments the analgesic is selected from the group consisting of oxycodone, gabapentin, pregabalin, hydrocodone, fentanyl, hydromorphone, levorphenol, morphine, methadone, tramadol, topiramate, diacetyl morphine, codeine, olanzapine, hydrocortisone, prednisone, sufentanyl, alfentanyl, carbamazapine, lamotrigine, doxepin, and haloperidol. In some embodiments analgesic is morphine. In some embodiments the morphine and the quercetin are present in a molar ratio of about 0.002:1 to 0.1:1. In some embodiments morphine is present at about 0.5-1000 mg and the quercetin is present at about 1-500 mg. In some embodiments the morphine is present at about 80 mg and the quercetin is present at about 500 mg. In some embodiments the analgesic is oxycodone. In some embodiments the oxycodone and the quercetin are present in a molar ratio of about 0.002:1 to 0.1:1. In some embodiments the oxycodone is present at about 1-1000 mg and the quercetin is present at about 1-5000 mg. In some embodiments the oxycodone is present at about 80 mg and the quercetin is present at about 500 mg. In some embodiments the analgesic is gabapentin. In some embodiments the gabapentin and the quercetin are present in a molar ratio of about 0.2:1 to 6:1. In some embodiments the gabapentin is present at about 100 to 800 mg and the quercetin is present at about 5-5000 mg. In some embodiments the gabapentin is present at about 300 mg and the quercetin is present at about 150 mg. In some embodiments the analgesic and the pyrone analog such as a flavonoid are present in a molar ratio of about 0.001:1 to about 10:1. In some embodiments the analgesic is present at about 0.001 to 500 mg and the pyrone analog such as a flavonoid is present at about 1 to 1000 mg.

In some embodiments the analgesic agent is present in an amount sufficient to produce an analgesic effect, and wherein the pyrone analog such as a flavonoid is present in an amount sufficient to reduce tissue specific exposure and unwanted adverse effects of the analgesic agent. In some embodiments the pyrone analog such as a flavonoid is a modulator of a blood tissue transport protein, such as P-glycoprotein (herein referred to as blood tissue barrier or BTB transport protein, and is present in an amount sufficient to reduce a side effect of the therapeutic agent. In some embodiments the BTB transport protein is an ABC transport protein. In some embodiments the ABC transport protein is a P-gP. In some embodiments the side effect includes an effect is selected from the group consisting of drowsiness, impaired concentration, sexual dysfunction, sleep disturbances, habituation, dependence, alteration of mood, respiratory depression, nausea, vomiting, dizziness memory impairment, neuronal dysfunction, neuronal death, visual disturbance, impaired mentation, tolerance, addiction, hallucinations, lethargy, myoclonic jerking, endocrinopathies, and combinations thereof.

In some embodiments a therapeutic effect of the therapeutic agent is increased at least about 10% compared to the therapeutic effect without the pyrone analog such as a flavonoid, when the composition is administered to an animal.

In some embodiments, the compositions and methods of the invention utilize an analgesic agent. In some embodiments, the analgesic agent is an opiate analgesic. In some embodiments, the analgesic is a non-opiate analgesic. In some embodiments, the compositions and methods of the invention utilize a non-analgesic therapeutic agent. In some embodiments, the compositions and methods of the invention utilize an antihypertensive agent. In some embodiments, the compositions and methods of the invention utilize an immunosuppressive agent. The therapeutic agent may also be a chemotherapeutic agent, an anti depressant, an anti psychotic, a vasodilator, a cardiac glycoside, a diuretic agent, a bronchodilator, a corticosteroid, a sedative-hypnotic, an antiepileptic drug, a general anesthetic, a skeletal muscle relaxant, an anti-hyperlipidemic agent, a non-steroidal antiinflammatory drug, an antidiabetic agent, an antimicrobial agent, an antifungal agent, an antiviral agent, or an antiprotozoal agent. It will be appreciated that there is some overlap between these groups, e.g., some agents that have primarily an analgesic effect also have other therapeutic effects, while some agents that have primarily a non-analgesic effect also provide some degree of analgesia. The invention encompasses these therapeutic agents as well. Additional suitable drugs may be found in Goodman and Gilman's “The Pharmacological Basis of Therapeutics” Tenth Edition edited by Hardman, Limbird and Gilman or the Physician's Desk Reference, both of which are incorporated herein by reference in their entirety.

In some embodiments the therapeutic agent is an immunomodulator, e.g., an immunosuppressive agent such as a calcineurin inhibitor. In some embodiments, the compositions and methods of the invention utilize cyclosporin A (CsA). In some embodiments, the compositions and methods of the invention utilize tacrolimus. In some embodiments, the calcineurin inhibitor is tacrolimus analog. In some embodiments, the tacrolimus analog is selected from the group consisting of meridamycin, 31-O-Demethyl-FK506; L-683,590, L-685,818; 32-O-(1-hydroxyethylindol-5-yl)ascomycin; ascomycin; C18-OH-ascomycin; 9-deoxo-31-O-demethyl-FK506; L-688,617; A-119435; AP1903; rapamycin; dexamethasone-FK506 heterodimer; 13-O-demethyl tacrolimus; and FK 506-dextran conjugate. In some embodiments the composition comprises a liquid. In some embodiments the composition comprises a solid. In some embodiments the solid is formed by a process comprising freeze drying or lyophilization. In some embodiments the composition is suitable for oral administration. In some embodiments the composition is suitable for administration by injection.

In some embodiments, the invention provides compositions that contains a sulfoalkyl ether cyclodextrin, e.g. sulfobutylether-7-β-cyclodextrin, quercetin or a quercetin derivative and tacrolimus, or FK-506 where the FK-506 is present in an amount sufficient to exert a therapeutic effect, e.g., an immunosuppressive effect, and the quercetin or a quercetin derivative is present in an amount sufficient to decrease a side effect of the FK-506, e.g. a CNS effect and/or a hyperglycemic effect, by a measurable amount, compared to the side effect without the quercetin or a quercetin derivative when the composition is administered to an animal. The measurable amount may be an average of at least about 1, 5, 10, 15, 20, 25, 30, 35, 40, 45, 50, 55, 60, 65, 70, 75, 80, 85, 90, 95, or more than 95%, compared to the side effect without the flavonoid-sulfobutylether-7-β-cyclodextrin composition. The side effect may be any side effect including those described herein.

One aspect of the invention is a method of treating an animal comprising administering to an animal an effective amount of a pharmaceutical formulation that is made using a method of the invention. In some embodiments pyrone analog such as a flavonoid is selected from the group consisting of quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, 5,7-dideoxyquercetin (3,3′,4′-trihydroxyflavone), and epicatechin. In some embodiments the pyrone analog such as a flavonoid is a quercetin. In some embodiments the composition is administered intravenously. In some embodiments the composition is administered orally.

One aspect of the invention is a method of treating an animal for pain comprising administering to an animal in pain an effective amount of an analgesic agent and an amount of a composition comprising a pyrone analog such as a flavonoid and a cyclodextrin sufficient to reduce a central nervous system effect of the analgesic agent. In some embodiments the cyclodextrin is a sulfobutylether-7-β-cyclodextrin. In some embodiments the pyrone analog such as a flavonoid is a BTB transport protein activator which is administered in an amount sufficient to substantially eliminate a central nervous system effect of the analgesic compound. In some embodiments the analgesic agent and the composition comprising a pyrone analog such as a flavonoid and cyclodextrin are co-administered. In some embodiments the analgesic compound and composition comprising a pyrone analog such as a flavonoid and cyclodextrin are administered admixed in a single composition.

In some embodiments the amount of analgesic agent is administered in an amount sufficient to produce an analgesic effect, and wherein said amount is different than the amount sufficient to produce an analgesic effect in the absence of administration of the composition comprising the pyrone analog such as a flavonoid and the cyclodextrin. In some embodiments the amount of analgesic agent administered is lower than the amount sufficient to produce an analgesic effect in the absence of administration of the composition comprising the pyrone analog such as a flavonoid and the cyclodextrin.

In some embodiments the administration is rectal, buccal, intranasal, transdermal, intravenous, intraperitoneal, parenteral, intramuscular, subcutaneous, orally, topical, as an inhalant, or via an impregnated or coated device such as a stent. In some embodiments the administration is intravenous. In some embodiments administration is transdermal. In some embodiments the administration is oral. In some embodiments the animal in pain suffers from chronic pain. In some embodiments the animal is a mammal. In some embodiments the animal is a human.

In some embodiments the pyrone analog such as a flavonoid and/or its metabolite is an activator of P-gP.

In some embodiments the pyrone analog such as a flavonoid is selected from the group consisting of quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, 5,7-dideoxyquercetin (3,3′,4′-trihydroxyflavone), and epicatechin. In some embodiments the pyrone analog such as a flavonoid is quercetin. In some embodiments the pyrone analog such as a flavonoid is fisetin. In some embodiments the pyrone analog such as a flavonoid is 5,7-dideoxyquercetin. In some embodiments the analgesic is selected from the group consisting of oxycodone, gabapentin, pregabalin, hydrocodone, fentanyl, hydromorphine, levorphenol, morphine, methadone, tramadol and topiramate. In some embodiments the analgesic is selected from the group consisting of morphine, oxycodone, and gabapentin. In some embodiments the analgesic is morphine. In some embodiments the analgesic is oxycodone. In some embodiments the analgesic is gabapentin.

In some embodiments the analgesic compound and the pyrone analog such as a flavonoid are administered together about once per day to about 6 times per day. In some embodiments the administration continues for less than about 7 days. In some embodiments the administration continues indefinitely.

In some embodiments the composition further comprises administering to the animal in pain another therapeutic agent. In some embodiments therapeutic agent is selected from the group consisting of antinausea agents, amphetamines, antianxiolytics, and hypnotics. In some embodiments the molar ratio of the amount of analgesic agent administered and the amount of pyrone analog such as a flavonoid administered is about 0.001:1 to about 10:1.

One aspect of the invention is a method of treating an animal comprising; administering an animal in need of treatment an effective amount of a therapeutic agent and a composition comprising a pyrone analog such as a flavonoid and a cyclodextrin. In some embodiments the cyclodextrin is sulfobutylether-7-β-cyclodextrin. In some embodiments the pyrone analog such as a flavonoid and/or its metabolite comprises a BTB transport protein modulator. In some embodiments the BTB transport protein modulator comprises a BTB transport protein activator. In some embodiments the BTB transport protein modulator comprises a modulator of P-gP. In some embodiments the pyrone analog such as a flavonoid and/or its metabolite comprises a side effect modulator. In some embodiments the side effect modulator is present in an amount sufficient to decrease a side effect of the therapeutic agent when the composition is administered to an animal. In some embodiments the side effect modulator is present in an amount sufficient to decrease a side effect of the therapeutic agent by an average of about 10% compared to the side effect without the side effect modulator.

In some embodiments the side effect is selected from the group consisting of hypogonadism and other forms of endocrinopathy, drowsiness, impaired concentration, sexual dysfunction, sleep disturbances, habituation, dependence, alteration of mood, respiratory depression, nausea, vomiting, lowered appetite, lassitude, lowered energy, dizziness, memory impairment, neuronal dysfunction, neuronal death, visual disturbance, impaired mentation, tolerance, addiction, hallucinations, lethargy, myoclonic jerking, and combinations thereof.

In some embodiments the therapeutic agent is selected from the group consisting of antidepressants, anti-psychotics, antihypertensives, vasodilators, barbiturates, membrane stabilizers, cardiac stabilizers, glucocorticoids, antiinfectives, immunomodulators and chemotherapeutic agents. In some embodiments the therapeutic agent is an immunomodulator. In some embodiments the therapeutic agent is tacrolimus. In some embodiments the administration is rectal, buccal, intranasal, transdermal, intravenous, intraperitoneal, parenteral, intramuscular, subcutaneous, orally, topical, as an inhalant, or via an impregnated or coated device such as a stent. In some embodiments the administration is intravenous. In some embodiments the administration is oral. In some embodiments the therapeutic agent is an analgesic and the composition comprising a pyrone analog such as a flavonoid and a cyclodextrin enhances the analgesic affect of the analgesic. In some embodiments the cyclodextrin is sulfobutylether-7-β-cyclodextrin. In some embodiments the pyrone analog such as a flavonoid is quercetin. In some embodiments the pyrone analog such as a flavonoid is fisetin. In some embodiments the pyrone analog such as a flavonoid is 5,7-dideoxyquercetin. In some embodiments wherein the analgesic is morphine.

One aspect of the invention is a pharmaceutical composition comprising a pyrone analog such as a flavonoid, a cyclodextrin, a basic amino acid or a sugar-amine and a pharmaceutically or veterinarily acceptable carrier. In some embodiments the basic amino acid is arginine. In some embodiments the basic amino acid is lysine. In some embodiments the sugar-amine is meglumine.

In some embodiments the pyrone analog such as a flavonoid is quercetin, galangin, fisetin, or kaempferol. In some embodiments the pyrone analog such as a flavonoid is quercetin. In some embodiments the pyrone analog such as a flavonoid is fisetin. In some embodiments the pyrone analog such as a flavonoid is 5,7-dideoxyquercetin.

In some embodiments the cyclodextrin is sulfobutylether-7-β-cyclodextrin.

In some embodiments the pyrone analog such as a flavonoid is quercetin, and the cyclodextrin is sulfobutylether-7-β-cyclodextrin. In some embodiments the pyrone analog such as a flavonoid is fisetin, and the cyclodextrin is sulfobutylether-7-β-cyclodextrin. In some embodiments the pyrone analog such as a flavonoid is 5,7-dideoxyquercetin, and the cyclodextrin is sulfobutylether-7-β-cyclodextrin.

In some embodiments the carrier comprises water. In some embodiments the sulfobutylether-7-β-cyclodextrin is present at a concentration of about 20% w/v or greater. In some embodiments the sulfobutylether-7-β-cyclodextrin is present at a concentration in a range of about 20% w/v to about 30% w/v. In some embodiments the quercetin is present in a range between about 1 mM to about 50 mM. In some embodiments the quercetin is present in a range between about 2 mM to about 40 mM. In some embodiments the amino acid is arginine. In some embodiments the amino acid is lysine. In some embodiments the pH is greater than about 8.5.

In some embodiments the composition is a dry powder formulation. In some embodiments the molar ratio of the quercetin to the sulfobutylether-7-β-cyclodextrin is between about 1:3 and 1:12.

One aspect of the invention is a method of preparing a solution of a pyrone analog such as a flavonoid comprising mixing a cyclodextrin, a pyrone analog such as a flavonoid, and a basic amino acid or a sugar-amine with water at a pH greater than 8.5. In some embodiments the method comprises dissolving the cyclodextrin in water to produce a cyclodextrin solution, then mixing the pyrone analog such as a flavonoid and the basic amino acid or sugar-amine with the cyclodextrin solution. In some embodiments the basic amino acid is arginine. In some embodiments the basic amino acid is lysine. In some embodiments the sugar-amine is meglumine. In some embodiments the pyrone analog such as a flavonoid is quercetin, galangin, or kaempferol. In some embodiments the pyrone analog such as a flavonoid is quercetin. In some embodiments the pyrone analog such as a flavonoid is fisetin. In some embodiments the pyrone analog such as a flavonoid is 5,7-dideoxyquercetin. In some embodiments the cyclodextrin is sulfobutylether-7-β-cyclodextrin. In some embodiments the pyrone analog such as a flavonoid is quercetin, and the cyclodextrin is sulfobutylether-7-β-cyclodextrin. In some embodiments the pyrone analog such as a flavonoid is fisetin, and the cyclodextrin is sulfobutylether-7-β-cyclodextrin. In some embodiments the pyrone analog such as a flavonoid is 5,7-dideoxyquercetin, and the cyclodextrin is sulfobutylether-7-β-cyclodextrin.

In some embodiments of the method the sulfobutylether-7-β-cyclodextrin is present at a concentration of about 20% w/v or greater. In some embodiments the sulfobutylether-7-β-cyclodextrin is present at a concentration in a range of about 20% w/v to about 30% w/v. In some embodiments the quercetin is present in a range between about 1 mM to about 50 mM. In some embodiments the quercetin is present in a range between about 2 mM to about 40 mM. In some embodiments the amino acid is arginine. In some embodiments the amino acid is lysine.

Other objects, features and advantages of the methods and compositions described herein will become apparent from the following detailed description. It should be understood, however, that the detailed description and the specific examples, while indicating specific embodiments, are given by way of illustration only, since various changes and modifications within the spirit and scope of the invention will become apparent to those skilled in the art from this detailed description.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a graph of Rat CWTF data showing the effectiveness of co-administration of a sulfobutylether-7-β-cyclodextrin-flavonoid and morphine for analgesia for a morphine dose of 6 mg/kg.

FIG. 2 is a graph of Rat CWTF data showing the effectiveness of co-administration of a sulfobutylether-7-β-cyclodextrin-flavonoid and morphine for analgesia for a morphine dose of 4 mg/kg.

FIG. 3 is a bar graph that shows stability data for aqueous solutions of quercetin with and without Captisol at about pH 9 with various bases or alkalinizers including basic lysine, arginine, and meglumine.

FIG. 4 is a graph of blood glucose measurements in rats showing attenuation of tacrolimus induced hyperglycemia by pyrone analog-cyclodextrins such as Q-Captisol and Fisetin-Captisol.

### DETAILED DESCRIPTION OF THE INVENTION

Reference will now be made in detail to particularly preferred embodiments of the invention. Examples of the preferred embodiments are illustrated in the following Examples section. Unless defined otherwise, all technical and scientific terms used herein have the same meaning as is commonly understood by one of skill in the art to which this invention belongs. All patents and publications referred to herein are incorporated by reference.

### I. Introduction

This invention relates to compositions and methods for making and using aqueous solutions of pyrone analogs such as flavonoids and cyclodextrins. In some embodiments, the invention provides useful methods for making aqueous solutions of pyrone analogs such as flavonoids and cyclodextrins that involve mixing the pyrone analogs such as flavonoids and cyclodextrins at high pH, then subsequently reducing the pH. The methods of the invention provide a route to make high-concentration aqueous compositions comprising pyrone analogs such as flavonoids and cyclodextrins, for example, comprising sulfobutylether-7-β-cyclodextrin. The compositions can be used as made, or can be further processed, for example by freeze-drying to create a powder composition. These compositions can be used as pharmaceutical compositions to be administered in a variety of ways, for example, intravenously or orally. The ability to have high concentration solutions of these compositions is useful both for the practical processing and manufacturing of pharmaceuticals based on these compositions, and for administering the compositions, where the solubility can be related to bioavailability of the compositions.

In some embodiments, the high solubility aqueous solutions of the invention are stable over time. The stability of the solutions allows them to be stored in some cases for days, weeks or months in liquid form. As used herein, stability with respect to solubility refers to stability with respect to precipitation from solution.

The pyrone analog-sulfoalkyl ether cyclodextrin such as flavonoid-sulfoalkyl ether cyclodextrin compositions of the invention are useful as compositions and method for co-administration with therapeutic agents. The compositions, for example comprising quercetin or a quercetin derivative, can be used to decrease side effects when co-administered with therapeutic agents. The compositions of the invention can be either administered separately, or concurrently with the therapeutic agents. In addition, the compositions of the present invention can be co-administered with therapeutic agents to enhance the effectiveness of the therapeutic agent. For example, a sulfobutylether-7-β-cyclodextrin-quercetin aqueous composition, or a sulfobutylether-7-β-cyclodextrin-quercetin derivative aqueous composition of the present invention can enhance analgesia when co-administered with an immunosuppressive agent such as tacrolimus.

This invention provides compositions and methods utilizing soluble pyrone analogs and/or their metabolites which act in combination with a therapeutic agent to enhance the effectiveness and/or reduce the side effects of the therapeutic agent.

In one aspect, the invention provides compositions and methods utilizing a soluble pyrone analog such as pyrone analog-cyclodextrin that act as a side effect modulator. A “side effect modulator” as used herein is an agent to reduce or eliminate one or more side effects of one or more substances. In some embodiments, the invention provides compositions and methods utilizing a combination of a therapeutic agent and a soluble pyrone analog such as pyrone analog-cyclodextrin that acts as an agent to reduce or eliminate a side effect of the therapeutic agent. Typically, the side effect modulator is a modulator of a blood tissue transport protein, such as P-glycoprotein (herein referred to as blood tissue barrier or BTB transport protein. The methods and compositions are useful in the treatment of an animal in need of treatment, where it is desired that one or more side effects of the substance, e.g., therapeutic agent be reduced or eliminated. In embodiments further utilizing a therapeutic agent, the methods and compositions are useful in the treatment of an animal in need of treatment, where it is desired that one or more side effects of the therapeutic agent be reduced or eliminated while one or more of the therapeutic effects (e.g., peripheral effects) of the agent are retained or enhanced.

In some embodiments of the invention, the therapeutic agent is an analgesic agent, such as an opiate or a non-opiate analgesic. In some embodiments of the invention, the therapeutic agent is a non-analgesic agent. In some embodiments the therapeutic agent is an immunosuppressant such as tacrolimus. The soluble pyrone analog such as pyrone analog-cyclodextrin and/or its metabolite, acting as an agent causing a decrease in the side effects of the therapeutic agent, e.g., a modulator of a BTB transport protein may be an activator or an inhibitor of the protein. The modulatory effect may be dose-dependent, e.g., some modulators act as activators in one dosage range and inhibitors in another. In some embodiments, a modulator of a BTB transport protein is used in a dosage wherein it acts primarily as an activator.

Typically, the use of a soluble pyrone analog such as pyrone analog-cyclodextrin and/or its metabolite as the BTB transport protein modulator, e.g., activator, results in a decrease in one or more side-effects of the therapeutic agent. The therapeutic effect(s) of the agent may be decreased, remain the same, or increase; however, in preferred embodiments, if the therapeutic effect is decreased, it is not decreased to the same degree as the side effects. It will be appreciated that a given therapeutic agent may have more than one therapeutic effect and or one or more side effects, and it is possible that the therapeutic ratio (in this case, the ratio of change in desired effect to change in undesired effect) may vary depending on which effect is measured. However, at least one therapeutic effect of the therapeutic agent is decreased to a lesser degree than at least one side effect of the therapeutic agent.

In addition, in some embodiments, one or more therapeutic effects of the agent is enhanced by use in combination with soluble pyrone analog such as pyrone analog-cyclodextrin phosphorylated polyphenol and/or its metabolite acting as a BTB and/or placental transport protein modulator, while one or more side effects of the therapeutic agent is reduced or substantially eliminated. For example, in some embodiments, the analgesic effect of an analgesic agent is enhanced while one or more side effects of the agent is reduced or substantially eliminated.

Without being bound by theory, and as an example only of a possible mechanism, it is thought that the methods and compositions of the invention operate by reducing or eliminating the concentration of the therapeutic agent from a compartment or compartments in which it causes a side effect, while retaining or even increasing the effective concentration of the agent in the compartment or compartments where it exerts its therapeutic effect.

It will be appreciated that the therapeutic and/or side effects of an therapeutic agent may be mediated in part or in whole by one or more metabolites of the therapeutic agent, and that a BTB modulator that reduces or eliminates the side effect compartment concentration of the therapeutic agent and/or of one or active metabolites of the therapeutic agent that produce side effects, while retaining or enhancing a therapeutic compartment concentration of the therapeutic agent and/or one or more metabolites producing a therapeutic effect, is also encompassed by the methods and compositions of the invention. In addition, a soluble pyrone analog such as pyrone analog-cyclodextrin may be converted in vivo to metabolites that have differing activities in the modulation of one or more BTB transport modulators, and these metabolites are also encompassed by the compositions and methods of the invention.

Hence, in some embodiments the invention provides compositions that include a therapeutic agent and a soluble pyrone analog such as pyrone analog-cyclodextrin, where the therapeutic agent is present in an amount sufficient to exert a therapeutic effect and the soluble pyrone analog such as pyrone analog-cyclodextrin is present in an amount sufficient to decrease side effect, e.g., a side effect of the therapeutic agent when compared to the side effect without the soluble pyrone analog such as pyrone analog-cyclodextrin, when the composition is administered to an animal. The decrease in the side effect can be measurable. The soluble pyrone analog such as pyrone analog-cyclodextrin and/or its metabolite is a BTB transport protein activator in some embodiments. In some embodiments the soluble pyrone analog such as pyrone analog-cyclodextrin is a modulator of ATP binding cassette (ABC) transport proteins. In some embodiments the soluble pyrone analog such as pyrone analog-cyclodextrin is a modulator of P-glycoprotein (P-gP).

In some embodiments, compositions of the invention include one or more than one therapeutic agent as well as one or more than one soluble pyrone analog such as pyrone analog-cyclodextrin. One or more of the therapeutic agents may have one or more side effects which are desired to be decreased.

Compositions of the invention may be prepared in any suitable form for administration to an animal. In some embodiments, the invention provides pharmaceutical compositions.

In some embodiments, the invention provides compositions suitable for oral administration. In some embodiments, compositions are suitable for transdermal administration. In some embodiments, compositions are suitable for injection by any standard route of injection, e.g., intravenous, subcutaneous, intramuscular, or intraperitoneal. Compositions suitable for other routes of administration are also encompassed by the invention, as described herein.

The soluble pyrone analogs such as pyrone analog-cyclodextrins of use in the invention include any soluble pyrone analog such as pyrone analog-cyclodextrin that results in the desired decrease in side effect of a therapeutic agent and/or the increased therapeutic effect of the therapeutic agent, for example, that is a suitable BTB transport protein modulator. In some embodiments, the soluble pyrone analog such as pyrone analog-cyclodextrin is one or more flavonoid-cyclodextrin. In some embodiments, the BTB transport protein modulator is quercetin-cyclodextrin. In some embodiments, the BTB transport protein modulator is fisetin-cyclodextrin. In some embodiments, the BTB transport protein modulator is a phosphorylated 5,7-dideoxyquercetin-cyclodextrin. In some embodiments the cyclodextrin is a sulfoalkylether cyclodextrin such as sulfobutylether-7-β-cyclodextrin.

Therapeutic agents of use in the invention include any suitable agent that produces a side effect that it is desired to reduce or eliminate, while retaining or enhancing a therapeutic effect of the agent. In some embodiments, the therapeutic agent is an analgesic agent. In some instances a side effect may be desirable in some cases and undesirable in others. For example, some analgesics also produce a sedating effect. In some instances, such a sedating effect may be desirable. For example, in the use of analgesics in terminal patients where the main object is to improve quality of the remaining period of life, a certain amount of sedation in addition to analgesia may be desirable. However, it is often desirable to decrease pain without altering mood or concentration, or with minimal alteration of mood or concentration. For example, in patients with chronic intractable pain who are otherwise in good health, it is often desired to achieve maximum alleviation of pain while having minimum sedation or effects on concentration. In the latter case, it is useful to decrease or eliminate the side effect of sedation while retaining the analgesic effect of the agent. It is within the invention to titrate the combination of dosage of therapeutic agent and the side effect modulator, e.g. BTB transport protein modulator, in such a way as to obtain a ratio of therapeutic effect to side effect that is considered optimal. Thus, in some embodiments, one or more side effects of the therapeutic agent is reduced but not eliminated. In other embodiments, one or more side effects of the therapeutic agent is substantially eliminated. In some embodiments, the analgesic agent is an opiate. In some embodiments, the analgesic agent is a non-opiate.

In some embodiments the invention provides methods of treatment. In certain embodiments, the invention provides a method of treating a condition by administering to an animal suffering from the condition an effective amount of a therapeutic agent and an amount of a soluble pyrone analog such as pyrone analog-cyclodextrin, such as such cyclodextrin-quercetin, cyclodextrin-fisetin, or cyclodextrin-5,7-dideoxyquercetin, sufficient to reduce or eliminate a side effect of the therapeutic agent. In some embodiments the soluble pyrone analog such as pyrone analog-cyclodextrin and/or its metabolite is a BTB transport protein activator. In some embodiments, the therapeutic agent is an analgesic agent, e.g., an opiate or a non-opiate analgesic. In certain embodiments the invention provides methods of treatment of pain, e.g., chronic pain, by administration of an analgesic, e.g., an opiate, without the development of tolerance and/or dependence to the analgesic, by co-administering a modulator of a BTB transport protein in combination with the analgesic, thereby partially or completely preventing or delaying development of tolerance and/or dependence to the analgesic. In some embodiments, the therapeutic agent is an immunomodulator such as an immunosuppressant. The compounds of the invention can improve the performance of the immunosuppressant, or reduce a side effect of the immunosuppressant such as nephrotoxicity, renal function impairment, creatinine increase, urinary tract infection, oliguria, cystitis haemorrhagic, hemolytic-uremic syndrome or micturition disorder. In some embodiments, the side effect is decrease in tissue metabolic function.

In some embodiments the invention provides methods of decreasing a side effect of an agent in an animal, e.g. a human, that has received an amount of the agent sufficient to produce a side effect by administering to the animal, e.g., human, an amount of a soluble pyrone analog such as pyrone analog-cyclodextrin sufficient to reduce or eliminate the side effect. In certain embodiments, the agent is an anesthetic, e.g., a general anesthetic. In certain embodiments, the agent is a therapeutic agent or drug of abuse that has be administered in excess, e.g., in an overdose.

### II. Methods of Making Aqueous Pyrone Analog-Cyclodextrin Such as Flavonoid-Cyclodextrin Solutions

One aspect of the invention is a method of making aqueous pyrone analog such as a flavonoid solutions comprising mixing a cyclodextrin and the pyrone analog such as a flavonoid at a pH greater than about 11 and subsequently lowering the pH to less than about 9. In some cases, the method allows for the preparation of aqueous solutions with high concentrations of pyrone analog such as a flavonoid. In some cases, the method allows for the production of aqueous compositions with high concentrations of pyrone analogs such as flavonoids.

One aspect of the invention is a method for forming an aqueous composition comprising a pyrone analog such as a flavonoid comprising: (a) dissolving cyclodextrin in an aqueous solution; (b) adding the flavonoid to the aqueous solution; (c) raising the pH of the aqueous solution to above about pH 11 while mixing the cyclodextrin and pyrone analog such as a flavonoid; and (d) lowering the pH of the aqueous solution to below about pH 9.

In some embodiments, the pH is raised to greater than about pH 11. For example, the pH can be raised to above about 11, 11.2, 11.4, 11.6, 11.8, 12, 12.2, 12.4, 12.6, 12.8, 13, 13.2, 13.4 or above pH 13.4. In general, the pH is raised to bring the pyrone analog such as a flavonoid into solution. In some embodiments the pH is raised to bring as much of the pyrone analog such as a flavonoid into solution as possible without causing significant degradation of the pyrone analog such as a flavonoid. In some embodiments substantially all of the pyrone analog such as a flavonoid is dissolved into solution at the high pH.

In some embodiments, after raising the pH to above pH 11, the pH of the solution is lowered below pH 9. In some embodiments the pH is lowered to below about 8.8, 8.6, 8.5, 8.4, 8.2, 7.8, 7.6, 7.4, 7.2, 7.0, 6.8, 6.5, 6, 5.5, 5, 4.5, 4, 3.5, 3, or less than pH 3. In general, after raising the pH, the pH is lowered to the level at which the aqueous composition will be used or stored. Where the composition is to be used as a pharmaceutical, the pH is lowered to a biologically acceptable pH, usually near neutral pH. In some embodiments, the pH is lowered to between 6 and 9, between 6.5 and 8.5, between about 7.2 and 8.4, between about 7.6 and 8.0, or about pH 7.8.

Some pyrone analogs such as flavonoids are known to be unstable and to degrade in basic solution. For instance, Zheng, et al. J. Pharm. Sci. 94(5), 2005 teaches that while quercetin is stable below pH 3, degradation of quercetin above pH 5 became apparent (see page 1084). Thus, complexation in aqueous solutions between pyrone analogs such as flavonoids and cyclodextrins has generally been carried out at or below neutral pH. For instance, Zheng et al. mix excess quercetin with various cyclodextrins in phosphate buffer at pH 3, mix the mixture for 24 hours, then filter off the undissolved material.

We have found that while pyrone analogs such as flavonoids can degrade in basic solution, aqueous pyrone analog-cyclodextrin such as flavonoid-cyclodextrin compositions can be prepared with the present invention with little to no degradation of the pyrone analog such as a flavonoid by keeping the time during which the pyrone analog such as a flavonoid is above pH 9 short. In some embodiments the time that the pyrone analog such as a flavonoid is above pH 9 is less than about 60, 40, 30, 20, 15, 10, 5, 4, 3, 2, or less than about one minute. In some embodiments, the time that the pyrone analog such as a flavonoid is above pH 9 is less than about 20 minutes. In some embodiments, the time that the pyrone analog such as a flavonoid is above pH 9 is less than about 15 minutes. In some embodiments, the time that the pyrone analog such as a flavonoid is above pH 9 is less than about 10 minutes. In some embodiments, the time that the pyrone analog such as a flavonoid is above pH 9 is less than about 5 minutes. In some embodiments, the time that the pyrone analog such as a flavonoid is above pH 9 is between about 30 and about 60, between about 20 and about 40, between about 15 and about 20, between about 10 and about 15, between about 5 and about 10, between about 1 and about 5, between about 1 and about 10, between about 2 and about 15, or between about 5 and about 15 minutes.

In the methods of the present invention, the temperature at which pyrone analog such as a flavonoid is above pH 9 is generally kept relatively low. In embodiments of the invention, the temperature at which the pyrone analog such as a flavonoid is above pH 9 is kept below about 50° C., below about 40° C., below about 30° C., below about 28° C., below about 26° C., below about 24° C., below about 22° C., below about 20° C., below about 18° C., below about 16° C., below about 15° C., below about 14° C., below about 12° C., or below about 10° C. In some embodiments the temperature at which the pyrone analog such as a flavonoid is above pH 9 is between about 20° C. and about 30° C., between about 10° C. and about 40° C., between about 20° C. and about 26° C., or between about 23° C. and about 25° C.

Any suitable pyrone analog such as a flavonoid can be used in the present invention. A detailed description of pyrone analogs such as flavonoids is provided below. In some embodiments of the method, the pyrone analog such as a flavonoid that is used in the method is selected from the group consisting of quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, 5,7-dideoxyquercetin (3,3′,4′-trihydroxyflavone), and epicatechin or mixtures thereof. In some embodiments of the methods, the pyrone analog such as a flavonoid is quercetin, kaempferol, or galangin or mixtures thereof. In some embodiments, the pyrone analog such as a flavonoid is quercetin. In some embodiments the pyrone analog such as a flavonoid is fisetin. In some embodiments the pyrone analog such as a flavonoid is 5,7-dideoxyquercetin. In some embodiments, the pyrone analog such as a flavonoid is a derivative of quercetin.

The methods of the present invention are useful for pyrone analogs such as flavonoids that are insoluble in water or that are sparingly soluble in water. A flavonoid that is sparingly soluble in water has a low solubility constant or Ks. An example of a sparingly soluble flavonoid is quercetin.

The methods of the present invention are useful for pyrone analogs such as flavonoids having acidic protons. An acidic proton can be removed by base in aqueous solution. In some embodiments, the pKa of the proton is less than 10. In some embodiments the acidic proton will be an OH group that is attached to an aromatic ring, or a phenol group. The pyrone analogs such as flavonoids can have multiple aromatic —OH groups. In some embodiments, the flavonoid has 3, 4, 5, or 6 acidic protons and/or aromatic —OH groups.

While not being bound by theory, it is known that pyrone analogs such as flavonoids with aromatic —OH protons that are substantially water insoluble or sparingly water soluble can be made more water soluble by raising the pH, due at least in part to the deprotonation of the acidic hydrogen(s), creating a pyrone analog such as a flavonoid anion that will tend to be more soluble in water than pyrone analog such as a flavonoid without the proton removed. Thus, raising the pH to above the pKa of the acidic proton on the pyrone analog such as a flavonoid, can result in higher solubility of the pyrone analog such as a flavonoid at the high pH. In the method of the present invention, the pyrone analog such as a flavonoid, at high pH, is mixed with the cyclodextrin, and then the pH of the aqueous solution is lowered. As the pH of the solution is lowered, the pyrone analog such as a flavonoid becomes less soluble, but instead of precipitating out of solution, the pyrone analog such as a flavonoid appears to form a complex with the cyclodextrin. This method is an effective method for rapidly obtaining a soluble pyrone analog-cyclodextrin such as flavonoid-cyclodextrin aqueous composition. Surprisingly, we have found that this method can produce a pyrone analog-cyclodextrin such as flavonoid-cyclodextrin aqueous composition in which the flavonoid is soluble at higher concentrations than obtained by conventional means such as sonicating the pyrone analog such as a flavonoid and cyclodextrin below pH 8. This method can be used to obtain high aqueous concentrations of pyrone analogs such as flavonoids with sulfobutylether-7-β-cyclodextrin. In some embodiments, high aqueous concentrations of quercetin or a quercetin derivative with sulfobutylether-7-β-cyclodextrin can be obtained with the methods of the invention.

The methods of the invention can be used with any suitable type of cyclodextrin. A more detailed description of cyclodextrins is provided below. The methods of the invention can be used with alpha, beta or gamma cyclodextrins. The methods of the invention can be used with modified cyclodextrins such as hydroxypropyl derivatives of alpha-, beta- and gamma-cyclodextrin, sulfoalkylether cyclodextrins such as sulfobutylether beta-cyclodextrin, alkylated cyclodextrins such as the randomly methylated beta.-cyclodextrin, and various branched cyclodextrins such as glucosyl- and maltosyl-beta.-cyclodextrin. In some embodiments, the method is directed at pharmaceutical compositions, in which hydroxypropyl cyclodextrins and sulfoalkyl cyclodextrins can be useful. In some embodiments, sulfobutylether-7-β-cyclodextrin is used.

In addition to pyrone analogs such as flavonoids, the methods of the invention can be used with other compounds that have acidic functional groups, for example for acidic compounds that are sparingly soluble at low pH, and have a higher solubility above about pH 11. For example, in one embodiment of the invention an organic compound with one or more acidic functional groups is mixed with a cyclodextrin at a pH above pH 11 in an aqueous medium, then the pH of the aqueous medium is lowered to below pH 9. In some embodiments the acidic organic compound has one or more groups with a pKa of less than 10. In some embodiments, the acidic organic compound is sparingly soluble or substantially insoluble below pH 9, and is more soluble above pH 11 than at pH 9. In some embodiments, the acidic organic compound is substantially soluble above pH 11. In some embodiments, the acidic organic compound is substantially soluble above pH 12.

Another aspect of the invention is a method of producing an aqueous solution of a pyrone analog such as a flavonoid comprising mixing a pyrone analog such as a flavonoid, a cyclodextrin, and a basic amino acid or sugar-amine at a pH of about 8.5 or greater. It has been found that the basic amino acid, such as lysine and arginine or a sugar-amine such as meglumine, can act, along with the cyclodextrin, to increase the solubility of the pyrone analog such as a flavonoid in water.

As used in the method, the cyclodextrin is generally present at a level between 10% w/v to 40% w/v in the aqueous solution. In some cases the cyclodextrin is present between 15% and 35%. In some cases the cyclodextrin is present between 20% and 35%. In some cases the cyclodextrin is present between 20% and 35%. In some cases the cyclodextrin is present between 25% and 35%. In some cases the cyclodextrin is present between 30% and 35%. In some cases the cyclodextrin is present at about 10%, about 12%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 38% and about 40% w/v in the aqueous solution. In some cases the cyclodextrin is present in a range of 10% to 15%, 15% to 20%, 20% to 25%, 25% to 30%, 30% to 35%, or 35% to 40% w/v in the aqueous solution. In some cases it is found that having a level of cyclodextrin greater than about 20%, greater than about 25%, or greater than about 30% w/v in the aqueous solution can be used to obtain high solubility of the pyrone analog such as a flavonoid. The cyclodextrin that works in this range can be, for example, a sulfoalkyl cyclodextrins such as sulfobutylether-β-cyclodextrin.

The pyrone analog such as a flavonoid used in the method of producing an aqueous solution comprising the pyrone analog such as a flavonoid, cyclodextrin and amino acid or sugar-amine can be a pyrone analog such as a flavonoid known and/or described herein. The pyrone analog such as a flavonoid can be, for example, quercetin or a quercetin derivative, galangin, fisetin, or kaempferol. In some cases, the method provides the pyrone analog such as a flavonoid, e.g. quercetin or a quercetin derivative at a concentration in a range between 1 mg/mL and 15 mg/mL, between 3 mg/mL and 14 mg/mL, between 5 mg/mL and 13 mg/mL, between 6 mg/mL and 12 mg/mL, between 8 mg/mL and 12 mg/mL, or between 9 mg/mL and 11 mg/mL. In some cases, the method provides the pyrone analog such as a flavonoid, e.g. quercetin or a quercetin derivative at a concentration of greater than 1 mg/mL, greater than 2 mg/mL, greater than 4 mg/mL, greater than 3 mg/mL, greater than 5 mg/mL, greater than 6 mg/mL, greater than 7 mg/mL, greater than 8 mg/mL, greater than 9 mg/mL, greater than 10 mg/mL, greater than 11 mg/mL, greater than 12 mg/mL, greater than 13 mg/mL, greater than 14 mg/mL, or greater than 15 mg/mL. In some cases, the method provides the pyrone analog such as a flavonoid e.g. quercetin or a quercetin derivative at a concentration of greater than about 3 mM, greater than about 6 mM, greater than about 9 mM, greater than about 12 mM, greater than about 15 mM, greater than about 18 mM, greater than about 21 mM, greater than about 24 mM, greater than about 27 mM, greater than about 30 mM, or greater than about 33 mM.

The basic amino acid can be any suitable amino acid having a basic group (in addition to the amine of the amino acid). The basic group can be, for example, an amine group or a guanidinium group. The pKa of the basic group will generally be greater than about 9.5, greater than about 10, greater than about 10.5, greater than about 11, or greater than about 11.5. The pKa of the basic group can be between about 9.5 and about 12, between about 10 and about 11.5, or between about 10.5 and 11.5. The amino acid can be a naturally occurring amino acid or a synthetic amino acid. In some cases it is desirable to use a naturally occurring basic amino acid in a pharmaceutical formulation. In some cases lysine is the amino acid. In some cases arginine is the amino acid. In some cases, both lysine and arginine are used in combination.

In some embodiments the methods of the invention use a pyrone analog such as a flavonoid such as quercetin or a quercetin derivative, a sulfobutylether-7-β-cyclodextrin, and a polyhydroxy amine or sugar-amine. For example, in some embodiments, a polyhydroxy compound having a basic group such as an amine, or a sugar having a basic group such as an amine group (a sugar amine) can be used. In some cases the sugar-amine can be 1-Deoxy-1-(methylamino)-D-galactitol, Deoxy-1-(octylamino)-D-glucitol, Deoxy-1-(2-hydroxyethylamino)-D-glucitol, Disorbitylamine, Galactosamine, Glucosamine, or Mannosamine. In some cases, for example, meglumine (N-Methyl-d-glucamine) can be used. While not being bound by theory, these compounds may provide solvation of the pyrone analogs such as flavonoids, e.g. quercetin in the presence of cyclodextrins e.g. sulfobutylether-β-cyclodextrin by having both a basic functional group which can assist in removing a proton from an acidic group on the pyrone analog such as a flavonoid, e.g. quercetin, and by having a hydrophilic portion (the polyhydroxy functionality) to assist in solvation with water.

The amount of the amino acid can be the amount required to bring the pH of the solution above about 8.5, above about 8.7, or above about 9.0.

In some cases, the cyclodextrin, e.g. sulfobutylether-β-cyclodextrin, is first dissolved in water, then subsequently, the pyrone analog such as a flavonoid and basic amino acid or sugar-amine are mixed to form the aqueous solution.

In some cases, the pyrone analog such as a flavonoid, e.g. quercetin or a quercetin derivative, will degrade in the basic medium. Therefore, the time of mixing to form the aqueous solution will in some cases be minimized. In some cases, the mixing is done in less than about 1 hour, less than about 30 minutes, less than about 20 minutes, less than about 15 minutes, less than about 10 minutes, or less than about 5 minutes.

The temperature at which the mixing is carried out is generally near room temperature. In some cases, the temperature is between about 20° C. and about 25° C., between about 18° C. and about 28° C., between about 15° C. and about 30° C., between about 10° C. and about 25° C., between about 5° C. and about 20° C.

After the aqueous solution is formed, the pH of the solution can be neutralized by the addition of acid or by the addition of a buffer solution. In some cases, the acid is hydrochloric acid (HCL). The neutralized solution is generally brought to below pH 8.5. In some cases, the pH of the neutralized solution is between 5 and 8.5, between 6 and 8.5, between 7 and 8.5, between 7 and 8, or between 7.5 and 8. In some cases, the pH of the neutralized solution is 8.5, 8.4, 8.3, 8.2, 8.1, 8.0, 7.9, 7.8, 7.7, 7.6, 7.5, 7.4, 7.3, 7.2, 7.1, or 7.0. In some cases, the pH of the neutralized solution is about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, or about 7.0.

The neutralized solution can then be dried to obtain a dry powder formulation comprising the pyrone analog such as a flavonoid such as quercetin or a quercetin derivative, the cyclodextrin such as sulfobutylether-β-cyclodextrin, and the basic amino acid or sugar-amine. The dry powder can be stored, and can then be re-dissolved in water, for example to produce an intravenous solution. The dry powder can also be formulated as described below into a pharmaceutical formulation suitable for administration via various routes. The powder can be packaged into kits.

In some embodiments of the invention, the pyrone analog such as a flavonoid, such as quercetin or a quercetin derivative, the cyclodextrin such as sulfobutylether-β-cyclodextrin, and the basic amino acid or sugar-amine are mixed in methanol. The methanol is then evaporated to yield a mixture which can be subsequently mixed in water to form an aqueous solution of pyrone analog such as a flavonoid of the present invention. While not being bound by theory, the dissolution of the pyrone analog such as a flavonoid in methanol and the subsequent precipitation of the pyrone analog such as a flavonoid along with the cyclodextrin such as sulfobutylether-β-cyclodextrin is believed in some cases to break up the crystallinity of the pyrone analog such as a flavonoid, promoting disruption of the crystalline lattice and fostering interaction with the other components in a manner that facilitates the subsequent dissolution of the pyrone analog such as a flavonoid in water or aqueous solution. In some embodiments, quercetin, for example in the form of quercetin dihydrate, Captisol, and either arginine, lysine, or meglumine are mixed with methanol, the mixture is filtered from undissolved solids, and the solution obtained from filtration is treated in order to remove the methanol to obtain a solid residue. The removal of methanol can be accomplished, for example, by treating with molecular sieves, distillation, evaporation, or lyophilization. The solid residue can be stored or used right away. The solid residue can then be dissolved in water or aqueous solution to produce an aqueous solution of quercetin.

### III. Pyrone Analogs and Flavonoids of the Invention

As used herein and in the appended claims, the singular forms “a,” “and,” and “the” include plural referents unless the context clearly dictates otherwise. Thus, for example, reference to “a compound” includes a plurality of such compounds, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range may vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

“Acyl” refers to a —(C═O)— radical which is attached to two other moieties through the carbon atom. Those groups may be chosen from alkyl, alkenyl, alkynyl, aryl, heterocylic, heteroaliphatic, heteroaryl, and the like. Unless stated otherwise specifically in the specification, an acyl group is optionally substituted by one or more substituents which independently are: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —OPO₃WY (where W and Y are hydrogen, methyl, ethyl, alkyl, carbohydrate, lithium, sodium or potassium) or —OPO₃Z (where Z is calcium, magnesium or iron) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Acyloxy” refers to a R(C═O)O— radical wherein R is alkyl, aryl, heteroaryl or heterocyclyl. Unless stated otherwise specifically in the specification, an acyloxy group is optionally substituted by one or more substituents which independently are: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2) —S(O)ₜN(R^{a})₂ (where t is 1 or 2), —OPO₃WY (where W and Y are hydrogen, methyl, ethyl, alkyl, carbohydrate, lithium, sodium or potassium) or —OPO₃Z (where Z is calcium, magnesium or iron) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Alkylaryl” refers to an (alkyl)aryl—radical, where alkyl and aryl are as defined herein.

“Aralkyl” refers to an (aryl)alkyl—radical where aryl and alkyl are as defined herein.

“Alkoxy” refers to a (alkyl)O—radical, where alkyl is as described herein and contains 1 to 10 carbons (e.g., C₁-C₁₀ alkyl). Whenever it appears herein, a numerical range such as “1 to 10” refers to each integer in the given range; e.g., “1 to 10 carbon atoms” means that the alkyl group may consist of 1 carbon atom, 2 carbon atoms, 3 carbon atoms, etc., up to and including 10 carbon atoms. In some embodiments, it is a C₁-C₄ alkoxy group. A alkoxy moiety is optionally substituted by one or more of the substituents described as suitable substituents for an alkyl radical.

“Alkyl” refers to a straight or branched hydrocarbon chain radical consisting solely of carbon and hydrogen atoms, containing no unsaturation, having from one to ten carbon atoms (e.g., C₁-C₁₀ alkyl). Whenever it appears herein, a numerical range such as “1 to 10” refers to each integer in the given range; e.g., “1 to 10 carbon atoms” means that the alkyl group may consist of 1 carbon atom, 2 carbon atoms, 3 carbon atoms, etc., up to and including 10 carbon atoms, although the present definition also covers the occurrence of the term “alkyl” where no numerical range is designated. Typical alkyl groups include, but are in no way limited to, methyl, ethyl, propyl, isopropyl, n-butyl, iso-butyl, sec-butyl isobutyl, tertiary butyl, pentyl, isopentyl, neopentyl, hexyl, septyl, octyl, nonyl, decyl, and the like. The alkyl is attached to the rest of the molecule by a single bond, for example, methyl (Me), ethyl (Et), n-propyl, 1-methylethyl (iso-propyl), n-butyl, n-pentyl, 1,1-dimethylethyl (t-butyl), 3-methylhexyl, 2-methylhexyl, and the like. Unless stated otherwise specifically in the specification, an alkyl group is optionally substituted by one or more substituents which independently are: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —OPO₃WY (where W and Y are hydrogen, methyl, ethyl, alkyl, carbohydrate, lithium, sodium or potassium) or —OPO₃Z (where Z is calcium, magnesium or iron) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

“Alkenyl” refers to a straight or branched hydrocarbon chain radical group consisting solely of carbon and hydrogen atoms, containing at least one double bond, and having from two to ten carbon atoms (i.e. C₂-C₁₀ alkenyl). Whenever it appears herein, a numerical range such as “2 to 10” refers to each integer in the given range; e.g., “2 to 10 carbon atoms” means that the alkenyl group may consist of 2 carbon atoms, 3 carbon atoms, etc., up to and including 10 carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more substituents which independently are: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —OPO₃WY (where W and Y are hydrogen, methyl, ethyl, alkyl, carbohydrate, lithium, sodium or potassium) or —OPO₃Z (where Z is calcium, magnesium or iron) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Alkynyl” refers to a straight or branched hydrocarbon chain radical group consisting solely of carbon and hydrogen atoms, containing at least one triple bond, having from two to ten carbon atoms (i.e. C₂-C₁₀ alkynyl). Whenever it appears herein, a numerical range such as “2 to 10” refers to each integer in the given range; e.g., “2 to 10 carbon atoms” means that the alkynyl group may consist of 2 carbon atoms, 3 carbon atoms, etc., up to and including 10 carbon atoms. In certain embodiments, an alkynyl comprises two to eight carbon atoms. In other embodiments, an alkynyl has two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more substituents which independently are: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —OPO₃WY (where W and Y are hydrogen, methyl, ethyl, alkyl, carbohydrate, lithium, sodium or potassium) or —OPO₃Z (where Z is calcium, magnesium or iron) where each R³ is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Amine” refers to a —N(R^{a})₂ radical group, where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl, unless stated otherwise specifically in the specification. Unless stated otherwise specifically in the specification, an amino group is optionally substituted by one or more substituents which independently are: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —OPO₃WY (where W and Y are hydrogen, methyl, ethyl, alkyl, carbohydrate, lithium, sodium or potassium) or —OPO₃Z (where Z is calcium, magnesium or iron) where each R³ is independently hydrogen, alkyl, fluoroalkyl, carbocyclyl, carbocyclylalkyl, aryl, aralkyl, heterocyclyl, heterocyclylalkyl, heteroaryl or heteroarylalkyl.

An “amide” refers to a chemical moiety with formula C(O)NHR or NHC(O)R, where R is selected from the group consisting of alkyl, cycloalkyl, aryl, heteroaryl (bonded through a ring carbon) and heteroalicyclic (bonded through a ring carbon). An amide may be an amino acid or a peptide molecule attached to a compound of Formula (I), thereby forming a prodrug. Any amine, hydroxy, or carboxyl side chain on the compounds described herein can be amidified. The procedures and specific groups to make such amides are known to those of skill in the art and can readily be found in reference sources such as Greene and Wuts, Protective Groups in Organic Synthesis, 3.sup.rd Ed., John Wiley & Sons, New York, N.Y., 1999, which is incorporated herein by reference in its entirety.

“Aromatic” or “aryl” refers to an aromatic radical with six to ten ring atoms (e.g., C₆-C₁₀ aromatic or C₆-C₁₀ aryl) which has at least one ring having a conjugated pi electron system and includes both carbocyclic aryl (e.g., phenyl, fluorenyl, and naphthyl) and heterocyclic aryl (or “heteroaryl” or “heteroaromatic”) groups (e.g., pyridine). Whenever it appears herein, a numerical range such as “6 to 10” refers to each integer in the given range; e.g., “6 to 10 ring atoms” means that the aryl group may consist of 6 ring atoms, 7 ring atoms, etc., up to and including 10 ring atoms. The term includes monocyclic or fused-ring polycyclic (i.e., rings which share adjacent pairs of ring atoms) groups. Unless stated otherwise specifically in the specification, an aryl moiety is optionally substituted by one or more substituents which are independently: hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —CN—OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —OPO₃WY (where W and Y are hydrogen, methyl, ethyl, alkyl, carbohydrate, lithium, sodium or potassium) or —OPO₃Z (where Z is calcium, magnesium or iron) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Carboxaldehyde” refers to a (C═O)H radical.

“Carboxyl” refers to a (C═O)OH radical.

“Carbohydrate” as used herein, includes, but not limited to, monosaccharides, disaccharides, oligosaccharides, or polysaccharides. Monosaccharide for example includes, but not limited to, aldotrioses such as glyceraldehyde, ketotrioses such as dihydroxyacetone, aldotetroses such as erythrose and threose, ketotetroses such as erythrulose, aldopentoses such as arabinose, lyxose, ribose and xylose, ketopentoses such as ribulose and xylulose, aldohexoses such as allose, altrose, galactose, glucose, gulose, idose, mannose and talose, ketohexoses such as fructose, psicose, sorbose and tagatose, heptoses such as mannoheptulose, sedoheptulose, octoses such as octolose, 2-keto-3-deoxy-manno-octonate, nonoses such as sialoseallose. Disaccharides for example includes, but not limited to, glucorhamnose, trehalose, sucrose, lactose, maltose, galactosucrose, N-acetyllactosamine, cellobiose, gentiobiose, isomaltose, melibiose, primeverose, hesperodinose, and rutinose. Oligosaccharides for example includes, but not limited to, raffinose, nystose, panose, cellotriose, maltotriose, maltotetraose, xylobiose, galactotetraose, isopanose, cyclodextrin (α-CD) or cyclomaltohexaose, β-cyclodextrin (β-CD) or cyclomaltoheptaose and γ-cyclodextrin (γ-CD) or cyclomaltooctaose. Polysaccharide for example includes, but not limited to, xylan, mannan, galactan, glucan, arabinan, pustulan, gellan, guaran, xanthan, and hyaluronan. Some examples include, but not limited to, starch, glycogen, cellulose, inulin, chitin, amylose and amylopectin.

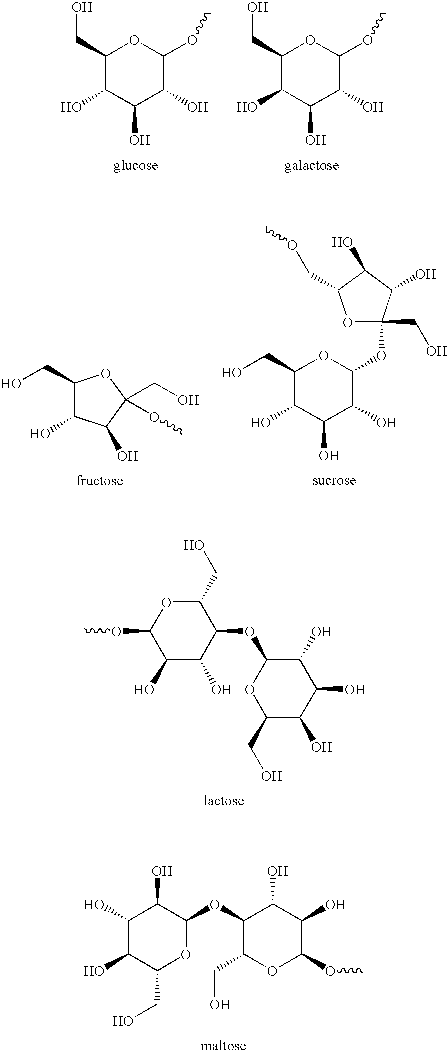

A compound of Formula I having a carbohydrate moeity can be referred to as the pyrone analog glycoside or the pyrone analog saccharide. As used herein, “carbohydrate” further encompasses the glucuronic as well as the glycosidic derivative of compounds of Formula I. Where the pyrone analog has no carbohydrate moeity, it can be referred to as the aglycone. Further, where a phenolic hydroxy is derivatized with any of the carbohydrates described above, the carbohydrate moiety is referred to as a glycosyl residue. Unless stated otherwise specifically in the specification, a carbohydrate group is optionally substituted by one or more substituents which are independently: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —OPO₃WY (where W and Y are hydrogen, methyl, ethyl, alkyl, carbohydrate, lithium, sodium or potassium) or —OPO₃Z (where Z is calcium, magnesium or iron) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Cyano” refers to a —CN moiety.

“Cycloalkyl” refers to a monocyclic or polycyclic radical that contains only carbon and hydrogen, and may be saturated, partially unsaturated, or fully unsaturated. Cycloalkyl groups include groups having from 3 to 10 ring atoms (i.e. C₂-C₁₀ cycloalkyl). Whenever it appears herein, a numerical range such as “3 to 10” refers to each integer in the given range; e.g., “3 to 10 carbon atoms” means that the cycloalkyl group may consist of 3 carbon atoms, etc., up to and including 10 carbon atoms illustrative examples of cycloalkyl groups include, but are not limited to the following moieties: cyclopropyl, cyclobutyl, cyclopentyl, cyclohexyl, cycloseptyl, cyclooctyl, cyclononyl, cyclodecyl, norbornyl, and the like. Unless stated otherwise specifically in the specification, a cycloalkyl group is optionally substituted by one or more substituents which are independently: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —OPO₃WY (where W and Y are hydrogen, methyl, ethyl, alkyl, carbohydrate, lithium, sodium or potassium) or —OPO₃Z (where Z is calcium, magnesium or iron) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Ester” refers to a chemical radical of formula COOR, where R is selected from the group consisting of alkyl, cycloalkyl, aryl, heteroaryl (bonded through a ring carbon) and heteroalicyclic (bonded through a ring carbon). Any amine, hydroxy, or carboxyl side chain on the compounds described herein can be esterified. The procedures and specific groups to make such esters are known to those of skill in the art and can readily be found in reference sources such as Greene and Wuts, Protective Groups in Organic Synthesis, 3.sup.rd Ed., John Wiley & Sons, New York, N.Y., 1999, which is incorporated herein by reference in its entirety. Unless stated otherwise specifically in the specification, an ester group is optionally substituted by one or more substituents which are independently: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —OPO₃WY (where W and Y are hydrogen, methyl, ethyl, alkyl, carbohydrate, lithium, sodium or potassium) or —OPO₃Z (where Z is calcium, magnesium or iron) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical may be optionally substituted as defined above for an alkyl group.

“Halo”, “halide”, or, alternatively, “halogen” means fluoro, chloro, bromo or iodo. The terms “haloalkyl,” “haloalkenyl,” “haloalkynyl” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

The terms “heteroalkyl” “heteroalkenyl” and “heteroalkynyl” include optionally substituted alkyl, alkenyl and alkynyl radicals and which have one or more skeletal chain atoms selected from an atom other than carbon, e.g., oxygen, nitrogen, sulfur, phosphorus or combinations thereof.

“Heteroaryl” or, alternatively, “heteroaromatic” refers to a 5- to 18-membered aryl group (e.g., C₅-C₁₃ heteroaryl) that includes one or more ring heteroatoms selected from nitrogen, oxygen and sulfur, and which may be a monocyclic, bicyclic, tricyclic or tetracyclic ring system. Whenever it appears herein, a numerical range such as “5 to 18” refers to each integer in the given range; e.g., “5 to 18 ring atoms” means that the heteroaryl group may consist of 5 ring atoms, 6 ring atoms, etc., up to and including 18 ring atoms. An N-containing “heteroaromatic” or “heteroaryl” moiety refers to an aromatic group in which at least one of the skeletal atoms of the ring is a nitrogen atom. The polycyclic heteroaryl group may be fused or non-fused. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, a heteraryl moiety is optionally substituted by one or more substituents which are independently: hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —CN, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —OPO₃WY (where W and Y are hydrogen, methyl, ethyl, alkyl, carbohydrate, lithium, sodium or potassium) or —OPO₃Z (where Z is calcium, magnesium or iron) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Heterocyclyl” refers to a stable 3- to 18-membered non-aromatic ring (e.g., C₃-C₁₈ heterocyclyl) radical that comprises two to twelve carbon atoms and from one to six heteroatoms selected from nitrogen, oxygen and sulfur. Whenever it appears herein, a numerical range such as “3 to 18” refers to each integer in the given range; e.g., “3 to 18 ring atoms” means that the heteroaryl group may consist of 3 ring atoms, 4 ring atoms, etc., up to and including 18 ring atoms. In some embodiments, it is a C₅-C₁₀ heterocyclyl. In some embodiments, it is a C₄-C₁₀ heterocyclyl. In some embodiments, it is a C₃-C₁₀heterocyclyl. Unless stated otherwise specifically in the specification, the heterocyclyl radical is a monocyclic, bicyclic, tricyclic or tetracyclic ring system, which may include fused or bridged ring systems. The heteroatoms in the heterocyclyl radical may be optionally oxidized. One or more nitrogen atoms, if present, are optionally quaternized. The heterocyclyl radical is partially or fully saturated. The heterocyclyl may be attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocylyl moiety is optionally substituted by one or more substituents which are independently: hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀heterocyclic, C₃-C₁₀ cycloalkyl, —CN, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —OPO₃WY (where W and Y are hydrogen, methyl, ethyl, alkyl, carbohydrate, lithium, sodium or potassium) or —OPO₃Z (where Z is calcium, magnesium or iron) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Heteroalicyclic” refers to a cycloalkyl radical that includes at least one heteroatom selected from nitrogen, oxygen and sulfur. The radicals may be fused with an aryl or heteroaryl. The term heteroalicyclic also includes all ring forms of the carbohydrates, including but not limited to the monosaccharides, the disaccharides and the oligosaccharides. Unless stated otherwise specifically in the specification, a heteroalicyclic group is optionally substituted by one or more substituents which independently are: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —OPO₃WY (where W and Y are hydrogen, methyl, ethyl, alkyl, carbohydrate, lithium, sodium or potassium) or —OPO₃Z (where Z is calcium, magnesium or iron) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Imino” refers to the ═N—H radical.

“Isocyanato” refers to a NCO radical.

“Isothiocyanato” refers to a NCS radical.

“Mercaptyl” refers to a (alkyl)S or (H)S radical.

“Moiety” refers to a specific segment or functional group of a molecule. Chemical moieties are often recognized chemical entities embedded in or appended to a molecule.

“Nitro” refers to the NO₂radical.

“Oxa” refers to the —O— radical.

“Oxo” refers to the ═O radical.

“Sulfinyl” refers to a S(═O)—R radical, where R is selected from the group consisting of alkyl, cycloalkyl, aryl, heteroaryl (bonded through a ring carbon) and heteroalicyclic (bonded through a ring carbon)

“Sulfonyl” refers to a S(═O)₂—R radical, where R is selected from the group consisting of alkyl, cycloalkyl, aryl, heteroaryl (bonded through a ring carbon) and heteroalicyclic (bonded through a ring carbon).

“Sulfonamidyl” refers to a S(═O)₂—NRR radical, where each R is selected independently from the group consisting of hydrogen, alkyl, cycloalkyl, aryl, heteroaryl (bonded through a ring carbon) and heteroalicyclic (bonded through a ring carbon).

“Sulfoxyl” refers to a S(═O)₂OH radical.

“Sulfonate” refers to a S(═O)₂—OR radical, where R is selected from the group consisting of alkyl, cycloalkyl, aryl, heteroaryl (bonded through a ring carbon) and heteroalicyclic (bonded through a ring carbon).

“Thiocyanato” refers to a CNS radical.

“Thioxo” refers to the ═S radical.

“Substituted” means that the referenced group may be substituted with one or more additional group(s) individually and independently selected from acyl, alkyl, alkylaryl, cycloalkyl, aralkyl, aryl, carbohydrate, heteroaryl, heterocyclic, hydroxy, alkoxy, aryloxy, mercapto, alkylthio, arylthio, cyano, halo, carbonyl, ester, thiocarbonyl, isocyanato, thiocyanato, isothiocyanato, nitro, perhaloalkyl, perfluoroalkyl, phosphate, silyl, sulfinyl, sulfonyl, sulfonamidyl, sulfoxyl, sulfonate, and amino, including mono- and di-substituted amino groups, and the protected derivatives thereof. The subsituents themselves may be substituted, for example, a cycloakyl substituent may have a halide substituted at one or more ring carbons, and the like. The protecting groups that may form the protective derivatives of the above substituents are known to those of skill in the art and may be found in references such as Greene and Wuts, above.

The compounds presented herein may possess one or more chiral centers and each center may exist in the R or S configuration. The compounds presented herein include all diastereomeric, enantiomeric, and epimeric forms as well as the appropriate mixtures thereof. Stereoisomers may be obtained, if desired, by methods known in the art as, for example, the separation of stereoisomers by chiral chromatographic columns.

The methods and formulations described herein include the use of N-oxides, crystalline forms (also known as polymorphs), or pharmaceutically acceptable salts of compounds having the structure of Formula (I), as well as active metabolites of these compounds having the same type of activity. In addition, the compounds described herein can exist in unsolvated as well as solvated forms with pharmaceutically acceptable solvents such as water, ethanol, and the like. The solvated forms of the compounds presented herein are also considered to be disclosed herein.

Pyrone analogs of Formula I and their pharmaceutically/veterinarily acceptable salt or esters are provided in this invention,

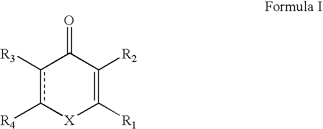

wherein X is O, S, or NR′, wherein R′ is hydrogen, C₁-C₁₀ alkyl, C₂-C₁₀ alkynyl, C₂-C₁₀ alkenyl, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, or C₃-C₁₀ cycloalkyl;

R₁, and R₂ are independently hydrogen, hydroxyl, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₄-C₁₀heterocyclyl, heteroaryl, C₃-C₁₀cycloalkyl, —OPO₃WY, —OCH₂PO₄WY, —OCH₂PO₄Z or —OPO₃Z;

R₃ and R₄ are independently hydrogen, hydroxyl, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl C₆-C₁₀ aralkyl acyl, C₆-C₁₀ alkylaryl acyl, alkoxy, amine, aryl, C₄-C₁₀ heterocyclyl, heteroaryl, C₃-C₁₀cycloalkyl, —OPO₃WY, —OCH₂PO₄WY, —OCH₂PO₄Z or —OPO₃Z;

or R₃ and R₄ are taken together to form a C₅-C₁₀heterocyclyl, C₅-C₁₀cycloalkyl, aryl, or heteroaryl; and

W and Y are independently hydrogen, methyl, ethyl, alkyl, carbohydrate, or a cation, and Z is a multivalent cation.

In some embodiments, X is O.

In other embodiments, X is S.

In yet other embodiments, X is NR′.

In some embodiments, R′ is hydrogen. In some embodiments, R′ is unsubstituted C₁-C₁₀ alkyl. In some embodiments, R′ is substituted C₁-C₁₀ alkyl. In some embodiments, R′ is unsubstituted C₂-C₁₀ alkynyl. In some embodiments, R′ is substituted C₂-C₁₀ alkynyl. In some embodiments, R′ is unsubstituted C₂-C₁₀ alkenyl. In some embodiments, R′ is substituted C₂-C₁₀ alkenyl. In some embodiments, R′ is unsubstituted C₁-C₁₀ aliphatic acyl. In some embodiments, R′ is substituted C₁-C₁₀ aliphatic acyl. In some embodiments, R′ is unsubstituted C₆-C₁₀ aromatic acyl. In some embodiments, R′ is substituted C₆-C₁₀ aromatic acyl. In some embodiments, R′ is unsubstituted C₆-C₁₀ aralkyl acyl. In some embodiments, R′ is substituted C₆-C₁₀ aralkyl acyl. In some embodiments, R′ is unsubstituted C₆-C₁₀ alkylaryl acyl. In some embodiments, R′ is substituted C₆-C₁₀ alkylaryl acyl. In some embodiments, R′ is unsubstituted aryl. In some embodiments, R′ is substituted aryl. In some embodiments, R′ is unsubstituted C₃-C₁₀ heterocyclyl. In some embodiments, R′ is substituted C₃-C₁₀ heterocyclyl. In some embodiments, R′ is unsubstituted heteroaryl. In some embodiments, R′ is substituted heteroaryl. In some embodiments, R′ is unsubstituted C₃-C₁₀cycloalkyl. In some embodiments, R′ is substituted C₃-C₁₀cycloalkyl.

In some embodiments, R₁ is hydrogen. In some embodiments, R₁ is optionally substituted C₁-C₁₀ alkyl. hydroxyl. In some embodiments, R₁ is unsubstituted C₁-C₁₀ alkyl. In some embodiments, R₁ is substituted C₁-C₁₀ alkyl. In some embodiments, R₁ is unsubstituted C₁-C₁₀ alkyl. In some other embodiments, R₁ is substituted C₁-C₁₀ alkyl. In some embodiments, R₁ is unsubstituted C₂-C₁₀ alkynyl. In some embodiments, R₁ is substituted C₂-C₁₀ alkynyl. In some embodiments, R₁ is unsubstituted C₂-C₁₀ alkenyl. In some embodiments, R₁ is substituted C₂-C₁₀ alkenyl. In some embodiments, R₁ is carboxyl. In some embodiments, R₁ is unsubstituted carbohydrate. In some embodiments, R₁ is substituted carbohydrate. In some embodiments, R₁ is unsubstituted ester. In some embodiments, R₁ is substituted ester. In some embodiments, R₁ is unsubstituted acyloxy. In some embodiments, R₁ is substituted acyloxy. In some embodiments, R₁ is nitro. In some embodiments, R₁ is halogen. In some embodiments, R₁ is unsubstituted C₁-C₁₀ aliphatic acyl. In some embodiments, R₁ is substituted C₁-C₁₀ aliphatic acyl. In some embodiments, R₁ is unsubstituted C₆-C₁₀ aromatic acyl. In some embodiments, R₁ is substituted C₆-C₁₀ aromatic acyl. In some embodiments, R₁ is unsubstituted C₆-C₁₀ aralkyl acyl. In some embodiments, R₁ is substituted C₆-C₁₀ aralkyl acyl. In some embodiments, R₁ is unsubstituted C₆-C₁₀ alkylaryl acyl. In some embodiments, R₁ is substituted C₆-C₁₀ alkylaryl acyl. In some embodiments, R₁ is unsubstituted alkoxy. In some embodiments, R₁ is substituted alkoxy. In some embodiments, R₁ is unsubstituted amine. In some embodiments, R₁ is substituted amine. In some embodiments, R₁ is unsubstituted aryl. In some embodiments, R₁ is substituted aryl. In some embodiments, R₁ is unsubstituted C₄-C₁₀heterocyclyl. In some embodiments, R₁ is substituted C₄-C₁₀ heterocyclyl. In some embodiments, R₁ is unsubstituted heteroaryl. In some embodiments, R₁ is substituted heteroaryl. In some embodiments, R₁ is unsubstituted C₃-C₁₀cycloalkyl. In some embodiments, R₁ is substituted C₃-C₁₀cycloalkyl. In some embodiments, R₁ is —OPO₃WY. In some embodiments, R₁ is —OCH₂PO₄WY. In some embodiments, R₁ is —OCH₂PO₄Z. In some embodiments, R₁ is —OPO₃Z.

In some embodiments, when R₁ is aryl, it is monocyclic. In some embodiments, when R₁ is aryl, it is bicyclic. In some embodiments, when R₁ is heteroaryl, it is monocyclic. In some embodiments, when R₁ is heteroaryl, it is bicyclic.

In some embodiments, R₂ is hydrogen. In some embodiments, R₂ is hydroxyl. In some embodiments, R₂ is optionally substituted C₁-C₁₀ alkyl. In some embodiments, R₂ is unsubstituted C₁-C₁₀ alkyl. In some embodiments, R₂ is substituted C₁-C₁₀alkyl. In some embodiments, R₂ is unsubstituted C₁-C₁₀ alkyl. In some other embodiments, R₂ is substituted C₁-C₁₀alkyl. In some embodiments, R₂ is unsubstituted C₂-C₁ alkynyl. In some embodiments, R₂ is substituted C₂-C₁₀ alkynyl. In some embodiments, R₂ is unsubstituted C₂-C₁₀ alkenyl. In some embodiments, R₂ is substituted C₂-C₁₀ alkenyl. In some embodiments, R₂ is carboxyl. In some embodiments, R₂ is unsubstituted carbohydrate. In some embodiments, R₂ is substituted carbohydrate. In some embodiments, R₂ is unsubstituted ester. In some embodiments, R₂ is substituted ester. In some embodiments, R₂ is unsubstituted acyloxy. In some embodiments, R₂ is substituted acyloxy. In some embodiments, R₂ is nitro. In some embodiments, R₂ is halogen. In some embodiments, R₂ is unsubstituted C₁-C₁₀ aliphatic acyl. In some embodiments, R₂ is substituted C₁-C₁₀ aliphatic acyl. In some embodiments, R₂ is unsubstituted C₆-C₁₀ aromatic acyl. In some embodiments, R₂ is substituted C₆-C₁₀ aromatic acyl. In some embodiments, R₂ is unsubstituted C₆-C₁₀ aralkyl acyl. In some embodiments, R₂ is substituted C₆-C₁₀ aralkyl acyl. In some embodiments, R₂ is unsubstituted C₆-C₁₀ alkylaryl acyl. In some embodiments, R₂ is substituted C₆-C₁₀ alkylaryl acyl. In some embodiments, R₂ is unsubstituted alkoxy. In some embodiments, R₂ is substituted alkoxy. In some embodiments, R₂ is unsubstituted amine. In some embodiments, R₂ is substituted amine. In some embodiments, R₂ is unsubstituted aryl. In some embodiments, R₂ is substituted aryl. In some embodiments, R₂ is unsubstituted C₄-C₁₀heterocyclyl. In some embodiments, R₂ is substituted C₄-C₁₀ heterocyclyl. In some embodiments, R₂ is unsubstituted heteroaryl. In some embodiments, R₂ is substituted heteroaryl. In some embodiments, R₂ is unsubstituted C₃-C₁₀cycloalkyl. In some embodiments, R₂ is substituted C₃-C₁₀cycloalkyl. In some embodiments, R₂ is —OPO₃WY. In some embodiments, R₂ is —OCH₂PO₄WY. In some embodiments, R₂ is —OCH₂PO₄Z. In some embodiments, R₂ is —OPO₃Z.

In some embodiments, R₃ is hydrogen. In some embodiments, R₃ is optionally substituted C₁-C₁₀ alkyl. hydroxyl. In some embodiments, R₃ is unsubstituted C₁-C₁₀ alkyl. In some embodiments, R₃ is substituted C₁-C₁₀ alkyl. In some embodiments, R₃ is unsubstituted C₁-C₁₀ alkyl. In some other embodiments, R₃ is substituted C₁-C₁₀ alkyl. In some embodiments, R₃ is unsubstituted C₂-C₁₀ alkynyl. In some embodiments, R₃ is substituted C₂-C₁₀ alkynyl. In some embodiments, R₃ is unsubstituted C₂-C₁₀ alkenyl. In some embodiments, R₃ is substituted C₂-C₁₀ alkenyl. In some embodiments, R₃ is carboxyl. In some embodiments, R₃ is unsubstituted carbohydrate. In some embodiments, R₃ is substituted carbohydrate. In some embodiments, R₃ is unsubstituted ester. In some embodiments, R₃ is substituted ester. In some embodiments, R₃ is unsubstituted acyloxy. In some embodiments, R₃ is substituted acyloxy. In some embodiments, R₃ is nitro. In some embodiments, R₃ is halogen. In some embodiments, R₃ is unsubstituted C₁-C₁₀ aliphatic acyl. In some embodiments, R₃ is substituted C₁-C₁₀ aliphatic acyl. In some embodiments, R₃ is unsubstituted C₆-C₁₀ aromatic acyl. In some embodiments, R₃ is substituted C₆-C₁₀ aromatic acyl. In some embodiments, R₃ is unsubstituted C₆-C₁₀ aralkyl acyl. In some embodiments, R₃ is substituted C₆-C₁₀ aralkyl acyl. In some embodiments, R₃ is unsubstituted C₆-C₁₀ alkylaryl acyl. In some embodiments, R₃ is substituted C₆-C₁₀ alkylaryl acyl. In some embodiments, R₃ is unsubstituted alkoxy. In some embodiments, R₃ is substituted alkoxy. In some embodiments, R₃ is unsubstituted amine. In some embodiments, R₃ is substituted amine. In some embodiments, R₃ is unsubstituted aryl. In some embodiments, R₃ is substituted aryl. In some embodiments, R₃ is unsubstituted C₄-C₁₀heterocyclyl. In some embodiments, R₃ is substituted C₄-C₁₀ heterocyclyl. In some embodiments, R₃ is unsubstituted heteroaryl. In some embodiments, R₃ is substituted heteroaryl. In some embodiments, R₃ is unsubstituted C₃-C₁₀cycloalkyl. In some embodiments, R₃ is substituted C₃-C₁₀cycloalkyl. In some embodiments, R₃ is —OPO₃WY. In some embodiments, R₃ is —OCH₂PO₄WY. In some embodiments, R₃ is —OCH₂PO₄Z. In some embodiments, R₃ is —OPO₃Z.

In some embodiments, R₄ is hydrogen. In some embodiments, R₄ is optionally substituted C₁-C₁₀ alkyl. hydroxyl. In some embodiments, R₄ is unsubstituted C₁-C₁₀ alkyl. In some embodiments, R₄ is substituted C₁-C₁₀ alkyl. In some embodiments, R₄ is unsubstituted C₁-C₁₀ alkyl. In some other embodiments, R₄ is substituted C₁-C₁₀ alkyl. In some embodiments, R₄ is unsubstituted C₂-C₁₀ alkynyl. In some embodiments, R₄ is substituted C₂-C₁₀ alkynyl. In some embodiments, R₄ is unsubstituted C₂-C₁₀ alkenyl. In some embodiments, R₄ is substituted C₂-C₁₀ alkenyl. In some embodiments, R₄ is carboxyl. In some embodiments, R₄ is unsubstituted carbohydrate. In some embodiments, R₄ is substituted carbohydrate. In some embodiments, R₄ is unsubstituted ester. In some embodiments, R₄ is substituted ester. In some embodiments, R₄ is unsubstituted acyloxy. In some embodiments, R₄ is substituted acyloxy. In some embodiments, R₄ is nitro. In some embodiments, R₄ is halogen. In some embodiments, R₄ is unsubstituted C₁-C₁₀ aliphatic acyl. In some embodiments, R₄ is substituted C₁-C₁₀ aliphatic acyl. In some embodiments, R₄ is unsubstituted C₆-C₁₀ aromatic acyl. In some embodiments, R₄ is substituted C₆-C₁₀ aromatic acyl. In some embodiments, R₄ is unsubstituted C₆-C₁₀ aralkyl acyl. In some embodiments, R₄ is substituted C₆-C₁₀ aralkyl acyl. In some embodiments, R₄ is unsubstituted C₆-C₁₀ alkylaryl acyl. In some embodiments, R₄ is substituted C₆-C₁₀ alkylaryl acyl. In some embodiments, R₄ is unsubstituted alkoxy. In some embodiments, R₄ is substituted alkoxy. In some embodiments, R₄ is unsubstituted amine. In some embodiments, R₄ is substituted amine. In some embodiments, R₄ is unsubstituted aryl. In some embodiments, R₄ is substituted aryl. In some embodiments, R₄ is unsubstituted C₄-C₁₀heterocyclyl. In some embodiments, R₄ is substituted C₄-C₁₀ heterocyclyl. In some embodiments, R₄ is unsubstituted heteroaryl. In some embodiments, R₄ is substituted heteroaryl. In some embodiments, R₄ is unsubstituted C₃-C₁₀cycloalkyl. In some embodiments, R₄ is substituted C₃-C₁₀cycloalkyl. In some embodiments, R₄ is —OPO₃WY. In some embodiments, R₄ is —OCH₂PO₄WY. In some embodiments, R₄ is —OCH₂PO₄Z. In some embodiments, R₄ is —OPO₃Z.

In some embodiments, R₃ and R₄ are taken together to form an unsubstituted C₅-C₁₀heterocyclyl. In other embodiments, R₃ and R₄ are taken together to form a substituted C₅-C₁₀heterocyclyl. In some embodiments, R₃ and R₄ are taken together to form an unsubstituted C₅-C₁₀cycloalkyl. In some embodiments, R₃ and R₄ are taken together to form a substituted C₅-C₁₀cycloalkyl. In some embodiments, R₃ and R₄ are taken together to form an unsubstituted aryl. In some embodiments, R₃ and R₄ are taken together to form a substituted aryl. In some embodiments, R₃ and R₄ are taken together to form an unsubstituted heteroaryl. In some embodiments, R₃ and R₄ are taken together to form a substituted heteroaryl.

In various embodiments, W is hydrogen. In various embodiments, W is unsubstituted methyl. In various embodiments, W is substituted methyl. In various embodiments, W is unsubstituted ethyl. In various embodiments, W is substituted ethyl. In various embodiments, W is unsubstituted alkyl. In various embodiments, W is substituted alkyl. In various embodiments, W is unsubstituted carbohydrate. In various embodiments, W is substituted carbohydrate. In various embodiments, W is potassium. In various embodiments, W is sodium. In various embodiments, W is lithium. In various embodiments, Y is hydrogen. In various embodiments, Y is unsubstituted methyl. In various embodiments, Y is substituted methyl. In various embodiments, Y is unsubstituted ethyl. In various embodiments, Y is substituted ethyl. In various embodiments, Y is unsubstituted alkyl. In various embodiments, Y is substituted alkyl. In various embodiments, Y is unsubstituted carbohydrate. In various embodiments, Y is substituted carbohydrate. In various embodiments, Y is potassium. In various embodiments, Y is sodium. In various embodiments, Y is lithium.

In various embodiments, Z is calcium. In various embodiments, Z is magnesium. In various embodiments, Z is iron.

The 2, 3 bond may be saturated or unsaturated in the compounds of Formula I.

In some embodiments of the invention, the pyrone analog of Formula I is of Formula II:

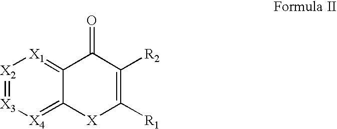

wherein X, R₁, R₂, W, Y, and Z are defined as in Formula I;

X₁, X₂, X₃, and X₄ are independently CR₅, O, S, or N;

each instance of R₅ is independently hydrogen, hydroxyl, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀alkylaryl acyl, alkoxy, amine, aryl, C₃-C₁₀heterocyclyl, heteroaryl, C₃-C₁₀cycloalkyl, —OPO₃WY, —OCH₂PO₄WY, —OCH₂PO₄Z or —OPO₃Z.

In some embodiments, X₁ is CR₅.

In other embodiments, X₁ is O.

In yet other embodiments, X₁ is S.

In further embodiments, X₁ is N.

In some embodiments, X₂ is CR₅.

In other embodiments, X₂ is O.

In yet other embodiments, X₂ is S.

In further embodiments, X₂ is N.

In some embodiments, X₃ is CR₅.

In other embodiments, X₃ is O.

In yet other embodiments, X₃ is S.

In further embodiments, X₃ is N.

In other embodiments, X₄ is CR₅.

In some embodiments, X₄ is O.

In yet other embodiments, X₄ is S.

In some embodiments, X₄ is N.

In some embodiments, X₁, X₂, X₃, and X₄ are CR₅.

In some embodiments, X₁ and X₃ are CR₅ and X₂ and X₄ are N.

In some embodiments, X₂ and X₄ are CR₅ and X₁ and X₃ are N.

In some embodiments, X₂ and X₃ are CR₅ and X₁ and X₄ are N.

In various embodiments, R₁ is one of the following formulae:

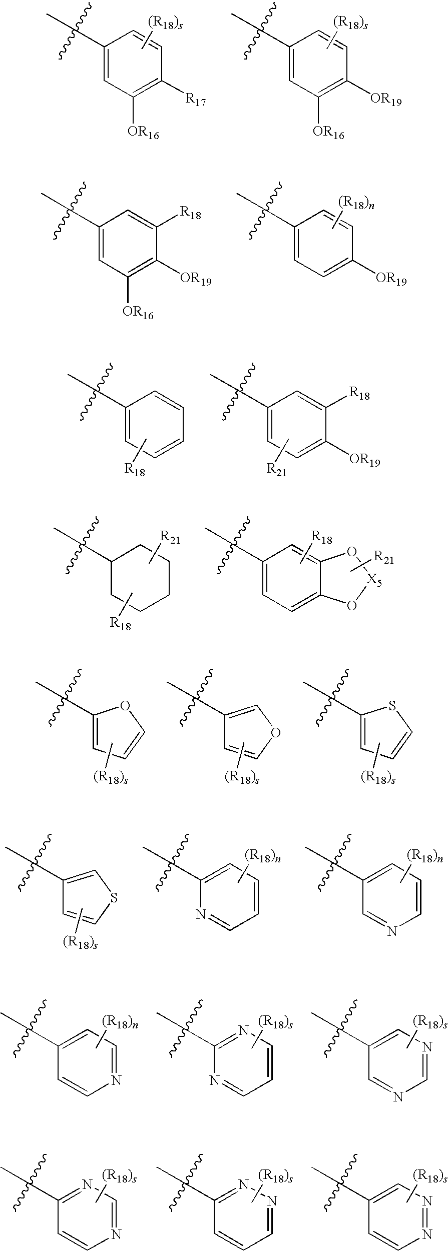

wherein R₁₆ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, C₃-C₁₀cycloalkyl, —PO₃WY, —CH₂PO₄WY, —CH₂PO₄Z or —PO₃Z;

R₁₇ is hydrogen, hydroxy, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, aryl, C₃-C₁₀ heterocyclyl, heteroaryl, or C₃-C₁₀cycloalkyl, —OPO₃WY, —OCH₂PO₄WY, —OCH₂PO₄Z or —OPO₃Z;

each instance of R₁₈ and R₂₁ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z;

R₁₉ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀heterocyclyl, heteroaryl, optionally substituted C₃-C₁₀cycloalkyl, —PO₃WY, —CH₂PO₄WY, —CH₂PO₄Z or —PO₃Z;

s is an integer of 0, 1, 2, or 3; and

n is an integer of 0, 1, 2, 3, or 4.

In some embodiments, R₁₆ is hydrogen. In some embodiments, R₁₆ is unsubstituted C₁-C₁₀ alkyl. In some embodiments, R₁₆ is substituted C₁-C₁₀ alkyl. In some embodiments, R₁₆ is unsubstituted C₂-C₁₀ alkynyl. In some embodiments, R₁₆ is substituted C₂-C₁₀ alkynyl. In some embodiments, R₁₆ is unsubstituted C₂-C₁₀ alkenyl. In some embodiments, R₁₆ is substituted C₂-C₁₀ alkenyl. In some embodiments, R₁₆ is unsubstituted carbohydrate 1. In some embodiments, R₁₆ is substituted carbohydrate. In some embodiments, R₁₆ is unsubstituted C₁-C₁₀ aliphatic acyl. In some embodiments, R₁₆ is substituted C₁-C₁₀ aliphatic acyl. In some embodiments, R₁₆ is unsubstituted C₆-C₁₀ aromatic acyl. In some embodiments, R₁₆ is substituted C₆-C₁₀ aromatic acyl. In some embodiments, R₁₆ is unsubstituted C₆-C₁₀ aralkyl acyl. In some embodiments, R₁₆ is substituted C₆-C₁₀ aralkyl acyl. In some embodiments, R₁₆ is unsubstituted C₆-C₁₀ alkylaryl acyl. In some embodiments, R₁₆ is substituted C₆-C₁₀ alkylaryl acyl. In some embodiments, R₁₆ is unsubstituted aryl. In some embodiments, R₁₆ is substituted aryl. In some embodiments, R₁₆ is unsubstituted C₃-C₁₀heterocyclyl. In some embodiments, R₁₆ is substituted C₃-C₁₀heterocyclyl. In some embodiments, R₁₆ is unsubstituted heteroaryl. In some embodiments, R₁₆ is substituted heteroaryl. In some embodiments, R₁₆ is unsubstituted C₃-C₁₀cycloalkyl. In some embodiments, R₁₆ is substituted C₃-C₁₀cycloalkyl. In some embodiments, R₁₆ is —PO₃WY. In some embodiments, R₁₆ is —CH₂PO₄WY. In some embodiments, R₁₆ is —CH₂PO₄Z. In some embodiments, R₁₆ is —PO₃Z.

In some embodiments, R₁₇ is hydrogen. In some embodiments, R₁₇ is hydroxy. In some embodiments, R₁₇ is carboxaldehyde. In some embodiments, R₁₇ is unsubstituted amine. In some embodiments, R₁₇ is substituted amine. In some embodiments, R₁₇ is unsubstituted C₁-C₁₀ alkyl. In some embodiments, R₁₇ is unsubstituted C₂-C₁₀ alkynyl. In some embodiments, R₁₇ is substituted C₂-C₁₀ alkynyl. In some embodiments, R₁₇ is unsubstituted C₂-C₁₀ alkenyl. In some embodiments, R₁₇ is substituted C₂-C₁₀ alkenyl. In some embodiments, R₁₇ is carboxyl. In some embodiments, R₁₇ is unsubstituted carbohydrate. In some embodiments, R₁₇ is substituted carbohydrate. In some embodiments, R₁₇ is unsubstituted ester. In some embodiments, R₁₇ is substituted ester. In some embodiments, R₁₇ is unsubstituted acyloxy. In some embodiments, R₁₇ is substituted acyloxy. In some embodiments, R₁₇ is nitro. In some embodiments, R₁₇ is halogen. In some embodiments, R₁₇ is unsubstituted C₁-C₁₀ aliphatic acyl. In some embodiments, R₁₇ is substituted C₁-C₁₀ aliphatic acyl. In some embodiments, R₁₇ is unsubstituted C₆-C₁₀ aromatic acyl. In some embodiments, R₁₇ is substituted C₆-C₁₀ aromatic acyl. In some embodiments, R₁₇ is unsubstituted C₆-C₁₀ aralkyl acyl. In some embodiments, R₁₇ is substituted C₆-C₁₀ aralkyl acyl. In some embodiments, R₁₇ is unsubstituted C₆-C₁₀ alkylaryl acyl. In some embodiments, R₁₇ is substituted C₆-C₁₀ alkylaryl acyl. In some embodiments, R₁₇ is unsubstituted alkoxy. In some embodiments, R₁₇ is substituted alkoxy. In some embodiments, R₁₇ is unsubstituted aryl. In some embodiments, R₁₇ is substituted aryl. In some embodiments, R₁₇ is unsubstituted C₃-C₁₀heterocyclyl. In some embodiments, R₁₇ is substituted C₃-C₁₀heterocyclyl. In some embodiments, R₁₇ is unsubstituted heteroaryl. In some embodiments, R₁₇ is substituted heteroaryl. In some embodiments, R₁₇ is unsubstituted C₃-C₁₀cycloalkyl. In some embodiments, R₁₇ is substituted C₃-C₁₀cycloalkyl. In some embodiments, R₁₇ is —OPO₃WY. In some embodiments, R₁₇ is —OCH₂PO₄WY. In some embodiments, R₁₇ is —OCH₂PO₄Z. In some embodiments, R₁₇ is —OPO₃Z.

In some embodiments, R₁₈ is hydrogen. In some embodiments, R₁₈ is hydroxy. In some embodiments, R₁₈ is carboxaldehyde. In some embodiments, R₁₈ is unsubstituted amine. In some embodiments, R₁₈ is substituted amine. In some embodiments, R₁₈ is unsubstituted C₁-C₁₀ alkyl. In some embodiments, R₁₈ is unsubstituted C₂-C₁₀ alkynyl. In some embodiments, R₁₈ is substituted C₂-C₁₀ alkynyl. In some embodiments, R₁₈ is unsubstituted C₂-C₁₀ alkenyl. In some embodiments, R₁₈ is substituted C₂-C₁₀ alkenyl. In some embodiments, R₁₈ is carboxyl. In some embodiments, R₁₈ is unsubstituted carbohydrate. In some embodiments, R₁₈ is substituted carbohydrate. In some embodiments, R₁₈ is substituted carbohydrate. In some embodiments, R₁₈ is unsubstituted ester. In some embodiments, R₁₈ is substituted ester. In some embodiments, R₁₈ is unsubstituted acyloxy. In some embodiments, R₁₈ is substituted acyloxy. In some embodiments, R₁₈ is nitro. In some embodiments, R₁₈ is halogen. In some embodiments, R₁₈ is unsubstituted C₁-C₁₀ aliphatic acyl. In some embodiments, R₁₈ is substituted C₁-C₁₀ aliphatic acyl. In some embodiments, R₁₈ is unsubstituted C₆-C₁₀ aromatic acyl. In some embodiments, R₁₈ is substituted C₆-C₁₀ aromatic acyl. In some embodiments, R₁₈ is unsubstituted C₆-C₁₀ aralkyl acyl. In some embodiments, R₁₈ is substituted C₆-C₁₀ aralkyl acyl. In some embodiments, R₁₈ is unsubstituted C₆-C₁₀ alkylaryl acyl. In some embodiments, R₁₈ is substituted C₆-C₁₀ alkylaryl acyl. In some embodiments, R₁₈ is unsubstituted alkoxy. In some embodiments, R₁₈ is substituted alkoxy. In some embodiments, R₁₈ is unsubstituted aryl. In some embodiments, R₁₈ is substituted aryl. In some embodiments, R₁₈ is unsubstituted C₃-C₁₀heterocyclyl. In some embodiments, R₁₈ is substituted C₃-C₁₀heterocyclyl. In some embodiments, R₁₈ is unsubstituted heteroaryl. In some embodiments, R₁₈ is substituted heteroaryl. In some embodiments, R₁₈ is unsubstituted C₃-C₁₀cycloalkyl. In some embodiments, R₁₈ is substituted C₃-C₁₀cycloalkyl. In some embodiments, R₁₈ is —OPO₃WY. In some embodiments, R₁₈ is —OCH₂PO₄WY. In some embodiments, R₁₈ is —OCH₂PO₄Z. In some embodiments, R₁₈ is —OPO₃Z.

In some embodiments, R₁₉ is hydrogen. In some embodiments, R₁₉ is unsubstituted C₁-C₁₀ alkyl. In some embodiments, R₁₉ is substituted C₁-C₁₀ alkyl. In some embodiments, R₁₉ is unsubstituted C₂-C₁₀ alkynyl. In some embodiments, R₁₉ is substituted C₂-C₁₀ alkynyl. In some embodiments, R₁₉ is unsubstituted C₂-C₁₀ alkenyl. In some embodiments, R₁₉ is substituted C₂-C₁₀ alkenyl. In some embodiments, R₁₉ is unsubstituted carbohydrate. In some embodiments, R₁₉ is substituted carbohydrate. In some embodiments, R₁₉ is unsubstituted C₁-C₁₀ aliphatic acyl. In some embodiments, R₁₉ is substituted C₁-C₁₀ aliphatic acyl. In some embodiments, R₁₉ is unsubstituted C₆-C₁₀ aromatic acyl. In some embodiments, R₁₉ is substituted C₆-C₁₀ aromatic acyl. In some embodiments, R₁₉ is unsubstituted C₆-C₁₀ aralkyl acyl. In some embodiments, R₁₉ is substituted C₆-C₁₀ aralkyl acyl. In some embodiments, R₁₉ is unsubstituted C₆-C₁₀ alkylaryl acyl. In some embodiments, R₁₉ is substituted C₆-C₁₀ alkylaryl acyl. In some embodiments, R₁₉ is unsubstituted aryl. In some embodiments, R₁₉ is substituted aryl. In some embodiments, R₁₉ is unsubstituted C₃-C₁₀heterocyclyl. In some embodiments, R₁₉ is substituted C₃-C₁₀heterocyclyl. In some embodiments, R₁₉ is unsubstituted heteroaryl. In some embodiments, R₁₉ is substituted heteroaryl. In some embodiments, R₁₉ is unsubstituted C₃-C₁₀cycloalkyl. In some embodiments, R₁₉ is substituted C₃-C₁₀cycloalkyl. In some embodiments, R₁₉ is —PO₃WY. In some embodiments, R₁₉ is —CH₂PO₄WY. In some embodiments, R₁₉ is —CH₂PO₄Z. In some embodiments, R₁₉ is —PO₃Z.

In some embodiments, R₂₁ is hydrogen. In some embodiments, R₂₁ is hydroxy. In some embodiments, R₂₁ is carboxaldehyde. In some embodiments, R₂₁ is unsubstituted amine. In some embodiments, R₂₁ is substituted amine. In some embodiments, R₂₁ is unsubstituted C₁-C₁₀ alkyl. In some embodiments, R₂₁ is unsubstituted C₂-C₁₀ alkynyl. In some embodiments, R₂₁ is substituted C₂-C₁₀ alkynyl. In some embodiments, R₂₁ is unsubstituted C₂-C₁₀ alkenyl. In some embodiments, R₂₁ is substituted C₂-C₁₀ alkenyl. In some embodiments, R₂₁ is carboxyl. In some embodiments, R₂₁ is unsubstituted carbohydrate. In some embodiments, R₂₁ is substituted carbohydrate. In some embodiments, R₂₁ is unsubstituted ester. In some embodiments, R₂₁ is substituted ester. In some embodiments, R₂₁ is unsubstituted acyloxy. In some embodiments, R₂₁ is substituted acyloxy. In some embodiments, R₂₁ is nitro. In some embodiments, R₂₁ is halogen. In some embodiments, R₂₁ is unsubstituted C₁-C₁₀ aliphatic acyl. In some embodiments, R₂₁ is substituted C₁-C₁₀ aliphatic acyl. In some embodiments, R₂₁ is unsubstituted C₆-C₁₀ aromatic acyl. In some embodiments, R₂₁ is substituted C₆-C₁₀ aromatic acyl. In some embodiments, R₂₁ is unsubstituted C₆-C₁₀ aralkyl acyl. In some embodiments, R₂₁ is substituted C₆-C₁₀ aralkyl acyl. In some embodiments, R₂₁ is unsubstituted C₆-C₁₀ alkylaryl acyl. In some embodiments, R₂₁ is substituted C₆-C₁₀ alkylaryl acyl. In some embodiments, R₂₁ is unsubstituted alkoxy. In some embodiments, R₂₁ is substituted alkoxy. In some embodiments, R₂₁ is unsubstituted aryl. In some embodiments, R₂₁ is substituted aryl. In some embodiments, R₂₁ is unsubstituted C₃-C₁₀heterocyclyl. In some embodiments, R₂₁ is substituted C₃-C₁₀heterocyclyl. In some embodiments, R₂₁ is unsubstituted heteroaryl. In some embodiments, R₂₁ is substituted heteroaryl. In some embodiments, R₂₁ is unsubstituted C₃-C₁₀cycloalkyl. In some embodiments, R₂₁ is substituted C₃-C₁₀cycloalkyl. In some embodiments, R₂₁ is —OPO₃WY. In some embodiments, R₂₁ is —OCH₂PO₄WY. In some embodiments, R₂₁ is —OCH₂PO₄Z. In some embodiments, R₂₁ is —OPO₃Z.

In some embodiments, s is an integer of 0. In some embodiments, s is an integer of 1. In some embodiments, s is an integer of 2. In some embodiments, s is an integer of 3.

In some embodiments, n is an integer of 0. In some embodiments, n is an integer of 1. In some embodiments, n is an integer of 2. In some embodiments, n is an integer of 3. In some embodiments, n is an integer of 4.

In various embodiments, W and Y are independently potassium, sodium, or lithium.

In various embodiments, Z is calcium, magnesium or iron.

In various embodiments of the invention, the pyrone analog is of Formulae III, IV, V, or VI as illustrated in Scheme I.

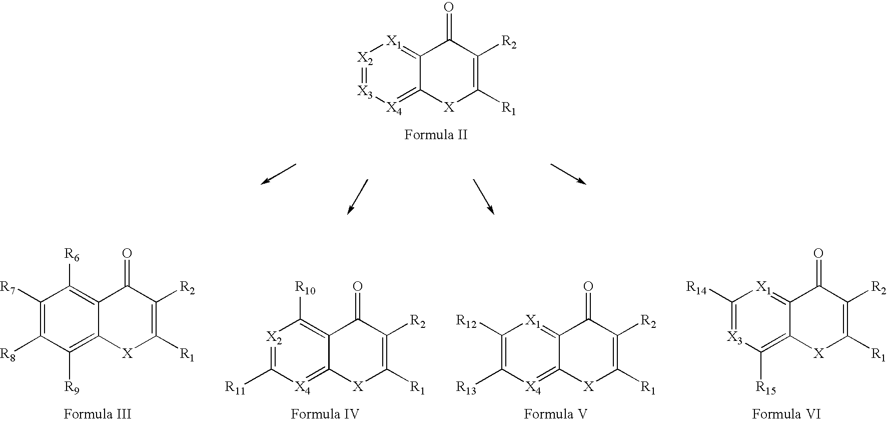

### Scheme I. Exemplary Subclasses of Formula II

In some embodiments of the invention where the X₁, X₂, X₃, and X₄ of the compounds of Formula II are CR₅, the compound is of Formula III:

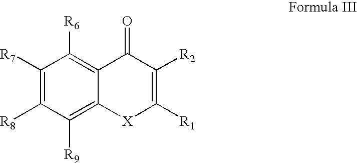

wherein X, R₁, R₂, W, Y, and Z are defined as in Formula I and Formula II;

R₆, R₇, R₈, and R₉ are independently hydrogen, hydroxyl, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀alkylaryl acyl, alkoxy, amine, aryl, C₃-C₁₀heterocyclyl, heteroaryl, C₃-C₁₀cycloalkyl, —OPO₃WY, —OCH₂PO₄WY, —OCH₂PO₄Z or —OPO₃Z.

In some embodiments, R₆ is hydrogen. In some embodiments, R₆ is hydroxyl. In some embodiments, R₆ is carboxaldehyde. In some embodiments, R₆ is unsubstituted amine. In some embodiments, R₆ is substituted amine. In some embodiments, R₆ is unsubstituted C₁-C₁₀ alkyl. In some embodiments, R₆ is substituted C₁-C₁₀ alkyl. In some embodiments, R₆ is unsubstituted C₂-C₁₀ alkynyl. In some embodiments, R₆ is substituted C₂-C₁₀ alkynyl. In some embodiments, R₆ is unsubstituted C₂-C₁₀ alkenyl. In some embodiments, R₆ is substituted C₂-C₁₀ alkenyl. In some embodiments, R₆ is carboxyl. In some embodiments, R₆ is unsubstituted carbohydrate. In some embodiments, R₆ is substituted carbohydrate. In some embodiments, R₆ is unsubstituted ester. In some embodiments, R₆ is substituted ester. In some embodiments, R₆ is unsubstituted acyloxy. In some embodiments, R₆ is substituted acyloxy. In some embodiments, R₆ is nitro. In some embodiments, R₆ is halogen. In some embodiments, R₆ is unsubstituted C₁-C₁₀ aliphatic acyl. In some embodiments, R₆ is substituted C₁-C₁₀ aliphatic acyl. In some embodiments, R₆ is unsubstituted C₆-C₁₀ aromatic acyl. In some embodiments, R₆ is substituted C₆-C₁₀ aromatic acyl. In some embodiments, R₆ is unsubstituted C₆-C₁₀ aralkyl acyl. In some embodiments, R₆ is substituted C₆-C₁₀ aralkyl acyl. In some embodiments, R₆ is unsubstituted C₆-C₁₀ alkylaryl acyl. In some embodiments, R₆ is substituted C₆-C₁₀ alkylaryl acyl. In some embodiments, R₆ is unsubstituted alkoxy. In some embodiments, R₆ is substituted alkoxy. In some embodiments, R₆ is unsubstituted aryl. In some embodiments, R₆ is substituted aryl. In some embodiments, R₆ is unsubstituted C₃-C₁₀heterocyclyl. In some embodiments, R₆ is substituted C₃-C₁₀ heterocyclyl. In some embodiments, R₆ is unsubstituted heteroaryl. In some embodiments, R₆ is unsubstituted C₃-C₁₀cycloalkyl. In some embodiments, R₆ is substituted C₃-C₁₀cycloalkyl. In some embodiments, R₆ is —OPO₃WY. In some embodiments, R₆ is —OCH₂PO₄WY. In some embodiments, R₆ is —OCH₂PO₄Z. In some embodiments, R₆ is —OPO₃Z.

In some embodiments, R₇ is hydrogen. In some embodiments, R₇ is hydroxyl. In some embodiments, R₇ is carboxaldehyde. In some embodiments, R₇ is unsubstituted amine. In some embodiments, R₇ is substituted amine. In some embodiments, R₇ is unsubstituted C₁-C₁₀ alkyl. In some embodiments, R₇ is substituted C₁-C₁₀ alkyl. In some embodiments, R₇ is unsubstituted C₂-C₁₀ alkynyl. In some embodiments, R₇ is substituted C₂-C₁₀ alkynyl. In some embodiments, R₇ is unsubstituted C₂-C₁₀ alkenyl. In some embodiments, R₇ is substituted C₂-C₁₀ alkenyl. In some embodiments, R₇ is carboxyl. In some embodiments, R₇ is unsubstituted carbohydrate. In some embodiments, R₇ is substituted carbohydrate. In some embodiments, R₇ is unsubstituted ester. In some embodiments, R₇ is substituted ester. In some embodiments, R₇ is unsubstituted acyloxy. In some embodiments, R₇ is substituted acyloxy. In some embodiments, R₇ is nitro. In some embodiments, R₇ is halogen. In some embodiments, R₇ is unsubstituted C₁-C₁₀ aliphatic acyl. In some embodiments, R₇ is substituted C₁-C₁₀ aliphatic acyl. In some embodiments, R₇ is unsubstituted C₆-C₁₀ aromatic acyl. In some embodiments, R₇ is substituted C₆-C₁₀ aromatic acyl. In some embodiments, R₇ is unsubstituted C₆-C₁₀ aralkyl acyl. In some embodiments, R₇ is substituted C₆-C₁₀ aralkyl acyl. In some embodiments, R₇ is unsubstituted C₆-C₁₀ alkylaryl acyl. In some embodiments, R₇ is substituted C₆-C₁₀ alkylaryl acyl. In some embodiments, R₇ is unsubstituted alkoxy. In some embodiments, R₇ is substituted alkoxy. In some embodiments, R₇ is unsubstituted aryl. In some embodiments, R₇ is substituted aryl. In some embodiments, R₇ is unsubstituted C₃-C₁₀heterocyclyl. In some embodiments, R₇ is substituted C₃-C₁₀ heterocyclyl. In some embodiments, R₇ is unsubstituted heteroaryl. In some embodiments, R₇ is unsubstituted C₃-C₁₀cycloalkyl. In some embodiments, R₇ is substituted C₃-C₁₀cycloalkyl. In some embodiments, R₇ is —OPO₃WY. In some embodiments, R₇ is —OCH₂PO₄WY. In some embodiments, R₇ is —OCH₂PO₄Z. In some embodiments, R₇ is —OPO₃Z.

In some embodiments, R₈ is hydrogen. In some embodiments, R₈ is hydroxyl. In some embodiments, R₈ is carboxaldehyde. In some embodiments, R₈ is unsubstituted amine. In some embodiments, R₈ is substituted amine. In some embodiments, R₈ is unsubstituted C₁-C₁₀ alkyl. In some embodiments, R₈ is substituted C₁-C₁₀ alkyl. In some embodiments, R₈ is unsubstituted C₂-C₁₀ alkynyl. In some embodiments, R₈ is substituted C₂-C₁₀ alkynyl. In some embodiments, R₈ is unsubstituted C₂-C₁₀ alkenyl. In some embodiments, R₈ is substituted C₂-C₁₀ alkenyl. In some embodiments, R₈ is carboxyl. In some embodiments, R₈ is unsubstituted carbohydrate. In some embodiments, R₈ is substituted carbohydrate. In some embodiments, R₈ is unsubstituted ester. In some embodiments, R₈ is substituted ester. In some embodiments, R₈ is unsubstituted acyloxy. In some embodiments, R₈ is substituted acyloxy. In some embodiments, R₈ is nitro. In some embodiments, R₈ is halogen. In some embodiments, R₈ is unsubstituted C₁-C₁₀ aliphatic acyl. In some embodiments, R₈ is substituted C₁-C₁₀ aliphatic acyl. In some embodiments, R₈ is unsubstituted C₆-C₁₀ aromatic acyl. In some embodiments, R₈ is substituted C₆-C₁₀ aromatic acyl. In some embodiments, R₈ is unsubstituted C₆-C₁₀ aralkyl acyl. In some embodiments, R₈ is substituted C₆-C₁₀ aralkyl acyl. In some embodiments, R₈ is unsubstituted C₆-C₁₀ alkylaryl acyl. In some embodiments, R₈ is substituted C₆-C₁₀ alkylaryl acyl. In some embodiments, R₈ is unsubstituted alkoxy. In some embodiments, R₈ is substituted alkoxy. In some embodiments, R₈ is unsubstituted aryl. In some embodiments, R₈ is substituted aryl. In some embodiments, R₈ is unsubstituted C₃-C₁₀heterocyclyl. In some embodiments, R₈ is substituted C₃-C₁₀ heterocyclyl. In some embodiments, R₈ is unsubstituted heteroaryl. In some embodiments, R₈ is unsubstituted C₃-C₁₀cycloalkyl. In some embodiments, R₈ is substituted C₃-C₁₀cycloalkyl. In some embodiments, R₈ is —OPO₃WY. In some embodiments, R₈ is —OCH₂PO₄WY. In some embodiments, R₈ is —OCH₂PO₄Z. In some embodiments, R₈ is —OPO₃Z.

In some embodiments, R₉ is hydrogen. In some embodiments, R₉ is hydroxyl. In some embodiments, R₉ is carboxaldehyde. In some embodiments, R₉ is unsubstituted amine. In some embodiments, R₉ is substituted amine. In some embodiments, R₉ is unsubstituted C₁-C₁₀ alkyl. In some embodiments, R₉ is substituted C₁-C₁₀ alkyl. In some embodiments, R₉ is unsubstituted C₂-C₁₀ alkynyl. In some embodiments, R₉ is substituted C₂-C₁₀ alkynyl. In some embodiments, R₉ is unsubstituted C₂-C₁₀ alkenyl. In some embodiments, R₉ is substituted C₂-C₁₀ alkenyl. In some embodiments, R₉ is carboxyl. In some embodiments, R₉ is unsubstituted carbohydrate. In some embodiments, R₉ is substituted carbohydrate. In some embodiments, R₉ is unsubstituted ester. In some embodiments, R₉ is substituted ester. In some embodiments, R₉ is unsubstituted acyloxy. In some embodiments, R₉ is substituted acyloxy. In some embodiments, R₉ is nitro. In some embodiments, R₉ is halogen. In some embodiments, R₉ is unsubstituted C₁-C₁₀ aliphatic acyl. In some embodiments, R₉ is substituted C₁-C₁₀ aliphatic acyl. In some embodiments, R₉ is unsubstituted C₆-C₁₀ aromatic acyl. In some embodiments, R₉ is substituted C₆-C₁₀ aromatic acyl. In some embodiments, R₉ is unsubstituted C₆-C₁₀ aralkyl acyl. In some embodiments, R₉ is substituted C₆-C₁₀ aralkyl acyl. In some embodiments, R₉ is unsubstituted C₆-C₁₀ alkylaryl acyl. In some embodiments, R₉ is substituted C₆-C₁₀ alkylaryl acyl. In some embodiments, R₉ is unsubstituted alkoxy. In some embodiments, R₉ is substituted alkoxy. In some embodiments, R₉ is unsubstituted aryl. In some embodiments, R₉ is substituted aryl. In some embodiments, R₉ is unsubstituted C₃-C₁₀heterocyclyl. In some embodiments, R₉ is substituted C₃-C₁₀ heterocyclyl. In some embodiments, R₉ is unsubstituted heteroaryl. In some embodiments, R₉ is unsubstituted C₃-C₁₀cycloalkyl. In some embodiments, R₉ is substituted C₃-C₁₀cycloalkyl. In some embodiments, R₉ is —OPO₃WY. In some embodiments, R₉ is —OCH₂PO₄WY. In some embodiments, R₉ is —OCH₂PO₄Z. In some embodiments, R₉ is —OPO₃Z.

In various embodiments of the invention, the pyrone analog of Formula III is of Formula VII:

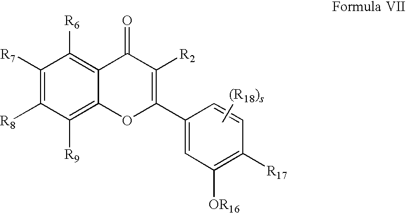

wherein R₂, R₁₆, R₁₇, R₁₈, and s are as defined in Formula II and R₆, R₇, R₈, and R₉ are as defined in Formula III.

In other embodiments of the invention, the pyrone analog of Formula III is a compound of Formula VIII:

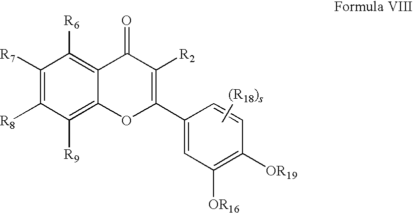

wherein R₂, R₁₆, R₁₈, R₁₉, and s are as defined in Formula II and R₆, R₇, R₈, and R₉ are as defined in Formula III.

In some embodiments of the invention, the pyrone analog of Formula II is of Formula IX:

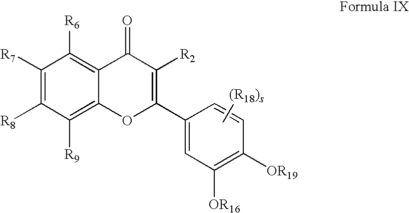

wherein R₂, R₁₆, R₁₈, R₁₉, and s are as defined in Formula II; and

R₆, R₇, R₈, and R₉ are independently hydrogen, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀alkylaryl acyl, alkoxy, amine, aryl, C₃-C₁₀heterocyclyl, heteroaryl, C₃-C₁₀cycloalkyl, —OPO₃WY, —OCH₂PO₄WY, —OCH₂PO₄Z or —OPO₃Z. In this embodiment, none of R₆-R₉ are OH.

In some embodiments, R₆ is hydrogen. In some embodiments, R₆ is carboxaldehyde. In some embodiments, R₆ is unsubstituted amine. In some embodiments, R₆ is substituted amine. In some embodiments, R₆ is unsubstituted C₁-C₁₀ alkyl. In some embodiments, R₆ is substituted C₁-C₁₀ alkyl. In some embodiments, R₆ is unsubstituted C₂-C₁₀ alkynyl. In some embodiments, R₆ is substituted C₂-C₁₀ alkynyl. In some embodiments, R₆ is unsubstituted C₂-C₁₀ alkenyl. In some embodiments, R₆ is substituted C₂-C₁₀ alkenyl. In some embodiments, R₆ is carboxyl. In some embodiments, R₆ is unsubstituted carbohydrate. In some embodiments, R₆ is substituted carbohydrate. In some embodiments, R₆ is unsubstituted ester. In some embodiments, R₆ is substituted ester. In some embodiments, R₆ is unsubstituted acyloxy. In some embodiments, R₆ is substituted acyloxy. In some embodiments, R₆ is nitro. In some embodiments, R₆ is halogen. In some embodiments, R₆ is unsubstituted C₁-C₁₀ aliphatic acyl. In some embodiments, R₆ is substituted C₁-C₁₀ aliphatic acyl. In some embodiments, R₆ is unsubstituted C₆-C₁₀ aromatic acyl. In some embodiments, R₆ is substituted C₆-C₁₀ aromatic acyl. In some embodiments, R₆ is unsubstituted C₆-C₁₀ aralkyl acyl. In some embodiments, R₆ is substituted C₆-C₁₀ aralkyl acyl. In some embodiments, R₆ is unsubstituted C₆-C₁₀ alkylaryl acyl. In some embodiments, R₆ is substituted C₆-C₁₀ alkylaryl acyl. In some embodiments, R₆ is unsubstituted alkoxy. In some embodiments, R₆ is substituted alkoxy. In some embodiments, R₆ is unsubstituted aryl. In some embodiments, R₆ is substituted aryl. In some embodiments, R₆ is unsubstituted C₃-C₁₀heterocyclyl. In some embodiments, R₆ is substituted C₃-C₁₀heterocyclyl. In some embodiments, R₆ is unsubstituted heteroaryl. In some embodiments, R₆ is unsubstituted C₃-C₁₀cycloalkyl. In some embodiments, R₆ is substituted C₃-C₁₀cycloalkyl. In some embodiments, R₆ is —OPO₃WY. In some embodiments, R₆ is —OCH₂PO₄WY. In some embodiments, R₆ is —OCH₂PO₄Z. In some embodiments, R₆ is —OPO₃Z.

In some embodiments, R₇ is hydrogen. In some embodiments, R₇ is carboxaldehyde. In some embodiments, R₇ is unsubstituted amine. In some embodiments, R₇ is substituted amine. In some embodiments, R₇ is unsubstituted C₁-C₁₀ alkyl. In some embodiments, R₇ is substituted C₁-C₁₀ alkyl. In some embodiments, R₇ is unsubstituted C₂-C₁₀ alkynyl. In some embodiments, R₇ is substituted C₂-C₁₀ alkynyl. In some embodiments, R₇ is unsubstituted C₂-C₁₀ alkenyl. In some embodiments, R₇ is substituted C₂-C₁₀ alkenyl. In some embodiments, R₇ is carboxyl. In some embodiments, R₇ is unsubstituted carbohydrate. In some embodiments, R₇ is substituted carbohydrate. In some embodiments, R₇ is unsubstituted ester. In some embodiments, R₇ is substituted ester. In some embodiments, R₇ is unsubstituted acyloxy. In some embodiments, R₇ is substituted acyloxy. In some embodiments, R₇ is nitro. In some embodiments, R₇ is halogen. In some embodiments, R₇ is unsubstituted C₁-C₁₀ aliphatic acyl. In some embodiments, R₇ is substituted C₁-C₁₀ aliphatic acyl. In some embodiments, R₇ is unsubstituted C₆-C₁₀ aromatic acyl. In some embodiments, R₇ is substituted C₆-C₁₀ aromatic acyl. In some embodiments, R₇ is unsubstituted C₆-C₁₀ aralkyl acyl. In some embodiments, R₇ is substituted C₆-C₁₀ aralkyl acyl. In some embodiments, R₇ is unsubstituted C₆-C₁₀ alkylaryl acyl. In some embodiments, R₇ is substituted C₆-C₁₀ alkylaryl acyl. In some embodiments, R₇ is unsubstituted alkoxy. In some embodiments, R₇ is substituted alkoxy. In some embodiments, R₇ is unsubstituted aryl. In some embodiments, R₇ is substituted aryl. In some embodiments, R₇ is unsubstituted C₃-C₁₀heterocyclyl. In some embodiments, R₇ is substituted C₃-C₁₀heterocyclyl. In some embodiments, R₇ is unsubstituted heteroaryl. In some embodiments, R₇ is unsubstituted C₃-C₁₀cycloalkyl. In some embodiments, R₇ is substituted C₃-C₁₀cycloalkyl. In some embodiments, R₇ is —OPO₃WY. In some embodiments, R₇ is —OCH₂PO₄WY. In some embodiments, R₇ is —OCH₂PO₄Z. In some embodiments, R₇ is —OPO₃Z.

In some embodiments, R₈ is hydrogen. In some embodiments, R₈ is hydroxyl. In some embodiments, R₈ is carboxaldehyde. In some embodiments, R₈ is unsubstituted amine. In some embodiments, R₈ is substituted amine. In some embodiments, R₈ is unsubstituted C₁-C₁₀ alkyl. In some embodiments, R₈ is substituted C₁-C₁₀ alkyl. In some embodiments, R₈ is unsubstituted C₂-C₁₀ alkynyl. In some embodiments, R₈ is substituted C₂-C₁₀ alkynyl. In some embodiments, R₈ is unsubstituted C₂-C₁₀ alkenyl. In some embodiments, R₈ is substituted C₂-C₁₀ alkenyl. In some embodiments, R₈ is carboxyl. In some embodiments, R₈ is unsubstituted carbohydrate. In some embodiments, R₈ is substituted carbohydrate. In some embodiments, R₈ is unsubstituted ester. In some embodiments, R₈ is substituted ester. In some embodiments, R₈ is unsubstituted acyloxy. In some embodiments, R₈ is substituted acyloxy. In some embodiments, R₈ is nitro. In some embodiments, R₈ is halogen. In some embodiments, R₈ is unsubstituted C₁-C₁₀ aliphatic acyl. In some embodiments, R₈ is substituted C₁-C₁₀ aliphatic acyl. In some embodiments, R₈ is unsubstituted C₆-C₁₀ aromatic acyl. In some embodiments, R₈ is substituted C₆-C₁₀ aromatic acyl. In some embodiments, R₈ is unsubstituted C₆-C₁₀ aralkyl acyl. In some embodiments, R₈ is substituted C₆-C₁₀ aralkyl acyl. In some embodiments, R₈ is unsubstituted C₆-C₁₀ alkylaryl acyl. In some embodiments, R₈ is substituted C₆-C₁₀ alkylaryl acyl. In some embodiments, R₈ is unsubstituted alkoxy. In some embodiments, R₈ is substituted alkoxy. In some embodiments, R₈ is unsubstituted aryl. In some embodiments, R₈ is substituted aryl. In some embodiments, R₈ is unsubstituted C₃-C₁₀heterocyclyl. In some embodiments, R₈ is substituted C₃-C₁₀ heterocyclyl. In some embodiments, R₈ is unsubstituted heteroaryl. In some embodiments, R₈ is unsubstituted C₃-C₁₀cycloalkyl. In some embodiments, R₈ is substituted C₃-C₁₀cycloalkyl. In some embodiments, R₈ is —OPO₃WY. In some embodiments, R₈ is —OCH₂PO₄WY. In some embodiments, R₈ is —OCH₂PO₄Z. In some embodiments, R₈ is —OPO₃Z.

In some embodiments, R₉ is hydrogen. In some embodiments, R₉ is carboxaldehyde. In some embodiments, R₉ is unsubstituted amine. In some embodiments, R₉ is substituted amine. In some embodiments, R₉ is unsubstituted C₁-C₁₀ alkyl. In some embodiments, R₉ is substituted C₁-C₁₀ alkyl. In some embodiments, R₉ is unsubstituted C₂-C₁₀ alkynyl. In some embodiments, R₉ is substituted C₂-C₁₀ alkynyl. In some embodiments, R₉ is unsubstituted C₂-C₁₀ alkenyl. In some embodiments, R₉ is substituted C₂-C₁₀ alkenyl. In some embodiments, R₉ is carboxyl. In some embodiments, R₉ is unsubstituted carbohydrate. In some embodiments, R₉ is substituted carbohydrate. In some embodiments, R₉ is unsubstituted ester. In some embodiments, R₉ is substituted ester. In some embodiments, R₉ is unsubstituted acyloxy. In some embodiments, R₉ is substituted acyloxy. In some embodiments, R₉ is nitro. In some embodiments, R₉ is halogen. In some embodiments, R₉ is unsubstituted C₁-C₁₀ aliphatic acyl. In some embodiments, R₉ is substituted C₁-C₁₀ aliphatic acyl. In some embodiments, R₉ is unsubstituted C₆-C₁₀ aromatic acyl. In some embodiments, R₉ is substituted C₆-C₁₀ aromatic acyl. In some embodiments, R₉ is unsubstituted C₆-C₁₀ aralkyl acyl. In some embodiments, R₉ is substituted C₆-C₁₀ aralkyl acyl. In some embodiments, R₉ is unsubstituted C₆-C₁₀ alkylaryl acyl. In some embodiments, R₉ is substituted C₆-C₁₀ alkylaryl acyl. In some embodiments, R₉ is unsubstituted alkoxy. In some embodiments, R₉ is substituted alkoxy. In some embodiments, R₉ is unsubstituted aryl. In some embodiments, R₉ is substituted aryl. In some embodiments, R₉ is unsubstituted C₃-C₁₀heterocyclyl. In some embodiments, R₉ is substituted C₃-C₁₀heterocyclyl. In some embodiments, R₉ is unsubstituted heteroaryl. In some embodiments, R₉ is unsubstituted C₃-C₁₀cycloalkyl. In some embodiments, R₉ is substituted C₃-C₁₀cycloalkyl. In some embodiments, R₉ is —OPO₃WY. In some embodiments, R₉ is —OCH₂PO₄WY. In some embodiments, R₉ is —OCH₂PO₄Z. In some embodiments, R₉ is —OPO₃Z.

In some embodiments of the invention, the pyrone analog of Formula III is of Formula X:

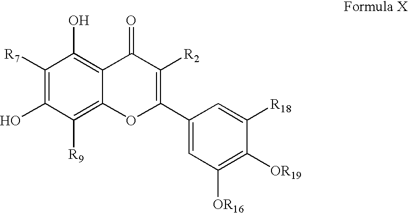

wherein R₂, R₁₆, R₁₈, and R₁₉ are as defined in Formula II and R₇ and R₉ are as defined in Formula III.

In other embodiments of the invention, the pyrone analog of Formula III is of Formula XI:

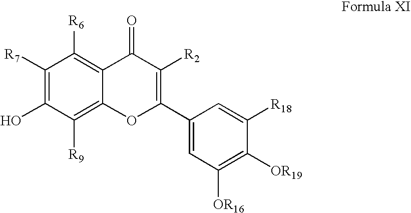

wherein R₂, R₁₆, R₁₈, and R₁₉ are as defined in Formula II and R₆, R₇, and R₉ are as defined in Formula III.

In some embodiments of the invention, compounds of the following Formulae VIII-A, VIII-B, and VIII-C, are useful in the methods of the invention, where each instance of R_{c} and R_{d} is independently hydrogen, —PO₃WY, —OPO₃Z, OCH₂OPOWY, or OCH₂OPO₃Z, where W and Y are hydrogen, methyl, ethyl, alkyl, carbohydrate, lithium, sodium or potassium and Z is calcium, magnesium or iron.

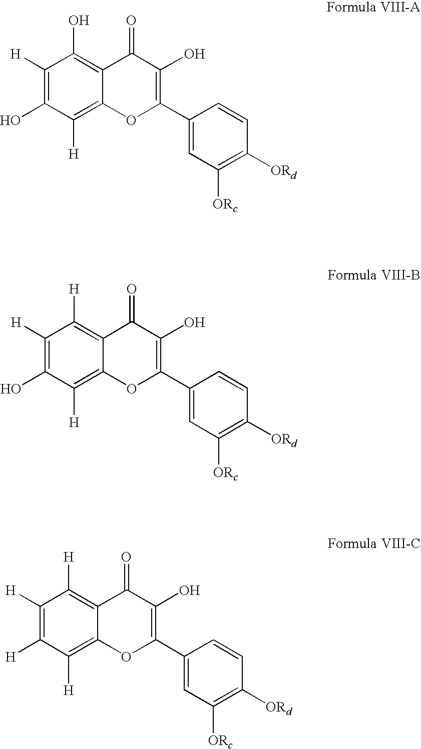

In some embodiments of the invention, for a compound of Formulae VIII-A, VIII-B, or VIII-C, Rc and Rd are hydrogen. In some embodiments of the invention, for a compound of Formulae VIII-A, VIII-B, or VIII-C, R_{c} is OPO₃WY and R_{d} is hydrogen. In some embodiments of the invention, for a compound of Formulae VIII-A, VIII-B, or VIII-C, R_{c} is OPO₃WY and R_{d} is OPO₃WY. In some embodiments of the invention, for a compound of Formulae VIII-A, VIII-B, or VIII-C, R_{c} is a mixture of hydrogen and OPO₃WY and R^{d} is OPO₃WY. In some embodiments of the invention, for a compound of Formulae VIII-A, VIII-B, or VIII-C, R_{c} is hydrogen and R_{d} is a mixture of hydrogen and OPO₃Z. In some embodiments of the invention, for a compound of Formulae VIII-A, VIII-B, or VIII-C, R_{c} is OPO₃Z and R_{d} is hydrogen. In some embodiments of the invention, for a compound of Formulae VIII-A, VIII-B, or VIII-C, R_{c} is —OPO₃Z and R_{d} is OPO₃Z. In some embodiments of the invention, for a compound of Formulae VIII-A, VIII-B, or VIII-C, R_{c} is a mixture of hydrogen and OPO₃Z and R_{d} is OPO₃Z. In some embodiments of the invention, for a compound of Formulae VIII-A, VIII-B, or VIII-C, R_{c} is hydrogen and R_{d} is a mixture of hydrogen and OPO₃Z. In some embodiments of the invention, for a compound of Formulae VIII-A, VIII-B, or VIII-C, R_{c} is CH₂OPO₃Z and R_{d} is hydrogen. In some embodiments of the invention, for a compound of Formulae VIII-A, VIII-B, or VIII-C, R_{c} is CH₂OPO₃Z and R_{d} is CH₂OPO₃Z. In some embodiments of the invention, for a compound of Formulae VIII-A, VIII-B, or VIII-C, R_{c} is a mixture of hydrogen and CH₂OPO₃Z and R_{d} is CH₂OPO₃Z. In some embodiments of the invention, for a compound of Formulae VIII-A, VIII-B, or VIII-C, R_{c} is hydrogen and R_{d} is a mixture of hydrogen and CH₂OPO₃Z.

In other embodiments of the invention, the pyrone analog of Formula III is of Formula XII:

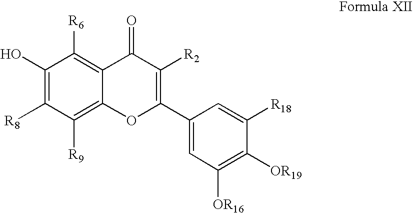

wherein R₂, R₁₆, R₁₈, and R₁₉ are as defined in Formula I₁ and R₆, R₈, and R₉ are as defined in Formula III.

In other embodiments of the invention, the pyrone analog of Formula III is of Formula XIII:

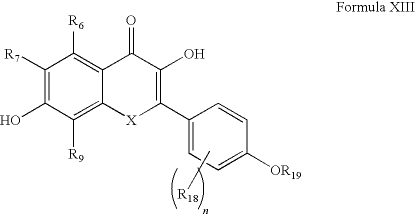

wherein X, R₁₈, and R₁₉ are as defined in Formula II and R₆, R₇, and R₉ are as defined in Formula III.

In some embodiments, the pyrone analog of Formula III is of Formula XIV:

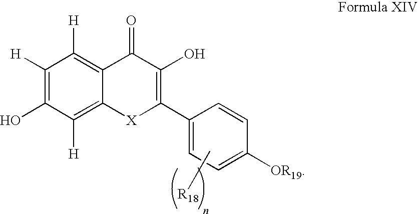

In some embodiments, the pyrone analog of Formula III is of Formula XV:

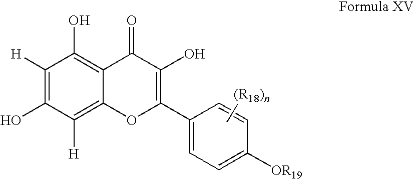

wherein R₁₈, R₁₉, and n are as defined in Formula II.

In some embodiments, the pyrone analog of Formula III is of Formula XVI:

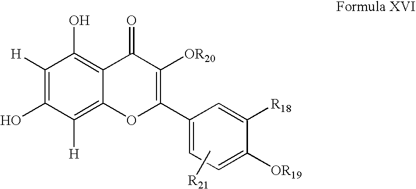

wherein R₁₈, R₁₉, R₂₁, and n are as defined in Formula II;

R₂₀ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀alkylaryl acyl, aryl, C₃-C₁₀heterocyclyl, heteroaryl, optionally substituted C₃-C₁₀cycloalkyl, —PO₃WY, —CH₂PO₄WY, —CH₂PO₄Z or —PO₃Z; and

W and Y are independently hydrogen, methyl, ethyl, alkyl, carbohydrate, or a cation, and Z is a multivalent cation.

In some embodiments, R₂₀ is hydrogen. In some embodiments, R₂₀ is unsubstituted C₁-C₁₀ alkyl. In some embodiments, R₂₀ is substituted C₁-C₁₀ alkyl. In some embodiments, R₂₀ is unsubstituted C₂-C₁₀ alkynyl. In some embodiments, R₂₀ is substituted C₂-C₁₀ alkynyl. In some embodiments, R₂₀ is unsubstituted C₂-C₁₀ alkenyl. In some embodiments, R₂₀ is substituted C₂-C₁₀ alkenyl. In some embodiments, R₂₀ is unsubstituted carbohydrate. In some embodiments, R₂₀ is substituted carbohydrate. In some embodiments, R₂₀ is unsubstituted C₁-C₁₀ aliphatic acyl. In some embodiments, R₂₀ is substituted C₁-C₁₀ aliphatic acyl. In some embodiments, R₂₀ is unsubstituted C₆-C₁₀ aromatic acyl. In some embodiments, R₂₀ is substituted C₆-C₁ aromatic acyl. In some embodiments, R₂₀ is unsubstituted C₆-C₁₀ aralkyl acyl. In some embodiments, R₂₀ is substituted C₆-C₁₀ aralkyl acyl. In some embodiments, R₂₀ is unsubstituted C₆-C₁₀ alkylaryl acyl. In some embodiments, R₂₀ is substituted C₆-C₁₀ alkylaryl acyl. In some embodiments, R₂₀ is unsubstituted aryl. In some embodiments, R₂₀ is substituted aryl. In some embodiments, R₂₀ is unsubstituted C₃-C₁₀heterocyclyl. In some embodiments, R₂₀ is substituted C₃-C₁₀heterocyclyl. In some embodiments, R₂₀ is unsubstituted heteroaryl. In some embodiments, R₂₀ is substituted heteroaryl. In some embodiments, R₂₀ is unsubstituted C₃-C₁₀cycloalkyl. In some embodiments, R₂₀ is substituted C₃-C₁₀cycloalkyl. In some embodiments, R₂₀ is —PO₃WY. In some embodiments, R₂₀ is —CH₂PO₄WY. In some embodiments, R₂₀ is —CH₂PO₄Z. In some embodiments, R₂₀ is —PO₃Z.

In some embodiments, the pyrone analog of Formula III is of Formula XVII:

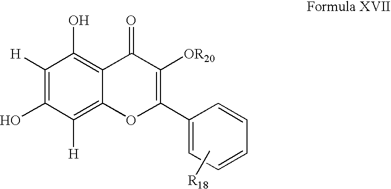

wherein R₁₈ is as defined in Formula II; and

R₂₀ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀heterocyclyl, heteroaryl, optionally substituted C₃-C₁₀cycloalkyl, —PO₃WY, —CH₂PO₄WY, —CH₂PO₄Z or —PO₃Z.

In some embodiments, R₂₀ is hydrogen. In some embodiments, R₂₀ is unsubstituted C₁-C₁₀ alkyl. In some embodiments, R₂₀ is substituted C₁-C₁₀ alkyl. In some embodiments, R₂₀ is unsubstituted C₂-C₁₀ alkynyl. In some embodiments, R₂₀ is substituted C₂-C₁₀ alkynyl. In some embodiments, R₂₀ is unsubstituted C₂-C₁₀ alkenyl. In some embodiments, R₂₀ is substituted C₂-C₁₀ alkenyl. In some embodiments, R₂₀ is unsubstituted carbohydrate. In some embodiments, R₂₀ is substituted carbohydrate. In some embodiments, R₂₀ is unsubstituted C₁-C₁₀ aliphatic acyl. In some embodiments, R₂₀ is substituted C₁-C₁₀ aliphatic acyl. In some embodiments, R₂₀ is unsubstituted C₆-C₁₀ aromatic acyl. In some embodiments, R₂₀ is substituted C₆-C₁₀ aromatic acyl. In some embodiments, R₂₀ is unsubstituted C₆-C₁₀ aralkyl acyl. In some embodiments, R₂₀ is substituted C₆-C₁₀ aralkyl acyl. In some embodiments, R₂₀ is unsubstituted C₆-C₁₀ alkylaryl acyl. In some embodiments, R₂₀ is substituted C₆-C₁₀ alkylaryl acyl. In some embodiments, R₂₀ is unsubstituted aryl. In some embodiments, R₂₀ is substituted aryl. In some embodiments, R₂₀ is unsubstituted C₃-C₁₀heterocyclyl. In some embodiments, R₂₀ is substituted C₃-C₁₀heterocyclyl. In some embodiments, R₂₀ is unsubstituted heteroaryl. In some embodiments, R₂₀ is substituted heteroaryl. In some embodiments, R₂₀ is unsubstituted C₃-C₁₀cycloalkyl. In some embodiments, R₂₀ is substituted C₃-C₁₀cycloalkyl. In some embodiments, R₂₀ is —PO₃WY. In some embodiments, R₂₀ is —CH₂PO₄WY. In some embodiments, R₂₀ is —CH₂PO₄Z. In some embodiments, R₂₀ is —PO₃Z.

In some embodiments, the pyrone analog of Formula III is of Formula XVIII:

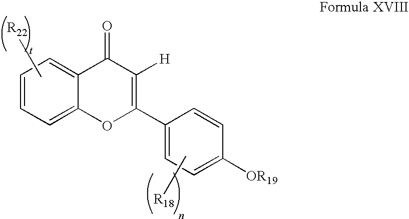

wherein R₁₈ and R₁₉ are as defined in Formula II;

wherein each instance of R₂₂ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z; and

t is an integer of 0, 1, 2, 3, or 4

In some embodiments, R₂₂ is hydrogen. In some embodiments, R₂₂ is hydroxy. In some embodiments, R₂₂ is carboxaldehyde. In some embodiments, R₂₂ is unsubstituted amine. In some embodiments, R₂₂ is substituted amine. In some embodiments, R₂₂ is unsubstituted C₁-C₁₀ alkyl. In some embodiments, R₂₂ is unsubstituted C₂-C₁₀ alkynyl. In some embodiments, R₂₂ is substituted C₂-C₁₀ alkynyl. In some embodiments, R₂₂ is unsubstituted C₂-C₁₀ alkenyl. In some embodiments, R₂₂ is substituted C₂-C₁₀ alkenyl. In some embodiments, R₂₂ is carboxyl. In some embodiments, R₂₂ is unsubstituted carbohydrate. In some embodiments, R₂₂ is substituted carbohydrate. In some embodiments, R₂₂ is unsubstituted ester. In some embodiments, R₂₂ is substituted ester. In some embodiments, R₂₂ is unsubstituted acyloxy. In some embodiments, R₂₂ is substituted acyloxy. In some embodiments, R₂₂ is nitro. In some embodiments, R₂₂ is halogen. In some embodiments, R₂₂ is unsubstituted C₁-C₁₀ aliphatic acyl. In some embodiments, R₂₂ is substituted C₁-C₁₀ aliphatic acyl. In some embodiments, R₂₂ is unsubstituted C₆-C₁₀ aromatic acyl. In some embodiments, R₂₂ is substituted C₆-C₁₀ aromatic acyl. In some embodiments, R₂₂ is unsubstituted C₆-C₁₀ aralkyl acyl. In some embodiments, R₂₂ is substituted C₆-C₁₀ aralkyl acyl. In some embodiments, R₂₂ is unsubstituted C₆-C₁₀ alkylaryl acyl. In some embodiments, R₂₂ is substituted C₆-C₁₀ alkylaryl acyl. In some embodiments, R₂₂ is unsubstituted alkoxy. In some embodiments, R₂₂ is substituted alkoxy. In some embodiments, R₂₂ is unsubstituted aryl. In some embodiments, R₂₂ is substituted aryl. In some embodiments, R₁₈ is unsubstituted C₃-C₁₀heterocyclyl. In some embodiments, R₂₂ is substituted C₃-C₁₀heterocyclyl. In some embodiments, R₂₂ is unsubstituted heteroaryl. In some embodiments, R₂₂ is substituted heteroaryl. In some embodiments, R₂₂ is unsubstituted C₃-C₁₀cycloalkyl. In some embodiments, R₂₂ is substituted C₃-C₁₀cycloalkyl. In some embodiments, R₂₂ is —OPO₃WY. In some embodiments, R₂₂ is —OCH₂PO₄WY. In some embodiments, R₂₂ is —OCH₂PO₄Z. In some embodiments, R₂₂ is —OPO₃Z.

In some embodiments, t is an integer of 0. In some embodiments, t is an integer of 1. In some embodiments, t is an integer of 2. In some embodiments, t is an integer of 3. In some embodiments, t is an integer of 4.

In some embodiments, the pyrone analog of Formula III is of Formula XIX:

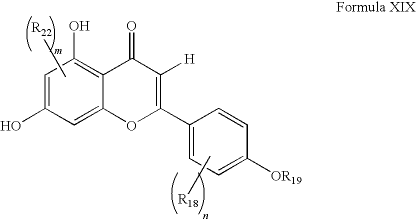

wherein R₁₈ and R₁₉ are as defined in Formula II;

wherein each instance of R₂₂ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z; and

m is an integer of 0, 1, or 2.

In some embodiments, m is an integer of 0. In some embodiments, m is an integer of 1. In some embodiments, m is an integer of 2.

In some embodiments, the pyrone analog of Formula III is of Formula XX:

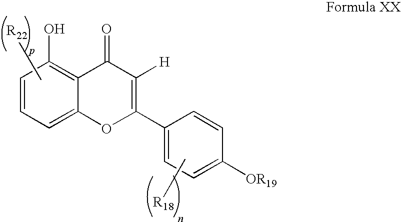

wherein R₁₈ and R₁₉ are as defined in Formula II;

wherein each instance of R₂₂ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z; and

p is an integer of 0, 1, 2 or 3.

In some embodiments, R₂₂ is hydrogen. In some embodiments, R₂₂ is hydroxy. In some embodiments, R₂₂ is carboxaldehyde. In some embodiments, R₂₂ is unsubstituted amine. In some embodiments, R₂₂ is substituted amine. In some embodiments, R₂₂ is unsubstituted C₁-C₁₀ alkyl. In some embodiments, R₂₂ is unsubstituted C₂-C₁₀ alkynyl. In some embodiments, R₂₂ is substituted C₂-C₁₀ alkynyl. In some embodiments, R₂₂ is unsubstituted C₂-C₁₀ alkenyl. In some embodiments, R₂₂ is substituted C₂-C₁₀ alkenyl. In some embodiments, R₂₂ is carboxyl. In some embodiments, R₂₂ is unsubstituted carbohydrate. In some embodiments, R₂₂ is substituted carbohydrate. In some embodiments, R₂₂ is unsubstituted ester. In some embodiments, R₂₂ is substituted ester. In some embodiments, R₂₂ is unsubstituted acyloxy. In some embodiments, R₂₂ is substituted acyloxy. In some embodiments, R₂₂ is nitro. In some embodiments, R₂₂ is halogen. In some embodiments, R₂₂ is unsubstituted C₁-C₁₀ aliphatic acyl. In some embodiments, R₂₂ is substituted C₁-C₁₀ aliphatic acyl. In some embodiments, R₂₂ is unsubstituted C₆-C₁₀ aromatic acyl. In some embodiments, R₂₂ is substituted C₆-C₁₀ aromatic acyl. In some embodiments, R₂₂ is unsubstituted C₆-C₁₀ aralkyl acyl. In some embodiments, R₂₂ is substituted C₆-C₁₀ aralkyl acyl. In some embodiments, R₂₂ is unsubstituted C₆-C₁₀ alkylaryl acyl. In some embodiments, R₂₂ is substituted C₆-C₁₀ alkylaryl acyl. In some embodiments, R₂₂ is unsubstituted alkoxy. In some embodiments, R₂₂ is substituted alkoxy. In some embodiments, R₂₂ is unsubstituted aryl. In some embodiments, R₂₂ is substituted aryl. In some embodiments, R₁₈ is unsubstituted C₃-C₁₀heterocyclyl. In some embodiments, R₂₂ is substituted C₃-C₁₀heterocyclyl. In some embodiments, R₂₂ is unsubstituted heteroaryl. In some embodiments, R₂₂ is substituted heteroaryl. In some embodiments, R₂₂ is unsubstituted C₃-C₁₀cycloalkyl. In some embodiments, R₂₂ is substituted C₃-C₁₀cycloalkyl. In some embodiments, R₂₂ is —OPO₃WY. In some embodiments, R₂₂ is —OCH₂PO₄WY. In some embodiments, R₂₂ is —OCH₂PO₄Z. In some embodiments, R₂₂ is —OPO₃Z.

In some embodiments, p is an integer of 0. In some embodiments, p is an integer of 1. In some embodiments, p is an integer of 2. In some embodiments, p is an integer of 3.

In some embodiments, the pyrone analog of Formula III is of Formula XXI:

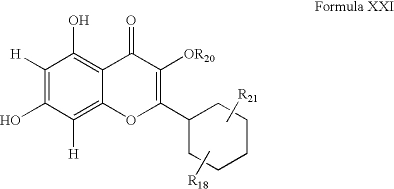

wherein R₁₈ and R₂₁ as defined in Formula II; and

R₂₀ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀heterocyclyl, heteroaryl, optionally substituted C₃-C₁₀cycloalkyl, —PO₃WY, —CH₂PO₄WY, —CH₂PO₄Z or —PO₃Z.

In some embodiments, R₂₀ is hydrogen. In some embodiments, R₂₀ is unsubstituted C₁-C₁₀ alkyl. In some embodiments, R₂₀ is substituted C₁-C₁₀ alkyl. In some embodiments, R₂₀ is unsubstituted C₂-C₁₀ alkynyl. In some embodiments, R₂₀ is substituted C₂-C₁₀ alkynyl. In some embodiments, R₂₀ is unsubstituted C₂-C₁₀ alkenyl. In some embodiments, R₂₀ is substituted C₂-C₁₀ alkenyl. In some embodiments, R₂₀ is unsubstituted carbohydrate. In some embodiments, R₂₀ is substituted carbohydrate. In some embodiments, R₂₀ is unsubstituted C₁-C₁₀ aliphatic acyl. In some embodiments, R₂₀ is substituted C₁-C₁₀ aliphatic acyl. In some embodiments, R₂₀ is unsubstituted C₆-C₁₀ aromatic acyl. In some embodiments, R₂₀ is substituted C₆-C₁₀ aromatic acyl. In some embodiments, R₂₀ is unsubstituted C₆-C₁₀ aralkyl acyl. In some embodiments, R₂₀ is substituted C₆-C₁₀ aralkyl acyl. In some embodiments, R₂₀ is unsubstituted C₆-C₁₀ alkylaryl acyl. In some embodiments, R₂₀ is substituted C₆-C₁₀ alkylaryl acyl. In some embodiments, R₂₀ is unsubstituted aryl. In some embodiments, R₂₀ is substituted aryl. In some embodiments, R₂₀ is unsubstituted C₃-C₁₀heterocyclyl. In some embodiments, R₂₀ is substituted C₃-C₁₀heterocyclyl. In some embodiments, R₂₀ is unsubstituted heteroaryl. In some embodiments, R₂₀ is substituted heteroaryl. In some embodiments, R₂₀ is unsubstituted C₃-C₁₀cycloalkyl. In some embodiments, R₂₀ is substituted C₃-C₁₀cycloalkyl. In some embodiments, R₂₀ is —PO₃WY. In some embodiments, R₂₀ is —CH₂PO₄WY. In some embodiments, R₂₀ is —CH₂PO₄Z. In some embodiments, R₂₀ is —PO₃Z.

In some embodiments, the pyrone analog of Formula III is of Formula XXII:

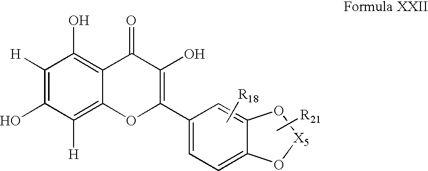

wherein R₁₈ and R₂₁ are as defined in Formula II;

wherein X₅ is a C₁ to C₄ group, optionally interrupted by O, S, NR₂₃, or NR₂₃R₂₃ as valency permits, forming a ring which is aromatic or nonaromatic;

each instance of R₂₃ is independently hydrogen, C₁-C₁₀ alkyl, C₂-C₁₀ alkynyl, C₂-C₁₀ alkenyl, carbohydrate, acyloxy, C₁-C₁₀ aliphatic acyl, C₆-C₁₀ aromatic acyl, C₆-C₁₀ aralkyl acyl, C₆-C₁₀ alkylaryl acyl, alkoxy, aryl, heteroaryl, C₅-C₁₀heterocyclyl, C₃-C₁₀cycloalkyl, —PO₃WY, —CH₂PO₄WY, —CH₂PO₄Z or —PO₃Z.

In some embodiments, R₂₃ is hydrogen. In some embodiments, R₂₃ is unsubstituted C₁-C₁₀ alkyl. In some embodiments, R₂₃ is substituted C₁-C₁₀ alkyl. In some embodiments, R₂₃ is unsubstituted C₂-C₁₀ alkynyl. In some embodiments, R₂₃ is substituted C₂-C₁₀ alkynyl. In some embodiments, R₂₃ is unsubstituted C₂-C₁₀ alkenyl. In some embodiments, R₂₃ is substituted C₂-C₁₀ alkenyl. In some embodiments, R₂₃ is unsubstituted acyloxy. In some embodiments, R₂₃ is substituted acyloxy. In some embodiments, R₂₃ is unsubstituted carbohydrate. In some embodiments, R₂₃ is substituted carbohydrate. In some embodiments, R₂₃ is unsubstituted acyloxy. In some embodiments, R₂₃ is substituted acyloxy. In some embodiments, R₂₃ is unsubstituted C₁-C₁₀ aliphatic acyl. In some embodiments, R₂₃ is substituted C₁-C₁₀ aliphatic acyl. In some embodiments, R₂₃ is unsubstituted C₆-C₁₀ aromatic acyl. In some embodiments, R₂₃ is substituted C₆-C₁₀ aromatic acyl. In some embodiments, R₂₃ is unsubstituted C₆-C₁₀ aralkyl acyl. In some embodiments, R₂₃ is substituted C₆-C₁₀ aralkyl acyl. In some embodiments, R₂₃ is unsubstituted C₆-C₁₀ alkylaryl acyl. In some embodiments, R₂₃ is substituted C₆-C₁₀ alkylaryl acyl. In some embodiments, R₂₃ is unsubstituted alkoxy. In some embodiments, R₂₃ is substituted alkoxy. In some embodiments, R₂₃ is unsubstituted aryl. In some embodiments, R₂₃ is substituted aryl. In some embodiments, R₂₃ is unsubstituted C₃-C₁₀heterocyclyl. In some embodiments, R₂₃ is substituted C₃-C₁₀heterocyclyl. In some embodiments, R₂₃ is unsubstituted heteroaryl. In some embodiments, R₂₃ is substituted heteroaryl. In some embodiments, R₂₃ is unsubstituted C₃-C₁₀cycloalkyl. In some embodiments, R₂₃ is substituted C₃-C₁₀cycloalkyl.

In some embodiments, the pyrone analog of Formula III is of Formula XXIII:

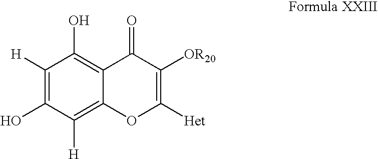

Wherein R₂₀ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, optionally substituted C₃-C₁₀cycloalkyl, —PO₃WY, —CH₂PO₄WY, —CH₂PO₄Z or —PO₃Z;

Het is a 3 to 10 membered optionally substituted monocyclic or bicyclic heteroaromatic or heteroalicyclic ring system containing 1, 2, 3, 4, or 5 heteroatoms selected from the group of O, S, and N, with the proviso that no two adjacent ring atoms are O or S, wherein the ring system is unsaturated, partially unsaturated or saturated, wherein any number of the ring atoms have substituents as valency permits which are hydrogen, hydroxyl, carboxyaldehyde, alkylcarboxaldehyde, imino, C₁-C₁₀ alkyl, C₁-C₁₀ alkynyl, C₁-C₁₀ alkenyl, carboxyl, carbohydrate, acyloxy, nitro, halogen, C₁-C₁₀ aliphatic acyl, C₅-C₁₀ aromatic acyl, C₆-C₁₀ aralkyl acyl, C₆-C₁₀ alkylaryl acyl, alkoxy, amine, aryl, heteroaryl, C₅-C₁₀heterocyclyl, C₅-C₁₀cycloalkyl, —OPO₃WY, —OCH₂PO₄WY, —OCH₂PO₄Z or —OPO₃Z; and

W and Y are independently hydrogen, methyl, ethyl, alkyl, carbohydrate, or a cation, and Z is a multivalent cation.

In some embodiments, R₂₀ is hydrogen. In some embodiments, R₂₀ is unsubstituted C₁-C₁₀ alkyl. In some embodiments, R₂₀ is substituted C₁-C₁₀ alkyl. In some embodiments, R₂₀ is unsubstituted C₂-C₁₀ alkynyl. In some embodiments, R₂₀ is substituted C₂-C₁₀ alkynyl. In some embodiments, R₂₀ is unsubstituted C₂-C₁₀ alkenyl. In some embodiments, R₂₀ is substituted C₂-C₁₀ alkenyl. In some embodiments, R₂₀ is unsubstituted carbohydrate. In some embodiments, R₂₀ is substituted carbohydrate. In some embodiments, R₂₀ is unsubstituted C₁-C₁₀ aliphatic acyl. In some embodiments, R₂₀ is substituted C₁-C₁₀ aliphatic acyl. In some embodiments, R₂₀ is unsubstituted C₆-C₁₀ aromatic acyl. In some embodiments, R₂₀ is substituted C₆-C₁₀ aromatic acyl. In some embodiments, R₂₀ is unsubstituted C₆-C₁₀ aralkyl acyl. In some embodiments, R₂₀ is substituted C₆-C₁₀ aralkyl acyl. In some embodiments, R₂₀ is unsubstituted C₆-C₁₀ alkylaryl acyl. In some embodiments, R₂₀ is substituted C₆-C₁₀ alkylaryl acyl. In some embodiments, R₂₀ is unsubstituted aryl. In some embodiments, R₂₀ is substituted aryl. In some embodiments, R₂₀ is unsubstituted C₃-C₁₀heterocyclyl. In some embodiments, R₂₀ is substituted C₃-C₁₀heterocyclyl. In some embodiments, R₂₀ is unsubstituted heteroaryl. In some embodiments, R₂₀ is substituted heteroaryl. In some embodiments, R₂₀ is unsubstituted C₃-C₁₀cycloalkyl. In some embodiments, R₂₀ is substituted C₃-C₁₀cycloalkyl. In some embodiments, R₂₀ is —PO₃WY. In some embodiments, R₂₀ is —CH₂PO₄WY. In some embodiments, R₂₀ is —CH₂PO₄Z. In some embodiments, R₂₀ is —PO₃Z.

In some embodiments, Het is one of the following formulae:

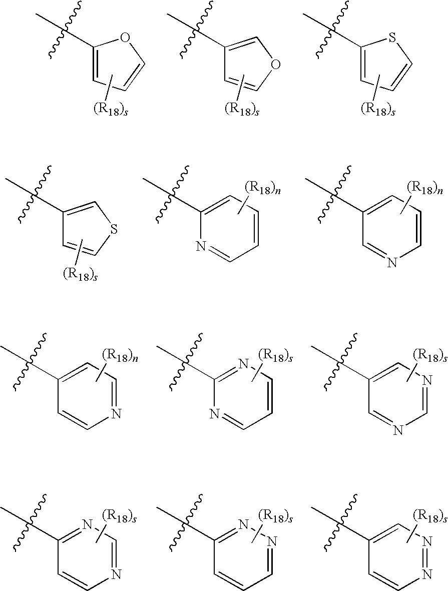

wherein each instance of R₁₈ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z;

s is an integer of 0, 1, 2, or 3; and

n is an integer of 0, 1, 2, 3, or 4.

In some embodiments, R₁₈ is hydrogen. In some embodiments, R₁₈ is hydroxy. In some embodiments, R₁₈ is carboxaldehyde. In some embodiments, R₁₈ is unsubstituted amine. In some embodiments, R₁₈ is substituted amine. In some embodiments, R₁₈ is unsubstituted C₁-C₁₀ alkyl. In some embodiments, R₁₈ is unsubstituted C₂-C₁₀ alkynyl. In some embodiments, R₁₈ is substituted C₂-C₁₀ alkynyl. In some embodiments, R₁₈ is unsubstituted C₂-C₁₀ alkenyl. In some embodiments, R₁₈ is substituted C₂-C₁₀ alkenyl. In some embodiments, R₁₈ is carboxyl. In some embodiments, R₁₈ is unsubstituted carbohydrate. In some embodiments, R₁₈ is substituted carbohydrate. In some embodiments, R₁₈ is substituted carbohydrate. In some embodiments, R₁₈ is unsubstituted ester. In some embodiments, R₁₈ is substituted ester. In some embodiments, R₁₈ is unsubstituted acyloxy. In some embodiments, R₁₈ is substituted acyloxy. In some embodiments, R₁₈ is nitro. In some embodiments, R₁₈ is halogen. In some embodiments, R₁₈ is unsubstituted C₁-C₁₀ aliphatic acyl. In some embodiments, R₁₈ is substituted C₁-C₁₀ aliphatic acyl. In some embodiments, R₁₈ is unsubstituted C₆-C₁₀ aromatic acyl. In some embodiments, R₁₈ is substituted C₆-C₁₀ aromatic acyl. In some embodiments, R₁₈ is unsubstituted C₆-C₁₀ aralkyl acyl. In some embodiments, R₁₈ is substituted C₆-C₁₀ aralkyl acyl. In some embodiments, R₁₈ is unsubstituted C₆-C₁₀ alkylaryl acyl. In some embodiments, R₁₈ is substituted C₆-C₁₀ alkylaryl acyl. In some embodiments, R₁₈ is unsubstituted alkoxy. In some embodiments, R₁₈ is substituted alkoxy. In some embodiments, R₁₈ is unsubstituted aryl. In some embodiments, R₁₈ is substituted aryl. In some embodiments, R₁₈ is unsubstituted C₃-C₁₀heterocyclyl. In some embodiments, R₁₈ is substituted C₃-C₁₀heterocyclyl. In some embodiments, R₁₈ is unsubstituted heteroaryl. In some embodiments, R₁₈ is substituted heteroaryl. In some embodiments, R₁₈ is unsubstituted C₃-C₁₀cycloalkyl. In some embodiments, R₁₈ is substituted C₃-C₁₀cycloalkyl. In some embodiments, R₁₈ is —OPO₃WY. In some embodiments, R₁₈ is —OCH₂PO₄WY. In some embodiments, R₁₈ is —OCH₂PO₄Z. In some embodiments, R₁₈ is —OPO₃Z.

In some embodiments, n is an integer of 0. In some embodiments, n is an integer of 1. In some embodiments, n is an integer of 2. In some embodiments, n is an integer of 3. In some embodiments, n is an integer of 4.

In some embodiments, s is an integer of 0. In some embodiments, s is an integer of 1. In some embodiments, s is an integer of 2. In some embodiments, s is an integer of 3.

In some embodiments of the invention, the pyrone analog of Formula II is of Formula IV:

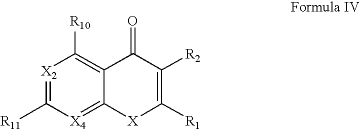

wherein X, X₂, X₄, R′, R₁, R₂, W, Y, and Z are as defined for Formula II; and

R₁₀ and R₁₁ are independently hydrogen, hydroxyl, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀alkylaryl acyl, alkoxy, amine, aryl, C₃-C₁₀heterocyclyl, heteroaryl, C₃-C₁₀cycloalkyl, —OPO₃WY, —OCH₂PO₄WY, —OCH₂PO₄Z or —OPO₃Z.

In some embodiments, R₁₀ is hydrogen. In some embodiments, R₁₀ is hydroxyl. In some embodiments, R₁₀ is carboxaldehyde. In some embodiments, R₁₀ is unsubstituted amine. In some embodiments, R₁₀ is substituted amine. In some embodiments, R₁₀ is unsubstituted C₁-C₁₀ alkyl. In some embodiments, R₁₀ is substituted C₁-C₁₀ alkyl. In some embodiments, R₁₀ is unsubstituted C₂-C₁₀ alkynyl. In some embodiments, R₁₀ is substituted C₂-C₁₀ alkynyl. In some embodiments, R₁₀ is unsubstituted C₂-C₁₀ alkenyl. In some embodiments, R₁₀ is substituted C₂-C₁₀ alkenyl. In some embodiments, R₁₀ is carboxyl. In some embodiments, R₁₀ is unsubstituted carbohydrate. In some embodiments, R₁₀ is substituted carbohydrate. In some embodiments, R₁₀ is unsubstituted ester. In some embodiments, R₁₀ is substituted ester. In some embodiments, R₁₀ is unsubstituted acyloxy. In some embodiments, R₁₀ is substituted acyloxy. In some embodiments, R₁₀ is nitro. In some embodiments, R₁₀ is halogen. In some embodiments, R₁₀ is unsubstituted C₁-C₁₀ aliphatic acyl. In some embodiments, R₁₀ is substituted C₁-C₁₀ aliphatic acyl. In some embodiments, R₁₀ is unsubstituted C₆-C₁₀ aromatic acyl. In some embodiments, R₁₀ is substituted C₆-C₁₀ aromatic acyl. In some embodiments, R₁₀ is unsubstituted C₆-C₁₀ aralkyl acyl. In some embodiments, R₁₀ is substituted C₆-C₁₀ aralkyl acyl. In some embodiments, R₁₀ is unsubstituted C₆-C₁₀ alkylaryl acyl. In some embodiments, R₁₀ is substituted C₆-C₁₀ alkylaryl acyl. In some embodiments, R₁₀ is unsubstituted alkoxy. In some embodiments, R₁₀ is substituted alkoxy. In some embodiments, R₁₀ is unsubstituted aryl. In some embodiments, R₁₀ is substituted aryl. In some embodiments, R₁₀ is unsubstituted C₃-C₁₀heterocyclyl. In some embodiments, R₁₀ is substituted C₃-C₁₀heterocyclyl. In some embodiments, R₁₀ is unsubstituted heteroaryl. In some embodiments, R₁₀ is unsubstituted C₃-C₁₀cycloalkyl. In some embodiments, R₁₀ is substituted C₃-C₁₀cycloalkyl. In some embodiments, R₁₀ is —OPO₃WY. In some embodiments, R₁₀ is —OCH₂PO₄WY. In some embodiments, R₁₀ is —OCH₂PO₄Z. In some embodiments, R₁₀ is —OPO₃Z.

In some embodiments, R₁₁ is hydrogen. In some embodiments, R₁₁ is hydroxyl. In some embodiments, R₁₁ is carboxaldehyde. In some embodiments, R₁₁ is unsubstituted amine. In some embodiments, R₁₁ is substituted amine. In some embodiments, R₁₁ is unsubstituted C₁-C₁₀ alkyl. In some embodiments, R₁₁ is substituted C₁-C₁₀ alkyl. In some embodiments, R₁₁ is unsubstituted C₂-C₁₀ alkynyl. In some embodiments, R₁₁ is substituted C₂-C₁₀ alkynyl. In some embodiments, R₁₁ is unsubstituted C₂-C₁₀ alkenyl. In some embodiments, R₁₁ is substituted C₂-C₁₀ alkenyl. In some embodiments, R₁₁ is carboxyl. In some embodiments, R₁₁ is unsubstituted carbohydrate. In some embodiments, R₁₁ is substituted carbohydrate. In some embodiments, R₁₁ is unsubstituted ester. In some embodiments, R₁₁ is substituted ester. In some embodiments, R₁₁ is unsubstituted acyloxy. In some embodiments, R₁₁ is substituted acyloxy. In some embodiments, R₁₁ is nitro. In some embodiments, R₁₁ is halogen. In some embodiments, R₁ is unsubstituted C₁-C₁₀ aliphatic acyl. In some embodiments, R₁ is substituted C₁-C₁₀ aliphatic acyl. In some embodiments, R₁₁ is unsubstituted C₆-C₁₀ aromatic acyl. In some embodiments, R₁₁ is substituted C₆-C₁₀ aromatic acyl. In some embodiments, R₁₁ is unsubstituted C₆-C₁₀ aralkyl acyl. In some embodiments, R₁₁ is substituted C₆-C₁₀ aralkyl acyl. In some embodiments, R₁₁ is unsubstituted C₆-C₁₀ alkylaryl acyl. In some embodiments, R₁₁ is substituted C₆-C₁₀ alkylaryl acyl. In some embodiments, R₁₁ is unsubstituted alkoxy. In some embodiments, R₁₁ is substituted alkoxy. In some embodiments, R₁₁ is unsubstituted aryl. In some embodiments, R₁₁ is substituted aryl. In some embodiments, R₁₁ is unsubstituted C₃-C₁₀heterocyclyl. In some embodiments, R₁₁ is substituted C₃-C₁₀heterocyclyl. In some embodiments, R₁₁ is unsubstituted heteroaryl. In some embodiments, R₁₁ is unsubstituted C₃-C₁₀cycloalkyl. In some embodiments, R₁₁ is substituted C₃-C₁₀cycloalkyl. In some embodiments, R₁₁ is —OPO₃WY. In some embodiments, R₁₁ is —OCH₂PO₄WY. In some embodiments, R₁₁ is —OCH₂PO₄Z. In some embodiments, R₁₁ is —OPO₃Z.

In some embodiments of the invention, the pyrone analog of Formula IV is of Formula XXIV or Formula XXV:

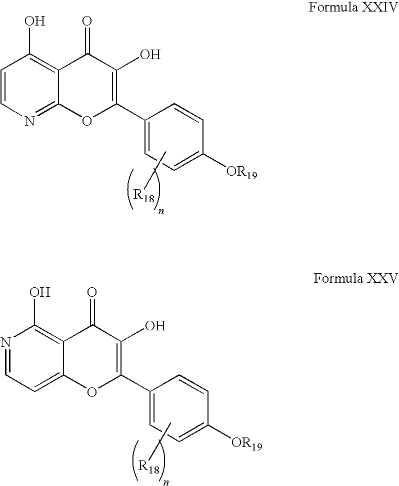

wherein R₁₈, R₁₉, and n are as defined in Formula II.

In some embodiments of the invention, the pyrone analog of Formula IV is of Formula XXVI or Formula XXVII:

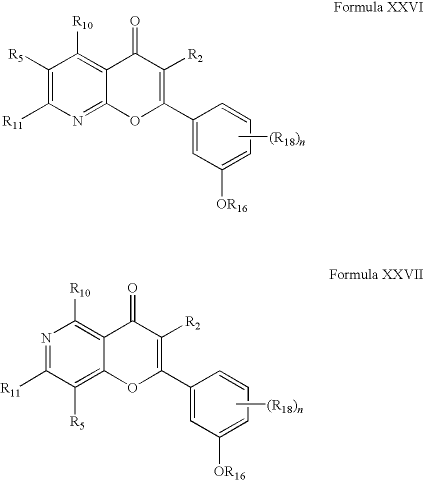

wherein R₂, R₅, W, Y, and Z are as defined for Formula II and R₁₀ and R₁₁ are as defined for Formula IV;

R₁₆ is hydrogen, —PO₃WY, —CH₂PO₄WY, —CH₂PO₄Z or —PO₃Z;

wherein each instance of R₁₈ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z; and

n is an integer of 0, 1, 2, 3, or 4.

In some embodiments of the invention, the pyrone analog of Formula IV is of Formula XXVIII:

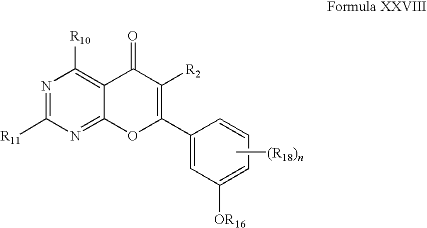

wherein R₂, W, Y, and Z are as defined for Formula II and R₁₀ and R₁₁ are as defined for Formula IV;

R₁₆ is hydrogen, —PO₃WY, —CH₂PO₄WY, —CH₂PO₄Z or —PO₃Z;

wherein each instance of R₁₈ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z; and

n is an integer of 0, 1, 2, 3, or 4.

In some embodiments of the invention, the pyrone analog of Formula II is of Formula V:

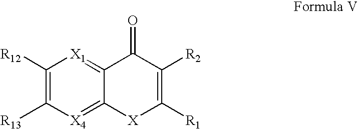

wherein X, X₁, X₄, R′, R₁, R₂, W, Y, and Z are as defined for Formula II; and

R₁₂ and R₁₃ are independently hydrogen, hydroxyl, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀alkylaryl acyl, alkoxy, amine, aryl, C₃-C₁₀heterocyclyl, heteroaryl, C₃-C₁₀cycloalkyl, —OPO₃WY, —OCH₂PO₄WY, —OCH₂PO₄Z or —OPO₃Z.

In some embodiments, R₁₂ is hydrogen. In some embodiments, R₁₂ is hydroxyl. In some embodiments, R₁₂ is carboxaldehyde. In some embodiments, R₁₂ is unsubstituted amine. In some embodiments, R₁₂ is substituted amine. In some embodiments, R₁₂ is unsubstituted C₁-C₁₀ alkyl. In some embodiments, R₁₂ is substituted C₁-C₁₀ alkyl. In some embodiments, R₁₂ is unsubstituted C₂-C₁₀ alkynyl. In some embodiments, R₁₂ is substituted C₂-C₁₀ alkynyl. In some embodiments, R₁₂ is unsubstituted C₂-C₁₀ alkenyl. In some embodiments, R₁₂ is substituted C₂-C₁₀ alkenyl. In some embodiments, R₁₂ is carboxyl. In some embodiments, R₁₂ is unsubstituted carbohydrate. In some embodiments, R₁₂ is substituted carbohydrate. In some embodiments, R₁₂ is unsubstituted ester. In some embodiments, R₁₂ is substituted ester. In some embodiments, R₁₂ is unsubstituted acyloxy. In some embodiments, R₁₂ is substituted acyloxy. In some embodiments, R₁₂ is nitro. In some embodiments, R₁₂ is halogen. In some embodiments, R₁₂ is unsubstituted C₁-C₁₀ aliphatic acyl. In some embodiments, R₁₂ is substituted C₁-C₁₀ aliphatic acyl. In some embodiments, R₁₂ is unsubstituted C₆-C₁₀ aromatic acyl. In some embodiments, R₁₂ is substituted C₆-C₁₀ aromatic acyl. In some embodiments, R₁₂ is unsubstituted C₆-C₁₀ aralkyl acyl. In some embodiments, R₁₂ is substituted C₆-C₁₀ aralkyl acyl. In some embodiments, R₁₂ is unsubstituted C₆-C₁₀ alkylaryl acyl. In some embodiments, R₁₂ is substituted C₆-C₁₀ alkylaryl acyl. In some embodiments, R₁₂ is unsubstituted alkoxy. In some embodiments, R₁₂ is substituted alkoxy. In some embodiments, R₁₂ is unsubstituted aryl. In some embodiments, R₁₂ is substituted aryl. In some embodiments, R₁₂ is unsubstituted C₃-C₁₀heterocyclyl. In some embodiments, R₁₂ is substituted C₃-C₁₀heterocyclyl. In some embodiments, R₁₂ is unsubstituted heteroaryl. In some embodiments, R₁₂ is unsubstituted C₃-C₁₀cycloalkyl. In some embodiments, R₁₂ is substituted C₃-C₁₀cycloalkyl. In some embodiments, R₁₂ is —OPO₃WY. In some embodiments, R₁₂ is —OCH₂PO₄WY. In some embodiments, R₁₂ is —OCH₂PO₄Z. In some embodiments, R₁₂ is —OPO₃Z.

In some embodiments, R₁₃ is hydrogen. In some embodiments, R₁₃ is hydroxyl. In some embodiments, R₁₃ is carboxaldehyde. In some embodiments, R₁₃ is unsubstituted amine. In some embodiments, R₁₃ is substituted amine. In some embodiments, R₁₃ is unsubstituted C₁-C₁₀ alkyl. In some embodiments, R₁₃ is substituted C₁-C₁₀ alkyl. In some embodiments, R₁₃ is unsubstituted C₂-C₁₀ alkynyl. In some embodiments, R₁₃ is substituted C₂-C₁₀ alkynyl. In some embodiments, R₁₃ is unsubstituted C₂-C₁₀ alkenyl. In some embodiments, R₁₃ is substituted C₂-C₁₀ alkenyl. In some embodiments, R₁₃ is carboxyl. In some embodiments, R₁₃ is unsubstituted carbohydrate. In some embodiments, R₁₃ is substituted carbohydrate. In some embodiments, R₁₃ is unsubstituted ester. In some embodiments, R₁₃ is substituted ester. In some embodiments, R₁₃ is unsubstituted acyloxy. In some embodiments, R₁₃ is substituted acyloxy. In some embodiments, R₁₃ is nitro. In some embodiments, R₁₃ is halogen. In some embodiments, R₁₃ is unsubstituted C₁-C₁₀ aliphatic acyl. In some embodiments, R₁₃ is substituted C₁-C₁₀ aliphatic acyl. In some embodiments, R₁₃ is unsubstituted C₆-C₁₀ aromatic acyl. In some embodiments, R₁₃ is substituted C₆-C₁₀ aromatic acyl. In some embodiments, R₁₃ is unsubstituted C₆-C₁₀ aralkyl acyl. In some embodiments, R₁₃ is substituted C₆-C₁₀ aralkyl acyl. In some embodiments, R₁₃ is unsubstituted C₆-C₁₀ alkylaryl acyl. In some embodiments, R₁₃ is substituted C₆-C₁₀ alkylaryl acyl. In some embodiments, R₁₃ is unsubstituted alkoxy. In some embodiments, R₁₃ is substituted alkoxy. In some embodiments, R₁₃ is unsubstituted aryl. In some embodiments, R₁₃ is substituted aryl. In some embodiments, R₁₃ is unsubstituted C₃-C₁₀heterocyclyl. In some embodiments, R₁₃ is substituted C₃-C₁₀heterocyclyl. In some embodiments, R₁₃ is unsubstituted heteroaryl. In some embodiments, R₁₃ is unsubstituted C₃-C₁₀cycloalkyl. In some embodiments, R₁₃ is substituted C₃-C₁₀cycloalkyl. In some embodiments, R₁₃ is —OPO₃WY. In some embodiments, R₁₃ is —OCH₂PO₄WY. In some embodiments, R₁₃ is —OCH₂PO₄Z. In some embodiments, R₁₃ is —OPO₃Z.

In some embodiments of the invention, the pyrone analog of Formula V is of Formula XXIX or Formula XXX:

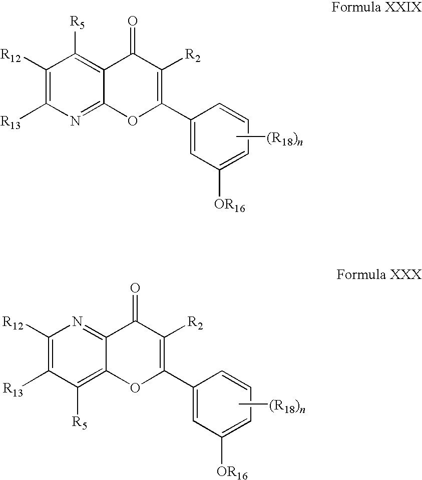

wherein R₂, R₅, R₁₈, n, W, Y, and Z are as defined for Formula II and R₁₂ and R₁₃ are as defined for Formula V; and

R₁₆ is hydrogen, —PO₃WY, —CH₂PO₄WY, —CH₂PO₄Z or —PO₃Z.

In some embodiments of the invention, the pyrone analog of Formula V is of Formula XXXI:

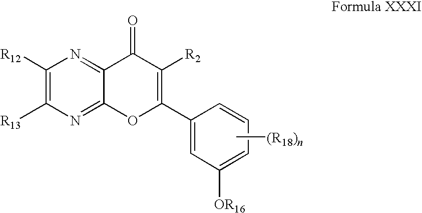

wherein R₂, R₁₈, n, W, Y, and Z are as defined for Formula II and R₁₂ and R₁₃ are as defined for Formula V; and

R₁₆ is hydrogen, —PO₃WY, —CH₂PO₄WY, —CH₂PO₄Z or —PO₃Z.

In some embodiments of the invention, the pyrone analog of Formula II is of Formula VI:

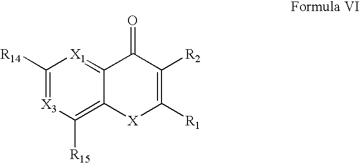

wherein X, X₁, X₃, R′, R₁, R₂, W, Y, and Z are as defined for Formula II; and

R₁₄ and R₁₅ are independently hydrogen, hydroxyl, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀alkylaryl acyl, alkoxy, amine, aryl, C₃-C₁₀heterocyclyl, heteroaryl, C₃-C₁₀cycloalkyl, —OPO₃WY, —OCH₂PO₄WY, —OCH₂PO₄Z or —OPO₃Z.

In some embodiments, R₁₄ is hydrogen. In some embodiments, R₁₄ is hydroxyl. In some embodiments, R₁₄ is carboxaldehyde. In some embodiments, R₁₄ is unsubstituted amine. In some embodiments, R₁₄ is substituted amine. In some embodiments, R₁₄ is unsubstituted C₁-C₁₀ alkyl. In some embodiments, R₁₄ is substituted C₁-C₁₀ alkyl. In some embodiments, R₁₄ is unsubstituted C₂-C₁₀ alkynyl. In some embodiments, R₁₄ is substituted C₂-C₁₀ alkynyl. In some embodiments, R₁₄ is unsubstituted C₂-C₁₀ alkenyl. In some embodiments, R₁₄ is substituted C₂-C₁₀ alkenyl. In some embodiments, R₁₄ is carboxyl. In some embodiments, R₁₄ is unsubstituted carbohydrate. In some embodiments, R₁₄ is substituted carbohydrate. In some embodiments, R₁₄ is unsubstituted ester. In some embodiments, R₁₄ is substituted ester. In some embodiments, R₁₄ is unsubstituted acyloxy. In some embodiments, R₁₄ is substituted acyloxy. In some embodiments, R₁₄ is nitro. In some embodiments, R₁₄ is halogen. In some embodiments, R₁₄ is unsubstituted C₁-C₁₀ aliphatic acyl. In some embodiments, R₁₄ is substituted C₁-C₁₀ aliphatic acyl. In some embodiments, R₁₄ is unsubstituted C₆-C₁₀ aromatic acyl. In some embodiments, R₁₄ is substituted C₆-C₁₀ aromatic acyl. In some embodiments, R₁₄ is unsubstituted C₆-C₁₀ aralkyl acyl. In some embodiments, R₁₄ is substituted C₆-C₁₀ aralkyl acyl. In some embodiments, R₁₄ is unsubstituted C₆-C₁₀ alkylaryl acyl. In some embodiments, R₁₄ is substituted C₆-C₁₀ alkylaryl acyl. In some embodiments, R₁₄ is unsubstituted alkoxy. In some embodiments, R₁₄ is substituted alkoxy. In some embodiments, R₁₄ is unsubstituted aryl. In some embodiments, R₁₄ is substituted aryl. In some embodiments, R₁₄ is unsubstituted C₃-C₁₀heterocyclyl. In some embodiments, R₁₄ is substituted C₃-C₁₀heterocyclyl. In some embodiments, R₁₄ is unsubstituted heteroaryl. In some embodiments, R₁₄ is unsubstituted C₃-C₁₀cycloalkyl. In some embodiments, R₁₄ is substituted C₃-C₁₀cycloalkyl. In some embodiments, R₁₄ is —OPO₃WY. In some embodiments, R₁₄ is —OCH₂PO₄WY. In some embodiments, R₁₄ is —OCH₂PO₄Z. In some embodiments, R₁₄ is —OPO₃Z.

In some embodiments, R₁₅ is hydrogen. In some embodiments, R₁₅ is hydroxyl. In some embodiments, R₁₅ is carboxaldehyde. In some embodiments, R₁₅ is unsubstituted amine. In some embodiments, R₁₅ is substituted amine. In some embodiments, R₁₅ is unsubstituted C₁-C₁₀ alkyl. In some embodiments, R₁₅ is substituted C₁-C₁₀ alkyl. In some embodiments, R₁₅ is unsubstituted C₂-C₁₀ alkynyl. In some embodiments, R₁₅ is substituted C₂-C₁₀ alkynyl. In some embodiments, R₁₅ is unsubstituted C₂-C₁₀ alkenyl. In some embodiments, R₁₅ is substituted C₂-C₁₀ alkenyl. In some embodiments, R₁₅ is carboxyl. In some embodiments, R₁₅ is unsubstituted carbohydrate. In some embodiments, R₁₅ is substituted carbohydrate. In some embodiments, R₁₅ is unsubstituted ester. In some embodiments, R₁₅ is substituted ester. In some embodiments, R₁₅ is unsubstituted acyloxy. In some embodiments, R₁₅ is substituted acyloxy. In some embodiments, R₁₃ is nitro. In some embodiments, R₁₃ is halogen. In some embodiments, R₁₃ is unsubstituted C₁-C₁₀ aliphatic acyl. In some embodiments, R₁₅ is substituted C₁-C₁₀ aliphatic acyl. In some embodiments, R₁₅ is unsubstituted C₆-C₁₀ aromatic acyl. In some embodiments, R₁₅ is substituted C₆-C₁₀ aromatic acyl. In some embodiments, R₁₅ is unsubstituted C₆-C₁₀ aralkyl acyl. In some embodiments, R₁₅ is substituted C₆-C₁₀ aralkyl acyl. In some embodiments, R₁₅ is unsubstituted C₆-C₁₀ alkylaryl acyl. In some embodiments, R₁₅ is substituted C₆-C₁₀ alkylaryl acyl. In some embodiments, R₁₅ is unsubstituted alkoxy. In some embodiments, R₁₅ is substituted alkoxy. In some embodiments, R₁₅ is unsubstituted aryl. In some embodiments, R₁₅ is substituted aryl. In some embodiments, R₁₅ is unsubstituted C₃-C₁₀heterocyclyl. In some embodiments, R₁₅ is substituted C₃-C₁₀heterocyclyl. In some embodiments, R₁₅ is unsubstituted heteroaryl. In some embodiments, R₁₅ is unsubstituted C₃-C₁₀cycloalkyl. In some embodiments, R₁₅ is substituted C₃-C₁₀cycloalkyl. In some embodiments, R₁₅ is —OPO₃WY. In some embodiments, R₁₅ is —OCH₂PO₄WY. In some embodiments, R₁₅ is —OCH₂PO₄Z. In some embodiments, R₁₅ is —OPO₃Z.

In some embodiments of the invention, the pyrone analog of Formula VI is of Formula XXXII or Formula XXXIII:

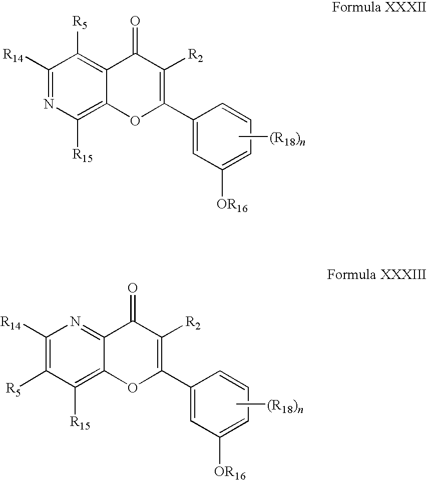

wherein R₂, R₅, R₁₈, n, W, Y, and Z are as defined for Formula II and R₁₄ and R₁₅ are as defined for Formula V; and

R₁₆ is hydrogen, —PO₃WY, —CH₂PO₄WY, —CH₂PO₄Z or —PO₃Z.

In some embodiments of the invention, the pyrone analog of Formula VI is of Formula XXXIV:

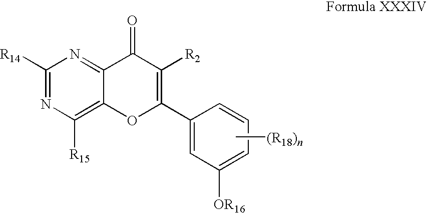

wherein R₂, R₁₈, n, W, Y, and Z are as defined for Formula II and R₁₄ and R₁₅ are as defined for Formula V; and

R₁₆ is hydrogen, —PO₃WY, —CH₂PO₄WY, —CH₂PO₄Z or —PO₃Z.

The compositions and methods of the present invention can involve flavonoids. Flavonoids can be classified into subgroups based on differences in their chemical structures. The basic flavonoid structure is shown below (formula XXXV):

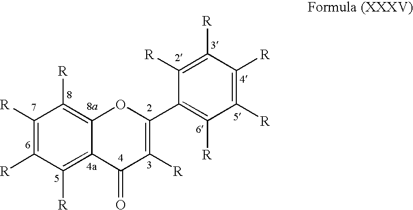

wherein the 2, 3 bond may be saturated or unsaturated, and wherein each R can be independently selected from the group consisting of hydrogen, optionally substituted hydroxyl, optionally substituted amine, optionally substituted thiol, optionally substituted C₁-C₁₀ alkyl, optionally substituted C₁-C₁₀ alkynyl, optionally substituted C₁-C₁₀ alkenyl, optionally substituted aryl, optionally substituted heteroaryl, optionally substituted C₅-C₁₀ cycloalkyl, optionally substituted C₅-C₁₀ heterocycloalkyl, optionally substituted C₁-C₁₀ aliphatic acyl, optionally substituted C₁-C₁₀ aromatic acyl, trialkyl silyl, optionally substituted ether, carbohydrate, substituted carbohydrate, amino acid, and substituted amino acid; and its pharmaceutically acceptable salts, esters, prodrugs, analogs, isomers, stereoisomers or tautomers thereof.

In some embodiments, the invention utilizes a flavonoid where the molecule is planar. In some embodiments, the invention utilizes a flavonoid where the 2-3 bond is unsaturated. In some embodiments, the invention utilizes a flavonoid where the 3-position is hydroxylated. In some embodiments, the invention utilizes a flavonoid where the 2-3 bond is unsaturated and the 3-position is hydroxylated (e.g., flavonols).

In some embodiments, the invention utilizes one or more flavonoids selected from the group consisting of quercetin or a quercetin derivative, isoquercetin, flavone, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, 5,7-dideoxyquercetin (3,3′,4′-trihydroxyflavone), and epicatechin. In some embodiments, the invention utilizes one or more flavonoids selected from the group consisting of quercetin or a quercetin derivative, isoquercetin, apigenin, rhoifolin, galangin, fisetin, morin, rutin, kaempferol, myricetin, naringenin, hesperetin, phloretin, and genistein. Structures of these compositions are well-known in the art. See, e.g., Critchfield et al. (1994) Biochem. Pharmacol 7:1437-1445.

In some embodiments, the invention utilizes a flavonol. In some embodiments, the flavonol is selected from the group consisting of quercetin or a quercetin derivative, fisetin, morin, rutin, myricetin, galangin, fisetin, and kaempherol, and combinations thereof. In some embodiments, the flavonol is selected from the group consisting of quercetin or a quercetin derivative, galangin, fisetin, and kaempherol, and combinations thereof. In some embodiments, the flavonol is quercetin. In some embodiments, the flavonol is galangin. In some embodiments, the flavonol is kaempherol.

A particularly useful flavonol is quercetin. Quercetin may be used to illustrate formulations and methods useful in the invention, however, it is understood that the discussion of quercetin applies equally to other flavonoids and flavonols useful in the invention, e.g., kaempferol and galangin. Quercetin in any suitable form and purity can be used in the invention. For example, in some cases quercetin in the dihydrate form of quercetin can be used.

The structure of quercetin is shown below (formula XXXVI):

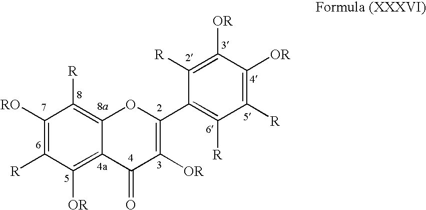

wherein each OR is an OH (i.e., 3-OH, 5-OH, 7-OH, 3′-OH, and 4′-OH) and each R is an H. Some embodiments of the invention comprise a derivative of quercetin. For example, derivatives of quercetin comprise compositions of formula II, wherein each R can be independently selected from the group consisting of hydrogen, optionally substituted C₁-C₁₀ alkyl, optionally substituted aryl, optionally substituted C₁-C₁₀ aliphatic acyl, optionally substituted C₁-C₁₀ aromatic acyl, trialkylsilyl, optionally substituted ether, carbohydrate, substituted carbohydrate, amino acid and substituted amino acid; and its pharmaceutically acceptable salts, esters, prod rugs, analogs, isomers, stereoisomers or tautomers thereof. In some embodiments, metabolites of quercetin, e.g., quercetin 3-O-glucouronide, are used.

In some embodiments, the quercetin is in a carbohydrate-derivatized form, e.g., a quercetin-O-saccharide. Quercetin-O-saccharides useful in the invention include, but are not limited to, quercetin 3-O-glycoside, quercetin 3-O-glucorhamnoside, quercetin 3-O-galactoside, quercetin 3-O-xyloside, and quercetin 3-O-rhamnoside. In some embodiments, the invention utilizes a quercetin 7-O-saccharide.

In some embodiments, the invention utilizes a quercetin aglycone. In some embodiments, a combination of aglycones and carbohydrate-derivatized quercetins is used. It will be appreciated that the various forms of quercetin may have different properties useful in the compositions and methods of the invention, and that the route of administration can determine the choice of forms, or combinations of forms, used in the composition or method. Choice of a single form, or of combinations, is a matter of routine experimentation.

Thus, in some embodiments the invention features a composition or method utilizing a quercetin-sulfobutylether-7-β-cyclodextrin composition to reduce or eliminate one or more side effects of a substance, such as a therapeutic agent, e.g., an analgesic.

In compositions for oral delivery of quercetin, carbohydrate-derivatized forms (also referred to herein as “quercetin saccharides”) are used with sulfobutylether-7-β-cyclodextrin in some embodiments. Various combinations of carbohydrate-derivatized forms and/or aglycone forms may be used in some embodiments. In some embodiments, quercetin-3-O-glycoside is used with sulfobutylether-7-β-cyclodextrin in an oral preparation of quercetin; in some embodiments, a pharmaceutically acceptable excipient is included in the composition. In some embodiments, quercetin 3-O-glucorhamnoside is used with sulfobutylether-7-β-cyclodextrin in an oral preparation of quercetin; in some embodiments, a pharmaceutically acceptable excipient is included in the composition. Other carbohydrate-derivatized forms of quercetin, or other forms of quercetin which are derivatives as described above, can also be used, based on their oral bioavailability, their metabolism, their incidence of gastrointestinal, other side effects, and other factors known in the art. Determining the bioavailability of quercetin in the form of derivatives including aglycones and glycosides is a matter of routine experimentation. See, e.g., Graefe et al., J. Clin. Pharmacol. (2001) 451:492-499; Arts et al. (2004) Brit. J. Nutr. 91:841-847; Moon et al. (2001) Free Rad. Biol. Med. 30:1274-1285; Hollman et al. (1995) Am. J. Clin. Nutr. 62:1276-1282; Jenaelle et al. (2005) Nutr. J. 4:1, and Cermak et al. (2003) J. Nutr. 133: 2802-2807, all of which are incorporated by reference herein in their entirety.

For further description of carbohydrate derivatives of polyphenols of the invention and their use, see U.S. Patent Publication No. 2006/0111308, in particular paragraphs [103]-[122] and PCT Publication No. WO0655672, in particular paragraphs [90]-[108], incorporated herein by reference.

In some embodiments of the invention, phosphorylated forms of flavonoids are used. As used herein, a compound that is “phosphorylated” includes a compound that has one or more phosphate groups covalently bound to it. In some embodiments, phosphorylation refers to the conversion of an OH group on a flavonoid to a phosphate group such as —OPO₃XY or —OPO₃Z group where X and Y can be hydrogen, an alkyl (such as methyl or ethyl), a carbohydrate, or a cation, and where Z is a multivalent cation. The chemistry for conversion of OH groups to phosphate groups is well known in the art and can be accomplished for example by reaction with phosphoric acid (see e.g. Organic Letters, 7(10), (2005), 1999-2002). In other embodiments, phosphorylation will involve the conversion of an H group or other group bound directly to a carbon to a phosphate group such as —OPO₃XY or —OPO₃Z group where X and Y can be hydrogen, an alkyl (such as methyl or ethyl), a carbohydrate, or a cation, and where Z is a multivalent cation. The phosphate group can also be referred to as a phosphonoxy group. Some phosphorylated flavonoids useful in the present invention are described in WO 93/09786, JP 01308476, and JP 01153695. In some cases, the phosphorylated compound will have a cyclic phosphate structure, such as a 5 membered ring that is formed when the phosphorous of the phosphate bridges two hydroxyl groups on adjacent carbons.

In some cases the phosphorylated flavonoids of the invention comprise polyphosphate derivatives. Polyphosphate derivatives are those in which more than one phosphate is connected in a linear chain. Suitable polyphosphate derivatives include, for example, diphosphates (pyrophosphates), and triphosphates.

“Phosphorylation” as used herein includes the addition of a sugar phosphate to the flavonoid. For example phosphorylation could be the addition of an inositol phosphate group. The addition of a sugar phosphate group to flavonoids is described in WO 96/21440.

In some embodiments, the flavonoids are derivatized with amino acid substituents. When R is substituted hydroxyl or substituted amine, substituents comprise both natural and unnatural amino acid moieties including, for example, glycine, dimethyl glycine, alanine, sarcosine, asparagine and arginine.

In some of these embodiments, a pharmaceutically acceptable excipient in addition to the cyclodextrin is also included.

### Cyclodextrins

The methods and compositions of the present invention involve cyclodextrins. Cyclodextrins and their derivatives can be used in liquid formulations to enhance the aqueous solubility of hydrophobic compounds. Cyclodextrins are cyclic carbohydrates derived from starch. The unmodified cyclodextrins differ by the number of glucopyranose units joined together in the cylindrical structure. The parent cyclodextrins typically contain 6, 7, or 8 glucopyranose units and are referred to as alpha-, beta-, and gamma-cyclodextrin respectively. Each cyclodextrin subunit has secondary hydroxyl groups at the 2 and 3-positions and a primary hydroxyl group at the 6-position. The cyclodextrins may be pictured as hollow truncated cones with hydrophilic exterior surfaces and hydrophobic interior cavities. In aqueous solutions, these hydrophobic cavities can incorporate hydrophobic organic compounds, which can fit all, or part of their structure into these cavities. This process, sometimes referred to as inclusion complexation, may result in increased apparent aqueous solubility and stability for the complexed drug. The complex is stabilized by hydrophobic interactions and does not generally involve the formation of any covalent bonds.

Cyclodextrins can be derivatized to improve their properties. Cyclodextrin derivatives that are particularly useful for pharmaceutical applications include the hydroxypropyl derivatives of alpha-, beta- and gamma-cyclodextrin, sulfoalkylether cyclodextrins such as sulfobutylether beta-cyclodextrin, alkylated cyclodextrins such as the randomly methylated beta.-cyclodextrin, and various branched cyclodextrins such as glucosyl- and maltosyl-beta.-cyclodextrin. Chemical modification of the parent cyclodextrins (usually at the hydroxyl moieties) has resulted in derivatives with sometimes improved safety while retaining or improving the complexation ability of the cyclodextrin. The chemical modifications, such as sulfoalkyl ether and hydroxypropyl, can result in rendering the cyclodextrins amorphous rather than crystalline, leading to improved solubility.

Particularly useful cyclodextrins for the present invention are the sulfoalkyl ether derivatives. The sulfoalkyl ether—CDs are a class of negatively charged cyclodextrins, which vary in the nature of the alkyl spacer, the salt form, the degree of substitution and the starting parent cyclodextrin. A particularly useful form of cyclodextrin is sulfobutylether-7-β-cyclodextrin, which is available under the trade name Captisol™ from CyDex, Inc. which has an average of about 7 substituents per cyclodextrin molecule. The anionic sulfobutyl ether substituents improve the aqueous solubility of the parent cyclodextrin. Reversible, non-covalent, complexation of flavonoids with the sulfobutylether-7-β-cyclodextrin can provide for increased solubility and stability in aqueous solutions.

The methods of the present invention provide for the formation of flavonoid-cyclodextrin compositions using, for example, the cyclodextrins and flavonoids described herein. In some embodiments, the methods of the invention provide for the preparation of flavonoid-cyclodextrin compositions where the flavonoid has a higher solubility in water than for a flavonoid-cyclodextrin composition formed without using the method of the invention.

### IV. Pyrone Analog-Sulfoalkyl Ether Cyclodextrin Such as Flavonoid Sulfoalkyl Ether Cyclodextrin Aqueous Compositions

The methods of the present invention can in some embodiments lead to compositions. In some embodiments, the compositions are aqueous solutions comprising a pyrone analog such as a flavonoid at a higher concentration than previously obtained. In some embodiments the invention comprises a composition with a pyrone analog such as a flavonoid and a sulfo-alkyl ether substituted cyclodextrin prepared by a process of mixing the cyclodextrin and the pyrone analog such as a flavonoid at a pH greater than about 11 and subsequently lowering the pH to less than about 9 where the concentration of the pyrone analog such as a flavonoid is higher than that obtained without using the process. In some embodiments the invention comprises a composition with a pyrone analog such as a flavonoid and a sulfo-alkyl ether substituted cyclodextrin prepared by a process of mixing the cyclodextrin and the pyrone analog such as a flavonoid at a pH greater than about 12 and subsequently lowering the pH to less than about 9 where the concentration of the pyrone analog such as a flavonoid is higher than that obtained without using the process. In some embodiments the invention comprises a composition with a pyrone analog such as a flavonoid and a sulfo-alkyl ether substituted cyclodextrin prepared by a process of mixing the cyclodextrin and the pyrone analog such as a flavonoid at a pH greater than about 11 and subsequently lowering the pH to less than about 8.5 where the concentration of the pyrone analog such as a flavonoid is higher than that obtained without using the process.

In some embodiments the invention allows for aqueous compositions in which the concentration of the pyrone analog such as a flavonoid is high at a pH below pH 9.

In some embodiments, the invention provides a composition comprising a pyrone analog such as a flavonoid and a sulfo-alkyl ether substituted cyclodextrin and an aqueous carrier wherein the pyrone analog such as a flavonoid is present in a concentration greater than 0.5 mM, 1 mM, 5 mM, 10 mM, 20 mM, 30 mM, 33 mM, 40 mM, 50 mM, 60 mM, 70 mM, 80 mM or greater than 80 mM. In some embodiments, the invention provides a composition comprising a pyrone analog such as a flavonoid and a sulfobutylether-7-β-cyclodextrin and an aqueous carrier wherein the pyrone analog such as a flavonoid is present in a concentration greater than 0.5 mM, 1 mM, 5 mM, 10 mM, 20 mM, 30 mM, 33 mM, 40 mM, 50 mM, 60 mM, 70 mM, 80 mM or greater than 80 mM. In some embodiments, the invention provides a composition comprising a quercetin or a quercetin derivative and a sulfobutylether-7-β-cyclodextrin and an aqueous carrier wherein the quercetin or a quercetin derivative is present in a concentration greater than 0.5 mM, 1 mM, 5 mM, 10 mM, 20 mM, 30 mM, 33 mM, 40 mM, 50 mM, 60 mM, 70 mM, 80 mM or greater than 80 mM.

In some embodiments, the invention provides a composition comprising a quercetin or a quercetin derivative and a sulfobutylether-7-β-cyclodextrin and an aqueous carrier wherein the quercetin or a quercetin derivative is present in a concentration greater than 0.5 mM. In some embodiments, the invention provides a composition comprising a quercetin or a quercetin derivative and a sulfobutylether-7-β-cyclodextrin and an aqueous carrier wherein the quercetin or a quercetin derivative is present in a concentration greater than 1 mM. In some embodiments, the invention provides a composition comprising a quercetin or a quercetin derivative and a sulfobutylether-7-β-cyclodextrin and an aqueous carrier wherein the quercetin or a quercetin derivative is present in a concentration greater than 5 mM. In some embodiments, the invention provides a composition comprising a quercetin or a quercetin derivative and a sulfobutylether-7-β-cyclodextrin and an aqueous carrier wherein the quercetin or a quercetin derivative is present in a concentration greater than 10 mM. In some embodiments, the invention provides a composition comprising a quercetin or a quercetin derivative and a sulfobutylether-7-β-cyclodextrin and an aqueous carrier wherein the quercetin or a quercetin derivative is present in a concentration greater than 20 mM. In some embodiments, the invention provides a composition comprising a quercetin or a quercetin derivative and a sulfobutylether-7-β-cyclodextrin and an aqueous carrier wherein the quercetin or a quercetin derivative is present in a concentration greater than 33 mM. In some embodiments, the invention provides a composition comprising a quercetin or a quercetin derivative and a sulfobutylether-7-β-cyclodextrin and an aqueous carrier wherein the quercetin or a quercetin derivative is present in a concentration greater than 40 mM. In some embodiments, the invention provides a composition comprising a quercetin or a quercetin derivative and a sulfobutylether-7-β-cyclodextrin and an aqueous carrier wherein the quercetin or a quercetin derivative is present in a concentration greater than 50 mM. In some embodiments, the invention provides a composition comprising a quercetin or a quercetin derivative and a sulfobutylether-7-β-cyclodextrin and an aqueous carrier wherein the quercetin or a quercetin derivative is present in a concentration greater than 60 mM. In some embodiments, the invention provides a composition comprising a quercetin or a quercetin derivative and a sulfobutylether-7-β-cyclodextrin and an aqueous carrier wherein the quercetin or a quercetin derivative is present in a concentration greater than 80 mM.

In some embodiments, the invention provides a composition comprising a pyrone analog such as a flavonoid and a sulfobutylether-7-β-cyclodextrin and an aqueous carrier wherein the pyrone analog such as a flavonoid is present in a concentration greater than 0.5 mM, 1 mM, 5 mM, 10 mM, 20 mM, 30 mM, 33 mM, 40 mM, 50 mM, 60 mM, 70 mM, 80 mM or greater than 80 mM, wherein the flavonoid is selected from the group consisting of quercetin or a quercetin derivative, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, 5,7-dideoxyquercetin (3,3′,4′-trihydroxyflavone), and epicatechin.

In the compositions of the invention, the molar ratio of pyrone analog such as a flavonoid, e.g. quercetin, to cyclodextrin, e.g. sulfobutylether-7-β-cyclodextrin is between 1:1 and 1:40. In some cases, the molar ratio of pyrone analog such as a flavonoid, e.g. quercetin, to cyclodextrin, e.g. sulfobutylether-7-β-cyclodextrin is between 1:1 and 1:20. In some cases, the molar ratio of pyrone analog such as a flavonoid, e.g. quercetin, to cyclodextrin, e.g. sulfobutylether-7-β-cyclodextrin is between 1:1 and 1:10. In some cases, the molar ratio of pyrone analog such as a flavonoid, e.g. quercetin, to cyclodextrin, e.g. sulfobutylether-7-β-cyclodextrin is between 1:1 and 1:5. In some cases, the molar ratio of pyrone analog such as a flavonoid, e.g. quercetin, to cyclodextrin, e.g. sulfobutylether-7-β-cyclodextrin is between 1:2 and 1:5. In some cases, the molar ratio of pyrone analog such as a flavonoid, e.g. quercetin, to cyclodextrin, e.g. sulfobutylether-7-β-cyclodextrin is between 1:10 and 1:40. In some cases, the molar ratio of pyrone analog such as a flavonoid, e.g. quercetin, to cyclodextrin, e.g. sulfobutylether-7-β-cyclodextrin is between 1:15 and 1:40. In some cases, the molar ratio of pyrone analog such as a flavonoid, e.g. quercetin, to cyclodextrin, e.g. sulfobutylether-7-β-cyclodextrin is between 1:3 and 1:12. In some cases, the molar ratio of pyrone analog such as a flavonoid, e.g. quercetin, to cyclodextrin, e.g. sulfobutylether-7-β-cyclodextrin is between 1:5 and 1:10.

The compositions of the invention can be pharmaceutical compositions. It can be desirable to have a pharmaceutical with a high concentration of active ingredient so that the active ingredient can be delivered without having to deliver a large amount of solution to the patient. In some embodiments, the pyrone analog such as a flavonoid and a sulfo-alkyl ether substituted cyclodextrin and an aqueous carrier wherein the pyrone analog such as a flavonoid is present in a concentration greater than 0.5 mM, 1 mM, 5 mM, 10 mM, 20 mM, 30 mM, 33 mM, 40 mM, 50 mM, 60 mM, 70 mM, 80 mM or greater than 80 mM is a pharmaceutical composition. The composition comprising a relatively high concentration of pyrone analog such as a flavonoid can be used for any suitable type of administration described herein. In some embodiments, such pharmaceutical composition is for oral administration. In some embodiments, such pharmaceutical composition is for intravenous administration.

Another aspect of the invention is an aqueous composition comprising a pyrone analog such as a flavonoid, a cyclodextrin, and a basic amino acid or sugar-amine. It has been found that the basic amino acid, such as lysine and arginine, or the sugar-amine, such as meglumine, can act, along with the cyclodextrin, to increase the solubility of the pyrone analog such as a flavonoid in water.

The composition comprising the pyrone analog such as a flavonoid such as quercetin or a quercetin derivative, cyclodextrin and a basic amino acid or sugar-amine, can comprise an aqueous solution. In some cases the cyclodextrin is present between 10% w/v to 40% w/v in the aqueous solution. In some cases the cyclodextrin is present between 15% and 35%. In some cases the cyclodextrin is present between 20% and 35%. In some cases the cyclodextrin is present between 20% and 35%. In some cases the cyclodextrin is present between 25% and 35%. In some cases the cyclodextrin is present between 30% and 35%. In some cases the cyclodextrin is present at about 10%, about 12%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 38% and about 40% w/v in the aqueous solution. In some cases the cyclodextrin is present in a range of 10% to 15%, 15% to 20%, 20% to 25%, 25% to 30%, 30% to 35%, or 35% to 40% w/v in the aqueous solution. In some cases it is found that having a level of cyclodextrin greater than about 20%, greater than about 25%, or greater than about 30% w/v in the aqueous solution can be used to obtain high solubility of the pyrone analog such as a flavonoid. The cyclodextrin that works in this range can be, for example, a sulfoalkyl cyclodextrins such as sulfobutylether-β-cyclodextrin.

The pyrone analog such as a flavonoid in the composition comprising cyclodextrin and a basic amino acid or sugar-amine is any suitable pyrone analog such as a flavonoid, for example, those known and/or described herein. The pyrone analog such as a flavonoid can be, for example, quercetin or a quercetin derivative, galangin, fisetin, or kaempferol. Where the composition comprises an aqueous solution, the composition can comprise a pyrone analog such as a flavonoid, e.g. quercetin or a quercetin derivative at a concentration in a range between 1 mg/mL and 15 mg/mL, between 3 mg/mL and 14 mg/mL, between 5 mg/mL and 13 mg/mL, between 6 mg/mL and 12 mg/mL, between 8 mg/mL and 12 mg/mL, or between 9 mg/mL and 11 mg/mL. In some cases, the composition comprises the pyrone analog such as a flavonoid, e.g. quercetin or a quercetin derivative at a concentration of greater than 1 mg/mL, greater than 2 mg/mL, greater than 4 mg/mL, greater than 3 mg/mL, greater than 5 mg/mL, greater than 6 mg/mL, greater than 7 mg/mL, greater than 8 mg/mL, greater than 9 mg/mL, greater than 10 mg/mL, greater than 11 mg/mL, greater than 12 mg/mL, greater than 13 mg/mL, greater than 14 mg/mL, or greater than 15 mg/mL. In all of the amounts and ranges described above, the pyrone analog such as a flavonoid can be quercetin or a quercetin derivative.

In some cases, in the composition comprising the pyrone analog such as a flavonoid, cyclodextrin and basic amino acid or sugar-amine, the pyrone analog such as a flavonoid e.g. quercetin is present at a concentration of greater than about 3 mM, greater than about 6 mM, greater than about 9 mM, greater than about 12 mM, greater than about 15 mM, greater than about 18 mM, greater than about 21 mM, greater than about 24 mM, greater than about 27 mM, greater than about 30 mM, greater than about 36 mM, greater than about 40 mM, or greater than about 45 mM. In some cases, the concentration of the pyrone analog such as a flavonoid, e.g. quercetin is between about 6 mM to about 36 mM. In some cases, the concentration of the pyrone analog such as a flavonoid, e.g. quercetin is between about 8 mM to about 30 mM. In some cases, the concentration of the pyrone analog such as a flavonoid, e.g. quercetin is between about 9 mM to about 30 mM. In some cases, the concentration of the pyrone analog such as a flavonoid, e.g. quercetin is between about 12 mM to about 18 mM. In some cases, the concentration of the pyrone analog such as a flavonoid, e.g. quercetin is between about 24 mM to about 33 mM. In some cases, the concentration of the pyrone analog such as a flavonoid, e.g. quercetin is between about 27 mM to about 30 mM.

In the compositions of the invention, the basic amino acid generally has a basic group (in addition to the amine of the amino acid). The basic group can be, for example, an amine group or a guanidinium group. The pKa of the basic group will generally be greater than about 8.5, greater than about 9.5, greater than about 10, greater than about 10.5, greater than about 11, or greater than about 11.5. The pKa of the basic group can be between about 9.5 and about 12, between about 10 and about 11.5, or between about 10.5 and 11.5. The pKa of the basic group can be about 9.5, about 10, about 10.5, about 11, about 11.5, or about 12. The amino acid can be a naturally occurring amino acid or a synthetic amino acid. In some cases it is desirable to use a naturally occurring basic amino acid in a pharmaceutical formulation. In some cases lysine is the amino acid. In some cases arginine is the amino acid. In some cases, both lysine and arginine are both in the composition.

In some embodiments the compositions of the invention comprise a pyrone analog such as a flavonoid such as quercetin or a quercetin derivative, a sulfobutylether-7-β-cyclodextrin, and a polyhydroxy amine or sugar-amine. For example, in some embodiments, a polyhydroxy compound having a basic group such as an amine, or a sugar having a basic group such as an amine group (a sugar amine) can be used. In some cases the sugar-amine can be 1-Deoxy-1-(methylamino)-D-galactitol, Deoxy-1-(octylamino)-D-glucitol, Deoxy-1-(2-hydroxyethylamino)-D-glucitol, Disorbitylamine, Galactosamine, Glucosamine, or Mannosamine. In some cases, for example, meglumine (N-Methyl-d-glucamine) can be used. While not being bound by theory, these compounds may provide salvation of the pyrone analogs such as flavonoids, e.g. quercetin in the presence of cyclodextrins e.g. sulfobutylether-β-cyclodextrin by having both a basic functional group which can assist in removing a proton from an acidic group on the pyrone analog such as a flavonoid, e.g. quercetin, and by having a hydrophilic portion (the polyhydroxy functionality) to assist in salvation with water.

The amount of the amino acid or sugar-amine in the composition can be the amount required to bring the pH of a solution above about 8.5, above about 8.7, or above about 9.0. Where the composition comprising the pyrone analog such as a flavonoid, e.g. quercetin, cyclodextrin, e.g. sulfobutylether-β-cyclodextrin and basic amino acid or sugar-amine comprises an aqueous solution, the amount of amino acid or sugar-amine can be, for example, between 10 mM and 200 mM, between 30 mM and 120 mM, between 40 mM and 100 mM, between 60 mM and 75 mM. In some cases, the amino acid is arginine present at a concentration between 50 mM and 90 mM. In some cases, the amino acid is arginine present at a concentration between 60 mM and 80 mM. In some cases, the amino acid is arginine present at a concentration of about 70 mM. In some cases, the amino acid is lysine present between about 50 mM and 80 mM. In some cases, the amino acid is lysine present between about 60 mM and 70 mM. In some cases, the amino acid is lysine present at about 65 mM. In some cases a sugar-acid is used rather than or in addition to the amino acid. In some cases the sugar-acid is meglumine which is present between 30 mM and 60 mM. In some cases the sugar-acid is meglumine which is present between 40 mM and 50 mM. In some cases the sugar-acid is meglumine which is present at about 44 mM.

In some embodiments, the composition is a neutralized solution. The neutralized solution can be formed by adding an acid to a basic solution comprising the pyrone analog such as a flavonoid, cyclodextrin, and basic amino acid or sugar-amine. The neutralized solution is generally brought to below pH 8.5. In some cases, the pH of the neutralized solution is between 5 and 8.5, between 6 and 8.5, between 7 and 8.5, between 7 and 8, or between 7.5 and 8. In some cases, the pH of the neutralized solution is 8.5, 8.4, 8.3, 8.2, 8.1, 8.0, 7.9, 7.8, 7.7, 7.6, 7.5, 7.4, 7.3, 7.2, 7.1, or 7.0. In some cases, the pH of the neutralized solution is about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, or about 7.0.

In some cases, the composition comprises an aqueous solution comprising quercetin or a quercetin derivative at about 4 mg/mL to about 12 mg/mL, sulfobutylether-β-cyclodextrin at about 15% w/v to about 35% w/v, and lysine at about 40 mM to about 90 mM. In some cases, the composition comprises an aqueous solution comprising quercetin or a quercetin derivative at about 4 mg/mL to about 12 mg/mL, sulfobutylether-β-cyclodextrin at about 15% w/v to about 35% w/v, and arginine at about 40 mM to about 90 mM. In some cases, the composition comprises an aqueous solution comprising quercetin or a quercetin derivative at about 4 mg/mL to about 12 mg/mL, sulfobutylether-β-cyclodextrin at about 15% w/v to about 35% w/v, and meglumine at about 20 mM to about 60 mM. In some cases, the composition comprises an aqueous solution comprising quercetin or a quercetin derivative at about 5 mg/mL to about 10 mg/mL, sulfobutylether-β-cyclodextrin at about 20% w/v to about 30% w/v, and lysine at about 50 mM to about 80 mM. In some cases, the composition comprises an aqueous solution comprising quercetin or a quercetin derivative at about 5 mg/mL to about 10 mg/mL, sulfobutylether-β-cyclodextrin at about 15% w/v to about 30% w/v, and arginine at about 40 mM to about 90 mM. In some cases, the composition comprises an aqueous solution comprising quercetin or a quercetin derivative at about 4 mg/mL to about 12 mg/mL, sulfobutylether-β-cyclodextrin at about 15% w/v to about 30% w/v, and meglumine at about 20 mM to about 60 mM. In some cases, the composition comprises an aqueous solution comprising quercetin or a quercetin derivative at about 10 mg/mL, sulfobutylether-β-cyclodextrin at about 30% w/v, and lysine at about 65 mM. In some cases, the composition comprises an aqueous solution comprising quercetin or a quercetin derivative at about 10 mg/mL, sulfobutylether-β-cyclodextrin at about 30% w/v, and arginine at about 70 mM. In some cases, the composition comprises an aqueous solution comprising quercetin or a quercetin derivative at about 10 mg/mL, sulfobutylether-β-cyclodextrin at about 30% w/v, and meglumine at about 44 mM.

One aspect of the invention is a dry powder formulation comprising the pyrone analog such as a flavonoid such as quercetin or a quercetin derivative, the cyclodextrin such as sulfobutylether-β-cyclodextrin, and the basic amino acid or sugar-amine. In some cases, in the dry powder formulation, the molar ratio of the pyrone analog such as a flavonoid, e.g. quercetin to the basic amino acid or sugar-amine is from about 3:1 to about 1:9. In some cases the molar ratio of the pyrone analog such as a flavonoid, e.g. quercetin to the basic amino acid or sugar-amine is from about 1:1 to about 1:5. In some cases the molar ratio of the pyrone analog such as a flavonoid, e.g. quercetin to the basic amino acid or sugar-amine is about 1:2. In some cases the molar ratio of the pyrone analog such as a flavonoid, e.g. quercetin to the basic amino acid or sugar-amine is from about 1:1 to about 1:5 and the molar ratio of the pyrone analog such as a flavonoid to the cyclodextrin such as sulfobutylether-β-cyclodextrin is about 1:12 to 1:2.

In some cases the molar ratio of the pyrone analog such as a flavonoid, e.g. quercetin to the basic amino acid or sugar-amine is from about 3:1 to about 1:9 and the molar ratio of the pyrone analog such as a flavonoid to the cyclodextrin such as sulfobutylether-β-cyclodextrin is about 1:1 to 1:40. In some cases the molar ratio of the pyrone analog such as a flavonoid, e.g. quercetin to the basic amino acid or sugar-amine is from about 1:1 to about 1:5 and the molar ratio of the pyrone analog such as a flavonoid to the cyclodextrin such as sulfobutylether-β-cyclodextrin is about 1:3 to 1:12. In some cases the molar ratio of the pyrone analog such as a flavonoid, e.g. quercetin to the basic amino acid or sugar-amine is from about 1:1 to about 1:5 and the molar ratio of the pyrone analog such as a flavonoid to the cyclodextrin such as sulfobutylether-β-cyclodextrin is about 1:5 to 1:10. The dry powder can be stored, and can then be re-dissolved in water, for example to produce an intravenous solution. The dry powder can also be formulated as described below into a pharmaceutical formulation suitable for administration via various routes. The powder can be packaged into kits.

In some embodiments the solutions of pyrone analog such as a flavonoid produced by the above method are stable for a long period of time. In some embodiments, by using the methods of the invention, pyrone analog such as a flavonoid solutions at relatively high concentrations can be stable to precipitation for about 5, 10, 20, 30, 45, or 60 minutes, for about 1, 2, 4, 8, 10, 12, 18, or 24 hours, for about 1, 2, 3, 5, 7, or 10 days, for 1, 2, 3, 4, 6 weeks, or for 1, 2, 3, 6, 9, or 12 months or 1, 2 3 or more years. The term “soluble” as used herein means that the pyrone analog such as a flavonoid does not precipitate from the solution. In some embodiments, the soluble solution is substantially clear. In some embodiments the compositions can be stored at low temperature, e.g. refrigerated, for the time periods described above without precipitation. For example, a composition of this invention with quercetin at 10 mg/ml in water with sulfobutylether-7-β-cyclodextrin is stable for more than two weeks without precipitation of the quercetin.

In some cases the method allows for the production of pyrone analog-sulfoalkyl ether cyclodextrin such as flavonoid sulfoalkyl ether cyclodextrin aqueous compositions that have such a high concentrations that they tend to precipitate out of solution over time. For instance, the compositions may be clear and homogeneous for hours after their production by the methods of the invention, but will tend to precipitate after several hours at room temperature. These meta-stable high concentration solutions can still be useful, for instance if they are used within the time of solubility, or if they are further processed after having been produced at high concentration, for example being freeze-dried, or being diluted into formulations having long shelf life. It is known in the art how to characterize the stability of the fluids under various conditions to determine their usefulness for a given application.

### V. Pharmaceutical Formulations Made Using Flavonoid-Sulfoalkyl Ether Cyclodextrin Aqueous Compositions

The compositions of the present invention can be used to make pharmaceutical formulations. In embodiments where the formulations provide a high concentration of the pyrone analog such as a flavonoid in solution, these high concentration solutions can be useful for making pharmaceutical formulations. For example, in some embodiments, a composition with a high concentration of pyrone analog such as a flavonoid and sulfoalkyl ether cyclodextrin can be dried, for example by freeze-drying or lyophilization in order to form a solid, powdered composition for use in a pharmaceutical formulation. The dried powder can then formulated with other components to make a pharmaceutical formulation for any suitable type of administration. For example, in some embodiments the dried powder can be mixed with other ingredients to create an oral formulation. In other embodiments, the dried powder can be made into a solid formulation that can be stored and then subsequently dissolved to produce a pharmaceutical formulation for injection.

In some embodiments, the high concentration form of pyrone analog such as a flavonoid and sulfoalkyl ether cyclodextrin can be made as concentrated stock solution, and subsequently diluted for administration. It can be advantageous to have a high concentration stock solution for ease of manufacturing, storage, and handling.

In some embodiments, the invention provides a pharmaceutical composition that is made using an aqueous composition comprising a pyrone analog such as a flavonoid and a sulfo-alkyl ether substituted cyclodextrin and an aqueous carrier wherein the pyrone analog such as a flavonoid is present in a concentration greater than 0.5 mM, 1 mM, 5 mM, 10 mM, 20 mM, 30 mM, 33 mM, 40 mM, 50 mM, 60 mM, 70 mM, 80 mM or greater than 80 mM.

In some embodiments, the invention provides a pharmaceutical composition made from an aqueous composition comprising a pyrone analog such as a flavonoid and a sulfobutylether-7-β-cyclodextrin and an aqueous carrier wherein the pyrone analog such as a flavonoid is present in a concentration greater than 0.5 mM, 1 mM, 5 mM, 10 mM, 20 mM, 30 mM, 33 mM, 40 mM, 50 mM, 60 mM, 70 mM, 80 mM or greater than 80 mM. In some embodiments, the invention provides a composition comprising a quercetin or a quercetin derivative and a sulfobutylether-7-β-cyclodextrin and an aqueous carrier wherein the quercetin or a quercetin derivative is present in a concentration greater than 0.5 mM, 1 mM, 5 mM, 10 mM, 20 mM, 30 mM, 33 mM, 40 mM, 50 mM, 60 mM, 70 mM, 80 mM or greater than 80 mM.

In some embodiments, the invention provides a pharmaceutical composition made from an aqueous composition comprising a quercetin or a quercetin derivative and a sulfobutylether-7-β-cyclodextrin and an aqueous carrier wherein the quercetin or a quercetin derivative is present in a concentration greater than 0.5 mM. In some embodiments, the invention provides a pharmaceutical composition made from an aqueous composition comprising a quercetin or a quercetin derivative and a sulfobutylether-7-β-cyclodextrin and an aqueous carrier wherein the quercetin or a quercetin derivative is present in a concentration greater than 1 mM. In some embodiments, the invention provides a pharmaceutical composition made from an aqueous composition comprising a quercetin or a quercetin derivative and a sulfobutylether-7-β-cyclodextrin and an aqueous carrier wherein the quercetin or a quercetin derivative is present in a concentration greater than 5 mM. In some embodiments, the invention provides a pharmaceutical composition made from an aqueous composition comprising a quercetin or a quercetin derivative and a sulfobutylether-7-β-cyclodextrin and an aqueous carrier wherein the quercetin or a quercetin derivative is present in a concentration greater than 10 mM. In some embodiments, the invention provides a pharmaceutical composition made from an aqueous composition comprising a quercetin or a quercetin derivative and a sulfobutylether-7-β-cyclodextrin and an aqueous carrier wherein the quercetin or a quercetin derivative is present in a concentration greater than 20 mM. In some embodiments, the invention provides a pharmaceutical composition made from an aqueous composition comprising a quercetin or a quercetin derivative and a sulfobutylether-7-β-cyclodextrin and an aqueous carrier wherein the quercetin or a quercetin derivative is present in a concentration greater than 33 mM. In some embodiments, the invention provides a pharmaceutical composition made from an aqueous composition comprising a quercetin or a quercetin derivative and a sulfobutylether-7-β-cyclodextrin and an aqueous carrier wherein the quercetin or a quercetin derivative is present in a concentration greater than 40 mM. In some embodiments, the invention provides a pharmaceutical composition made from an aqueous composition comprising a quercetin or a quercetin derivative and a sulfobutylether-7-β-cyclodextrin and an aqueous carrier wherein the quercetin or a quercetin derivative is present in a concentration greater than 50 mM. In some embodiments, the invention provides a pharmaceutical composition made from an aqueous composition comprising a quercetin or a quercetin derivative and a sulfobutylether-7-β-cyclodextrin and an aqueous carrier wherein the quercetin or a quercetin derivative is present in a concentration greater than 60 mM. In some embodiments, the invention provides a pharmaceutical composition made from an aqueous composition comprising a quercetin or a quercetin derivative and a sulfobutylether-7-β-cyclodextrin and an aqueous carrier wherein the quercetin or a quercetin derivative is present in a concentration greater than 60 mM. In some embodiments, the invention provides a pharmaceutical composition made from an aqueous composition comprising a quercetin or a quercetin derivative and a sulfobutylether-7-β-cyclodextrin and an aqueous carrier wherein the quercetin or a quercetin derivative is present in a concentration greater than 80 mM.

In some embodiments, the invention provides a pharmaceutical composition made from an aqueous composition comprising a pyrone analog such as a flavonoid and a sulfo-alkyl ether substituted cyclodextrin and an aqueous carrier wherein the pyrone analog such as a flavonoid is present in a concentration greater than 0.5 mM, 1 mM, 5 mM, 10 mM, 20 mM, 30 mM, 33 mM, 40 mM, 50 mM, 60 mM, 70 mM, 80 mM or greater than 80 mM, wherein the flavonoid is selected from the group consisting of quercetin or a quercetin derivative, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, 5,7-dideoxyquercetin (3,3′,4′-trihydroxyflavone), and epicatechin.

In some embodiments, the invention provides a pharmaceutical composition made from an aqueous composition comprising a pyrone analog such as a flavonoid and a sulfobutylether-7-β-cyclodextrin and an aqueous carrier wherein the pyrone analog such as a flavonoid is present in a concentration greater than 0.5 mM, 1 mM, 5 mM, 10 mM, 20 mM, 30 mM, 33 mM, 40 mM, 50 mM, 60 mM, 70 mM, 80 mM or greater than 80 mM, wherein the flavonoid is selected from the group consisting of quercetin or a quercetin derivative, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, 5,7-dideoxyquercetin (3,3′,4′-trihydroxyflavone), and epicatechin.

In some embodiments, the invention provides a pharmaceutical composition made from an aqueous composition comprising a pyrone analog such as a flavonoid and a sulfo-alkyl ether substituted cyclodextrin and an aqueous carrier wherein the pyrone analog such as a flavonoid is present in a concentration greater than 0.5 mM, 1 mM, 5 mM, 10 mM, 20 mM, 30 mM, 33 mM, 40 mM, 50 mM, 60 mM, 70 mM, 80 mM or greater than 80 mM, wherein the administration is rectal, buccal, intranasal, transdermal, intravenous, intraperitoneal, parenteral, intramuscular, subcutaneous, orally, topical, as an inhalant, or via an impregnated or coated device such as a stent. In some embodiments, the invention provides pharmaceutical composition for intravenous administration made from an aqueous composition comprising a pyrone analog such as a flavonoid and a sulfo-alkyl ether substituted cyclodextrin and an aqueous carrier wherein the pyrone analog such as a flavonoid is present in a concentration greater than 0.5 mM, 1 mM, 5 mM, 10 mM, 20 mM, 30 mM, 33 mM, 40 mM, 50 mM, 60 mM, 70 mM, 80 mM or greater than 80 mM. In some embodiments, the pharmaceutical composition for intravenous administration is a solid. In some embodiments, the pharmaceutical composition for intravenous administration is made by removal of water, for example by freeze drying or lyophilization. In some embodiments the pharmaceutical composition for intravenous administration is a liquid.

The pharmaceutical formulation produced from the compositions can be processed and formulated as described herein.

VI. Methods of Reducing or Eliminating Side Effects of a Therapeutic Agent with a Composition Comprising a Flavonoid, and a Sulfoalkyl-Ether Cyclodextrin

In some embodiments, the invention provides a pyrone analog-sulfobutylether-7-β-cyclodextrin such as flavonoid-sulfobutylether-7-β-cyclodextrin composition that reduces or eliminates a side effect. In some embodiments, the invention provides compositions and methods utilizing a combination of a therapeutic agent and a pyrone analog-sulfobutylether-7-β-cyclodextrin such as flavonoid-sulfobutylether-7-β-cyclodextrin composition that acts as a side effect-decreasing agent that reduces or eliminates a side effect of the therapeutic agent. Typically, the side effect-decreasing agent is a modulator of a blood tissue barrier (BTB) such as exists at the blood brain barrier (BBB) or a placental barrier transport protein. The methods and compositions are useful in the treatment of an animal in need of treatment, where it is desired that one or more side effects of the substance, e.g., therapeutic agent, be reduced or eliminated. In embodiments further utilizing a therapeutic agent, the methods and compositions are useful in the treatment of an animal in need of treatment, where it is desired that one or more side effects of the therapeutic agent be reduced or eliminated while one or more of the therapeutic effects (e.g., peripheral effects) of the agent are retained or enhanced.

In some embodiments of the invention, the therapeutic agent is an analgesic agent, such as an opiate or a non-opiate analgesic. In some embodiments of the invention, the therapeutic agent is a non-analgesic agent. The pyrone analog-sulfobutylether-7-β-cyclodextrin such as flavonoid-sulfobutylether-7-β-cyclodextrin composition causing a decrease in side effects of the therapeutic agent, e.g., a modulator of a BTB, BBB, or placental barrier transport protein may be an activator or an inhibitor of the protein. The modulatory effect may be dose-dependent, e.g., some modulators act as activators in one dosage range and inhibitors in another. In some embodiments, a modulator of a BTB transport protein is used in a dosage wherein it acts primarily as an activator.

Typically, the use of the BTB protein modulator, e.g., activator, results in a decrease in one or more side effects of the therapeutic agent. The therapeutic effect(s) of the agent may be decreased, remain the same, or increase; however, in preferred embodiments, if the therapeutic effect is decreased, it is not decreased to the same degree as the side effect. It will be appreciated that a given therapeutic agent may have more than one therapeutic effect and or one or more side effect, and it is possible that the therapeutic ratio (in this case, the ratio of change in desired effect to change in undesired effect) may vary depending on which effect is measured. However, at least one therapeutic effect of the therapeutic agent is decreased to a lesser degree than at least one side effect of the therapeutic agent.

In addition, in some embodiments, one or more therapeutic effects of the agent is enhanced by use in combination with a pyrone analog-sulfobutylether-7-β-cyclodextrin such as flavonoid-sulfobutylether-7-β-cyclodextrin composition, while one or more side effect of the therapeutic agent is reduced or substantially eliminated. For example, in some embodiments, the analgesic effect of an analgesic agent is enhanced while one or more side effect of the agent is reduced or substantially eliminated.

Without being bound by theory, and as an example only of a possible mechanism, it is thought that the methods and compositions of the invention operate by reducing or eliminating the concentration of the therapeutic agent from a compartment such as the kidney, the pancreas, the liver, the CNS (e.g., brain) and/or fetal compartment, while retaining or even increasing the effective concentration of the agent in the circulation/periphery. Agents that act at least in part at peripheral targets may thus retain some or all of their activity, or even display enhanced therapeutic activity, while at the same time side are reduced or eliminated.

Without being bound by theory, it is believed that in the pyrone analog-sulfobutylether-7-β-cyclodextrin such as flavonoid-sulfobutylether-7-β-cyclodextrin formulations, some of the pyrone analog such as a flavonoid is bound within the hydrophobic cavity of the sulfobutylether-7-β-cyclodextrin in aqueous solution. The pyrone analog such as a flavonoid is believed to be bound in a reversible manner such that it can be delivered to the body while remaining in solution, then later released from the cyclodextrin to act on the body. In some cases the pyrone analog such as a flavonoid may remain bound to the cyclodextrin until it reaches the active region of the body (e.g. the blood brain barrier), then be released to act in the active site free of the cyclodextrin host. In some cases, pyrone analog such as a flavonoid may be released from the cyclodextrin in the body (e.g. in the bloodstream), and subsequently move through the body to the active site in an un-complexed form.

It will be appreciated that the therapeutic and/or side effects of an therapeutic agent may be mediated in part or in whole by one or metabolites of the therapeutic agent, and that a pyrone analog-sulfobutylether-7-β-cyclodextrin such as flavonoid-sulfobutylether-7-β-cyclodextrin composition that reduces or eliminates the kidney, islet cell, CNS or fetal concentration of the therapeutic agent and/or of one or active metabolites of the therapeutic agent that produce side effect, while retaining or enhancing a peripheral concentration of the therapeutic agent and/or one or more metabolites producing a therapeutic effect, is also encompassed by the methods and compositions of the invention. In addition, a pyrone analog-sulfobutylether-7-β-cyclodextrin such as flavonoid-sulfobutylether-7-β-cyclodextrin composition itself may be metabolized to metabolites that have differing activities in the modulation of one or more BTB transport receptors, and these metabolites are also encompassed by the compositions and methods of the invention.

Hence, in some embodiments the invention provides compositions that include a therapeutic agent and a pyrone analog-sulfobutylether-7-β-cyclodextrin such as flavonoid-sulfobutylether-7-β-cyclodextrin composition that acts as a BTB, where the therapeutic agent is present in an amount sufficient to exert a therapeutic effect and the BTB is present in an amount sufficient to decrease a side effect of the therapeutic agent when compared to the side effect without the BTB, for example BBB and/or placental modulator when the composition is administered to an animal. The decrease in the side effect can be measurable. The BTB transport protein modulator is a BTB protein activator in some embodiments. In some embodiments the BTB transport protein modulator is a modulator of ATP binding cassette (ABC) transport proteins. In some embodiments the BTB protein modulator is a modulator of P-glycoprotein (P-gP).

In some embodiments, compositions of the invention include one or more than one therapeutic agent as well as one or more than one pyrone analog-sulfobutylether-7-β-cyclodextrin such as flavonoid-sulfobutylether-7-β-cyclodextrin composition that acts as a BTB transport protein modulators. One or more of the therapeutic agents may have one or more side effects such as tissue specific adverse effects which are desired to be decreased.

Compositions of the invention may be prepared in any suitable form for administration to an animal. In some embodiments, the invention provides pharmaceutical compositions.

In some embodiments, the invention provides compositions suitable for oral administration. In some embodiments, compositions are suitable for transdermal administration. In some embodiments, compositions are suitable for injection by any suitable standard route of injection, e.g., intravenous, subcutaneous, intramuscular, or intraperitoneal. Compositions suitable for other routes of administration are also encompassed by the invention, as described herein. In some embodiments, the compositions of the invention provide for a higher concentration of aqueous soluble pyrone analog such as a flavonoid, which can be desirable for example, for an oral or intravenous formulation where a higher solubility can result in more effective delivery of the pyrone analog such as a flavonoid and increase its effectiveness.

Therapeutic agents of use in the invention include any suitable agent that produces a side effect that it is desired to reduce or eliminate, while retaining or enhancing a therapeutic effect of the agent. In some embodiments, the therapeutic agent is an analgesic agent. In some instances an effect, e.g., a side effect may be desirable in some cases and undesirable in others. For example, some analgesics also produce a sedating effect. In some instances, such a sedating effect may be desirable. For example, in the use of analgesics in terminal patients where the main object is to improve quality of the remaining period of life, a certain amount of sedation in addition to analgesia may be desirable. However, it is often desirable to decrease pain without altering mood or concentration, or with minimal alteration of mood or concentration. For example, in patients with chronic intractable pain who are otherwise in good health, it is often desired to achieve maximum alleviation of pain while having minimum sedation or effects on concentration. In the latter case, it is useful to decrease or eliminate the side effect of sedation while retaining the analgesic effect of the agent. It is within the invention to titrate the combination of dosage of therapeutic agent and of BTB and/or placental transport protein modulator in such a way as to obtain a ratio of therapeutic effect to side effect that is considered optimal. Thus, in some embodiments, one or more side effect of the therapeutic agent is reduced but not eliminated. In other embodiments, one or more side effects of the therapeutic agent is substantially eliminated. In some embodiments, the analgesic agent is an opiate. In some embodiments, the analgesic agent is a non-opiate.

In some embodiments the methods of the invention are used to reduce the side effect and/or increase the effectiveness of an immunosuppressant. The immunosuppressant can be a cyclosporin (Neoral, Sandimmune, SangCya), an azathioprine (Imuran), a corticosteroid such as prednisolone (Deltasone, Orasone), basiliximab (Simulect), daclizumab (Zenapax), muromonab CD3 (Orthoclone OKT3), tacrolimus (Prograf), ascomycin, pimecrolimus (Elidel), azathioprine (Imuran), cyclosporin (Sandimmune, Neoral), glatiramer acetate (Copaxone), mycopehnolate (CellCept), sirolimus (Rapamune), or voclosporin

In some embodiments methods of the invention are used to reduce the side effect and/or increase the effectiveness of a calcineurin inhibitor such as tacrolimus (Prograf),

The methods of the invention can be used to reduce the side effect and/or increase the effectiveness of a selective estrogen receptor modulator (SERM), such as tamoxifen.

The methods of the invention can be used to reduce the side effect and/or increase the effectiveness of an antilipedimic agent such as an HMG-CoA inhibitor such as lovastatin, simvastatin, pravastatin, fluvastatin, or atorvastatin

The methods of the invention can be used to reduce the side effect and/or increase the effectiveness of an antihyperglycemic agent (antiglycemic agent, hypoglycemic agent) such as glyburide, glipizide, gliclazide, or glimepride; a meglitinide such as repaglinide or netaglinide, a biguanide such as metformin, a thiazolidinedione, an α-glucosidase inhibitor such as acarbose or miglitol, glucagon, somatostatin, or diazoxide.

The methods of the invention can be used to reduce the side effect and/or increase the effectiveness of a cannabinoid.

The methods of the invention can be used to reduce the side effect and/or increase the effectiveness of an antidepressant. In some embodiments, antidepressants cause the side effects of high blood sugar and diabetes. The methods of the invention can be used, for example to reduce these side effects. In some embodiments the therapeutic agent is an antidepressant selected from the group of aripiprazone (Abilify), nefazodone (Serzone), escitalopram oxalate (Lexapro), sertraline (Zoloft), escitalopram (Lexapro), fluoxetine (Prozac), bupropion (Wellbutrin, Zyban), paroxetine (Paxil), venlafaxine (Effexor), trazodone (Desyrel), amitriptyline (Elavil), citalopram (Celexa), duloxetine (Cymbalta), mirtazapine (Remeron), nortriptyline (Pamelor), imipramine (Tofranil), amitriptyline (Elavil), clomipramine (Anafranil), doxepin (Adapin), trimipramine (Surmontil), amoxapine (Asenidin), desipramine (Norpramin), maprotiline (Ludiomil), protryptiline (Vivactil), citalopram (Celexa), fluvoxamine (Luvox), phenelzine (Nardil), trancylpromine (Parnate), selegiline (Eldepryl).

The methods of the invention can be used to reduce the side effect and/or increase the effectiveness of an antineuropathic agent such as gabapentin.

The methods of the invention can be used to reduce the side effect and/or increase the effectiveness of an anticonvulsant. In some cases, it can be an anticonvulsant that also has efficacy in the treatment of pain. The therapeutic agent can be, for example, acetazolamide (Diamox), carbamazepine (Tegretol), clobazam (Frisium), clonazepam (Klonopin/Rivotril), clorazepate (Tranxene-SD), diazepam (Valium), divalproex sodium (Depakote), ethosuximide (Zarontin), ethotoin (Peganone), felbamate (Felbatol), fosphenyloin (Cerebyx), gabapentin (Neurontin), lamotrigine (Lamictal), levetiracetam (Keppra), lorezepam (Ativan), mephenyloin (Mesantoin), metharbital (Gemonil), methsuximide (Celontin). Methazolamide (Neptazane), oxcarbazepine (Trileptal), phenobarbital, phenyloin (Dilantin/Epanutin), phensuximide (Milontin), pregabalin (Lyrica), primidone (Mysoline), sodium valproate (Epilim), stiripentol (Diacomit), tiagabine (Gabitril), topiramate (Topamax), trimethadione (Tridione), valproic acid (Depakene/Convulex), vigabatrin (Sabril), zonisamide (Zonegran), or cefepime hydrochloride (Maxipime).

In some embodiments the invention provides methods of treatment. In certain embodiments, the invention provides a method of treating a condition by administering to an animal suffering from the condition an effective amount of a therapeutic agent and an amount of a pyrone analog-sulfobutylether-7-β-cyclodextrin such as flavonoid-sulfobutylether-7-β-cyclodextrin composition acting as a BTB transport protein modulator, e.g., activator, sufficient to reduce or eliminate a side effect of the therapeutic agent. In some embodiments, the therapeutic agent is an analgesic agent, e.g., an opiate or a non-opiate analgesic. In certain embodiments the invention provides methods of treatment of pain, e.g., chronic pain, by administration of an analgesic, e.g., an opiate, without the development of tolerance and/or dependence to the analgesic, by co-administering a modulator of a pyrone analog-sulfobutylether-7-β-cyclodextrin such as flavonoid-sulfobutylether-7-β-cyclodextrin composition in combination with the analgesic, thereby preventing or delaying development of tolerance and/or dependence to the analgesic.

In some embodiments the invention provides methods of decreasing tissue residence and localized undesired side effect of an agent in an animal, e.g. a human, that has received an amount of the agent sufficient to produce a side effect by administering to the animal, e.g., human, an amount of a pyrone analog-sulfobutylether-7-β-cyclodextrin such as flavonoid-sulfobutylether-7-β-cyclodextrin composition modulator sufficient to reduce or eliminate the side effect such as side effect. In certain embodiments, the agent is an anesthetic, e.g., a general anesthetic. In certain embodiments, the agent is a therapeutic agent or drug of abuse that has be administered in excess, e.g., in an overdose.

A. Blood-Tissue Barrier

In some embodiments, the invention provides methods and compositions that modulate a blood tissue barrier (BTB) transport protein. BTB transport proteins play a role in the maintenance of barrier to foreign molecules and/or removal of substances from spaces (e.g. cells) by selectively permitting or reducing substance presence in the cell. The barrier can be a boundary between blood and a physiological compartment such as a cell, an organ, or a tissue. The barrier may be a cell membrane, a layer of cells, or a vascular structure. One example of such a barrier is the blood brain barrier, a capillary endothelial structure that regulates substrate entry and exit. In some embodiments, the polyphenol and/or its metabolite act as a modulator of a BTB transport protein on the endothelial cell. In some embodiments, the pyrone analog and/or its metabolite acts as a modulator of a BTB transport protein that is an ABC transport protein (see below). In some embodiments, the pyrone analog and/or its metabolite act as a BTB transport protein activator. In some embodiments, the pyrone analog and/or its metabolite is a modulator of P-gP, e.g., an activator of P-gP (see below).

1. Blood-Tissue Barrier Transporters

Without being limited by theory, it is thought that the compositions and methods of the invention operate by modulating transport of substrates across blood-tissue barriers, thus altering their concentration in one or more physiological compartments. There are many different types of BTB transporters, and it will be understood that compositions and methods of the invention may involve one or more than one BTB transporter. Other mechanisms may also be involved.

In some embodiments, the invention provides methods and compositions that modulate ATP Binding Cassette (ABC) transport proteins. ABC transport proteins is a superfamily of membrane transporters with similar structural features. These transport proteins are widely distributed in prokaryotic and eukaryotic cells. They are critical in the maintenance of barrier to foreign molecules and removal of waste from privileged spaces, and may be overexpressed in certain glial tumors conferring drug resistance to cytotoxic drugs. 48 members of the superfamily are described. There are 7 major subfamilies, which include ABC A-G. Subfamilies C, B, and G play a role in transport activity at blood brain barrier and blood-CSF barrier. ABC A substrates include lipids and cholesterol; ABC B transporters include P-glycoprotein (P-gP) and other multi drug resistance proteins (MRPs); ABC C contains MRP proteins; ABC E are expressed in ovary, testis and spleen; and ABC G contains breast cancer resistance protein (BCRP).

Other examples of blood-tissue barrier transporters that can be modulated by methods and compositions of the invention include organic anion transport systems (OAT), P-gP, and the GABA transporters—GAT-1 and GAT2/BGT-1. Substrate compounds for OATs include opiate peptides, including enkephalin and deltorphin II, anionic compounds, indomethacin, salicylic acid and cimetidine. OATs are inhibited by baclofen, tagamet, indomethacin, etc. and transport HVA (dopamine metabolite) and metabolites of norepinephrine, epinephrine, 5-HT3, and histamine.

GABA transporters are Na and Cl dependent, and are specific for GABA, taurine, β alanine, betaine, and nipecotic acid. GAT2 transporters are localized to abluminal and luminal surfaces of capillary endothelial cells. GAT-1 is localized to the outside of neurons and glia. GABA-transporter substrates include lorazepam, midazolam, diazepam, klonazepam and baclofen. Probenicid inhibits luminal membrane GABA transporters from capillary endothelial cells. GAT-1 is inhibited by Tiagabine.

### P-Glycoprotein

In some embodiments, the invention provides methods and compositions that modulate P-gP, e.g., that activate P-gP. P-gP, also known as ABCB 1, forms a protective barrier to pump away by excreting compounds into bile, urine, and intestinal lumen. Three isoforms have been identified in rodents (mdr1a, mdr1b, mdr2) and two in humans (MDR₁ and MDR₂). It is expressed in epithelium of the brain choroid plexus (which forms the blood cerebrospinal fluid barrier), as well as on the luminal surface of blood capillaries of the brain (blood-brain barrier) and other tissues known to have blood-tissue barriers, such as the placenta, the ovaries, and the testes.

In the brain, P-gP is expressed in multiple cell types within brain parenchyma including astrocytes and microglia and in luminal plasma membrane of capillary endothelium where it acts as a barrier to entry and efflux pump activity. P-gP transports a wide range of substrates out of cerebral endothelial cells into vascular lumen. P-gP is also expressed in the apical membrane of the choroid plexus and may transport substances into CSF.

P-gP substrates include molecules that tend to be lipophilic, planar molecules or uncharged or positively charged molecules. Non-limiting examples include organic cations, weak organic bases, organic anions and other uncharged compounds, including polypeptides and peptide derivatives, aldosterone, anthracyclines, colchicine, dexamethasone, digoxin, diltiazem, HIV protease inhibitors, loperamide, MTX, morphine, ondansetron, phenyloin and β-blockers. Inhibitors of P-gP include quinidine, verapamil, rifampin, PSC 833 (see Schinkel, J. Clin Invest., 1996, herein incorporated by reference in its entirety) cyclosporine A, carbamazepine, and amitryptiline.

Multi-drug resistance protein (MRP) substrates include acetaminophen glucuronide, protease inhibitors, methotrexate and ampicillin. Inhibitors of MRP include buthionine sulphoximine, an inhibitor of glutathione biosynthesis.

Breast Cancer Resistant Protein (BCRP)

BCRP, an ATP-driven transporter, is highly expressed, e.g., in the placenta. Allikmets R., et al., Cancer

Res. 58:5337-5339 (1998), herein incorporated by reference. BCRP is responsible for rendering tumor cells resistant to chemotherapeutic agents, such as topotecan, mitoxantrone, doxorubicin and daunorubicin. Allen J D, et al., Cancer Res. 59:4237-4241 (1999). BCRP has also been shown to restrict the passage of topotecan and mitoxantrone to the fetus in mice. Jonker J W et al., J. Natl. Cancer Inst. 92:1651-1656 (2000), herein incorporated by reference.

Monoamine Transporters

Monoamine transporters include serotonin transporter (SERT), norepinephrine transporter (NET) and the extraneuronal monoamine transporter (OCT3). Ramamoorthy S, et al., Placenta 14:449-461 (1993); Ramamoorthy S., et al., Biochem. 32:1346-1353 (1993); Kekuda R., et al., J. Biol. Chem. 273:15971-15979 (1998), all herein incorporated by reference.

Organic Cation Transporters

Organic Cation Transporters also exist, e.g., in the placenta. Placental Na+-driven organic cation transporter 2 (OCTN2) has been identified and localized to the basal membrane of the synctiotrophoblast. Wu X et al., J. Pharmacol. Exp. Ther. 290:1482-1492 (1999), herein incorporated by reference. Placental OCTN2 transports camitine across the placenta in the direction of the matemal-to-fetal transfer. Ohashi R., et al., J. Pharmacol. Exp. Ther. 291:778-784 (1999), herein incorporated by reference. Studies have identified methamphetamine, quinidine, verapamil, pyrilamine, desipramine, dimethylamiloride, cimetidine, and procainimide as drug substrates for OCTN2. Wu X, et al., Biochem. Biophys. Res. Commun. 246:589-595 (1998); Wu X, et al., Biochim. Biophys. Acta 1466:315-327 (2000), herein incorporated by reference.

Monocarboxylate Transporters and the Dicarboxylate Transporters

Another type of BTB transporters include monocarboxylate (MCT) and dicarboxylate (NaDC3 transporters. Both MCT (e.g. lactate transport) and NaDC3 (e.g. succinate transport), which utilize electrochemical gradients for transport, are localized to the brush border membrane of the placenta, with MCT being expressed in the basal membrane to a lesser extent. Price N T, et al., Biochem. J. 329:321-328 (1998); Ganaphthy V, et al., Biochem J. 249:179-184 (1988); Balkovetz D F, et al., 263:13823-13830 (1988), all incorporated by reference herein. Valproic acid, a teratogenic substance, may be a substrate for MCT transfer, and compete with lactate for transport across the placental barrier. Nakamura H. et al., Pharm. Res. 19:154-161 (2002), herein incorporated by reference.

Further information on exemplary transporters that can be modulated in embodiments of the methods and compositions of the invention are provided in Table 1 below.

TABLE 1
Active Transporters found, e.g. in the Blood-Brain Barrier.

| Active Transporter | Physiological Function in Blood-Brain Barrier | Exemplary Substrates |
|---|---|---|
| P-glycoprotein (P-gP) | Limits accumulation in KIDNEY, PANCREAS, | Loperamide, morphine, β endorphin, |
| | CNS of phospholipids, xenobiotics and other | phenytoin, elavil, depakote, cyclosporine, |
| | drugs; regulates absorption, distribution and | protease inhibitors, digoxin, calcium |
| | elimination of drug substances. | channel blockers, vinca alkaloids, |
| | | anthracyclines, ivermectin, aldosterone, |
| | | hydrocortisone, dexamethasone, taxanes, |
| | | domperidone, ondansetron |
| Multidrug Resistance | MRP family members mediate ATP dependent | Acetaminophen glucuronide, protease |
| (MRP) Protein Family | transport of unconjugated, amphillic anions, and | inhibitors, methotrexate, ampicillin |
| | lipophillic compounds conjugated to glutathione, | |
| | glucoronic acid, and sulfate; detoxification | |
| | function includes extrusion of leukotriene | |
| | metabolites; folate transport. | |
| GABA transporters (GAT- | GAT1 drives GABA into neurons; mediates | Lorazepam, midazolam, diazepam, |
| 1 and GAT-2, BGT-1) | clearance of GABA from the brain | klonazepam, baclofen |
| Organic Anion Transport | Limits thiopurine uptake; transports HVA | Opiate peptides, including enkephalin and |
| (OAT) Systems | (dopamine metabolite), and metabolites of | deltorphin II, anionic compounds, |
| | norepinephrine, epinephrine, serotonin and | indomethacin, salicylic acid, cimetide |
| | histamine | |

B. Blood Brain Barrier

Blood-tissue barriers may be illustrated by the blood brain barrier (BBB) and its mechanisms for controlling access to the CNS; however, it will be understood that the mechanisms described herein for the BBB are applicable, where appropriate, to other BTBs (especially in terms of transport proteins), and that the BBB is used as an illustrative example.

The access to the brain is controlled by at least two barriers, i.e., blood brain barrier (BBB) and blood-cerebrospinal fluid (CSF) barrier. As used herein, the term “blood brain-barrier” can encompass the blood-brain and blood-CSF barriers, unless otherwise indicated. The methods and compositions described herein are suitable for modulating the access of drugs and other substances into the brain. In some embodiments, the methods and compositions involve the modification of the blood brain barrier and/or blood-CSF barrier to prevent or reduce the entry of drugs into the central nervous system (CNS), e.g., by promoting efflux of the drugs from the CNS. In some embodiments, the compositions and methods of the invention utilize a modulator of a blood brain-barrier transport protein. In some embodiments, the compositions and methods of the invention utilize an activator of a blood brain-barrier transport protein.

The blood brain barrier regulates the transfer of substances between circulating blood and brain by facilitated transport and/or facilitated efflux. The interface on both luminal and abluminal surfaces contain physical and metabolic transporter components.

The exchange of substances between circulating blood and brain can be determined by evaluating octanol/H₂0 partition coefficient, facilitated transport, and/or facilitated efflux. The methods of measuring blood brain barrier integrity can be used to identify suitable central nervous system modulators for use in the methods and compositions described herein.

Various transporters exist to regulate rate of brain permeation for compounds with varying lipophilicity. Generally, hydrophilic nutrients, such as glucose and amino acids, are allowed entry into the physiological compartments of the methods and compositions disclosed herein. Conversely, compounds with low lipophilicity are pumped away from the physiological compartments by, for example, xenobiotic efflux transporters. These transporters are preferably modulated by the methods and compositions described herein to prevent entry of compounds and drugs into the central nervous system.

The blood CSF barrier is formed by the tight junctions of the epithelium of the choroid plexus and arachnoid membrane surrounding the brain and spinal cord. It is involved in micronutrient extraction, clearance of metabolic waste, and transport of drugs.

Mechanisms and routes of compounds into and out of brain include paracellular aqueous pathway for water soluble agents, transcellular lipophilic pathway for lipid soluble agents, transport proteins for glucose, amino acids, purines, etc., specific receptor mediated endocytosis for insulin, transferrin, etc., adsorptive endocytosis for albumin, other plasma proteins, etc., and transporters (e.g., blood-brain barrier transport proteins) such as P-glycoprotein (P-gP), multi-drug resistance proteins (MRP), organic anion transporter (OAT) efflux pumps, gamma-aminobutyric acid (GABA) transporters and other transporters that modulate transport of drugs and other xenobiotics. Methods and compositions of the invention may involve modulation of one or more of these transporters. Preferably, the central nervous system modulators affect one or more of these mechanisms and routes to extrude drugs from the central nervous system.

The methods and compositions described herein also modulate other barriers, such as neuronal transport barriers, as well as other barriers.

### Active Transporters

Another embodiment of the methods and compositions disclosed herein is use of modulators or therapeutic agents in manipulating active transport of drugs, chemicals and other substances across the placental barrier. Active transport across the placental barrier, as opposed to facilitated diffusion or passive transport, requires energy, usually in the form of adenosine triphosphate (ATP) or through energy stored in the transmembrane electrochemical gradient provided by Na⁺, Cl⁻ or H⁺. Because of the input of energy, active transport systems may work against a concentration gradient, however, saturation of the transporters can occur.

Extensive studies have been conducted regarding placental transport systems of nutrients, such as amino acids, vitamins and glucose. See Hahn T, et al., Early Pregnancy 2:168-182 (1996); Moe A J, Am. J. Physiol. 268:C1321-1331 (1995); Bissonnette J M, Mead Johnson Symp. Perinat. Dev. Med., 18:21-23 (1981), all incorporated herein by reference. Active transport of drugs occurs through the same transport systems, most likely due to structurally similarities between the transported drugs and endogenous substrates. Syme et al. (2004).

Active drug transporters are located either in the maternal-facing brush border (apical) membrane or the fetal-facing basolateral (basal) membrane where they pump drugs into or out of the synctiotrophoblast. Table 2 summarizes the active transporters that have been identified in the placenta.

TABLE 2
Active transporters found, e.g. in Placenta.

| Active Transporter | Physiological Function in Placenta | Exemplary Substrates |
|---|---|---|
| P-glycoprotein (P-gP) | Fetal-to-maternal transfer of hydrophobic | Digoxin, cyclosporine, saquinavir, |
| | cationic compounds | vincristine, vinblastine, paclitaxel, |
| | | dexamethasone, terfenadine, |
| | | sirolimus, quinidine, ondansetron, |
| | | loperamide |
| Multidrug resistance protein | Fetal-to-maternal transfer of glutathione, | Methotrexate, etoposide, vincristine, |
| 1 (MRP1) | sulfate and glucoronide conjugates (dianionic | cisplatin, vinblastine, HIV protease |
| | sulfated bile salts) | inhibitors |
| Multidrug resistance protein | Fetal-to-maternal transfer of glutathione, | Etoposide, cisplatin, doxorubicin, |
| 2 (MRP2) | sulfate and glucuronide conjugates (dianionic | vincristine, vinblastine, methotrexate, |
| | sulfated bile salts, bilirubin glucuronide, | paracetamol, glucuronide, |
| | estradiol glucuronide) | grepafloxacin, ampilicillin |
| Multidrug resistance protein | Fetal-to-maternal transfer of anionic | Methotrexate, etoposide |
| 3 (MRP3) | conjugates | |
| Breast cancer resistant | Unknown | Topotecan, mitoxantrone, |
| protein (BCRP) | | doxorubicin, daunorubicin |
| Serotonin transporter (SERT) | Serotonin transfer | Amphetamines |
| Norepinephrine transporter | Dopamine and norepinephrine transfer | Amphetamines |
| (NET) | | |
| Extraneuronal monoamine | Serotonin, dopamine, norepinephrine, | Amphetamines, imipramine, |
| transporter (OCT3) | histamine transfer | desipramine, clonidine, cimetidine |
| Organic cation transporters | Maternal-to-fetal transfer of carnitine | Metamphetamine, quinidine, |
| (OCTN) | | verapamil, pyrilamine |
| Monocarboxylate | Fetal-to-maternal transfer of lactate and | Valproic acid |
| transporters | pyruvate | |
| Dicarboxylate transporters | Maternal-to-fetal transfer of succinate and α- | Unknown |
| | ketoglutarate | |
| Sodium/multivitamin | Maternal-to-fetal transfer of biotin and | Carbamazepine, primidone |
| transporter (SMVT) | pantothenate | |

VII. Substances Whose Side Effects are Enhanced, or Whose Side Effects are Reduced when Combined with a Soluble Pyrone Analog—Therapeutic Agents

In one aspect, the invention provides compositions and methods to reduce or eliminate one or more side effects of a substance. The substance may be produced in the subject in a normal or abnormal condition (e.g., beta amyloid in Alzheimer's disease). The substance may be an agent that is introduced into an animal, e.g., a therapeutic agent (e.g., an analgesic for pain relief or an immunosuppressant to decrease rejection in organ transplant). It will be appreciated that some therapeutic agents are also agents produced naturally in an animal, and the two groups are not mutually exclusive. In some embodiments, the compositions and methods retain or enhance a desired effect of the substance, e.g., a peripheral effect. The methods and compositions of the invention apply to any therapeutic agent for which it is desired to reduce one or more side effects of the agent and/or enhance one or more of the therapeutic effects of the agent. In some embodiments, the compositions and methods of the invention utilize an analgesic agent. In some embodiments, the analgesic agent is an opiate analgesic. In some embodiments, the analgesic is a non-opiate analgesic. In some embodiments, the compositions and methods of the invention utilize a non-analgesic therapeutic agent, e.g., an immunosuppressant. It will be appreciated that there is some overlap between these groups, as some agents that have primarily an analgesic effect also have other therapeutic effects, while some agents that have primarily a non-analgesic effect also provide some degree of analgesia. The invention encompasses these therapeutic agents as well.

Hence, in some embodiments, the methods and compositions of the present invention can be used to modulate the effects of one or more of a variety of therapeutic agents. In some embodiments, the dosage of the therapeutic agent will be modulated according to the effect of the side effect modulator. For instance, less therapeutic agent may be needed to reach optimal effect when co-administered with the side effect modulator. In other embodiments co-administering the side effect modulator with a therapeutic agent will allow for chronically administering the drug without drug escalation and/or without dependence on the drug. In another embodiment co-administering the side effect modulator will allow for the elimination of a therapeutic agent from a physiological compartment, i.e. wash out drug in an overdose situation or to wake up a patient faster after anesthesia. In some embodiments, the physiological compartment is a central nervous system. In some embodiments, the physiological compartment is a fetal compartment.

The “side effect” of the therapeutic agent for which modulation is sought may be any effect associated with the agent that occurs in addition to the therapeutic effect. In some embodiments, the compositions and methods of the invention are used to decrease undesirable side effects and or increase desirable side effects or therapeutic effects of a therapeutic agent. Side effects are often specific to the agent, and are well-known in the art for various therapeutic agents. The effect may be acute or chronic. The effect may be biochemical, cellular, at the tissue level, at the organ level, at the multi-organ level, or at the level of the entire organism. The effect may manifest in one or more objective or subjective manners, any of which may be used to measure the effect.

An exemplary side effect, associated with many types of therapeutic agents, e.g., opiates, is a central nervous system (CNS) effect. The term “central nervous system (CNS) effect,” as used herein, encompasses any effect of a substance in the CNS. For some substances that may be normally or abnormally produced in the CNS, such as amyloid beta, the effect may be a pathological effect. In some embodiments, the side effect of a substance can be drowsiness, impaired concentration, sexual dysfunction, sleep disturbances, habituation, dependence, alteration of mood, respiratory depression, nausea, vomiting, lowered appetite, lassitude, lowered energy, dizziness, memory impairment, neuronal dysfunction, neuronal death, visual disturbances, impaired mentation, tolerance, addiction, hallucinations, lethargy, myoclonic jerking, or endocrinopathies, or combinations thereof.

Other exemplary side effects include hypogonadism (e.g., lowered testosterone) associated with some therapeutic agents, e.g., opiates, and hyperglycemia associated with immunosuppressants, e.g., tacrolimus. See U.S. published Patent Applications US2006/0111308 and US2008/0161248; and PCT published Patent Applications WO/06055672 and WO/08083160, all of which are incorporated by reference herein in their entirety.

A “therapeutic effect,” as that term is used herein, encompasses a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. A prophylactic effect includes delaying or eliminating the appearance of a disease or condition, delaying or eliminating the onset of symptoms of a disease or condition, slowing, halting, or reversing the progression of a disease or condition, or any suitable combination thereof.

The term “physiological compartment” as used herein includes physiological structures, such as organs or organ groups or the fetal compartment, or spaces whereby a physiological or chemical barrier exists to exclude compounds or agents from the internal portion of the physiological structure or space. Such physiological compartments include the central nervous system, the fetal compartment and internal structures contained within organs, such as the ovaries and testes.

Therapeutic agents that may be used in compositions and methods of the invention include analgesic agents, such as opiates, e.g. morphine, oxycodone, and the like, immunomodulators such as immunosuppressants, e.g., tacrolimus, cyclosporine, and the like, antineoplastics, amphetamines, antihypertensives, vasodilators, barbiturates, membrane stabilizers, cardiac stabilizers, glucocorticoids, chemotherapeutic agents, antiinfectives, tolerogen, immunostimulants, drug acting on the blood and the blood-forming organs, hematopoietic agent, growth factor, mineral, and vitamin, anticoagulant, thrombolytic, antiplatelet drug, hormone, hormone antagonist, pituitary hormone, thyroid and antithyroid drug, estrogen and progestin, androgen, adrenocorticotropic hormone; adrenocortical steroid and synthetic analogs, insulin, oral hypoglycemic agents, calcium, phosphate, parathyroid hormone, vitamin D, calcitonin, and other compounds. Therapeutic agents of use in the invention are further described in U.S. Patent Publication No. US2006/0111308, in particular at paragraphs [0123]-[0164]; and PCT Publication No. WO/06055672, in particular at paragraphs [00109]-[00145].

Thus compositions and methods of the invention encompass the use of one or more therapeutic agents in combination with a pyrone analog such as a flavonoid, such as quercetin, fisetin, or 5,7-dideoxyquercetin, that reduces a side effect of the therapeutic agent.

A. Analgesic Agents

The compositions and methods of the invention encompass the use of one or more analgesic agents in combination with a pyrone analog-sulfobutylether-7-β-cyclodextrin such as flavonoid-sulfobutylether-7-β-cyclodextrin composition that acts as an agent that reduces a side effect of the analgesic, such as a BTB transport protein modulator.

Analgesic agents are agents used to reduce or eliminate pain. An analgesic (colloquially known as painkiller) is any suitable member of the diverse group of drugs used to relieve pain and to achieve analgesia (“absence of pain”). Analgesic drugs act in various ways on the peripheral and central nervous system; analgesics may be employed for symptomatic relief and include broadly two major groups: 1) opiate analgesics; 2) nonopiate analgesics, including analgesics and antipyretics, nonsteroidal antiinflammatory drugs, acetominophen, paracetamol, indomethacin, tricyclic antidepressants (for example desipramine, imipramine, amytriptiline, nortriptile), anticonvulsants (for example, carbamazepine, valproate), and serotonin reuptake inhibitors (for example, fluoxetine, paraoxetine, sertraline), mixed serotonin-norepinephrine reuptake inhibitors (for example venlafaxine, duloxetine), serotonin receptor agonists and antagonists, cholinergic (muscarinic and nicotinic) analgesics, adrenergic agents, and neurokinin antagonists.

In one embodiment analgesic agents are selected from the group consisting of oxycodone, gabapentin, pregabalin, hydrocodone, fentanyl, hydromorphine, levorphenol, morphine, methadone, tramadol and topiramate.

1. Opiate Analgesics

In some embodiments of the invention utilizing an analgesic agent, the analgesic agent is an opiate. Opiates bind stereospecific receptors predominantly in the CNS and peripheral nervous system. The mu, kappa, and delta opiate receptors are the receptors most responsible for the analgesic effects. Mu activation produces analgesia but also has the usually undesired effects of respiratory depression, addiction, and euphoria. Kappa receptors are generally located in the spinal cord and help with spinal analgesia but also cause meiosis and sedation. Delta sites are also involved in analgesia. There is no ceiling effect with the analgesia provided by additional amounts of opiates. Thus side-effects also tend to increase with increasing dosage. Most common are gastrointestinal side-effects such as constipation, nausea and gastric distress. Sedation is also common.

Should the pain still prove debilitating, the clinician may choose to use stronger narcotics. Morphine is a pure agonist and makes for an excellent analgesic. Other mixed agonist/antagonist opiates, such as pantazocine, nalbuphine, and butorphanol, will selectively block mu receptors and activate kappa receptors. These drugs do exhibit a ceiling effect. Partial agonists act similarly by activating the mu receptor and block occupation of the kappa site.

Opioid alkaloids used in pain treatment and useful in embodiments of the invention include morphine (morphine sulfate), codeine, and thebaine. Semisynthetic derivatives include diamorphine (heroin), oxycodone, hydrocodone, dihydrocodeine, hydromorphone, oxymorphone, and nicomorphine. Synthetic opioids include phenylheptylamines such as methadone and levomethadyl acetate hydrochloride (LAAM); phenylpiperidines such as pethidine (meperidine), fentanyl, alfentanyl, sufentanil, remifentanil, ketobemidone, and carfentanyl; diphenylpropylamine derivatives such as propoxyphene, dextropropoxyphene, dextromoramide, bezitramide, and piritramide; benzomorphan derivatives such as pentazocine and phenazocine; oripavine derivatives such as buprenorphine; and morphinan derivatives such as butorphanol and nalbufine; and other opioids such as dezocine, etorphine, tilidine, tramadol, loperamide, nalbuphine, dextromethorphan, and diphenoxylate. Analgesic combinations that include opioids include analgesic combinations such as codeine/acetaminophen, codeine/aspirin, hydrocodone/acetaminophen, hydrocodone/ibuprofen, oxycodone/acetaminophen, oxycodone/aspirin, propoxyphene/aspirin or acetaminophen.

Opioid analgesics include, without limitation: alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene fentanyl, heroin, hydrocodone, hydromorphone, hydroxypethidine, isomethadone, ketobemidone, levallorphan, levomethadyl acetate hydrochloride (LAAM), levorphanol, levophenacyl morphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papavereturn, pentazocine, phenadoxone, phenomorphan, phenazocine, phenoperidine, piminodine, piritramide, propheptazine, promedol, properidine, propiram, propoxyphene, sufentanil, tramadol, tilidine, salts thereof and mixtures thereof.

In some embodiments, compositions and methods of the invention encompass the use of an opioid analgesic in combination with an agent that reduces a side effect of the opioid analgesic, such as a BTB transport protein modulator. In some embodiments, the opioid is oxycodone, hydrocodone, fentanyl, hydromorphine, levorphenol, morphine, methadone, or tramadol. In some embodiments, the opioid is oxycodone, hydrocodone, methadone, or tramadol. In some embodiments, the opioid is oxycodone. In some embodiments, the opioid is hydrocodone. In some embodiments, the opioid is methadone. In some embodiments, the opioid is tramadol.

2. Non-Opiate Analgesics

In some embodiments, the invention encompasses the use of a non-opiate analgesic. In some embodiments, the non-opiate analgesic is used in combination with a pyrone analog-sulfobutylether-7-β-cyclodextrin such as flavonoid-sulfobutylether-7-β-cyclodextrin composition that reduces a side effect of the non-opiate analgesic. In some embodiments, the non-opiate analgesic is used in addition to another analgesic, in combination with an agent that reduces a CNS side effect of the non-opiate analgesic and/or a side effect of the other analgesic.

Antidepressants and anticonvulsants In neuropathic and other opioid-insensitive pain conditions, antidepressants, e.g., tricyclic antidepressants (“TCAs”) and anticonvulsant therapy is typically used.

TCAs have been hypothesized to have their own analgesic effect, potentiate narcotics, and treat neuropathic pain as their modes of action for analgesia. Exemplary TCAs include Amitriptyline, Amoxapine, Clomipramine, Desipramine, Doxepin, Imipramine, Nortriptyline, Protriptyline, and Trimipramine.

In addition, other types of antidepressants may be used in treatment of, e.g., chronic pain. These include Escitalopram, Sertraline, Citalopram, Paroxetine, Paroxetin, controlled release, Fluoxetine, Venlafaxine; Reboxetine, Milnacipran, Mirtazapine, Nefazodone, Duloxetin Bupropion, Maprotiline, Mianserin, Trazodone, Dexmethylphenidate, Methyphenidate, and Amineptine, Fluoxetine weekly, Fluvoxamine, olanzapine/fluoxetine combination.

Anticonvulsants such as carbamazapine, topiramate, gabapentin, and pregabalin are used in neuropathic pains such as trigeminal neuralgia. Mexiletine and clonazepam have also been shown to be effective in other neuronally mediated types of pain. Further anticonvulsant agents include clorazepate dipotassium, diazepam, ethosuximide, ethotoin, felbamate, fosphenyloin, lamotrigine, levetiracetam, lorazepam, mephenyloin, mephobarbital, oxycarbazepine, pentobarbital sodium, phenobarbital, phenyloin, primidone, tiagabine, trimethadione, and valproic acid.

In some embodiments, compositions and methods of the invention encompass the use of an anticonvulsant in combination with a pyrone analog-sulfobutylether-7-β-cyclodextrin such as flavonoid-sulfobutylether-7-β-cyclodextrin composition that reduces a side effect of the anticonvulsant, such as a BTB transport protein modulator. In some embodiments, the anticonvulsant is gabapentin, pregabalin, or topiramate. In some embodiments, the anticonvulsant is gabapentin. In some embodiments, the anticonvulsant is pregabalin. In some embodiments, the anticonvulsant is topiramate.

Antiinflammatory compositions both steroidal and non-steroidal, also find use in pain relief, and may be used in the compositions and methods of the invention.

Non-limiting examples of steroidal anti-inflammatory agents suitable for use herein include corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof may be used. The preferred steroidal anti-inflammatory for use is hydrocortisone.

Additional nonopiate analgesics of use in the invention include the non-steroidal antiinflammatory compositions. NSAIDS are typically used as analgesics, antipyretics and anti-inflammatories. Acetaminophen, while not normally classified as an NSAID because it is not anti-inflammatory, has similar analgesic effects and is often used similarly. Salicylates are hydrolyzed by the body into salicylic acid whereas salicylamide and diflunisal have structural and functional similarities but do not get hydrolyzed. At sites of inflammation, NSAIDS typically inhibit prostaglandin synthesis by irreversibly acetylating cyclooxygenase and may inhibit nitric oxide synthetase, TNF-alpha, IL-1 and change other lymphocytic activity decreasing inflammation. Diclofenac, ibuprofen, indomethacin, and ketoprofen have been shown to have direct analgesic activity as well. Clinically, NSAIDs are typically used for mild to moderate pain, and are generally considered for some types of pain, most notably post-surgical pain, as being more effective than opioids.

NSAIDS used in pain treatment include salicylates such as aspirin, methyl salicylate, and diflunisal; arylalkanoic acids such as indomethacin, sulindac, diclofenac, and tolmetin; N-arylanthranilic acids (fenamic acids) such as mefenamic acid and mecflofenamate; oxicams such as piroxicam and meloxicam; coxibs such as celecoxib, rofecoxib, valdecoxib, parecoxib, and etoricoxib; sulphonanilides such as nimesulide; naphthylalkanones such as nabumetone; anthranilic acids such as pyrazolidinediones and phenylbutazone; proprionic acids such as fenoprofen, flurbiprofen, ibuprofen, ketoprofen, naproxen, and oxaprozin; pyranocarboxylic acids such as etodolac; pyrrolizine carboxylic acids such as ketorolac; and carboxylic acids.

Sedative-Hypnotic Drugs, may also be used, and include drugs that bind to the GABAA receptor such as the benzodiazepines (including alprazolam, chlordiazepoxide, clorazepate, clonazepam, diazepam, estazolam, flurazepam, halazepam, lorazepam, midazolam, oxazepam, quazepam, temazepam, triazolam), the barbiturates (such as amobarbital, pentobarbital, phenobarbital, secobarbita), and non-benzodiazepines (such as zolpidem and zaleplon), as well as the benzodiazepine antagonists (such as flumazenil). Other sedative-hypnotic drugs appear to work through non-GABA-ergic mechanisms such as through interaction with serotonin and dopaminergic receptors, and include buspirone, isapirone, geprirone, and tandospirone. Older drugs work through mechanisms that are not clearly elucidated, and include chloral hydrate, ethchlorvynol, meprobamate, and paraldehyde.

Ergot alkaloids are useful in the treatment of, e.g., migraine headache, and act on a variety of targets, including alpha adrenoceptors, serotonin receptors, and dopamine receptors. They include bromocriptine, cabergoline, pergolide, ergonovine, ergotamine, lysergic acid diethylamide, and methysergide. Available preparations include dihydroergotamine, ergonovine, ergotamine, ergotamine tartrate, and methylergonovine.

3. Other Pain-Reducing Modalities

In some embodiments, the compositions and methods of the invention encompass the use of an analgesic agent in combination with a pyrone analog-sulfobutylether-7-β-cyclodextrin such as flavonoid-sulfobutylether-7-β-cyclodextrin composition that acts as a modulator of a BTB transport protein, and further in combination with another pain-reducing modality. Treatment may also be by mechanical modalities of massage, ultrasound, stretching, traction, hydrotherapy or application of heat and cold. Electrical modalities of transcutaneous electrical nerve stimulation (TENS) or microcurrent electrical therapy (MET) might be used. Other therapies such as magnetic biostimulation, acupuncture, pulsed signal therapy, physical therapy, and electromedicine have all been used to treat pain conditions. Alternative and Eastern approaches have also been utilized. As part of a pain treatment or diagnosis plan, neural blockade by the introduction of local anesthetic or, rarely, a neurolytic can be used, usually combined with a steroid.

B. Non-Analgesic Agents

The methods and compositions of the invention are also useful in relation to non-analgesic therapeutic agents.

Therapeutic agents that may be used in compositions and methods of the invention include immunosuppressive agents, such as calcineurin inhibitors, e.g. tacrolimus, sirolimus, and the like, other immunomodulators, antineoplastics, amphetamines, antihypertensives, vasodilators, barbiturates, membrane stabilizers, cardiac stabilizers, glucocorticoids, antilipedimic, antiglycemics, cannabinoids, antidipressants, antineuroleptics, chemotherapeutic agents, antiinfectives, tolerogen, immunostimulants, drug acting on the blood and the blood-forming organs, hematopoietic agent, growth factor, mineral, and vitamin, anticoagulant, thrombolytic, antiplatelet drug, hormone, hormone antagonist, pituitary hormone, thyroid and antithyroid drug, estrogen and progestin, androgen, adrenocorticotropic hormone; adrenocortical steroid and synthetic analogs, insulin, oral hypoglycemic agents, calcium, phosphate, parathyroid hormone, vitamin D, calcitonin, and other compounds. Therapeutic agents of use in the invention are further described in U.S. Patent Publication No. US2006/0111308, in particular at paragraphs [0123]-[0164]; and PCT Publication No. WO/06055672, in particular at paragraphs [00109]-[00145].

In some embodiments the therapeutic agent whose side effect is reduced and/or whose effectiveness is improved in the presence of the phosphorylated pyrone analog is an immunosuppressant. The immunosuppressants can be a cyclosporin (Neoral, Sandimmune, SangCya), an azathioprine (Imuran), a corticosteroid such as prednisolone (Deltasone, Orasone), basiliximab (Simulect), daclizumab (Zenapax), muromonab CD3 (Orthoclone OKT3), tacrolimus (Prograf), ascomycin, pimecrolimus (Elidel), azathioprine (Imuran), cyclosporin (Sandimmune, Neoral), glatiramer acetate (Copaxone), mycopehnolate (CellCept), sirolimus (Rapamune), voclosporin

In some embodiments the therapeutic agent is a calcineurin inhibitor such as tacrolimus (Prograf),

The therapeutic agent can be a selective estrogen receptor modulator (SERM), such as tamoxifen.

The therapeutic agent can be an antilipedimic agent such as an HMG-CoA inhibitor such as lovastatin, simvastatin, pravastatin, fluvastatin, or atorvastatin

The therapeutic agent can be an antihyperglycemic agent (antiglycemic agent, hypoglycemic agent) such as glyburide, glipizide, gliclazide, or glimepride; a meglitinide such as repaglinide or netaglinide, a biguanide such as metformin, a thiazolidinedione, an α-glucosidase inhibitor such as acarbose or miglitol, glucagon, somatostatin, or diazoxide.

The therapeutic agent can be, in some embodiments, a cannabinoid.

The therapeutic agent can be an antidepressant. In some embodiments, antidepressants cause the side effects of high blood sugar and diabetes. The compounds and methods of the invention can be used, for example to reduce these side effects. In some embodiments the therapeutic agent is an antidepressant selected from the group of aripiprazone (Abilify), nefazodone (Serzone), escitalopram oxalate (Lexapro), sertraline (Zoloft), escitalopram (Lexapro), fluoxetine (Prozac), bupropion (Wellbutrin, Zyban), paroxetine (Paxil), venlafaxine (Effexor), trazodone (Desyrel), amitriptyline (Elavil), citalopram (Celexa), duloxetine (Cymbalta), mirtazapine (Remeron), nortriptyline (Pamelor), imipramine (Tofranil), amitriptyline (Elavil), clomipramine (Anafranil), doxepin (Adapin), trimipramine (Surmontil), amoxapine (Asenidin), desipramine (Norpramin), maprotiline (Ludiomil), protryptiline (Vivactil), citalopram (Celexa), fluvoxamine (Luvox), phenelzine (Nardil), trancylpromine (Pamate), selegiline (Eldepryl).

In some embodiments the therapeutic agent is an antineuropathic agent such as gabapentin.

The therapeutic agent can be an anticonvulsant. In some cases, it can be an anticonvulsant that also has efficacy in the treatment of pain. The therapeutic agent can be, for example, acetazolamide (Diamox), carbamazepine (Tegretol), clobazam (Frisium), clonazepam (Klonopin/Rivotril), clorazepate (Tranxene-SD), diazepam (Valium), divalproex sodium (Depakote), ethosuximide (Zarontin), ethotoin (Peganone), felbamate (Felbatol), fosphenyloin (Cerebyx), gabapentin (Neurontin), lamotrigine (Lamictal), levetiracetam (Keppra), lorezepam (Ativan), mephenyloin (Mesantoin), metharbital (Gemonil), methsuximide (Celontin). Methazolamide (Neptazane), oxcarbazepine (Trileptal), phenobarbital, phenyloin (Dilantin/Epanutin), phensuximide (Milontin), pregabalin (Lyrica), primidone (Mysoline), sodium valproate (Epilim), stiripentol (Diacomit), tiagabine (Gabitril), topiramate (Topamax), trimethadione (Tridione), valproic acid (Depakene/Convulex), vigabatrin (Sabril), zonisamide (Zonegran), or cefepime hydrochloride (Maxipime).

Thus, other suitable drugs for use herein include diuretics, vasopressin, agents affecting the renal conservation of water, rennin, angiotensin, agents useful in the treatment of myocardial ischemia, anti-hypertensive agents, angiotensin converting enzyme inhibitors, β-adrenergic receptor antagonists, agents for the treatment of hypercholesterolemia, and agents for the treatment of dyslipidemia.

Additional suitable drugs include drugs used for control of gastric acidity, agents for the treatment of peptic ulcers, agents for the treatment of gastroesophageal reflux disease, prokinetic agents, antiemetics, agents used in irritable bowel syndrome, agents used for diarrhea, agents used for constipation, agents used for inflammatory bowel disease, agents used for biliary disease, agents used for pancreatic disease. The compositions and methods of the invention can be used to modulate transport of drugs used to treat protozoal infections, drugs used to treat Malaria, Amebiasis, Giardiasis, Trichomoniasis, Trypanosomiasis, and/or Leishmaniasis, and/or drugs used in the chemotherapy of helminthiasis. Other drugs include antimicrobial agents, sulfonamides, trimethoprim-sulfamethoxazole quinolones, and agents for urinary tract infections, penicillins, cephalosporins, and other, A Lactam antibiotics, an agent comprising an aminoglycoside, protein synthesis inhibitors, drugs used in the chemotherapy of tuberculosis, mycobacterium avium complex disease, and leprosy, antifungal agents, antiviral agents including nonretroviral agents and antiretroviral agents.

In addition, drugs used for immunomodulation, such as immunomodulators, antivirals, antibiotics, immunosuppressive agents, tolerogens, and immunostimulants can be modulated. In addition, drugs acting on the blood and the blood-forming organs, hematopoietic agents, growth factors, minerals, and vitamins, anticoagulant, thrombolytic, and antiplatelet drugs can also be modulated. The invention can be used to modulate transport of hormones and hormone antagonists, pituitary hormones and their hypothalamic releasing factors, thyroid and antithyroid drugs, estrogens and progestins, androgens, adrenocorticotropic hormone; adrenocortical steroids and their synthetic analogs; inhibitors of the synthesis and actions of adrenocortical hormones, insulin, oral hypoglycemic agents, and the pharmacology of the endocrine pancreas, agents affecting calcification and bone turnover: calcium, phosphate, parathyroid hormone, vitamin D, calcitonin, and other compositions. Further transport of vitamins such as water-soluble vitamins, vitamin B complex, ascorbic acid, fat-soluble vitamins, vitamins A, K, and E can be modulated.

Additional suitable drugs may be found in Goodman and Gilman's “The Pharmacological Basis of Therapeutics” Tenth Edition edited by Hardman, Lombard and Gilman or the Physician's Desk Reference, both of which are incorporated herein by reference in their entirety.

Antihypertensives In some embodiments, compositions and methods of the invention encompass the use of an antihypertensive in combination with a pyrone analog-sulfobutylether-7-β-cyclodextrin such as flavonoid-sulfobutylether-7-β-cyclodextrin composition that reduces a side effect the antihypertensive, such as a BTB transport protein modulator.

Examples of antihypertensives useful in the methods and compositions of the invention include but are not limited to: atenolol, captopril, clonidine, guanethidine, hydralazine, hydrochorothiazide, lisinopril, losartan, methyldopa, minoxidil, nifedipine, prazosin, propranolol, reserpine, verapamil; centrally acting sympathoplegic drugs such as methyldopa, clonidine, guanabenz, guanfacine; ganglion-blocking agents such as mecamylamine (inversine); adrenergic neuron-blocking agents such as guanethidine, guanadrel, bethanidine, debrisoquin, reserpine; adrenoceptor antagonists such as propranolol; other beta-adrenoceptor-blocking agents such as metoprolol, nadolol, carteolol, atenolol, betaxolol, bisoprolol, pindolol, acebutolol, penbutolol, labetalol, carvedilol, esmolol, timolol; prazosin and other alpha blockers such as prazosin, terazosin, doxazosin; other alpha adrenoceptor-blocking agents such as pinacidil, urapidil, cromakalim; nonselective agents, phentolamine and phenoxybenzamine; vasodilators such as hydralazine and minoxidil; sodium nitroprusside, diazoxide, fenoldopam; calcium channel blockers such as verapamil, diltiazem and dihydrophyridine family (amlodipine, felodipine, isradipine, nicardipine, nifedipine, and nisoldipine); inhibitors of angiotensin such as renin, angiotensin, aldosterone; angiotensin-converting enzyme (ACE) inhibitors such as captopril, enalapril, lisinopril, benazepril, fosinopril, moexipril, perindopril, quinapril, ramipril, trandolapril; angiotensin receptor-blocking agents such as losartan, valsartan, candesartan, eprosartan, irbesartan and telmisartan, and olmisartan.

Antineoplastic agents or anti-cancer drugs. In some embodiments, compositions and methods of the invention encompass the use of an antineoplastic agent in combination with soluble pyrone analog. In some embodiments compositions and methods of the invention encompass the use of an antineoplastic agent or anti-cancer drugs in combination with a soluble pyrone analog such as pyrone analog-cyclodextrin e.g. flavonoid-cyclodextrin such as quercetin-cyclodextrin, fisetin-cyclodextrin or 5,7-dideoxyquercetin-cyclodextrin, wherein the soluble pyrone analog or its metabolite reduces a side effect of the antineoplastic agent, such as by acting as a BTB transport protein modulator.

Examples of antineoplastic agent or anti-cancer drugs useful in the methods and compositions of the invention include but are not limited to: capecitabine, cladribine, cytarabine, fludarabine, fluorouracil, gemcitabine, mercaptopurine, methotrexate, thioguanine; antitumor antibiotics such as: bleomycin; epipodophyllotoxins such as: etoposide, teniposide; taxanes such as: docetaxel, paclitaxel, vinca alkaloids such as: vinblastine, vincristine, vinorelbine; alkylating agents, such as: busulfan, carmustine, cyclophosphamide, lomustine, mechlorethamine, melphalan, thiotepa; anthracyclines, such as: daunorubicin, doxorubicin, epirubicin, idarubicin, mitoxantrone, Antitumor antibiotics such as: dactinomycin, mitomycin, camptothecins such as: irinotecan, topotecan, and platinum analogs such as: carboplatin, cisplatin, oxaliplatin,

Antiinfectives In some embodiments, compositions and methods of the invention encompass the use of an antiinfective agent in combination with a pyrone analog-sulfobutylether-7-β-cyclodextrin such as flavonoid-sulfobutylether-7-β-cyclodextrin composition that reduces a side effect of the antibacterial agent, such as a BTB transport protein modulator.

Non-limiting examples of antiinfective agents useful in the invention include 4-lactam drugs, quinolone drugs, ciprofloxacin, norfloxacin, tetracycline, amikacin, 2,4,4′-trichloro-2′-hydroxy diphenyl ether, 3,4,4′-trichlorocarbanilide, phenoxyethanol, phenoxy propanol, phenoxyisopropanol, doxycycline, capreomycin, chlorhexidine, chlortetracycline, oxytetracycline, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, octopirox, parachlorometa xylenol, nystatin, tolnaftate, zinc pyrithione and clotrimazole

VIII. Compositions Comprising a Therapeutic Agent and a Flavonoid with a Sulfoalkyl Ether Cyclodextrin

In one aspect the invention provides compositions that include a pyrone analog-sulfobutylether-7-β-cyclodextrin such as flavonoid-sulfobutylether-7-β-cyclodextrin as described herein that acts as an agent to reduce or eliminate a side effect of one or more substances. In some embodiments, the substance is a therapeutic agent with which the pyrone analog-sulfobutylether-7-β-cyclodextrin such as flavonoid-sulfobutylether-7-β-cyclodextrin composition is co-administered. “Co-administration,” “administered in combination with,” and their grammatical equivalents, as used herein, encompasses administration of two or more agents to an animal so that both agents and/or their metabolites are present in the animal at the same time. Co-administration includes simultaneous administration in separate compositions, administration at different times in separate compositions, or administration in a composition in which both agents are present, and combinations thereof.

In some embodiments, the invention provides compositions containing a combination of a therapeutic agent and a pyrone analog-sulfobutylether-7-β-cyclodextrin such as flavonoid-sulfobutylether-7-β-cyclodextrin composition that reduces or eliminates a side effect of the therapeutic agent. In some embodiments the invention provides pharmaceutical compositions that further include a pharmaceutically acceptable excipient. In some embodiments, the pharmaceutical compositions are suitable for oral administration. In some embodiments, the pharmaceutical compositions are suitable for transdermal administration. In some embodiments, the pharmaceutical compositions are suitable for injection. Other forms of administration are also compatible with embodiments of the pharmaceutical compositions of the invention, as described herein.

In some embodiments the pyrone analog such as a flavonoid is selected from the group consisting of quercetin or a quercetin derivative, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, 5,7-dideoxyquercetin (3,3′,4′-trihydroxyflavone), and epicatechin. In some embodiments the pyrone analog such as a flavonoid is a flavonol. In certain embodiments, the flavonol is selected from the group consisting of quercetin or a quercetin derivative, galangin, fisetin, and kaempferol, or combinations thereof. In some embodiments, the flavonol is quercetin. In some embodiments, the flavonol is galangin. In some embodiments, the flavonol is kaempferol.

In some embodiments, the side effect of the therapeutic agent that is reduced is selected from the group consisting of drowsiness, impaired concentration, sexual dysfunction, sleep disturbances, habituation, dependence, alteration of mood, respiratory depression, nausea, vomiting, dizziness, memory impairment, neuronal dysfunction, neuronal death, visual disturbance, impaired mentation, tolerance, addiction, hallucinations, lethargy, myoclonic jerking, endocrinopathies, and combinations thereof. In some embodiments, the side effect of the therapeutic agent that is reduced is selected from the group consisting of impaired concentration and sleep disturbances. In some embodiments, the side effect of the therapeutic agent that is reduced is impaired concentration. In some embodiments, the side effect of the therapeutic agent that is reduced is sleep disturbances.

In some embodiments, the side effect is a renal and/or urogenital side effect selected, e.g. from the group consisting of nephrotoxicity, renal function impairment, creatinine increase, urinary tract infection, oliguria, cystitis haemorrhagic, hemolytic-uremic syndrome or micturition disorder, as well as other effects mention herein, or combinations thereof. In some embodiments, the hepatic, pancreatic and/or gastrointestinal side effect is selected from the group consisting of hepatic necrosis, hepatotoxicity, liver fatty, venooclusive liver disease, diarrhea, nausea, constipation, vomiting, dyspepsia, anorexia, or LFT abnormal, as well as other effects mention herein, or combinations thereof.

In some embodiments the therapeutic agent is an immunosuppressant. In some embodiments, the immunosuppressant is selected from the group consisting of sirolimus, tacrolimus, mycophenolate, methadone, cyclosporin, cyclosporine, prednisone, or voclosporin.

In some embodiments the therapeutic agent is an analgesic agent. In some embodiments, the analgesic agent is selected from the group consisting of oxycodone, gabapentin, pregabalin, hydrocodone, fentanyl, hydromorphone, levorphenol, morphine, methadone, tramadol, topiramate, diacetyl morphine, codeine, olanzapine, hydrocortisone, prednisone, sufentanyl, alfentanyl, carbamazapine, lamotrigine, doxepin, and haloperidol. In some embodiments, the analgesic agent is selected from the group consisting of oxycodone, gabapentin, pregabalin, hydrocodone, fentanyl, hydromorphone, levorphenol, morphine, methadone, topiramate, diacetyl morphine, codeine, olanzapine, hydrocortisone, prednisone, sufentanyl, alfentanyl, carbamazapine, lamotrigine, doxepin, and haloperidol. In some embodiments, the analgesic agent is selected from the group consisting of oxycodone, gabapentin, pregabalin, hydrocodone, fentanyl, hydromorphine, levorphenol, morphine, methadone, tramadol and topiramate. In some embodiments, the analgesic is selected from the group consisting of oxycodone and gabapentin. In some embodiments, the analgesic is oxycodone. In some embodiments, the analgesic is gabapentin. In some embodiments, the analgesic is hydrocodone.

In some embodiments, the analgesic is an opiate analgesic. Opiate analgesics are as described herein. In some embodiments, the composition includes an opiate analgesic selected from the group consisting of oxycodone, hydrocodone, fentanyl, hydromorphone, levorphenol, morphine, methadone, tramadol, diacetyl morphine, codeine, sufentanyl, and alfentanyl. In some embodiments, the composition includes an opiate analgesic selected from the group consisting of oxycodone, hydrocodone, methadone, and tramadol. In some embodiments, the composition includes an opiate analgesic selected from the group consisting of oxycodone, hydrocodone, and methadone. In some embodiments, the opiate analgesic is oxycodone. In some embodiments, the opiate analgesic is hydrocodone. In some embodiments, the opiate analgesic is methadone.

In some embodiments, the analgesic is a non-opiate analgesic. Non-opiate analgesics are as described herein. In some embodiments, the composition includes a non-opiate analgesic selected from the group consisting of gabapentin, pregabalin, topiramate, olanzapine, hydrocortisone, prednisone, carbamazapine, lamotrigine, doxepin, and haloperidol. In some embodiments, the non-opiate analgesic is gabapentin. In some embodiments, the non-opiate analgesic is pregabalin.

Combinations of analgesics, such as combinations of an opiate and non-opiate analgesic, as are known in the art, may also be used in compositions of the invention.

In some embodiments, the composition includes a non-analgesic therapeutic agent. In some embodiments, the non-analgesic therapeutic agent is selected from the group consisting of antihypertensives, vasodilators, barbiturates, membrane stabilizers, cardiac stabilizers, glucocorticoids, antiinfectives. In some embodiments, the non-analgesic therapeutic agent is an antihypertensive. In some embodiments, the non-analgesic therapeutic agent is an antiinfective.

In some embodiments, the invention provides a composition containing a therapeutic agent and a pyrone analog-sulfobutylether-7-β-cyclodextrin such as flavonoid-sulfobutylether-7-β-cyclodextrin composition that acts as a BTB such as a blood-brain barrier (BBB) transport protein modulator, where the therapeutic agent is present in an amount sufficient to exert a therapeutic effect and the pyrone analog-sulfobutylether-7-β-cyclodextrin such as flavonoid-sulfobutylether-7-β-cyclodextrin is present in an amount sufficient to decrease a side effect of the therapeutic agent by a measurable amount, compared to the side effect without the pyrone analog-sulfobutylether-7-β-cyclodextrin such as flavonoid-sulfobutylether-7-β-cyclodextrin, when the composition is administered to an animal. In some embodiments, a side effect of the therapeutic agent is decreased by an average of at least about 5, 10, 15, 20, 25, 30, 35, 40, 45, 50, 55, 60, 65, 70, 75, 80, 85, 90, 95, or more than 95%, compared to the side effect without the pyrone analog-sulfobutylether-7-β-cyclodextrin such as flavonoid-sulfobutylether-7-β-cyclodextrin composition. In some embodiments, a side effect of the therapeutic agent is decreased by an average of at least about 5%, compared to the side effect without the pyrone analog-sulfobutylether-7-β-cyclodextrin such as flavonoid-sulfobutylether-7-β-cyclodextrin composition. In some embodiments, a side effect of the therapeutic agent is decreased by an average of at least about 10%, compared to the side effect without the pyrone analog-sulfobutylether-7-β-cyclodextrin such as flavonoid-sulfobutylether-7-β-cyclodextrin composition. In some embodiments, a side effect of the therapeutic agent is decreased by an average of at least about 15%, compared to the side effect without the pyrone analog-sulfobutylether-7-β-cyclodextrin such as flavonoid-sulfobutylether-7-β-cyclodextrin composition. In some embodiments, a side effect of the therapeutic agent is decreased by an average of at least about 20%, compared to the side effect without the pyrone analog-sulfobutylether-7-β-cyclodextrin such as flavonoid-sulfobutylether-7-β-cyclodextrin. In some embodiments, a side effect is substantially eliminated compared to the side effect without the pyrone analog such as a flavonoid-sulfobutylether-7-β-cyclodextrin composition. “Substantially eliminated” as used herein encompasses no measurable or no statistically significant side effect (one or more side effects) of the therapeutic agent, when administered in combination with the BTB transport protein modulator.

Thus, in some embodiments, the invention provides compositions that contain a sulfoalkyl ether cyclodextrin, e.g. sulfobutylether-7-β-cyclodextrin, a pyrone analog such as a flavonoid, and an analgesic agent, where the analgesic agent is present in an amount sufficient to exert an analgesic effect and the pyrone analog such as a flavonoid, e.g., a flavonol is present in an amount sufficient to decrease a side effect of the analgesic agent by a measurable amount, compared to the side effect without the pyrone analog such as a flavonoid, e.g., a flavonol when the composition is administered to an animal. The measurable amount may be an average of at least about 1, 5, 10, 15, 20, 25, 30, 35, 40, 45, 50, 55, 60, 65, 70, 75, 80, 85, 90, 95, or more than 95% as described herein. The side effect may be any side effect as described herein. In some embodiments, the side effect is disturbance of concentration. In some embodiments, the side effect is sleep disturbances.

In some embodiments, the invention provides compositions that contain a sulfoalkyl ether cyclodextrin, e.g. sulfobutylether-7-β-cyclodextrin, a flavonol and an opiate analgesic agent, where the opiate analgesic agent is present in an amount sufficient to exert an analgesic effect and the flavonol is present in an amount sufficient to decrease a side effect such as a side effect of the opiate analgesic agent by a measurable amount, compared to the side effect without the flavonol when the composition is administered to an animal. The measurable amount may be an average of at least about 1, 5, 10, 15, 20, 25, 30, 35, 40, 45, 50, 55, 60, 65, 70, 75, 80, 85, 90, 95, or more than 95% as described herein. For further description of analgesic agents that may be used in the compositions of the invention, see U.S. Patent Publication No. US2006/0111308, particularly at paragraphs [0130]-[0154], and PCT published Patent Application WO/06055672, particularly at paragraphs [00116]-[00136]. The side effect may be any side effect including those described herein. In some embodiments, the side effect is loss of concentration. In some embodiments, the side effect is sleep disturbances.

In some embodiments, the invention provides compositions that contains a sulfoalkyl ether cyclodextrin, e.g. sulfobutylether-7-β-cyclodextrin, quercetin or a quercetin derivative and an immunosuppressant such as tacrolimus, or FK-506 where the FK-506 is present in an amount sufficient to exert an analgesic effect and the quercetin or a quercetin derivative is present in an amount sufficient to decrease a side effect of the immunosuppressant such as FK-506 by a measurable amount, compared to the side effect without the quercetin or a quercetin derivative when the composition is administered to an animal. The measurable amount may be an average of at least about 1, 5, 10, 15, 20, 25, 30, 35, 40, 45, 50, 55, 60, 65, 70, 75, 80, 85, 90, 95, or more than 95%, compared to the side effect without the pyrone analog-sulfobutylether-7-β-cyclodextrin such as flavonoid-sulfobutylether-7-β-cyclodextrin composition. The side effect may be any side effect as described herein. In some embodiments, the side effect is hyperglycemia. In some embodiments, the side effect is sleep disturbances. In some embodiments, the side effect is nausea and/or vomiting. In some embodiments, the side effect is cognitive depression.

In some embodiments, the invention provides compositions that contains pyrone analog-sulfobutylether-7-β-cyclodextrin such as flavonoid-sulfobutylether-7-β-cyclodextrin composition and Tamoxifen where the Tamoxifen is present in an amount sufficient to exert an analgesic effect and the quercetin or a quercetin derivative is present in an amount sufficient to decrease a side effect of the Tamoxifen by a measurable amount, compared to the side effect without the quercetin or a quercetin derivative when the composition is administered to an animal. The measurable amount may be an average of at least about 1, 5, 10, 15, 20, 25, 30, 35, 40, 45, 50, 55, 60, 65, 70, 75, 80, 85, 90, 95, or more than 95%, compared to the side effect without the pyrone analog-sulfobutylether-7-β-cyclodextrin such as flavonoid-sulfobutylether-7-β-cyclodextrin composition. The side effect may be any side effect as described herein. In some embodiments, the side effect is loss of concentration. In some embodiments, the side effect is sleep disturbances. In some embodiments, the side effect is nausea and/or vomiting. In some embodiments, the side effect is cognitive depression.

In some embodiments, a therapeutic effect of the therapeutic agent is increased by an average of at least about 5%, compared to the therapeutic effect without the pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin. In some embodiments, a therapeutic effect of the therapeutic agent is increased by an average of at least about 10%, compared to the therapeutic effect without the pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin. In some embodiments, a therapeutic effect of the therapeutic agent is increased by an average of at least about 15%, compared to the therapeutic effect without pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin. In some embodiments, a therapeutic effect of the therapeutic agent is increased by an average of at least about 20%, compared to the therapeutic effect without the pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin. In some embodiments, a therapeutic effect of the therapeutic agent is increased by an average of at least about 30%, compared to the therapeutic effect without the pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin. In some embodiments, a therapeutic effect of the therapeutic agent is increased by an average of at least about 40%, compared to the therapeutic effect without the pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin. In some embodiments, a therapeutic effect of the therapeutic agent is increased by an average of at least about 50%, compared to the therapeutic effect without the pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin.

Thus, in some embodiments, the invention provides compositions containing a pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin present in an amount sufficient to decrease a side effect of a therapeutic agent by an average of at least about 5% and to increase a therapeutic effect of the therapeutic agent by an average of at least about 5%, compared to the side effect and therapeutic effect without the pyrone analog sulfoalkyl cyclodextrin such as flavonoid-sulfoalkyl cyclodextrin, when the composition is administered to an animal in combination with the therapeutic agent. In some embodiments, the invention provides compositions containing a pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin present in an amount sufficient to decrease a side effect of a therapeutic agent by an average of at least about 10% and to increase a therapeutic effect of the therapeutic agent by an average of at least about 10%, compared to the side effect and therapeutic effect without pyrone analog sulfoalkyl cyclodextrin such as flavonoid-sulfoalkyl cyclodextrin, when the composition is administered to an animal in combination with the therapeutic agent. In some embodiments, the invention provides compositions containing a pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin present in an amount sufficient to decrease a side effect of a therapeutic agent by an average of at least about 20% and to increase a therapeutic effect of the therapeutic agent by an average of at least about 20%, compared to the side effect and therapeutic effect without the pyrone analog sulfoalkyl cyclodextrin such as flavonoid-sulfoalkyl cyclodextrin, when the composition is administered to an animal in combination with the therapeutic agent. In some embodiments, the invention provides compositions containing a pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin is present in an amount sufficient to decrease a side effect of a therapeutic agent by an average of at least about 10% and to increase a therapeutic effect of the therapeutic agent by an average of at least about 20%, compared to the side effect and therapeutic effect without the pyrone analog sulfoalkyl cyclodextrin such as flavonoid-sulfoalkyl cyclodextrin, when the composition is administered to an animal in combination with the therapeutic agent. In some embodiments, the invention provides compositions containing a pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin present in an amount sufficient to decrease a side effect of a therapeutic agent by an average of at least about 10% and to increase a therapeutic effect of the therapeutic agent by an average of at least about 30%, compared to the side effects and therapeutic effect without the pyrone analog sulfoalkyl cyclodextrin such as flavonoid-sulfoalkyl cyclodextrin, when the composition is administered to an animal in combination with the therapeutic agent. In some embodiments, the invention provides compositions containing a pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin present in an amount sufficient to decrease a side effect of a therapeutic agent by an average of at least about 10% and to increase a therapeutic effect of the therapeutic agent by an average of at least about 40%, compared to the side effect and therapeutic effect without the pyrone analog sulfoalkyl cyclodextrin such as flavonoid-sulfoalkyl cyclodextrin, when the composition is administered to an animal in combination with the therapeutic agent. In some embodiments, the invention provides compositions containing a pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin present in an amount sufficient to decrease a side effect of a therapeutic agent by an average of at least about 10% and to increase a therapeutic effect of the therapeutic agent by an average of at least about 50%, compared to the side effect and therapeutic effect without the pyrone analog sulfoalkyl cyclodextrin such as flavonoid-sulfoalkyl cyclodextrin, when the composition is administered to an animal in combination with the therapeutic agent.

In exemplary embodiments, the invention provides a composition that contains a pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin that wherein the pyrone analog such as a flavonoid is quercetin or a quercetin derivative, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, 5,7-dideoxyquercetin (3,3′,4′-trihydroxyflavone), or epicatechin, or combinations thereof, and an analgesic, such as an opiate or nonopiate analgesic agent, where the analgesic is present in an amount sufficient to exert an analgesic effect, and the pyrone analog sulfoalkyl cyclodextrin such as flavonoid-sulfoalkyl cyclodextrin is present in an amount effective to decrease a side effect of the analgesic agent by a measurable amount (e.g., an average of at least about 5, 10, 15, 20, or more than 20%, as described herein) and to increase the analgesic effect of the analgesic agent by a measurable amount (e.g., an average of at least about 5, 10, 15, 20, or more than 20%, as described herein). The side effect may be any side effect as described herein. In some embodiments, the side effect is loss of concentration. In some embodiments, the side effect is sleep disturbances.

In exemplary embodiments, the invention provides a composition that contains a pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin wherein the pyrone analog such as a flavonoid is quercetin or a quercetin derivative, galangin, fisetin, or kaempferol and an analgesic that is oxycodone, gabapentin, pregabalin, hydrocodone, fentanyl, hydromorphone, levorphenol, morphine, methadone, tramadol, topiramate, diacetyl morphine, codeine, olanzapine, hydrocortisone, prednisone, sufentanyl, alfentanyl, carbamazapine, lamotrigine, doxepin, or haloperidol, where the analgesic is present in an amount sufficient to exert an analgesic effect, and the pyrone analog sulfoalkyl cyclodextrin such as flavonoid-sulfoalkyl cyclodextrin is present in an amount effective to decrease a side effect of the analgesic agent by a measurable amount (e.g., an average of at least about 5, 10, 15, 20, or more than 20%, as described herein) and to increase the analgesic effect of the analgesic agent by a measurable amount (e.g., an average of at least about 5, 10, 15, 20, or more than 20%, as described herein). The side effect may be any side effect as described herein. In some embodiments, the side effect is loss of concentration. In some embodiments, the side effect is sleep disturbances.

In exemplary embodiments, the invention provides a composition that contains a pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin, where the flavonoid is quercetin or a quercetin derivative, galangin, or kaempferol and an analgesic that is oxycodone, gabapentin, pregabalin, hydrocodone, fentanyl, hydromorphone, levorphenol, morphine, methadone, tramadol, topiramate, diacetyl morphine, codeine, olanzapine, hydrocortisone, prednisone, sufentanyl, alfentanyl, carbamazapine, lamotrigine, doxepin, or haloperidol, where the analgesic is present in an amount sufficient to exert an analgesic effect, and the pyrone analog sulfoalkyl cyclodextrin such as flavonoid-sulfoalkyl cyclodextrin is present in an amount effective to decrease a side effect of the analgesic agent by a measurable amount (e.g., an average of at least about 5, 10, 15, 20, or more than 20%, as described herein) and to increase the analgesic effect of the analgesic agent by a measurable amount (e.g., an average of at least about 5, 10, 15, 20, or more than 20%, as described herein). The side effect may be any side effect as described herein. In some embodiments, the side effect is loss of concentration. In some embodiments, the side effect is sleep disturbances.

In further exemplary embodiments, the invention provides a composition that contains a pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin, where the pyrone analog such as a flavonoid is quercetin or a quercetin derivative, galangin, fisetin, or kaempferol and an analgesic that is oxycodone, hydrocodone, methadone, tramadol, gabapentin, lorazepam, cyclobenzaprine hydrochloride, or carisoprodol, where the analgesic is present in an amount sufficient to exert an analgesic effect, and the pyrone analog such as a flavonoid is present in an amount effective to decrease a side effects of the analgesic agent by a measurable amount (e.g., an average of at least about 5, 10, 15, 20, or more than 20%, as described herein) and to increase the analgesic effect of the analgesic agent by a measurable amount (e.g., an average of at least about 5, 10, 15, 20, or more than 20%, as described herein). The side effect may be any side effect as described herein. In some embodiments, the side effect is loss of concentration. In some embodiments, the side effect is sleep disturbances.

In yet further exemplary embodiments, the invention provides a composition that contains a pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin, where the pyrone analog such as a flavonoid is quercetin or a quercetin derivative, galangin, fisetin, or kaempferol and an analgesic that is oxycodone or gabapentin, where the analgesic is present in an amount sufficient to exert an analgesic effect, and the flavonol is present in an amount effective to decrease side effect of the analgesic agent by a measurable amount (e.g., an average of at least about 5, 10, 15, 20, or more than 20%, as described herein) and to increase the analgesic effect of the analgesic agent by a measurable amount (e.g., an average of at least about 5, 10, 15, 20, or more than 20%, as described herein). The side effect may be any side effect as described herein. In some embodiments, the side effect is loss of concentration. In some embodiments, the side effect is sleep disturbances.

In still yet further exemplary embodiments, the invention provides a composition that contains a sulfoalkyl ether cyclodextrin-quercetin, e.g. sulfobutylether-7-β-cyclodextrin-quercetin and oxycodone, where the oxycodone is present in an amount sufficient to exert an analgesic effect, and the quercetin is present in an amount effective to decrease a side effect of the oxycodone by a measurable amount (e.g., an average of at least about 5, 10, 15, 20, or more than 20%, as described herein) and to increase the analgesic effect of the oxycodone by a measurable amount (e.g., an average of at least about 5, 10, 15, 20, or more than 20%, as described herein). The side effect may be any side effect as described herein. In some embodiments, the side effect is loss of concentration. In some embodiments, the side effect is sleep disturbances.

In still yet further exemplary embodiments, the invention provides a composition that contains a sulfoalkyl ether cyclodextrin-quercetin, e.g. sulfobutylether-7-β-cyclodextrin-quercetin and gabapentin, where the gabapentin is present in an amount sufficient to exert an analgesic effect, and the quercetin is present in an amount effective to decrease a side effect of the gabapentin by a measurable amount (e.g., an average of at least about 5, 10, 15, 20, or more than 20%, as described herein) and to increase the analgesic effect of the gabapentin by a measurable amount (e.g., an average of at least about 5, 10, 15, 20, or more than 20%, as described herein). The side effect may be any side effect as described herein. In some embodiments, the side effect is loss of concentration. In some embodiments, the side effect is sleep disturbances.

In still yet further exemplary embodiments, the invention provides a composition that contains a sulfoalkyl ether cyclodextrin-quercetin, e.g. sulfobutylether-7-β-cyclodextrin-quercetin and pregabalin, where the pregabalin is present in an amount sufficient to exert an analgesic effect, and the quercetin is present in an amount effective to decrease a side effect of the pregabalin by a measurable amount (e.g., an average of at least about 5, 10, 15, 20, or more than 20%, as described herein) and to increase the analgesic effect of the pregabalin by a measurable amount (e.g., an average of at least about 5, 10, 15, 20, or more than 20%, as described herein). The side effect may be any side effect as described herein. In some embodiments, the side effect is loss of concentration. In some embodiments, the side effect is sleep disturbances.

An “average” as used herein is preferably calculated in a set of normal human subjects, this set being at least about 3 human subjects, preferably at least about 5 human subjects, preferably at least about 10 human subjects, even more preferably at least about 25 human subjects, and most preferably at least about 50 human subjects.

In some embodiments, the invention provides a composition that contains a therapeutic agent and a pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin. In some embodiments, the concentration of the therapeutic agent is less than 90%, 80%, 70%, 60%, 50%, 40%, 30%, 20%, 19%, 18%, 17%, 16%, 15%, 14%, 13%, 12%, 1%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, or 0.01% w/w, w/v or v/v; and the concentration of the pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin is less than 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, or 0.01% w/w, w/v or v/v.

In some embodiments, a concentration of one or more of the therapeutic agents and/or pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01% w/w, w/v, or v/v.

In some embodiments, a concentration of the therapeutic agent is in the range from approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v. v/v; and the concentration of the pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin, is in the range from approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v. v/v.

In some embodiments, a concentration of the therapeutic agent is in the range from approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v and the sulfoalkyl ether cyclodextrin-flavonoid, e.g. sulfobutylether-7-β-cyclodextrin-flavonoid is in the range from approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some embodiments, a amount of the therapeutic agent is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g; and the pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, an amount of the therapeutic agent is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, , 0.25 g, 0.3 g, , 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g; and the amount of the pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

In some embodiments, a concentration of the therapeutic agents is in the range of 0.0001-10 g, 0.0005-9 g, 0.001-8 g, 0.005-7 g, 0.01-6 g, 0.05-5 g, 0.1-4 g, 0.5-4 g, or 1-3 g; and the concentration of the pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin is in the range of 0.0001-10 g, 0.0005-9 g, 0.001-8 g, 0.005-7 g, 0.01-6 g, 0.05-5 g, 0.1-4 g, 0.5-4 g, or 1-3 g.

In liquid preparations, the morphine can be present at about 5-500 mg/ml, or about 100-500 mg/ml, or about 250 mg/ml, and sulfobutylether-7-β-cyclodextrin-quercetin is present such that quercetin is present at about 1-1000 mg/ml, or about 10-1000 mg/ml, or about 50-1000 mg/ml, or about 100-1000 mg/ml, or about 1-500 mg/ml, or about 5-500 mg/ml, or about 50-500 mg/ml, or about 100-500 mg/ml, or about 200-1000 mg/ml, or about 200-800 mg/ml, or about 200-700 mg/ml, or about 10 mg/ml, or about 25 mg/ml, or about 50 mg/ml, or about 100 mg/ml, or about 200 mg/ml, or about 250 mg/ml, or about 300 mg/ml, or about 400 mg/ml, or about 500 mg/ml, or about 600 mg/ml, or about 700 mg/ml, or about 800 mg/ml, or about 900 mg/ml, or about 1000 mg/ml At higher levels of quercetin, solubility can be enhanced by adjusting the type of diluent.

In some embodiments, a molar ratio of the therapeutic agent to the pyrone analog such as a flavonoid in the pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin can be 0.0001:1 to 1:1. Without limiting the scope of the invention, the molar ratio of one or more of the therapeutic agents to the pyrone analog such as a flavonoid in the pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin can be about 0.0001:1 to about 10:1, or about 0.001:1 to about 5:1, or about 0.01:1 to about 5:1, or about 0.1:1 to about 2:1, or about 0.2:1 to about 2:1, or about 0.5:1 to about 2:1, or about 0.1:1 to about 1:1.

Without limiting the scope of the present invention, the molar ratio of one or more of the therapeutic agents to the pyrone analog such as a flavonoid in the pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin can be about 0.03×10⁻⁵:1, 0.1×10⁻⁵:1, 0.04×10⁻³:1, 0.03×10⁻⁵:1, 0.02×10⁻³:1, 0.01×10⁻³:1, 0.1×10⁻³:1, 0.15×10⁻³:1, 0.2×10⁻³:1, 0.3×10⁻³:1, 0.4×10⁻³:1, 0.5×10⁻³:1, 0.15×10⁻²:1, 0.1×10⁻²:1, 0.2×10⁻²:1, 0.3×10⁻²:1, 0.4×10⁻²:1, 0.5×10⁻²:1, 0.6×10⁻²:1, 0.8×10⁻²:1, 0.01:1, 0.1:1; or 0.2:1 per dose. In one embodiment, the therapeutic agent is oxycodone. In one embodiment, the sulfoalkyl ether cyclodextrin-flavonoid is sulfobutylether-7-β-cyclodextrin-quercetin.

Without limiting the scope of the present invention, the molar ratio of one or more of the therapeutic agents to the pyrone analog such as a flavonoid in the pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin can be about 0.03×10⁻⁵:1, 0.1×10⁻⁵:1, 0.04×10⁻³:1, 0.03×10⁻⁵:1, 0.02×10⁻⁵:1, 0.01×10⁻³:1, 0.1×10⁻³:1, 0.15×10⁻³:1, 0.2×10⁻³:1, 0.3×10⁻³:1, 0.4×10⁻³:1, 0.5×10⁻³:1, 0.15×10⁻²:1, 0.1×10⁻²:1, 0.2×10⁻²:1, 0.3×10⁻²:1, 0.4×10⁻²:1, 0.5×10⁻²:1, 0.6×10⁻²:1, 0.8×10⁻²:1, 0.01:1, 0.1:1; or 0.2:1 per dose. In one embodiment, the therapeutic agent is fentanyl. In one embodiment, the sulfoalkyl ether cyclodextrin-flavonoid is sulfobutylether-7-β-cyclodextrin-quercetin.

Without limiting the scope of the present invention, the molar ratio of one or more of the therapeutic agents to the pyrone analog such as a flavonoid in the pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin, can be about 0.001:1, 0.002:1, 0.003:1, 0.004:1, 0.005:1, 0.006:1, 0.007:1, 0.008:1, 0.009:1, 0.01:1, 0.02:1, 0.03:1, 0.04:1, 0.05:1, 0.06:1, 0.07:1, 0.08:1, 0.09:1, 0.1:1, 0.2:1, 0.3:1, 0.4:1, 0.5:1, 0.6:1, 0.7:1, 0.8:1, 0.9:1, 1:1, 2:1, 3:1, 4:1, or 5:1 per dose. In one embodiment, the therapeutic agent is Gabapentin or pregabalin. In one embodiment, the sulfoalkyl ether cyclodextrin-flavonoid is sulfobutylether-7-β-cyclodextrin-quercetin.

### IX. Pharmaceutical Compositions

The pyrone analog-sulfoalkyl cyclodextrins such as flavonoid-sulfoalkyl cyclodextrins of the invention are usually administered in the form of pharmaceutical compositions. The drugs described above are also administered in the form of pharmaceutical compositions. When the pyrone analog-sulfoalkyl cyclodextrins such as flavonoid-sulfoalkyl cyclodextrins and the drugs are used in combination, both components may be mixed into a preparation or both components may be formulated into separate preparations to use them in combination separately or at the same time.

This invention therefore provides pharmaceutical compositions that contain, as the active ingredient, a pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin or a pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants.

This invention further provides pharmaceutical compositions that contain, as the active ingredient, a pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin or a pharmaceutically acceptable salt and/or coordination complex thereof, a therapeutic agent or a pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants.

Such compositions are prepared in a manner well known in the pharmaceutical art.

Pharmaceutical compositions for oral administration In some embodiments, the invention provides a pharmaceutical composition for oral administration.

In some embodiments, the oral formulation is an aqueous formulation comprising a pyrone analog such as a flavonoid and a sulfo-alkyl ether substituted cyclodextrin and a pharmaceutically or veterinarily acceptable aqueous carrier wherein the pyrone analog such as a flavonoid is present in a concentration greater than 0.5 mM, 1 mM, 5 mM, 10 mM, 20 mM, 30 mM, 33 mM, 40 mM, 50 mM, 60 mM, 70 mM, 80 mM or greater than 80 mM.

In some embodiments, the oral formulation is an aqueous formulation comprising a pyrone analog such as a flavonoid and a sulfobutylether-7-β-cyclodextrin and a pharmaceutically or veterinarily acceptable aqueous carrier wherein the pyrone analog such as a flavonoid is present in a concentration greater than 0.5 mM. In some embodiments, the oral formulation is an aqueous formulation comprising a pyrone analog such as a flavonoid and a sulfobutylether-7-β-cyclodextrin and a pharmaceutically or veterinarily acceptable aqueous carrier wherein the pyrone analog such as a flavonoid is present in a concentration greater than 1 mM. In some embodiments, the oral formulation is an aqueous formulation comprising a pyrone analog such as a flavonoid and a sulfobutylether-7-β-cyclodextrin and a pharmaceutically or veterinarily acceptable aqueous carrier wherein the pyrone analog such as a flavonoid is present in a concentration greater than 5 mM. In some embodiments, the oral formulation is an aqueous formulation comprising a pyrone analog such as a flavonoid and a sulfobutylether-7-β-cyclodextrin and a pharmaceutically or veterinarily acceptable aqueous carrier wherein the pyrone analog such as a flavonoid is present in a concentration greater than 10 mM. In some embodiments, the oral formulation is an aqueous formulation comprising a pyrone analog such as a flavonoid and a sulfobutylether-7-β-cyclodextrin and a pharmaceutically or veterinarily acceptable aqueous carrier wherein the pyrone analog such as a flavonoid is present in a concentration greater than 15 mM. In some embodiments, the oral formulation is an aqueous formulation comprising a pyrone analog such as a flavonoid and a sulfobutylether-7-β-cyclodextrin and a pharmaceutically or veterinarily acceptable aqueous carrier wherein the pyrone analog such as a flavonoid is present in a concentration greater than 20 mM. In some embodiments, the oral formulation is an aqueous formulation comprising a pyrone analog such as a flavonoid and a sulfobutylether-7-β-cyclodextrin and a pharmaceutically or veterinarily acceptable aqueous carrier wherein the pyrone analog such as a flavonoid is present in a concentration greater than 30 mM. In some embodiments, the oral formulation is an aqueous formulation comprising a pyrone analog such as a flavonoid and a sulfobutylether-7-β-cyclodextrin and a pharmaceutically or veterinarily acceptable aqueous carrier wherein the pyrone analog such as a flavonoid is present in a concentration greater than 33 mM. In some embodiments, the oral formulation is an aqueous formulation comprising a pyrone analog such as a flavonoid and a sulfobutylether-7-β-cyclodextrin and a pharmaceutically or veterinarily acceptable aqueous carrier wherein the pyrone analog such as a flavonoid is present in a concentration greater than 40 mM. In some embodiments, the oral formulation is an aqueous formulation comprising a pyrone analog such as a flavonoid and a sulfobutylether-7-β-cyclodextrin and a pharmaceutically or veterinarily acceptable aqueous carrier wherein the pyrone analog such as a flavonoid is present in a concentration greater than 50 mM. In some embodiments, the oral formulation is an aqueous formulation comprising a pyrone analog such as a flavonoid and a sulfobutylether-7-β-cyclodextrin and a pharmaceutically or veterinarily acceptable aqueous carrier wherein the pyrone analog such as a flavonoid is present in a concentration greater than 60 mM. In some embodiments, the oral formulation is an aqueous formulation comprising a pyrone analog such as a flavonoid and a sulfobutylether-7-β-cyclodextrin and a pharmaceutically or veterinarily acceptable aqueous carrier wherein the pyrone analog such as a flavonoid is present in a concentration greater than 70 mM. In some embodiments, the oral formulation is an aqueous formulation comprising a pyrone analog such as a flavonoid and a sulfobutylether-7-β-cyclodextrin and a pharmaceutically or veterinarily acceptable aqueous carrier wherein the pyrone analog such as a flavonoid is present in a concentration greater than 80 mM.

In some embodiments, the oral formulation is made using an aqueous composition comprising quercetin or a quercetin derivative, a sulfo-alkyl ether substituted cyclodextrin and a pharmaceutically or veterinarily acceptable aqueous carrier wherein the pyrone analog such as a flavonoid is present in a concentration of greater than 0.5 mM, 1 mM, 5 mM, 10 mM, 20 mM, 30 mM, 33 mM, 40 mM, 50 mM, 60 mM, 70 mM, 80 mM or greater than 80 mM.

In some embodiments, the oral formulation is made using an aqueous composition comprising quercetin or a quercetin derivative, a sulfobutylether-7-β-cyclodextrin, and a pharmaceutically or veterinarily acceptable aqueous carrier wherein the quercetin or a quercetin derivative is present in a concentration of greater than 0.5 mM in the composition used to make the formulation. In some embodiments, the oral formulation is made using an aqueous composition comprising quercetin or a quercetin derivative, a sulfobutylether-7-β-cyclodextrin, and a pharmaceutically or veterinarily acceptable aqueous carrier wherein the quercetin or a quercetin derivative is present in a concentration of greater than 1 mM in the composition used to make the formulation. In some embodiments, the oral formulation is made using an aqueous composition comprising quercetin or a quercetin derivative, a sulfobutylether-7-β-cyclodextrin, and a pharmaceutically or veterinarily acceptable aqueous carrier wherein the quercetin or a quercetin derivative is present in a concentration of greater than 5 mM in the composition used to make the formulation. In some embodiments, the oral formulation is made using an aqueous composition comprising quercetin or a quercetin derivative, a sulfobutylether-7-β-cyclodextrin, and a pharmaceutically or veterinarily acceptable aqueous carrier wherein the quercetin or a quercetin derivative is present in a concentration of greater than 10 mM in the composition used to make the formulation. In some embodiments, the oral formulation is made using an aqueous composition comprising quercetin or a quercetin derivative, a sulfobutylether-7-β-cyclodextrin, and a pharmaceutically or veterinarily acceptable aqueous carrier wherein the quercetin or a quercetin derivative is present in a concentration of greater than 15 mM in the composition used to make the formulation. In some embodiments, the oral formulation is made using an aqueous composition comprising quercetin or a quercetin derivative, a sulfobutylether-7-β-cyclodextrin, and a pharmaceutically or veterinarily acceptable aqueous carrier wherein the quercetin or a quercetin derivative is present in a concentration of greater than 20 mM in the composition used to make the formulation. In some embodiments, the oral formulation is made using an aqueous composition comprising quercetin or a quercetin derivative, a sulfobutylether-7-β-cyclodextrin, and a pharmaceutically or veterinarily acceptable aqueous carrier wherein the quercetin or a quercetin derivative is present in a concentration of greater than 30 mM in the composition used to make the formulation. In some embodiments, the oral formulation is made using an aqueous composition comprising quercetin or a quercetin derivative, a sulfobutylether-7-β-cyclodextrin, and a pharmaceutically or veterinarily acceptable aqueous carrier wherein the quercetin or a quercetin derivative is present in a concentration of greater than 33 mM in the composition used to make the formulation. In some embodiments, the oral formulation is made using an aqueous composition comprising quercetin or a quercetin derivative, a sulfobutylether-7-β-cyclodextrin, and a pharmaceutically or veterinarily acceptable aqueous carrier wherein the quercetin or a quercetin derivative is present in a concentration of greater than 40 mM in the composition used to make the formulation. In some embodiments, the oral formulation is made using an aqueous composition comprising quercetin or a quercetin derivative, a sulfobutylether-7-β-cyclodextrin, and a pharmaceutically or veterinarily acceptable aqueous carrier wherein the quercetin or a quercetin derivative is present in a concentration of greater than 50 mM in the composition used to make the formulation. In some embodiments, the oral formulation is made using an aqueous composition comprising quercetin or a quercetin derivative, a sulfobutylether-7-β-cyclodextrin, and a pharmaceutically or veterinarily acceptable aqueous carrier wherein the quercetin or a quercetin derivative is present in a concentration of greater than 60 mM in the composition used to make the formulation. In some embodiments, the oral formulation is made using an aqueous composition comprising quercetin or a quercetin derivative, a sulfobutylether-7-β-cyclodextrin, and a pharmaceutically or veterinarily acceptable aqueous carrier wherein the quercetin or a quercetin derivative is present in a concentration of greater than 80 mM in the composition used to make the formulation.

Where the oral formulation is made from the aqueous composition of sulfoalkyl ether cyclodextrin-pyrone analog such as a flavonoid, the oral formulation can be a solid formulation that is produced by drying the aqueous composition, for example by freeze-drying or lyophilization. Lyophilization is a freeze-drying process in which water is sublimed from the composition after it is frozen. The particular advantages of the lyophilization process are that biologicals and pharmaceuticals that are relatively unstable in aqueous solution can be dried without elevated temperatures (thereby eliminating the adverse thermal affects) and then stored in the dry state where there are few stability problems. Once the aqueous composition is dried, it can be handled, for example, as a dried powder. The dried powder can be further formulated into oral pharmaceutical compositions as described herein.

In some embodiments, the oral pharmaceutical composition comprises a therapeutic agent, a pyrone analog such as a flavonoid, a sulfo-alkyl ether substituted cyclodextrin e.g. sulfobutylether-7-β-cyclodextrin and a carrier. In some embodiments, the oral composition comprises a combination of a therapeutic agent and a pyrone analog sulfoalkyl cyclodextrin such as flavonoid-sulfoalkyl cyclodextrin, that acts as an agent to reduce or eliminate a side effect of the therapeutic agent, and a pharmaceutical excipient suitable for oral administration. In some embodiments, the agent that reduces or eliminates the side effect of the therapeutic agent is sulfobutylether-7-β-cyclodextrin-quercetin.

In some embodiments, the pharmaceutical composition may be a liquid pharmaceutical composition suitable for oral consumption.

In some embodiments, the therapeutic agent is an analgesic agent. In some embodiments, the therapeutic agent is a non-analgesic agent. In some embodiments, the therapeutic agent is an opiate analgesic agent. In some embodiments, the therapeutic agent is an nonopiate analgesic agent. In some embodiments, the pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin that is capable of reducing or eliminating one or more side effect of the therapeutic agent is a BTB transport protein modulator, e.g., a BTB transport protein activator. In some embodiments, the agent capable of reducing or eliminating one or more side effects of the therapeutic agent is sulfobutylether-7-β-cyclodextrin-quercetin.

In some embodiments, the pharmaceutical composition may be a liquid pharmaceutical composition suitable for oral consumption.

In some embodiments, the invention provides a solid pharmaceutical composition for oral administration containing an effective amount of oxycodone, an amount of a sulfoalkyl ether cyclodextrin-quercetin, e.g. sulfobutylether-7-β-cyclodextrin-quercetin that is effective in reducing or eliminating a side effect of oxycodone, and a pharmaceutically acceptable excipient. In some embodiments, the composition further includes an effective amount of acetaminophen. In some embodiments, the invention provides a liquid pharmaceutical composition for oral administration containing an effective amount of oxycodone, an amount of a sulfoalkyl ether cyclodextrin-quercetin, e.g. sulfobutylether-7-β-cyclodextrin-quercetin that is effective in reducing or eliminating a side effect of oxycodone, and a pharmaceutically acceptable excipient. In some embodiments, the composition further includes an effective amount of acetaminophen.

In some embodiments, the invention provides a solid pharmaceutical composition for oral administration containing oxycodone at about 1-160 mg, quercetin as a sulfobutylether-7-β-cyclodextrin-quercetin composition at about 10-1000 mg and a pharmaceutically acceptable excipient. In some embodiments, the composition further includes acetaminophen at about 200-750 mg. In some embodiments, the invention provides a liquid pharmaceutical composition for oral administration containing oxycodone at about 1-200 mg/ml, quercetin at about 10-1000 mg/ml and a pharmaceutically acceptable excipient. In some embodiments, the composition further includes acetaminophen at about 10-750 mg/ml.

In some embodiments, the invention provides a solid pharmaceutical composition for oral administration containing an effective amount of gabapentin, an amount of sulfobutylether-7-β-cyclodextrin-quercetin that is effective in reducing or eliminating a side effects of gabapentin, and a pharmaceutically acceptable excipient. In some embodiments, the invention provides a liquid pharmaceutical composition for oral administration containing an effective amount of gabapentin, an amount of quercetin that is effective in reducing or eliminating a side effect of gabapentin, and a pharmaceutically acceptable excipient.

In some embodiments, the invention provides a solid pharmaceutical composition for oral administration containing gabapentin at about 100-800 mg, sulfobutylether-7-β-cyclodextrin-quercetin such that quercetin is present at about 10-1000 mg and a pharmaceutically acceptable excipient. In some embodiments, the invention provides a liquid pharmaceutical composition for oral administration containing gabapentin at about 5-500 mg/ml, quercetin at about 10-1000 mg/ml and a pharmaceutically acceptable excipient.

Pharmaceutical compositions of the invention suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered composition moistened with an inert liquid diluent.

This invention further encompasses anhydrous pharmaceutical compositions and dosage forms comprising an active ingredient, since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the invention to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the compositions disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the essential active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof, lysophospholipids and derivatives thereof; camitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, preferred ionic surfactants include, by way of example: lecithins, lysolecithin, phospholipids, lysophospholipids and derivatives thereof; camitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl camitines, palmitoyl camitines, myristoyl camitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition may include a solubilizer to ensure good solubilization and/or dissolution of the therapeutic agent and/or pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin and to minimize precipitation of the therapeutic agent and/or pyrone analog sulfoalkyl cyclodextrin such as flavonoid-sulfoalkyl cyclodextrin. This can be especially important for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, .epsilon.-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other solubilizers known in the art, such as dimethyl acetamide, dimethyl isosorbide, N-methylpyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol, propylene glycol, and other additional cyclodextrins.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

Pharmaceutical compositions for injection In some embodiments, the invention provides a pharmaceutical composition for injection.

In some embodiments, the pharmaceutical composition for injection is an aqueous formulation comprising a pyrone analog such as a flavonoid and a sulfo-alkyl ether substituted cyclodextrin and a pharmaceutically or veterinarily acceptable aqueous carrier wherein the pyrone analog such as a flavonoid is present in a concentration greater than 0.5 mM, 1 mM, 5 mM, 10 mM, 20 mM, 30 mM, 33 mM, 40 mM, 50 mM, 60 mM, 70 mM, 80 mM or greater than 80 mM.

In some embodiments, the pharmaceutical composition for injection is an aqueous formulation comprising a pyrone analog such as a flavonoid and a sulfobutylether-7-β-cyclodextrin and a pharmaceutically or veterinarily acceptable aqueous carrier wherein the pyrone analog such as a flavonoid is present in a concentration greater than 0.5 mM. In some embodiments, the pharmaceutical composition for injection is an aqueous formulation comprising a pyrone analog such as a flavonoid and a sulfobutylether-7-β-cyclodextrin and a pharmaceutically or veterinarily acceptable aqueous carrier wherein the pyrone analog such as a flavonoid is present in a concentration greater than 1 mM. In some embodiments, the pharmaceutical composition for injection is an aqueous formulation comprising a pyrone analog such as a flavonoid and a sulfobutylether-7-β-cyclodextrin and a pharmaceutically or veterinarily acceptable aqueous carrier wherein the pyrone analog such as a flavonoid is present in a concentration greater than 5 mM. In some embodiments, the pharmaceutical composition for injection is an aqueous formulation comprising a pyrone analog such as a flavonoid and a sulfobutylether-7-β-cyclodextrin and a pharmaceutically or veterinarily acceptable aqueous carrier wherein the pyrone analog such as a flavonoid is present in a concentration greater than 10 mM. In some embodiments, the pharmaceutical composition for injection is an aqueous formulation comprising a pyrone analog such as a flavonoid and a sulfobutylether-7-β-cyclodextrin and a pharmaceutically or veterinarily acceptable aqueous carrier wherein the pyrone analog such as a flavonoid is present in a concentration greater than 15 mM. In some embodiments, the pharmaceutical composition for injection is an aqueous formulation comprising a pyrone analog such as a flavonoid and a sulfobutylether-7-β-cyclodextrin and a pharmaceutically or veterinarily acceptable aqueous carrier wherein the pyrone analog such as a flavonoid is present in a concentration greater than 20 mM. In some embodiments, the pharmaceutical composition for injection is an aqueous formulation comprising a pyrone analog such as a flavonoid and a sulfobutylether-7-β-cyclodextrin and a pharmaceutically or veterinarily acceptable aqueous carrier wherein the pyrone analog such as a flavonoid is present in a concentration greater than 30 mM. In some embodiments, the pharmaceutical composition for injection is an aqueous formulation comprising a pyrone analog such as a flavonoid and a sulfobutylether-7-β-cyclodextrin and a pharmaceutically or veterinarily acceptable aqueous carrier wherein the pyrone analog such as a flavonoid is present in a concentration greater than 33 mM. In some embodiments, the pharmaceutical composition for injection is an aqueous formulation comprising a pyrone analog such as a flavonoid and a sulfobutylether-7-β-cyclodextrin and a pharmaceutically or veterinarily acceptable aqueous carrier wherein the pyrone analog such as a flavonoid is present in a concentration greater than 40 mM. In some embodiments, the pharmaceutical composition for injection is an aqueous formulation comprising a pyrone analog such as a flavonoid and a sulfobutylether-7-β-cyclodextrin and a pharmaceutically or veterinarily acceptable aqueous carrier wherein the pyrone analog such as a flavonoid is present in a concentration greater than 50 mM. In some embodiments, the pharmaceutical composition for injection is an aqueous formulation comprising a pyrone analog such as a flavonoid and a sulfobutylether-7-β-cyclodextrin and a pharmaceutically or veterinarily acceptable aqueous carrier wherein the pyrone analog such as a flavonoid is present in a concentration greater than 60 mM. In some embodiments, the pharmaceutical composition for injection is an aqueous formulation comprising a pyrone analog such as a flavonoid and a sulfobutylether-7-β-cyclodextrin and a pharmaceutically or veterinarily acceptable aqueous carrier wherein the pyrone analog such as a flavonoid is present in a concentration greater than 80 mM.

In some embodiments, the pharmaceutical composition for injection is made using an aqueous composition comprising a quercetin, a sulfo-alkyl ether substituted cyclodextrin and a pharmaceutically or veterinarily acceptable aqueous carrier wherein the quercetin is present in a concentration of greater than 0.5 mM, 1 mM, 5 mM, 10 mM, 20 mM, 30 mM, 33 mM, 40 mM, 50 mM, 60 mM, 70 mM, 80 mM or greater than 80 mM.

In some embodiments, the pharmaceutical composition for injection is made using an aqueous composition comprising quercetin or a quercetin derivative, a sulfobutylether-7-β-cyclodextrin, and a pharmaceutically or veterinarily acceptable aqueous carrier wherein the quercetin or a quercetin derivative is present in a concentration of greater than 0.5 mM in the composition used to make the formulation. In some embodiments, the pharmaceutical composition for injection is made using an aqueous composition comprising quercetin or a quercetin derivative, a sulfobutylether-7-β-cyclodextrin, and a pharmaceutically or veterinarily acceptable aqueous carrier wherein the quercetin or a quercetin derivative is present in a concentration of greater than 1 mM in the composition used to make the formulation. In some embodiments, the pharmaceutical composition for injection is made using an aqueous composition comprising quercetin or a quercetin derivative, a sulfobutylether-7-β-cyclodextrin, and a pharmaceutically or veterinarily acceptable aqueous carrier wherein the quercetin or a quercetin derivative is present in a concentration of greater than 5 mM in the composition used to make the formulation. In some embodiments, the pharmaceutical composition for injection is made using an aqueous composition comprising quercetin or a quercetin derivative, a sulfobutylether-7-β-cyclodextrin, and a pharmaceutically or veterinarily acceptable aqueous carrier wherein the quercetin or a quercetin derivative is present in a concentration of greater than 10 mM in the composition used to make the formulation. In some embodiments, the pharmaceutical composition for injection is made using an aqueous composition comprising quercetin or a quercetin derivative, a sulfobutylether-7-β-cyclodextrin, and a pharmaceutically or veterinarily acceptable aqueous carrier wherein the quercetin or a quercetin derivative is present in a concentration of greater than 15 mM in the composition used to make the formulation. In some embodiments, the pharmaceutical composition for injection is made using an aqueous composition comprising quercetin or a quercetin derivative, a sulfobutylether-7-β-cyclodextrin, and a pharmaceutically or veterinarily acceptable aqueous carrier wherein the quercetin or a quercetin derivative is present in a concentration of greater than 20 mM in the composition used to make the formulation. In some embodiments, the pharmaceutical composition for injection is made using an aqueous composition comprising quercetin or a quercetin derivative, a sulfobutylether-7-β-cyclodextrin, and a pharmaceutically or veterinarily acceptable aqueous carrier wherein the quercetin or a quercetin derivative is present in a concentration of greater than 30 mM in the composition used to make the formulation. In some embodiments, the pharmaceutical composition for injection is made using an aqueous composition comprising quercetin or a quercetin derivative, a sulfobutylether-7-β-cyclodextrin, and a pharmaceutically or veterinarily acceptable aqueous carrier wherein the quercetin or a quercetin derivative is present in a concentration of greater than 33 mM in the composition used to make the formulation. In some embodiments, the pharmaceutical composition for injection is made using an aqueous composition comprising quercetin or a quercetin derivative, a sulfobutylether-7-β-cyclodextrin, and a pharmaceutically or veterinarily acceptable aqueous carrier wherein the quercetin or a quercetin derivative is present in a concentration of greater than 40 mM in the composition used to make the formulation. In some embodiments, the pharmaceutical composition for injection is made using an aqueous composition comprising quercetin or a quercetin derivative, a sulfobutylether-7-β-cyclodextrin, and a pharmaceutically or veterinarily acceptable aqueous carrier wherein the quercetin or a quercetin derivative is present in a concentration of greater than 50 mM in the composition used to make the formulation. In some embodiments, the pharmaceutical composition for injection is made using an aqueous composition comprising quercetin or a quercetin derivative, a sulfobutylether-7-β-cyclodextrin, and a pharmaceutically or veterinarily acceptable aqueous carrier wherein the quercetin or a quercetin derivative is present in a concentration of greater than 60 mM in the composition used to make the formulation. In some embodiments, the pharmaceutical composition for injection is made using an aqueous composition comprising quercetin or a quercetin derivative, a sulfobutylether-7-β-cyclodextrin, and a pharmaceutically or veterinarily acceptable aqueous carrier wherein the quercetin or a quercetin derivative is present in a concentration of greater than 80 mM in the composition used to make the formulation.

Where the pharmaceutical composition for injection is made from the aqueous composition of pyrone analog sulfoalkyl cyclodextrin such as flavonoid-sulfoalkyl cyclodextrin, pharmaceutical composition for injection can be made into a solid formulation that is produced by drying the aqueous composition, for example by freeze drying or lyophilization. Having a dried, solid formulation can be advantageous for increasing the shelf-life. The solid formulation can then be re-dissolved into solution for injection. The dried powder can be further formulated into pharmaceutical composition for injection as described herein.

In some embodiments, the pharmaceutical composition for injection comprises a combination of a therapeutic agent and a pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin, and a pharmaceutical excipient suitable for injection. Components and amounts of agents in the compositions are as described herein.

In some embodiments, the pharmaceutical composition for injection containing a combination of a therapeutic agent and a pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin that reduces or eliminates a side effect of the therapeutic agent, and a pharmaceutical excipient suitable for injection. Components and amounts of agents in the compositions are as described herein.

The forms in which the compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the transport protein modulator and/or the therapeutic agent in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Pharmaceutical compositions for topical (e.g. transdermal) delivery In some embodiments, the invention provides a pharmaceutical composition for transdermal delivery is an aqueous formulation comprising a pyrone analog such as a flavonoid and a sulfo-alkyl ether substituted cyclodextrin and a pharmaceutically or veterinarily acceptable aqueous carrier wherein the pyrone analog such as a flavonoid is present in a concentration greater than 0.5 mM, 1 mM, 5 mM, 10 mM, 20 mM, 30 mM, 33 mM, 40 mM, 50 mM, 60 mM, 70 mM, 80 mM or greater than 80 mM.

In some embodiments, the invention provides a pharmaceutical composition for transdermal delivery is an aqueous formulation comprising quercetin or a quercetin derivative and a sulfobutylether-7-β-cyclodextrin and a pharmaceutically or veterinarily acceptable aqueous carrier wherein the pyrone analog such as a flavonoid is present in a concentration greater than 0.5 mM, 1 mM, 5 mM, 10 mM, 20 mM, 30 mM, 33 mM, 40 mM, 50 mM, 60 mM, 70 mM, 80 mM or greater than 80 mM.

In some embodiments, the pharmaceutical composition for transdermal delivery is made using an aqueous composition comprising a pyrone analog such as a flavonoid, a sulfo-alkyl ether substituted cyclodextrin and a pharmaceutically or veterinarily acceptable aqueous carrier wherein the pyrone analog such as a flavonoid is present in a concentration of greater than 0.5 mM, 1 mM, 5 mM, 10 mM, 20 mM, 30 mM, 33 mM, 40 mM, 50 mM, 60 mM, 70 mM, 80 mM or greater than 80 mM.

In some embodiments, the pharmaceutical composition for transdermal delivery is made using an aqueous composition comprising a pyrone analog such as a flavonoid, e.g. quercetin and a sulfobutylether-7-β-cyclodextrin and a pharmaceutically or veterinarily acceptable aqueous carrier wherein the pyrone analog such as a flavonoid is present in a concentration of greater than 0.5 mM, 1 mM, 5 mM, 10 mM, 20 mM, 30 mM, 33 mM, 40 mM, 50 mM, 60 mM, 70 mM, 80 mM or greater than 80 mM.

In some embodiments, the pharmaceutical composition for transdermal delivery is a combination of a therapeutic agent and sulfoalkyl ether cyclodextrin-pyrone analog such as a flavonoid, e.g. sulfobutylether-7-β-cyclodextrin-pyrone analog such as a flavonoid, and a pharmaceutical excipient suitable for transdermal delivery. Components and amounts of agents in the compositions are as described herein.

In some embodiments, the pharmaceutical composition for transdermal delivery is a combination of a therapeutic agent and pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin that reduces or eliminates a side effect of the therapeutic agent, and a pharmaceutical excipient suitable for transdermal delivery. In some embodiments, the pyrone analog sulfoalkyl cyclodextrin such as flavonoid-sulfoalkyl cyclodextrin that reduces or eliminates the side effect of the therapeutic agent is a BTB transport protein modulator. Components and amounts of agents in the compositions are as described herein.

Compositions of the present invention can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another preferred formulation for use in the methods of the present invention employs transdermal delivery devices (“patches”). Such transdermal patches may be used to provide continuous or discontinuous infusion of the transport protein modulator in controlled amounts, either with or without therapeutic agent. Thus, in some embodiments the invention provides a transdermal patch incorporating a pyrone analog sulfoalkyl cyclodextrin such as flavonoid-sulfoalkyl cyclodextrin in combination with a therapeutic agent. In some embodiments the invention provides a transdermal patch incorporating a pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin in combination with a therapeutic agent, e.g. an analgesic such as an opioid analgesic.

The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

Pharmaceutical compositions for inhalation. Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

Other pharmaceutical compositions Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety.

### X. Kits

The invention also provides kits. In some embodiments the kits include a container comprising pharmaceutical formulation that is made using an aqueous composition comprising a pyrone analog such as a flavonoid, a sulfo-alkyl ether substituted cyclodextrin and a pharmaceutically or veterinarily acceptable aqueous carrier wherein the pyrone analog such as a flavonoid is present in a concentration of greater than 0.5 mM, 1 mM, 5 mM, 10 mM, 20 mM, 30 mM, 33 mM, 40 mM, 50 mM, 60 mM, 70 mM, 80 mM or greater than 80 mM in the composition used to make the formulation, and instructions for using the formulation to treat a disorder. In some embodiments, the kits can include a sulfobutylether-7-β-cyclodextrin-pyrone analog such as a flavonoid for example sulfobutylether-7-β-cyclodextrin-quercetin, in suitable packaging, and written material that can include instructions for use, discussion of clinical studies, listing of side effects, and the like. The kits can include pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin, for example sulfobutylether-7-β-cyclodextrin-quercetin that act as an agent to reduce or eliminate a side effect of a therapeutic agent, in suitable packaging, and written material that can include instructions for use, discussion of clinical studies, listing of side effects, and the like. The kits may further contain a therapeutic agent that has a side effect. In some embodiments, the therapeutic agent and the agent that reduces or eliminates a side effect of the therapeutic agent are provided as separate compositions in separate containers within the kit. In some embodiments, the therapeutic agent and the agent that reduces or eliminates a side effect of the therapeutic agent are provided as a single composition within a container in the kit. Suitable packaging and additional articles for use (e.g., measuring cup for liquid preparations, foil wrapping to minimize exposure to air, and the like) are known in the art and may be included in the kit.

### XI. Methods of Treatment

In another aspect, the invention provides methods, including methods of treatment, methods of decreasing the concentration of a substance in a physiological compartment (e.g., methods of delaying the onset or preventing chronic neurodegenerative diseases), methods of enhancing a therapeutic effect of a substance, methods of delaying, preventing, reducing or eliminating tolerance or dependence in an animal that is administered a substance, methods of drug wash-out, and methods for identifying modulators of blood-brain barrier transport proteins.

For simplicity, methods will be described in terms of reduction of a side effect of a substance. It is understood that the methods apply equally to exclusion of a substance from the fetal compartment, or reduction of fetal effects of a substance.

The term “animal” or “animal subject” as used herein includes humans as well as other mammals. The methods generally involve the administration of one or more drugs for the treatment of one or more diseases. Combinations of agents can be used to treat one disease or multiple diseases or to modulate the side-effects of one or more agents in the combination.

The term “treating” and its grammatical equivalents as used herein includes achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

A. Methods of Treating Conditions

In some embodiments, the invention provides a method of treating a condition by administering to an animal suffering from the condition an effective amount of a therapeutic agent and an amount of a pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin that is a BTB transport protein activator sufficient to reduce or eliminate a side effect of the therapeutic agent. In some embodiments, the activator reduces or eliminates a plurality of side effect of the therapeutic agent. In some embodiments the animal is a mammal, e.g., a human.

In some embodiments of the methods of the invention, the invention provides a method of treating a condition by administering to an animal suffering from the condition an effective amount of tacrolimus and an amount of a BTB transport protein modulator sufficient to change the concentration of tacrolimus in a physiological compartment. In some embodiments of the methods of the invention the physiological compartment is selected from the group consisting of blood, lymph nodes, spleen, peyer's patches, lungs, heart kidney, pancreas liver, and gull bladder. In some embodiments of the methods of the invention the BTB transport modulator decrease the clearance of tacrolimus from a compartment where the drug is exerting therapeutic effect.

The therapeutic agent and the pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin are co-administered. “Co-administration,” “administered in combination with,” and their grammatical equivalents, as used herein, encompasses administration of two or more agents to an animal so that both agents and/or their metabolites are present in the animal at the same time. Co-administration includes simultaneous administration in separate compositions, administration at different times in separate compositions, or administration in a composition in which both agents are present. Thus, in some embodiments, the pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin are administered in a single composition. In some embodiments, the therapeutic agent and the pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin are admixed in the composition. Typically, the therapeutic agent is present in the composition in an amount sufficient to produce a therapeutic effect, and the pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin is present in the composition in an amount sufficient to reduce a central nervous system effect of the therapeutic agent. In some embodiments, the therapeutic agent is present in an amount sufficient to exert a therapeutic effect and the pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin is present in an amount sufficient to decrease a side effect of the therapeutic agent by an average of at least about 5, 10, 15, 20, 25, 30, 40, 50, 60, 70, 80, 90, more than 90%, or substantially eliminate a side effect compared to the effect without the pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin.

Administration of the therapeutic agent and the pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin may be any suitable means. If the agents are administered as separate compositions, they may be administered by the same route or by different routes. If the agents are administered in a single composition, they may be administered by any suitable route. In some embodiments, the agents are administered as a single composition by oral administration. In some embodiments, the agents are administered as a single composition by transdermal administration. In some embodiments, the agents are administered as a single composition by injection.

In some embodiments, in the pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin, the pyrone analog such as a flavonoid is quercetin or a quercetin derivative, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, 5,7-dideoxyquercetin (3,3′,4′-trihydroxyflavone), or epicatechin. In some embodiments, the pyrone analog such as a flavonoid is quercetin or a quercetin derivative, kaempferol, or galangin. In some embodiments, the pyrone analog such as a flavonoid is quercetin. In some embodiments, the pyrone analog such as a flavonoid is fisetin. In some embodiments, the pyrone analog such as a flavonoid is 5,7-dideoxyquercetin. Dosages are as provided for compositions. Typically, the daily dosage of the pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin will be about 0.5-100 mg/kg.

The therapeutic agent may be any suitable therapeutic agent described herein. In some embodiments, the therapeutic agent is an antihypertensive, vasodilator, barbiturate, membrane stabilizer, cardiac stabilizer, glucocorticoid, or antiinfectives, as described herein.

In some embodiments the therapeutic agent is an analgesic agent, e.g., an opiate such as morphine or oxycodone. In some embodiments, the therapeutic agent is an immunosuppressant such as a calcineurin inhibitor, e.g., tacrolimus or a tacrolimus analog. Further therapeutic agents are described in U.S. Patent Publication No. US2006/0111308 and US2008/0161248; and PCT Publication No. WO/06055672 and WO/08083160, incorporated by reference herein in their entirety.

The methods of the invention may be used for treatment of any suitable condition, e.g., diseases of the heart, circulation, lipoprotein metabolism, hemostasis and thrombosis, respiratory system, kidney, gastrointestinal tract, endocrine system, reproductive system, or hemopoeitic system, where one or more therapeutic agents are used that have side effect.

For example, in some embodiments, the methods of the invention include the treatment of hypertension in an animal by administering to an animal in need of treatment an effective amount of an antihypertensive and an effective amount pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin that reduces or eliminates a side effect of the hypertensive. Another exemplary embodiment is the treatment or prevention of infection in an animal by administering to an animal in need of treatment or prevention of infection an effective amount of an antiinfective agent and an effective amount of pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin that reduces or eliminates a side effect of the antiinfective agent.

When a therapeutic agent and an agent that reduces or eliminates a side effect of the therapeutic agent are used in combination, any suitable ratio of the two agents, e.g., molar ratio, wt/wt ration, wt/volume ratio, or volume/volume ratio, as described herein, may be used.

B. Methods of Decreasing the Concentration of a Substance in a Physiological Compartment

The invention provides methods for reducing the concentration of a substance in a physiological compartment by selectively increasing efflux of the substance from the physiological compartment to an external environment. The physiological compartment can be a central nervous system or a fetal compartment. In some embodiments of the compositions of the invention, the physiological compartment includes blood, lymph nodes, spleen, peyer's patches, lungs, and heart.

In some embodiments, compositions of the invention may be administered chronically to an individual in order to prevent, delay the appearance, or slow or halt the progression of a chronic neurodegenerative condition. In some embodiments, compositions of the invention may be administered chronically to an individual in order to remove from the CNS one or more substances associated with a chronic neurodegenerative condition. In some embodiments, the neurodegenerative condition is prion disease, Alzheimer's disease (AD), Parkinson's disease (PD), Huntington's disease (HD), ALS, multiple sclerosis, transverse myelitis, motor neuron disease, Pick's disease, tuberous sclerosis, lysosomal storage disorders, Canavan's disease, Rett's syndrome, spinocerebellar ataxias, Friedreich's ataxia, optic atrophy, or retinal degeneration. In some embodiments, the neurodegenerative disease is AD. In some embodiments, the substance associated with a neurodegenerative disease is amyloid beta. In some embodiments, a pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin is administered to the individual where the pyrone analog such as a flavonoid is, for example quercetin or a quercetin derivative, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, 5,7-dideoxyquercetin (3,3′,4′-trihydroxyflavone), or epicatechin. In some embodiments, the individual is a human and is chronically administered an amount of quercetin or a quercetin derivative effective in removing amyloid beta from the CNS. In some embodiments, the quercetin is administered in a pharmaceutical composition with a pharmaceutically acceptable excipient at a dose of 100 mg-10,000 mg per day. Other dosages of quercetin, as described herein, may also be used.

C. Methods of Treating Pain.

The invention provides methods of treating pain such as acute or chronic pain, using therapeutic agents and the compositions of the invention. Any suitable type of pain, whether acute or chronic, may be treated by the methods of the invention. Thus, in some embodiments, the invention provides a method of treating an animal for pain by administering to an animal in pain an effective amount of an analgesic agent, e.g. an opiate such as morphine or oxycodone and an amount of a pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin sufficient to reduce a side effect of the analgesic agent. Further description of types of pain, analgesic agents and treatment of pain may be found in U.S. Patent Publication No. US2006/0111308 and PCT Publication No. WO/06055672, incorporated by reference herein in their entirety.

D. Wash-Out Methods

The invention further provides methods of reversing one or more side effect of a substance by administering a pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin to an animal that has received an amount of the substance sufficient to produce one or more side effect. The methods are especially useful in a situation where it is desired to rapidly reverse one or more side effect of a substance, e.g., in an overdose situation or to enhance recovery from general anesthesia. Any suitable sulfoalkyl ether cyclodextrin-pyrone analog such as sulfoalkyl ether flavonoid, such as described herein may be used.

In some embodiments, the invention provides a method for reversing a side effect of an agent in a human by administering to the human an amount of a pyrone analog sulfoalkyl cyclodextrin such as flavonoid-sulfoalkyl cyclodextrin, e.g. sulfobutylether-7-β-cyclodextrin-quercetin sufficient to partially or completely reverse a central nervous system effect of the agent, where the human has received an amount of said agent sufficient to produce a central nervous system effect. In some embodiments, the agent is a general anesthetic. Examples of general anesthetics include, but not limited to, desflurane, dexmedetomidine, diazepam, droperidol, enflurane, etomidate, halothane, isoflurane, ketamine, lorazepam, methohexital, methoxyflurane, midazolam, nitrous Oxide propofol, sevoflurane, and thiopental. In some embodiments, the human has received an overdose of the agent producing the side effect. In some embodiments, the individual continues to experience peripheral effects of the agent. In some embodiments, the flavonoid in the pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin is quercetin or a quercetin derivative, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, 5,7-dideoxyquercetin (3,3′,4′-trihydroxyflavone), or epicatechin. In some embodiments, the pyrone analog such as a flavonoid is quercetin or a quercetin derivative. Typically, the pyrone analog such as a flavonoid will be administered by injection, e.g., intravenously or intraperitoneally, in a dose sufficient to partially or completely reverse a side effect of the substance. Such a dose in a human can be, e.g., about 0.1-100 gm, or about 0.5-50 gm, or about 1-20 gm, or 1, 2, 3, 4, 5, 6, 7, 8, 9, 10, 12, 14, 16, 18, or 20 gm. In general, the dose can be 0.01-1.5 gm/kg.

E. Administration

The methods involve the administration of an agent that reduces or eliminates a side effect of a substance. In some embodiments, a therapeutic agent that produces a side effect is administered in combination with a pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin that reduces the effects of a side effect of the therapeutic agent. In some embodiments, other agents are also administered, e.g., other therapeutic agents. When two or more agents are co-administered, they may be co-administered in any suitable manner, e.g., as separate compositions, in the same composition, by the same or by different routes of administration.

In some embodiments, the pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin is administered in a single dose. This may be the case, e.g., in wash-out methods where the agent is introduced into an animal to quickly lower the side effect of a substance already present in the body. Typically, such administration will be by injection, e.g., intravenous injection, in order to introduce the agent quickly. However, other routes may be used as appropriate. A single dose of an agent that reduces or eliminates a side effect of a substance may also be used when it is administered with the substance (e.g., a therapeutic agent that produces a side effect) for treatment of an acute condition.

In some embodiments, the pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin and/or the therapeutic agent is administered in multiple doses. Dosing may be about once, twice, three times, four times, five times, six times, or more than six times per day. Dosing may be about once a month, once every two weeks, once a week, or once every other day. In one embodiment the drug is an analgesic. In another embodiment the analgesic compound and the transport protein activator are administered together about once per day to about 6 times per day. In some embodiments the pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin and/or the therapeutic agent are administered 2 to 3 times per day. In some embodiments the pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin and the therapeutic agent are administered each 2 to 3 times per day and the pyrone analog sulfoalkyl cyclodextrin such as flavonoid-sulfoalkyl cyclodextrin is administered about 20, 30, 45, 60, or 90 minutes before administering the analgesic. In another embodiment the administration of the analgesic compound and the transport protein activator continues for less than about 7 days. In yet another embodiment the administration continues for more than about 6, 10, 14, 28 days, two months, six months, or one year. In some cases, continuous dosing is achieved and maintained as long as necessary, e.g., intravenous administration of analgesic in a post-operative situation or for a terminally ill patient, or transdermal dosing for chronic pain.

In some embodiments, the pyrone analog sulfoalkyl cyclodextrin such as flavonoid-sulfoalkyl cyclodextrin, e.g. sulfobutylether-7-β-cyclodextrin-quercetin or a quercetin derivative is administered two to three times a day with an oral dose of about 500 mg or an intravenous dose of about 150 mg. In some embodiments the pyrone analog sulfoalkyl cyclodextrin such as flavonoid-sulfoalkyl cyclodextrin, e.g. sulfobutylether-7-β-cyclodextrin-quercetin or a quercetin derivative is administered about one hour or about 30 minutes prior to administration of the therapeutic agent. In some embodiments the pyrone analog sulfoalkyl cyclodextrin such as flavonoid-sulfoalkyl cyclodextrin, e.g. sulfobutylether-7-β-cyclodextrin-quercetin or a quercetin derivative is administered such that it is in the bloodstream 30 minutes prior to administration of the therapeutic agent. This timing may be accomplished by administering the pyrone analog sulfoalkyl cyclodextrin such as flavonoid-sulfoalkyl cyclodextrin and the therapeutic agent separately, or by administering the quercetin or a quercetin derivative and agent in the same composition that is formulated such that quercetin or a quercetin derivative reaches the bloodstream before the therapeutic agent.

Administration of the agents of the invention may continue as long as necessary. In some embodiments, an agent of the invention is administered for more than 1, 2, 3, 4, 5, 6, 7, 14, or 28 days. In some embodiments, an agent of the invention is administered for less than 28, 14, 7, 6, 5, 4, 3, 2, or 1 day. In some embodiments, an agent of the invention is administered chronically on an ongoing basis, e.g., for the treatment of chronic pain.

An effective amount of a transport protein modulator and an effective amount of a drug may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

The pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin and the therapeutic agent may be administered in dosages as described herein (see, e.g., Compositions). Dosing ranges for therapeutic agents are known in the art. Dosing for the pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin may be found by routine experimentation. For a pyrone analog sulfoalkyl cyclodextrin such as flavonoid-sulfoalkyl cyclodextrin, e.g. sulfobutylether-7-β-cyclodextrin-quercetin, typical daily dose ranges based on the weight of quercetin are, e.g. about 1-5000 mg, or about 1-3000 mg, or about 1-2000 mg, or about 1-1000 mg, or about 1-500 mg, or about 1-100 mg, or about 10-5000 mg, or about 10-3000 mg, or about 10-2000 mg, or about 10-1000 mg, or about 10-500 mg, or about 10-200 mg, or about 10-100 mg, or about 20-2000 mg or about 20-1500 mg or about 20-1000 mg or about 20-500 mg, or about 20-100 mg, or about 50-5000 mg, or about 50-4000 mg, or about 50-3000 mg, or about 50-2000 mg, or about 50-1000 mg, or about 50-500 mg, or about 50-100 mg, about 100-5000 mg, or about 100-4000 mg, or about 100-3000 mg, or about 100-2000 mg, or about 100-1000 mg, or about 100-500 mg. In some embodiments, the daily dose of sulfobutylether-7-β-cyclodextrin-quercetin has an amount of quercetin of about 100, 200, 300, 400, 500, 600, 700, 800, 900, 1000, 1500, 2000, 3000, 5000 or 10000 mg. In some embodiments, the daily dose of quercetin is 500 mg. In some embodiments, the daily dose of quercetin is 900 mg. In some embodiments, the daily dose of quercetin is about 2100 mg. Daily dose range may depend on the form of pyrone analog such as a flavonoid, e.g., the carbohydrate moieties attached to the pyrone analog such as a flavonoid, and/or factors with which the pyrone analog such as a flavonoid is administered, as described herein. The serum half-life for, e.g., quercetin, is about 19-25 hours, so single dose accuracy is not crucial.

The single dose levels and daily dose levels can depend on the route of administration. For example, for intravenous administration, a single dose of sulfobutylether-7-β-cyclodextrin-quercetin may contain about 50 mg to about 200 mg of quercetin, about 100 mg to about 150 mg of quercetin, about 50 mg to about 100 mg of quercetin, about 50 mg to about 200 mg of quercetin, or about 150 mg of quercetin, which is given 2 to 3 times a day for a daily dose of about 100 mg to 600 mg of quercetin, about 200 mg to 400 mg of quercetin, about 300 mg to 500 mg of quercetin, about 400 mg to 500 mg of quercetin, or about 450 mg of quercetin per day.

For oral administration, a single dose of sulfobutylether-7-β-cyclodextrin-quercetin may contain about 200 mg to about 800 mg of quercetin, about 300 mg to about 600 mg of quercetin, about 400 mg to about 600 mg of quercetin, or about 500 mg of quercetin, which is given 2 to 3 times a day for a daily dose of about 400 mg to 2400 mg of quercetin, about 800 mg to 1800 mg of quercetin, about 1200 mg to 1800 mg of quercetin, about 1400 mg to 1600 mg of quercetin, or about 1500 mg of quercetin per day.

When a pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin, is administered in a composition that comprises one or more therapeutic agents, and the therapeutic agent has a shorter half-life than the sulfoalkyl ether cyclodextrin-pyrone analog such as a flavonoid (e.g., tramadol, hydrocodone, and the like have shorter half-lives than quercetin), unit dose forms of the therapeutic agent and the pyrone analog sulfoalkyl cyclodextrin such as flavonoid-sulfoalkyl cyclodextrin, e.g. pyrone analog-sulfobutylether-7-β-cyclodextrin such as flavonoid-sulfobutylether-7-β-cyclodextrin may be adjusted accordingly. Thus, for example, if sulfobutylether-7-β-cyclodextrin-quercetin is given in a composition also containing, e.g., tramadol, a typical unit dose form may be, e.g., 50 mg tramadol/100 mg quercetin, or 50 mg tramadol/500 mg quercetin. See e.g., Compositions.

Table 3 below, provides exemplary dosing schemes for selected analgesic agents and quercetin. These dosages are provided by way of example only and do not limit the invention.

TABLE 3
Exemplary dosing schemes

| Therapeutic Agent (A) + | Per Dose (A:Q)* | | Per Day(A:Q) | |
|---|---|---|---|---|
| Quercetin dihydrate (Q) | ~mole:mole | ~mg:mg | ~mole:mole | ~mg:mg |
| Vicodin | 0.006:1 | 10:1000 | 0.01:1 | 30:2000 |
| TID(hydrocodone | | | | |
| bitartrate - 5 mg per | | | | |
| tablet) | | | | |
| Tramadol | 0.1:1 | 100:1000 | 0.2:1-0.3:1 | 400-600:2000 |
| OxyContin | 0.07:1 | 80:1000 | 0.1:1 | 240:2000 |
| Methadone | 0.04:1 | 40:1000 | 0.2:1 | 400:2000 |
| Gabapentin | 0.6:1 | 300:1000 | 0.8:1 | 900:2000 |
| | 1.75:1 | 900:1000 | 2.6:1 | 2700:2000 |
| Lorazepam (Ativan) | 0.001:1 | 1:1000 | 0.001:1 | 3:2000 |
| Cyclobnzaprine | 0.01:1 | 10:1000 | 0.01:1 | 30:2000 |
| hydrochloride | | | | |
| (Flexeril) | | | | |
| Carisoprodol (Soma) | 0.4:1 | 350:1000 | 0.6:1 | 1050:2000 |

*2000 mg quercetin daily, given in two divided doses, e.g., with two doses of the analgesic. Some doses of analgesic were given without quercetin.

### EXAMPLES

### Example 1

### Preparation of a Sulfobutylether-7-β-Cyclodextrin Aqueous Composition

Under an inert atmosphere, 18.7 g of sulfobutylether-7-β-cyclodextrin (Captisol™, CyDex) is dissolved in about 50 ml of deionized (DI) water in a round-bottomed flask with magnetic stirring. The flask is placed in an ice bath. When all of the Captisol is dissolved, 1.24 g of quercetin (Micron Technologies) (equivalent to about 1 g of anhydrous quercetin) is added with stirring. Into the flask, 12 ml of 1 N sodium hydroxide is added over about 5 to 10 minutes. The appearance of the reaction should be clear indicating that both the Captisol and the quercetin are dissolved. Into the flask is then added 10.5 ml of hydrochloric acid over 5 to 10 minutes at a slow enough rate to avoid precipitation. During the addition of the sodium hydroxide and the hydrochloric acid, the temperature is maintained at less than 20° C. DI water is then added to give total volume of 100 ml. This procedure results in a sulfobutylether-7-β-cyclodextrin-quercetin aqueous composition at a concentration of 10 mg/ml (33 mM) in quercetin at a pH of about 7.8. This solution was found to be stable on storage for weeks without precipitation.

In a variation of the above method, 9 ml rather than 10.5 ml of hydrochloric acid is added to make a solution with a pH of about 8.4.

### Example 2

### In-Vivo Study of Sulfobutylether-7-β-Cyclodextrin-Quercetin Aqueous Composition

### Rat CWTF

This experiment demonstrates the effectiveness of the high concentration aqueous compositions of the present invention for analgesia when co-administered with an analgesic. The rat cold water tail flick (CETF) test is used to determine the maximum percent analgesia (% MPA) by the following procedure:A 1:1 mix of ethylene glycol/water is maintained at −3 degrees C. in a circulating water bath. Each rat is held over the bath with its tail submerged approximately half way into the liquid. The nociceptive threshold is taken as the latency before removal or flicking of the tail. For each animal, the first reading is discarded and the mean of a further three readings (at least 30 minutes apart) is noted. Only rats whose baseline values fall within the 10-20 sec range are used. A quercetin solution (Q-Captisol) that was made by the base then acid process described herein at a concentration of 25 mg/ml (83 mM) in quercetin was used. The quercetin solution was injected i.p. into rats for a dose of 25 mg/kg (0.20 ml/200 g rat) 30 min before morphine (6 mg/kg, s.c., 0.20 ml/200 g rat). Tail flick values are again taken 30, 60, 90 and 120 min after morphine administration. The % maximal possible analgesia (% MPA) is calculated for each rat at each time interval using the formula:

[(Test latency−baseline latency)/(60−baseline latency)]×100 where 60 seconds is used as the cutoff limit.

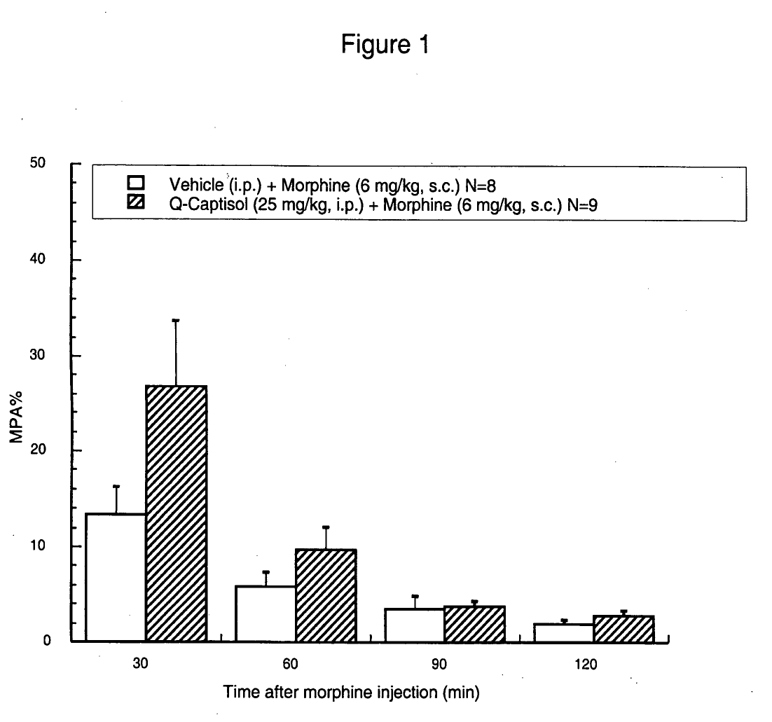

FIG. 1 shows the CWTF results for a study in which one set of rats (N=8) was given morphine at 6 mg/kg

s.c. along with vehicle, and another set of rats (N=9) was given morphine at 6 mg/kg s.c. along with sulfobutylether-7-β-cyclodextrin-quercetin aqueous composition (Q-Captisol) at 25 mg/kg, i.p. The results show that the injection of the sulfobutylether-7-β-cyclodextrin-quercetin aqueous composition 30 min prior to the morphine injection can improve the analgesic effect of the morphine.

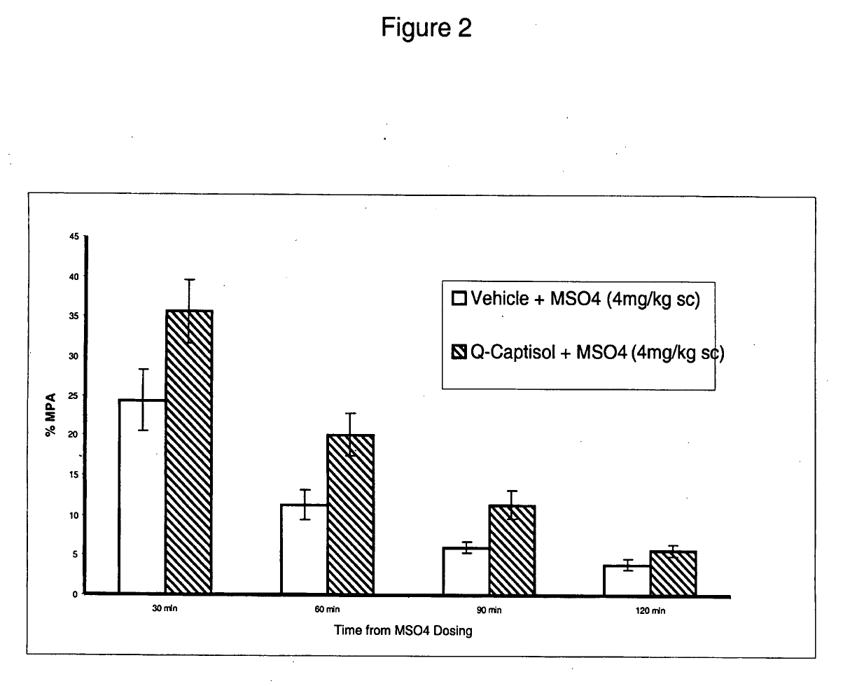

FIG. 2 shows the CWTF results for a similar study in which one set of rats was given morphine at 4 mg/kg s.c. along with vehicle, and another set of rats was given morphine at 4 mg/kg s.c. along with sulfobutylether-7-β-cyclodextrin-quercetin aqueous composition (Q-Captisol) at 25 mg/kg, i.p. The results show that the injection of the sulfobutylether-7-β-cyclodextrin-quercetin aqueous composition 30 min prior to the morphine injection can improve the analgesic effect of the morphine even at morphine doses as low as 4 mg/kg.

### Example 3

### Reversal Effect of Modulator, Sulfobutylether-7-β-Cyclodextrin-Quercetin, on Sedative Effects in Rodents

This example shows how An anesthetic wake up test is used to assess the reversal effect of modulator, quercetin, on the sedative effects of barbiturates, opioids, and benzodiazepines when administered as a high concentration aqueous solution of sulfobutylether-7-β-cyclodextrin-quercetin. This is a single blind, randomized, controlled animal trial. Approximately 48 rodents are utilized throughout the study. Animals may be reused. However, a washout of 24 hours is required between exposures.

Twelve rodents are utilized in each portion of this trial. Intravenous barbiturate (e.g. diprivan, pentobarbital, or phenobarbital) anesthesia is induced and titrated to spontaneous but slow respirations and lack of response to painful stimulation. Supplemental oxygen is delivered. A maximum of 3 doses of intraperitoneal sulfobutylether-7-β-cyclodextrin-quercetin are tested (low, medium, high) along with placebo. The sulfobutylether-7-β-cyclodextrin-quercetin is administered 30 minutes prior to administration of the barbiturate. Once administered rodents are monitored with the help of stopwatch for time to awakening and return to normal respiratory rate. Once awakened, rodents are tested for time to withdrawal from painful stimulus and performance on rotarod.

This study is repeated as a single agent trial with opioids (remifentanyl, fentanyl, morphine, etc) and benzodiazepines (diazepam, midazolam, lorazepam). This study is also repeated as a multi agent trial utilizing one opioid, one benzodiazepine, and one barbiturate.

### Example 4

### Sulfobutylether-7-α-Cyclodextrin-Quercetin

### CNS Effects of Oxycodone in an Acute Pain Model

Opioids frequently produce adverse CNS side effects in ambulatory settings, providing a rationale for improving opioid analgesia by minimizing adverse effects. Quercetin is a flavonol which is shown herein to activate PgP efflux of pump ligands at the blood brain barrier. In this pilot study, the hypothesis that quercetin, administered as a sulfobutylether-7-β-cyclodextrin-quercetin, improves the tolerability and safety of oxycodone without impairing analgesia is tested. Healthy subjects (N=65) undergoing third molar removal are randomly allocated to receive 500 mg oral quercetin in the form of sulfobutylether-7-β-cyclodextrin-quercetin or matching placebo at 1 hr prior to surgery. All subjects receive 10 mg oral oxycodone immediately prior to surgery. Decrease in pupil size is measured. Oxycodone concentrations are measured prior to surgery, at 1 hr, or at 4 hr. Total Nausea and Vomiting Score (TNVS) is calculated for the time from dosing through 24 hr. The test will also determine whether more subjects in the quercetin group than in the placebo group experience “no” nausea and vomiting. The tendency of subject to request an anti-emetic is also monitored. The patients are interviewed to determine whether the quercetin treated group have consistently lower mean pain intensity than the placebo group. Sulfobutylether-7-β-cyclodextrin-quercetin given 1 hr prior to oxycodone may ameliorate the severity of nausea and vomiting while not interfering with analgesic efficacy.

### Example 5

### Solubility of Quercetin with Sulfobutylether-7-β-cyclodextrin and Arginine

Sulfobutylether-7-β-cyclodextrin (Captisol™) is dissolved in water to form a solution at 30% w/v. To the Captisol solution is added Arginine at a concentration of 70 mM and Captisol™ at a concentration of about 20 mg/ml. The solution is stirred at room temperature for about 10 minutes. The solution is separated from any excess solids (e.g. by filtration). The concentration of quercetin in the solution is about 10.2 mg/mL.

### Example 6

### Solubility of Quercetin with Sulfobutylether-7-β-cyclodextrin and Lysine

Sulfobutylether-7-β-cyclodextrin (Captisol™) is dissolved in water to form a solution at 30% w/v. To the Captisol solution is added Lysine at a concentration of 65 mM and Captisol™ at a concentration of about 20 mg/ml. The solution is stirred at room temperature for about 10 minutes. The solution is separated from any excess solids (e.g. by filtration). The concentration of quercetin in the solution is about 10.4 mg/mL.

### Example 7

### Solubility of Quercetin with Sulfobutylether-7-β-cyclodextrin and Meglumine

Sulfobutylether-7-β-cyclodextrin (Captisol™) is dissolved in water to form a solution at 30% w/v. To the Captisol solution is added Meglumine at a concentration of 44 mM and Captisol™ at a concentration of about 20 mg/ml. The solution is stirred at room temperature for about 10 minutes. The solution is separated from any excess solids (e.g. by filtration). The concentration of quercetin in the solution is about 9.2 mg/mL.

### Example 8

### Stability of Quercetin with and without Sulfobutylether-7-α-cyclodextrin

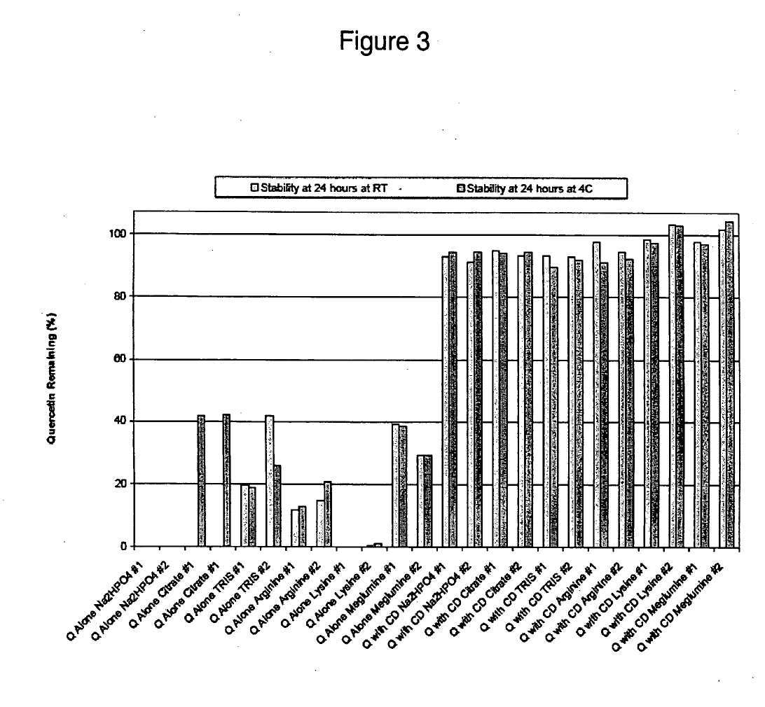

100 mL of various concentrations of Captisol solution are stirred in a beaker with a magnetic stir bar. Excess amounts (20 mg/mL) of quercetin are suspended. After 30 seconds, the alkalinzer, e.g. the amino acid, is added to achieve a target pH and the sample equilibrated for 10 minutes at RT. After the equilibration, the test solutions are filtered through 0.22 micron Millipore PVDF Millex-GV syringe filters. The pH is measured. The quercetin in the aliquots is assayed by high performance liquid chromatography (HPLC). The filtered samples are kept at RT and 4° C. for 24 and 72 hours. The quercetin in the aliquots is assayed by HPLC. Refractive index detection was used for the HPLC assay of Captisol. For the HPLC assay, a PolySep-GFC-P3000 300 mm×7.8 mm ID is used. The mobile phase is 80% 0.1M potassium nitrate/20% acetonitrile and the flow rate is 1.0 mL/min. Stability data can be obtained, for example, with the samples at pH 9. FIG. 3 shows stability data after 24 hours at room temperature (RT) and after 24 hours at 4° C. The stability is represented as the percent of quercetin remaining in the sample as determined by HPLC analysis. The quercetin formulations containing Captisol (Q with CD) were significantly more stable than the quercetin samples without Captisol (Q Alone).

### Example 9

### Preparation of Fisetin-Sulfobutylether-7-β-Cyclodextrin

Captisol™ (17.9 g) is dissolved in 55 ml of distilled (or deionized) water. Fisetin (anhydrous) (965 mg) is then added to the above solution. The mixture is stirred vigorously followed by sonication if necessary to break up any clumps so that a homogeneous suspension is obtained.

While stirring vigorously, a 1 N NaOH solution (12.0 ml) is added slowly and for the formation of the fisetin-Captisol inclusion complex. The resulting solution will be at about pH 12.5 and may have an orange-brown color. The solution is not kept long at this pH, and is quickly neutralized in the following step.

While stirring, add 1N HCl solution (9 ml) dropwise to attain pH to 7.8-7.9. The final solution may be light-yellow to light orange in color. In some cases, the pH can be brought to below pH 7.8. We have found, for example, that the pH can be lowered to about 7.2, at which point, no precipitation is observed. Add water to a final volume of 100 ml.

This solution will contain approximately a 1:2.5 molar ratio of fisetin to Captisol. This procedure is for preparing an approximately 9.47 mg/ml fisetin solution. In some cases, the solution can be further diluted to make a solution for injection or for oral administration, for example, by diluting with either 0.9% saline solution and/or with a solution containing dextrose.

### Example 10

### Blood Glucose Levels in Rats Co-Administered with Tacrolimus and Flavonoid-Sulfobutylether-7-β-Cyclodextrin

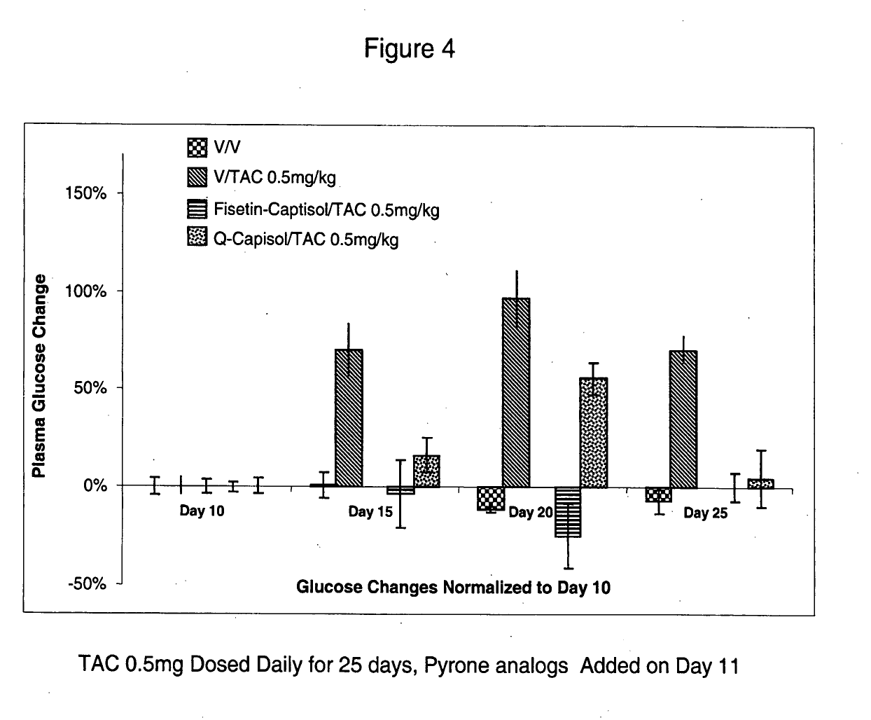

One set of 5 rats is treated from day 1 to day 25 with inert vehicle 2 intraperitoneally and treated from day 11 to day 25 with inert vehicle 1 intraperitoneally. A second set of 5 rats is treated from day 1 to day 25 intraperitoneally with tacrolimus (Prograf®) at 0.5 mg/kg, and treated from day 11 to day 25 intraperitoneally with inert vehicle 2. A third set 5 of rats is treated from day 1 to day 25 intraperitoneally with tacrolimus (Prograf®) at 0.5 mg/kg, and treated from day 11 to day 25 with sulfobutylether-7-β-cyclodextrin (Q-Captisol) at 100 mg/kg. A fourth set 5 of rats is treated from day 1 to day 25 intraperitoneally with tacrolimus (Prograft®) at 0.5 mg/kg, and treated from day 11 to day 25 with fisetin-sulfobutylether-7-β-cyclodextrin (Fisetin-Captisol) at 85 mg/kg. The blood glucose level in the rats was measured on days 1, 10, 15, 20, and 25. The results are shown in Tables 4-7 below and in FIG. 4. The results show that pyrone-analog-sulfobutylether-7-β-cyclodextrins such as Q-Captisol and Fisetin-Captisol attenuate tacrolimus induced hyperglycemia.

TABLE 4
Blood glucose levels in rats administered vehicle 1 and vehicle 2

| Treatment (mg/kg) | Treatment (mg/kg) | | Blood glucose | | | | |
|---|---|---|---|---|---|---|---|
| i.p. once daily | i.p. once daily | Rat | (g/l) | | | | |
| from Day 11 to Day 25 | from Day 1 to Day 25 | number | Day 1 | Day 10 | Day 15 | Day 20 | Day 25 |
| Vehicle 1 | Vehicle 2 | 1 | 1.26 | 1.32 | 1.21 | 1.01 | 1.21 |
| | | 2 | 1.43 | 1.27 | 1.30 | 0.93 | 1.13 |
| | | 3 | 1.20 | 0.95 | 1.26 | 1.27 | 1.16 |
| | | 4 | 1.51 | 1.36 | 1.30 | 1.12 | 1.08 |
| | | 5 | 1.44 | 1.39 | 1.30 | 1.26 | 1.27 |

| | | | | | |
|---|---|---|---|---|---|
| Mean ± | 1.37 | 1.26 | 1.27 | 1.12 | 1.17 |
| s.e.m. | 0.06 | 0.08 | 0.02 | 0.07 | 0.03 |
| Mean change from Day 1 | | −0.11 | −0.10 | −0.25 | −0.20 |

TABLE 5
Blood glucose levels in rats administered vehicle 1 and tacrolimus

| Treatment (mg/kg) | Treatment (mg/kg) | | Blood glucose | | | | |
|---|---|---|---|---|---|---|---|
| i.p. once daily | i.p. once daily | Rat | (g/l) | | | | |
| from Day 11 to Day 25 | from Day 1 to Day 25 | number | Day 1 | Day 10 | Day 15 | Day 20 | Day 25 |
| Vehicle 1 | Prograf ® | 6 | 1.16 | 1.40 | 2.31 | 2.97 | 1.69 |
| | 0.5 | 7 | 1.22 | 1.09 | 1.97 | 2.41 | 2.98 |
| | | 8 | 1.47 | 2.25 | 3.02 | 2.77 | 3.96 |
| | | 9 | 1.16 | 1.30 | 3.72 | 2.79 | 0.97 |
| | | 10 | 1.34 | 1.39 | 1.67 | 3.70 | 3.16 |

| | | | | | |
|---|---|---|---|---|---|
| Mean ± | 1.27 | 1.49 | 2.54 | 2.93 | 2.55 |
| s.e.m. | 0.06 | 0.20 | 0.37 | 0.21 | 0.54 |
| Mean change from Day 1 | | +0.22 | +1.27 | +1.66 | +1.28 |
| Mean change from all vehicle control | −0.10 | +0.23 | +1.27 | +1.81 | +1.38 |

TABLE 6
Blood glucose levels in rats administered Fisetin-Captisol and tacrolimus

| Treatment (mg/kg) | Treatment (mg/kg) | | Blood glucose | | | | |
|---|---|---|---|---|---|---|---|
| i.p. once daily | i.p. once daily | Rat | (g/l) | | | | |
| from Day 11 to Day 30 | from Day 1 to Day 30 | number | Day 1 | Day 10 | Day 15 | Day 20 | Day 25 |
| Fisetin-Captisol | Prograf ® | 16 | 1.32 | 2.36 | 2.93 | 1.75 | 1.85 |
| 85 | 0.5 | 17 | 1.21 | 1.35 | 1.40 | 1.18 | 1.25 |
| | | 18 | 1.23 | 1.27 | 1.41 | 1.25 | 1.64 |
| | | 19 | 1.22 | 2.86 | 1.69 | 1.35 | 2.86 |
| | | 20 | 1.35 | 1.40 | 1.50 | 1.41 | 1.66 |

| | | | | | |
|---|---|---|---|---|---|
| Mean ± | 1.27 | 1.85 | 1.79 | 1.39 | 1.85 |
| s.e.m. | 0.03 | 0.32 | 0.29 | 0.10 | 0.27 |
| Mean change from Day 1 | | +0.58 | +0.52 | +0.12 | +0.58 |
| Mean change from Prograf control | 0.00 | +0.36 | −0.75 | −1.54 | −0.70 |

TABLE 7
Blood glucose levels in rats administered Q-Captisol and tacrolimus

| Treatment (mg/kg) | Treatment (mg/kg) | | Blood glucose | | | | |
|---|---|---|---|---|---|---|---|
| i.p. once daily | i.p. once daily | Rat | (g/l) | | | | |
| from Day 11 to Day 30 | from Day 1 to Day 30 | number | Day 1 | Day 10 | Day 15 | Day 20 | Day 25 |
| Q-Captisol | Prograf ® | 21 | 1.62 | 1.39 | 2.05 | 2.23 | 1.64 |
| 100 | 0.5 | 22 | 1.36 | 1.48 | 1.74 | 3.22 | 1.32 |
| | | 23 | 1.41 | 2.16 | 1.85 | 2.50 | 1.82 |
| | | 24 | 1.40 | 1.46 | 2.48 | 3.60 | 1.86 |
| | | 25 | 1.28 | 1.83 | 1.53 | 1.39 | 2.06 |

| | | | | | |
|---|---|---|---|---|---|
| Mean ± | 1.41 | 1.66 | 1.93 | 2.59 | 1.74 |
| s.e.m. | 0.06 | 0.15 | 0.16 | 0.39 | 0.12 |
| Mean change from Day 1 | | +0.25 | +0.52 | +1.18 | +0.33 |
| Mean change from Prograf control | +0.14 | +0.17 | −0.61 | −0.34 | −0.81 |

### Example 11

### Pharmacokinetic Parameters of Tacrolimus in the Presence of Quercetin-Sulfobutylether-7-β-Cyclodextrin (Q-Captisol) in Rats Showing Tacrolimus Redistribution

Male Lewis rats (Charles River Laboratories; 8-9 weeks old; mean weights 282-285 gm) are randomly assigned to treatment groups by a computerized body weight stratification procedure. Quercetin-Sulfobutylether-7-β-cyclodextrin (Q-Captisol) is prepared as a dosing solution using Captisol (B-cyclodextrin-sulfobutyl ether sodium salt; CyDex Inc.) along with 1 molar equivalent of sodium hydroxide. Q-Captisol is administered intraperitoneally (i.p.) at doses of 0, 25, 150, or 300 mg/kg (N=9 rats/dose). Tacrolimus (1 mg/kg i.v.) is dosed at 0.2 ml/kg directly from Prograf 5 mg/ml clinical dosing solutions. Tacrolimus is dosed 30 min after administration of quercetin. Whole blood is collected in EDTA tubes from the retro-orbital sinus under 60:40 CO₂:O₂ anesthesia at 9 time periods using N=3 animals per time period: 5, 15, and 30 min; 1, 2, 3, 6, 8, and 24 hr after administration of tacrolimus. To avoid excessive blood loss from individual animals, 3 blood samples per rat are collected at specified time intervals, so the final samples for analysis contained N=3 blood samples per time period. Whole blood samples are froze on dry ice and stored frozen at −80° C. prior to analysis.

Thawed whole blood (200 μl) is transferred to a separate glass tube with 100 μl of ascomycin (Internal Standard: 100 ng/ml in 10% methanol) and 20 μl of 10% methanol. Samples are treated with 400 μl of 0.1 M zinc sulfate and then 100 μl of acetonitrile to precipitate proteins. Samples are vortexed, then centrifuged at 4000 rpm for 5 min. 500 μl of supernatant is transferred to a clean glass tube containing 1000 μl of water and vortexed thoroughly. Mixtures are loaded onto a 100 mg C₁₈ cartridge (SPE column) preconditioned with 1 ml of methanol, followed by 1 ml of water. The cartridge is washed 2× with 1 ml water, followed by 100 μl methanol. Tacrolimus and Internal Standard are eluted with 500 μl of methanol into an HPLC vial containing 1000 μl of 10 mM ammonium acetate. Samples are vortexed, then centrifuged at 4000 rpm for 10 min. A 10 μl aliquot of the clarified supernatant is injected directly onto LC/MS/MS for analysis. Calibration standards are prepared in blank rat whole blood at 1, 2, 5, 5, 10, 25, 50, 62.5, and 75 ng/ml and processed as described for the samples from rats dosed with tacrolimus.

The tacrolimus concentrations in plasma are determined using validated assay methods via an LC/MS/MS analytical technique. Major pharmacokinetic parameters, including area under the concentration versus time curve from time zero to the time of last measurable concentration (AUCₜ), area under the concentration versus time curve from time zero to infinite time (AUC_{inf}), maximum observed concentration (Cₘₐₓ), time to reach maximum observed blood or plasma concentration (Tₘₐₓ), apparent terminal elimination rate constant (K_{cl}), clearance (CL), and half-life (T_{1/2}), are determined from the plasma concentration data. Table 8 shows the results of the measurements. The results show that the Q-Captisol causes a significant decrease in tacrolimus volume of distribution (V_{d}), elevation of AUC and Cₘₐₓ, and reduced clearance. These changes are believed to be caused by drug re-distribution from non-targeted organs to systemic circulation.

TABLE 8
Pharmacokinetic parameters of i.v. tacrolimus
in plasma in male Lewis rats

| | | | AUC | | CL | |
|---|---|---|---|---|---|---|
| Q-Captisol | Cₘₐₓ | Tₘₐₓ | (ng- | T_{1/2} | (mL/ | V_{d} |
| Dose (mg/kg) | (ng/mL) | (hr) | hr/mL) | (hr) | hr/kg) | (L/kg) |
| 0 | 418 | 0.083 | 645 | 9.4 | 1376 | 18.6 |
| 25 | 426 | 0.083 | 635 | 7.9 | 1399 | 16.0 |
| 150 | 781 | 0.083 | 980 | 8.1 | 932 | 10.9 |
| 300 | 877 | 0.083 | 1242 | 7.7 | 733 | 8.2 |

### Example 12

### Pharmacokinetic Parameters of Tacrolimus in the Presence of Quercetin-Sulfobutylether-7-β-Cyclodextrin in Healthy Human Volunteers Showing Tacrolimus Redistribution

Tacrolimus-quercetin pharmacokinetic interactions are evaluated in N=15 healthy male subjects in an open-label, three-way crossover study of i.v. or oral quercetin-sulfobutylether-7-β-cyclodextrin (Q-Captisol) administered in combination with oral tacrolimus. Q-Captisol stock dosing solutions are prepared in aqueous Captisol (CyDex Inc.) using sodium hydroxide to facilitate dissolution. The final solution is adjusted to a final pH=7.5 to 8.0 using dilute sodium hydroxide or HCl. Stock solutions are aseptically filtered into a sterile IV bag and diluted to final volume with sterile saline. Q-Captisol oral dosing solutions are prepared by transferring the stock solution to a dosing cup and diluting to the final volume with deionized water. Oral tacrolimus is administered using commercially-available Prograf® capsules, with each capsule containing 1 mg tacrolimus and the excipient lactose.

In Period 1, all subjects received only a single dose of oral tacrolimus (1 mg).

In Period 2, all subjects received 3× daily oral doses of Q-Captisol on Days 12, 13, and 14. On Day 14, subjects received a single dose of oral tacrolimus (1 mg)

In Period 3, i.v. infusions of Q-Captisol are administered over 20 min via an indwelling cannula. Tacrolimus (1 mg) is administered orally 60 min after the start of the administration of the i.v. Q-Captisol dose.

The dosing regimen is as follows:

Period 1: Day 1: single oral dose of 1 mg tacrolimus

Period 2: Day 12: three times per day (Q 8 hrs) single oral dose of 100 mg Q-Captisol

Day 13: three times per day (Q 8 hrs) single oral dose of 100 mg Q-Captisol

Day 14: three times per day (Q 8 hrs) single oral dose of 100 mg Q-Captisol, with a single oral dose of 1 mg tacrolimus following the first dose of Q-Captisol.

Period 3: Day 25: single IV infusion of 100 mg Q-Captisol followed by a single oral dose of 1 mg tacrolimus.

In Period 1, blood samples for determination of tacrolimus pharmacokinetics are obtained at 0 min (pre-dose), then at 0.25, 0.5, 1, 2, 4, 8, 12, 24, 48, and 72 hr. Similar time periods are used to obtain tacrolimus blood sampled in Periods 2 and 3. Blood samples (0.4 ml) are withdrawn into K2-EDTA tubes from either an indwelling cannula or via venipuncture, then processed to yield plasma. Plasma samples are transferred to fresh tubes and frozen prior to shipment to the analytical laboratory.

The tacrolimus concentrations in plasma are determined using validated assay methods via an LC/MS/MS analytical technique. Major pharmacokinetic parameters, including area under the concentration versus time curve from time zero to the time of last measurable concentration (AUCₜ), area under the concentration versus time curve from time zero to infinite time (AUC_{inf}), maximum observed concentration (Cₘₐₓ), time to reach maximum observed blood or plasma concentration (Tₘₐₓ), apparent terminal elimination rate constant (K_{cl}), clearance (CL), and half-life (T_{1/2}), are determined from the plasma concentration data. Samples are analysed from all 15 subjects; one subject is a clear outlier and is excluded from the final analysis (N=14 subjects per dose regimen). Table 9 shows results from the study. Oral Q-Captisol produced shortened time to onset of tacrolimus, increased plasma exposure, and delayed hepatic elimination. Intravenous Q-Captisol dose produced the largest change in volume of distribution.

TABLE 9
Pharmacokinetic parameters of oral tacrolimus
in plasma in healthy human volunteers

| | | | AUC | | CL | |
|---|---|---|---|---|---|---|
| | Cₘₐₓ | Tₘₐₓ | (ng- | T_{1/2} | (mL/ | V_{d} |
| Period | (ng/mL) | (hr) | hr/mL) | (hr) | hr/kg) | (L/kg) |
| 1: tacrolimus | 5.8 | 1.56 | 48.8 | 20.8 | 285 | 8.5 |
| alone | | | | | | |
| 2: tacrolimus + | 6.5 | 1.28 | 65.7 | 27.7 | 212 | 8.4 |
| Q-Captisol po | | | | | | |
| 3: tacrolimus + | 6.4 | 1.55 | 56.3 | 19.9 | 245 | 7.1 |
| Q-Captisol iv | | | | | | |

All of the methods disclosed and claimed herein can be made and executed without undue experimentation in light of the present disclosure. It will be apparent to those of skill in the art that variations may be applied without departing from the concept, spirit and scope of the invention. More specifically, it will be apparent that certain agents that both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A method for producing a stable aqueous composition comprising a pyrone analog comprising mixing a cyclodextrin and the pyrone analog such as a flavonoid in an aqueous medium at a pH greater than about 11 and subsequently lowering the pH to less than about 9.

2. The method of claim 1 wherein the mixing of the cyclodextrin and the pyrone analog is at a pH greater than 12, and the pH is subsequently lowered to less than 8.5

3. The method of claim 1 wherein the cyclodextrin is sulfobutylether-7-β-cyclodextrin.

4. The method of claim 1 wherein the pyrone analog comprises a flavonoid is quercetin, galangin, fisetin, or kaempferol.

5. The method of claim 4 wherein the flavonoid comprises quercetin, galangin, fisetin, or kaempferol.

6. The method of claim 4 wherein the wherein the flavonoid comprises quercetin.

7. The method of claim 4 wherein the flavonoid has 3, 4, 5 or 6 acidic protons.

8. The method of claim 4 wherein the flavonoid has 5 or 6 acidic protons.

9. The method of claim 4 wherein the flavonoid has an aromatic hydroxyl (—OH) group.

10. The method of claim 4 wherein the flavonoid has 3, 4, or 5 aromatic hydroxyl (—OH) groups.

11. The method of claim 4 wherein the flavonoid has 4 or 5 aromatic hydroxyl (—OH) groups.

12. The method of claim 1 wherein the pyrone analog is substantially insoluble in water.

13. The method of claim 1 wherein the pyrone analog is sparingly soluble in water.

14. The method of claim 1 wherein sodium hydroxide is added in order to bring the pH of the aqueous solution to greater than about 11.

15. The method of claim 1 wherein hydrochloric acid is used to lower the pH.

16. The method of claim 1 wherein the method is carried out at a temperature below 30° C.

17. The method of claim 16 wherein the method is carried out at a temperature below 26° C.

18. The method of claim 1 wherein the time that the reaction is above pH 9 is less than 20 minutes.

19. The method of claim 1 wherein the time that the reaction is above pH 9 is less than 10 minutes.

20. The method of claim 1 wherein the time that the reaction is above pH 9 is less than 5 minutes.

21. The method of claim 1 wherein the concentration of the pyrone analog in the aqueous composition is greater than about 0.5 mM.

22. The method of claim 1 wherein the concentration of the pyrone analog in the aqueous composition is greater than about 5 mM.

23. The method of claim 1 wherein the concentration of the pyrone analog in the aqueous composition is greater than about 10 mM.

24. The method of claim 1 wherein the concentration of the pyrone analog in the aqueous composition is greater than about 30 mM.

25. The method of claim 1 wherein the concentration of the pyrone analog in the aqueous composition is greater than about 50 mM.

26. The method of claim 1 wherein the concentration of the pyrone analog in the aqueous composition is greater than about 80 mM.

27. A method for producing a composition comprising a pyrone analog in an aqueous solution comprising:
(a) dissolving a cyclodextrin in an aqueous solution;
(b) adding the pyrone analog to the aqueous solution;
(c) raising the pH of the aqueous solution to above about pH 11 while mixing the cyclodextrin and pyrone analog such as a flavonoid; and
(d) lowering the pH of the aqueous solution to below about pH 9.

28. The method of claim 27 wherein the steps are carried out in the order listed.

29. The method of claim 27 wherein the cyclodextrin is sulfobutylether-7-β-cyclodextrin.

30. The method of claim 27 wherein the pyrone analog comprises a flavonoid.

31. (canceled)

32. The method of claim 27 wherein the cyclodextrin is sulfobutylether-7-β-cyclodextrin, and the pyrone analog such as a flavonoid is quercetin.

33-39. (canceled)

40. A composition produced by carrying out the method of claim 1.

41. A composition comprising a pyrone analog and a sulfo-alkyl ether substituted cyclodextrin and an aqueous carrier wherein the pyrone analog is present in a concentration greater than 0.5 mM.

42-76. (canceled)

77. A solid pharmaceutical formulation that is made using the method of claim 1.

78. The formulation of claim 77 wherein the cyclodextrin is sulfobutylether-7-β-cyclodextrin.

79-81. (canceled)

82. The formulation of claim 77 wherein the pyrone analog comprises quercetin.

83-90. (canceled)

91. A kit comprising a) a container comprising the formulation of claim 77, and b) instructions for using the formulation to treat a disorder.

92. The kit of claim 91 wherein the formulation is suitable for intravenous administration.

93. The kit of claim 91 wherein the formulation is suitable for oral administration.

94. A pharmaceutical composition comprising a therapeutic agent, a pyrone analog, a sulfo-alkyl ether substituted cyclodextrin and a carrier.

95-115. (canceled)

116. The composition of claim 94 wherein the pyrone analog is a BTB transport modulator and is present in an amount sufficient to reduce a side effect such as a side effect of the therapeutic agent.

117-120. (canceled)

121. The composition of claim 94 wherein the therapeutic agent is selected from the group consisting of antihypertensives, vasodilators, barbiturates, membrane stabilizers, cardiac stabilizers, glucocorticoids, antiinfectives, immunomodulators and chemotherapeutic agents.

122. The composition of claim 121 wherein the therapeutic agent is an immunomodulator.

123. The composition of claim 121 wherein the therapeutic agent is tacrolimus.

124-128. (canceled)

129. A method of treating an animal comprising administering to an animal an effective amount of a pharmaceutical formulation that is made using the method of claim 1.

130-133. (canceled)

134. A method of treating an animal for pain comprising administering to an animal in pain an effective amount of an analgesic agent and an amount of a composition comprising a pyrone analog and a cyclodextrin sufficient to reduce a side effect of the analgesic agent.

135-161. (canceled)

162. A method of treating an animal comprising; administering an animal in need of treatment an effective amount of a therapeutic agent and a composition comprising a pyrone analog and a cyclodextrin.

163-170. (canceled)

171. The method of claim 162 wherein the therapeutic agent is selected from the group consisting of antihypertensives, vasodilators, barbiturates, membrane stabilizers, cardiac stabilizers, glucocorticoids, antiinfectives, immunomodulators and chemotherapeutic agents.

172. The method of claim 162 wherein the therapeutic agent is an immunomodulator.

173. The method of claim 162 wherein the therapeutic agent is tacrolimus.

174-180. (canceled)

181. A pharmaceutical composition comprising a pyrone analog such as a flavonoid, a cyclodextrin, and a basic amino acid or a sugar-amine and a pharmaceutically or veterinarily acceptable carrier.

182. The pharmaceutical composition of claim 181 wherein the basic amino acid is arginine.

183. The pharmaceutical composition of claim 181 wherein the basic amino acid is lysine.

184. The pharmaceutical composition of claim 181 wherein the sugar-amine is meglumine.

185. The pharmaceutical composition of claim 181 wherein the pyrone analog is quercetin, galangin, fisetin, or kaempferol.

186. The pharmaceutical composition of claim 181 wherein the pyrone analog is quercetin.

187. The pharmaceutical composition of claim 181 wherein the cyclodextrin is sulfobutylether-7-β-cyclodextrin.

188. The pharmaceutical composition of claim 181 wherein the pyrone analog is quercetin, and the cyclodextrin is sulfobutylether-7-β-cyclodextrin.

189. The pharmaceutical composition of claim 188 wherein the carrier comprises water.

190. The pharmaceutical composition of claim 189 wherein the sulfobutylether-7-β-cyclodextrin is present at a concentration of about 20% w/v or greater.

191. The pharmaceutical composition of claim 189 wherein the sulfobutylether-7-β-cyclodextrin is present at a concentration in a range of about 20% w/v to about 30% w/v.

192-198. (canceled)

199. A method of preparing a solution of a pyrone analog comprising mixing a cyclodextrin, a pyrone analog such as a flavonoid, and a basic amino acid or a sugar-amine with water at a pH greater than 8.5.

200. The method of claim 199 comprising dissolving the cyclodextrin in water to produce a cyclodextrin solution, then mixing the pyrone analog and the basic amino acid or sugar-amine with the cyclodextrin solution.

201. The method of claim 199 wherein the basic amino acid is arginine.

202. The method of claim 199 wherein the basic amino acid is lysine.

203. The method of claim 199 wherein the sugar-amine is meglumine.

204. The method of claim 199 wherein the pyrone analog is quercetin, galangin, fisetin, or kaempferol

205. The method of claim 199 wherein the pyrone analog is quercetin.

206. The method of claim 199 wherein the cyclodextrin is sulfobutylether-7-β-cyclodextrin.

207. The method of claim 199 wherein the pyrone analog is quercetin, and the cyclodextrin is sulfobutylether-7-β-cyclodextrin.

208-213. (canceled)
